(19) United States
(12) Patent Application Publication
(10) Pub. No.: US 2025/0104800 A1
(43) Pub. Date: Mar. 27, 2025

(54) COMPUTATIONAL ANALYSIS OF BIOLOGICAL DATA USING MANIFOLD AND A HYPERPLANE

(71) Applicant: MeMed Diagnostics Ltd., Tirat HaCarmel (IL)

(72) Inventors: Eran EDEN, Haifa (IL); Kfir OVED, Hof HaCarmel (IL); Roy NAVON, Tel-Aviv (IL); Assaf COHEN-DOTAN, Natania (IL); Olga BOICO, Atlit (IL)

(73) Assignee: MeMed Diagnostics Ltd., Tirat HaCarmel (IL)

(21) Appl. No.: 18/928,332

(22) Filed: Oct. 28, 2024

Related U.S. Application Data
(63) Continuation of application No. 18/230,718, filed on Aug. 7, 2023, now Pat. No. 12,131,807.
Application No. 18/230,718 is a continuation of application No. 17/875,467, filed on Jul. 28, 2022, now Pat. No. 11,776,658.
Application No. 17/875,467 is a continuation of application No. 16/998,006, filed on Aug. 20, 2020, now Pat. No. 11,450,406.
Application No. 16/998,006 is a division of application No. 16/355,984, filed on Mar. 18, 2019, now Pat. No. 11,081,206.
Application No. 16/355,984 is a continuation of application No. 15/503,439, filed on Feb. 13, 2017, now Pat. No. 10,303,846.
(60) Provisional application No. 62/105,938, filed on Jan. 21, 2015.
(60) Provisional application No. 62/037,180, filed on Aug. 14, 2014.

(51) Int. Cl. G16B 5/20; G01N 33/569; G06F 17/18; G16B 5/00; G16B 20/00; G16B 20/20; G16B 25/00; G16B 25/10; G16B 40/00; G16B 40/20; G16B 40/30; G16H 50/50

(52) U.S. Cl. CPC G16B 5/20; G06F 17/18; G16B 5/00; G16B 20/00; G16B 20/20; G16B 25/10; G16B 40/00; G16B 40/20; G16B 40/30; G16H 50/50; G01N 33/56911; G01N 33/56983; G16B 25/00; Y02A 90/10

(57) ABSTRACT

A method of analyzing biological data containing expression values of a plurality of polypeptides in the blood of a subject. The method comprises: calculating a distance between a segment of a curved line and an axis defined by a direction, the distance being calculated at a point over the curved line defined by a coordinate along the direction. The method further comprises correlating the distance to the presence of, absence of, or likelihood that the subject has, a bacterial infection. The coordinate is defined by a combination of the expression values, wherein at least 90% of the segment is between a lower bound line and an upper bound line.

### RELATED APPLICATIONS

This application is a continuation of U.S. patent application Ser. No. 18/230,718, filed on Aug. 7, 2023, now U.S. Pat. No. 12,131,807, which is a continuation of U.S. patent application Ser. No. 17/875,467, filed on Jul. 28, 2022, now U.S. Pat. No. 11,776,658, which is a continuation of U.S. patent application Ser. No. 16/998,006, filed on Aug. 20, 2020, now U.S. Pat. No. 11,450,406, which is a division of U.S. patent application Ser. No. 16/355,984 filed on Mar. 18, 2019, now U.S. Pat. No. 11,081,206 which is a U.S. continuation of U.S. patent application Ser. No. 15/503,439 filed on Feb. 13, 2017, now U.S. Pat. No. 10,303,846, which is a National Phase of PCT Patent Application No. PCT/IL2015/050823 having International Filing Date of Aug. 12, 2015, which claims the benefit of priority under 35 USC § 119 (e) of U.S. Provisional Patent Application Nos. 62/105,938 filed on Jan. 21, 2015 and 62/037,180 filed on Aug. 14, 2014. The contents of the above applications are all incorporated by reference as if fully set forth herein in their entirety.

### SEQUENCE LISTING STATEMENT

The XML file, entitled 101774ReplacementSequenceListing.xml, created on Nov. 30, 2024, comprising 162,407 bytes, submitted concurrently with the filing of this application is incorporated herein by reference. The sequence listing submitted herewith is identical to the sequence listing forming part of the international application.

### FIELD AND BACKGROUND OF THE INVENTION

The present invention, in some embodiments thereof, relates to computational analysis, and, more particularly, but not exclusively, to computational analysis of biological data, e.g., for the purpose of distinguishing between bacterial infection and non-bacterial disease, and/or between a bacterial infection and viral infection, and/or between an infectious and non-infectious disease.

Antibiotics (Abx) are the world's most prescribed class of drugs with a 25-30 billion $US global market. Abx are also the world's most misused drug with a significant fraction of all drugs (40-70%) being wrongly prescribed (Linder, J. A. and R. S. Stafford 2001; Scott, J. G. and D. Cohen, et al. 2001; Davey, P. and E. Brown, et al. 2006; Cadieux, G. and R. Tamblyn, et al. 2007; Pulcini, C. and E. Cua, et al. 2007), (“CDC-Get Smart: Fast Facts About Antibiotic Resistance” 2011).

One type of Abx misuse is when the drug is administered in case of a non-bacterial disease, such as a viral infection, for which Abx is ineffective. For example, according to the USA center for disease control and prevention CDC, over 60 Million wrong Abx prescriptions are given annually to treat flu in the US. The health-care and economic consequences of the Abx over-prescription include: (i) the cost of antibiotics that are unnecessarily prescribed globally, estimated at >$10 billion annually; (ii) side effects resulting from unnecessary Abx treatment are reducing quality of healthcare, causing complications and prolonged hospitalization (e.g. allergic reactions, Abx associated diarrhea, intestinal yeast etc.) and (iii) the emergence of resistant strains of bacteria as a result of the overuse (the CDC has declared the rise in antibiotic resistance of bacteria as “one of the world's most pressing health problems in the 21^{st} century” (Arias, C. A. and B. E. Murray 2009; “CDC—About Antimicrobial Resistance” 2011).

Antibiotics under-prescription is not uncommon either. For example up to 15% of adult bacterial pneumonia hospitalized patients in the US receive delayed or no Abx treatment, even though in these instances early treatment can save lives and reduce complications (Houck, P. M. and D. W. Bratzler, et al. 2002).

Technologies for infectious disease diagnosis have the potential to reduce the associated health and financial burden associated with Abx misuse. Ideally, such a technology should: (i) accurately differentiate between a bacterial and viral infections; (ii) be rapid (within minutes); (iii) be able to differentiate between pathogenic and non-pathogenic bacteria that are part of the body's natural flora; (iv) differentiate between mixed co-infections and pure viral infections and (v) be applicable in cases where the pathogen is inaccessible (e.g. sinusitis, pneumonia, otitis-media, bronchitis, etc).

Current solutions (such as culture, PCR and immunoassays) do not fulfill all these requirements: (i) Some of the assays yield poor diagnostic accuracy (e.g. low sensitivity or specificity) (Uyeki et al. 2009), and are restricted to a limited set of bacterial or viral strains; (ii) they often require hours to days; (iii) they do not distinguish between pathogenic and non-pathogenic bacteria (Del Mar, C 1992), thus leading to false positives; (iv) they often fail to distinguish between a mixed and a pure viral infections and (v) they require direct sampling of the infection site in which traces of the disease causing agent are searched for, thus prohibiting the diagnosis in cases where the pathogen resides in an inaccessible tissue, which is often the case.

Consequentially, there still a diagnostic gap, which in turn often leads physicians to either over-prescribe Abx (the “Just-in-case-approach”), or under-prescribe Abx (the “Wait-and-see-approach”) (Little, P. S. and I. Williamson 1994; Little, P. 2005; Spiro, D. M. and K. Y. Tay, et al. 2006), both of which have far reaching health and financial consequences.

Accordingly, a need exists for a rapid method that accurately differentiates between bacterial (including mixed bacterial plus viral infection), viral and non-bacterial, non-viral disease patients that addresses these challenges.

WO 2013/117746 teaches signatures and determinants for distinguishing between a bacterial and viral infection.

### SUMMARY OF THE INVENTION

According to an aspect of some embodiments of the present invention there is provided a method of analyzing biological data, the biological data containing expression values of a plurality of polypeptides in the blood of a subject. The method comprises: calculating a distance between a segment of a curved line and an axis defined by a direction, the distance being calculated at a point over the curved line defined by a coordinate δ₁ along the direction. The method further comprises correlating the distance to the presence of, absence of, or likelihood that the subject has a bacterial infection. The coordinate δ₁ is defined by a combination of the expression values, wherein at least 90% of the segment is between a lower bound line f(δ₁)−ε₀ and an upper bound line f(δ₁)+ε₁, wherein the g(δ₀) equals 1/(1+exp (δ₁)), and wherein each of the ε₀ and the ε₁ is less than 0.5.

According to some embodiments of the invention the method comprises obtaining the likelihood based on the distance, comparing the likelihood to a predetermined threshold, and, treating the subject for the bacterial infection when the likelihood is above the predetermined threshold.

According to an aspect of some embodiments of the present invention there is provided a method of analyzing biological data, the biological data containing expression values of a plurality of polypeptides in the blood of a subject. The method comprises: calculating a distance between a segment of a curved line and an axis defined by a direction, the distance being calculated at a point over the curved line defined by a coordinate δ₀ along the direction. The method further comprises correlating the distance to the presence of, absence of, or likelihood that the subject has a viral infection. The coordinate δ₀ is defined by a combination of the expression values, wherein at least 90% of the segment is between a lower bound line g(δ₀)−ε₀ and an upper bound line g(δ₀)+ε₁, wherein the f(δ₀) equals 1/(1+exp (δ₀)), and wherein each of the ε₀ and the ε₁ is less than 0.5.

According to some embodiments of the invention the method comprises obtaining the likelihood based on the distance, comparing the likelihood to a predetermined threshold, and, treating the subject for the viral infection when the likelihood is above the predetermined threshold.

According to some embodiments of the invention the combination of the expression values comprises a linear combination of the expression values.

According to some embodiments of the invention the combination of the expression values includes at least one nonlinear term corresponding to at least one of the expression values.

According to an aspect of some embodiments of the present invention there is provided a method of analyzing biological data, the biological data containing expression values of a plurality of polypeptides in the blood of a subject. The method comprises: calculating a first distance between a segment of a curved surface and a plane defined by a first direction and a second direction. The first distance being calculated at a point over the surface defined by first coordinate δ₀ along the first direction and a second coordinate δ₁ along the second direction. The method further comprises correlating the first distance to the presence of, absence of, or likelihood that the subject has a bacterial infection. Each of the coordinates is defined by a different combination of the expression values, wherein at least 90% of the segment is between a lower bound surface f(δ₀, δ₁)−ε₀ and an upper bound surface f(δ₀, δ₁)+ε₁, wherein the f(δ₀, δ₁) equals exp (δ₁)/(1+exp (δ₀)+exp (δ₁)), and wherein each of the ε₀ and the ε₁ is less than 0.5.

According to some embodiments of the invention for at least one of the coordinates, the combination of the expression values comprises a linear combination of the expression values.

According to some embodiments of the invention for at least one of the coordinates, the combination of the expression values includes at least one nonlinear term corresponding to at least one of the expression values.

According to some embodiments of the invention the method comprises obtaining the likelihood based on the first distance, comparing the likelihood to a predetermined threshold, and, treating the subject for the bacterial infection when the likelihood is above the predetermined threshold.

According to some embodiments of the invention the method comprises calculating a second distance between a segment of second curved surface and the plane; and correlating the second distance to the presence of, absence of, or likelihood that the subject has a viral infection. According to some embodiments of the invention at least 90% of the segment of the second surface is between a second lower bound surface g(δ₀, δ₁)−ε₂ and a second upper bound surface g(δ₀, δ₁)+ε₃, wherein the g(δ₀, δ₁) equals exp(δ₀)/(1+exp (δ₀)+exp (δ₁)), and wherein each of the ε₂ and the ε₃ is less than 0.5.

According to some embodiments of the invention the method comprises obtaining the likelihood based on the second distance, comparing the likelihood to a second predetermined threshold, and, treating the subject for the viral infection when the likelihood is above the second predetermined threshold.

According to some embodiments of the invention the method comprises obtaining the likelihood that the subject has a bacterial infection based on the distance, obtaining the likelihood that the subject has a viral infection based on the second distance, comparing each of the likelihoods to a respective predetermined threshold, and, when each of the likelihoods is below the respective predetermined threshold, then determining that the patient is likely to have a non-infectious disease.

According to an aspect of some embodiments of the present invention there is provided a method of analyzing biological data, the biological data containing expression values of a plurality of polypeptides in the blood of a subject. The method comprises: calculating a distance between a segment of a curved surface and a plane defined by a first direction and a second direction. The distance is calculated at a point over the surface defined by first coordinate δ₀ along the first direction and a second coordinate δ₁ along the second direction. The method comprises correlating the distance to the presence of, absence of, or likelihood that the subject has, a viral infection; wherein each of the coordinates is defined by a different combination of the expression values, wherein at least 90% of the segment is between a lower bound surface g(δ₀, δ₁)−ε₀ and an upper bound surface g(δ₀, δ₁)+ε₁, wherein the g(δ₀, δ₁) equals exp (δ₀)/(1+exp (δ₀)+exp (δ₁)), and wherein each of the ε₀ and the ε₁ is less than 0.5.

According to some embodiments of the invention each of the plurality of polypeptides is selected from the group consisting of CRP, IP-10, TRAIL, IL1ra, PCT and SAA.

According to some embodiments of the invention the plurality of polypeptides comprises at least three polypeptides.

According to some embodiments of the invention the plurality of polypeptides comprises at least three polypeptides selected from the group consisting of CRP, IP-10, TRAIL, IL1ra, PCT and SAA.

According to some embodiments of the invention the plurality of polypeptides comprises at least CRP and TRAIL.

According to some embodiments of the invention the plurality of polypeptides comprises at least CRP, TRAIL and IP-10.

According to some embodiments of the invention the method comprises generating an output of the likelihood, the output is presented as text.

According to some embodiments of the invention the method comprises generating an output of the likelihood, the output is presented graphically.

According to some embodiments of the invention the method comprises generating an output of the likelihood, the output is presented using a color index.

According to some embodiments of the invention the blood sample is whole blood.

According to some embodiments of the invention the blood sample is a fraction of whole blood.

According to some embodiments of the invention the blood fraction comprises serum or plasma.

According to some embodiments of the invention the method comprises determining the expression values, and wherein at least one of the expression values is determined electrophoretically or immunochemically.

According to some embodiments of the invention the immunochemical determination is effected by flow cytometry, radioimmunoassay, immunofluorescence or by an enzyme-linked immunosorbent assay.

According to some embodiments of the invention the calculating and the correlating is executed by a computer remote from the subject.

According to some embodiments of the invention the calculating and the correlating is executed by a computer near the subject.

According to some embodiments of the invention the calculating and the correlating is executed by a cloud computing resource of a cloud computing facility.

According to some embodiments of the invention the expression values are measured by a measuring system performing at least one automated assay selected from the group consisting of an automated ELISA, an automated immunoassay, and an automated functional assay, and the method comprises receiving said the biological data from said measuring system.

According to some embodiments of the invention the receiving is over an internet network via a network interface.

According to an aspect of some embodiments of the present invention there is provided a computer-implemented method for analyzing biological data. The method comprises: displaying on a display device a graphical user interface (GUI) having a calculation activation control; receiving expression values of polypeptides in the blood of a subject; responsively to an activation of the control by a user, automatically calculating a score based on the expression values; generating on the GUI a graphical scale having a first end identified as corresponding to a viral infection of the subject, and a second end identified as corresponding to a bacterial infection the subject; and generating a mark on the scale at a location corresponding to the score.

According to some embodiments of the invention the expression values are received by communicating with an external machine that measures the expression values. According to some embodiments of the invention the GUI comprises a communication control, wherein the communication with the external machine is in response to an activation of the communication control by the user.

According to some embodiments of the invention the GUI comprises a plurality of an expression value input fields, wherein the expression values are received via the input fields.

According to some embodiments of the invention the score is a likelihood that the subject has bacterial infection. According to some embodiments of the invention the score is a likelihood that the subject has viral infection.

According to an aspect of some embodiments of the present invention there is provided a computer software product, comprising a computer-readable medium in which program instructions are stored, which instructions, when read by a hardware processor, cause the hardware processor to receive expression values of a plurality of polypeptides in the blood of a subject who has an unknown disease, and to execute the method as delineated above and optionally as further detailed below.

According to an aspect of some embodiments of the present invention there is provided a system for analyzing biological data. The system comprises: a user interface configured to receive expression values of a plurality of polypeptides in the blood of a subject who has an unknown disease; and a hardware processor having a computer-readable medium storing the computer software product.

According to an aspect of some embodiments of the present invention there is provided a system for analyzing biological data. The system comprises: a first compartment configured to measure expression values of a plurality of polypeptides in the blood of a subject who has an unknown disease; a second compartment comprising a hardware processor having a computer-readable storing the computer software product.

According to some embodiments of the invention the first compartment, the second compartment and the display are mounted on or integrated with a body of a hand-held device.

According to an aspect of some embodiments of the present invention there is provided a method of analyzing a dataset. The method comprises: (a) accessing a dataset comprising classification groups based on expression values of a plurality of polypeptides in the blood of a subject who has an unknown disease in blood samples of multiple subjects, wherein the classification groups comprise a bacterial infection, a viral infection and a non-viral, non bacterial disease; and (b) analyzing the classification groups to provide at least a first probabilistic classification function f(δ₀, δ₁) representing the likelihood that a particular subject has a bacterial infection, the first classification function being a function of a first coordinate δ₀ and a second coordinate δ₁, and wherein each of the coordinates is defined by a different combination of the expression values.

According to some embodiments of the invention the method further comprising calculating a second classification function g(♦₀, δ₁) representing the likelihood that a particular subject has a viral infection, the second classification function being also a function of the first and the second coordinates.

According to some embodiments of the invention the method comprises calculating a third classification function h(δ₀, δ₁) representing the likelihood that a particular subject has a non-viral, non bacterial disease, the third classification function being also a function of the first and the second coordinates.

According to some embodiments of the invention, for at least one of the coordinates, the combination of the expression values comprises a linear combination of the expression values.

According to some embodiments of the invention for at least one of the coordinates, the combination of the expression values includes at least one nonlinear term corresponding to at least one of the expression values.

According to some embodiments of the invention the method comprises generating an output of the analyzing.

According to some embodiments of the invention the dataset comprises one or more multidimensional entries.

According to some embodiments of the invention the method wherein each entry in the dataset comprises at least one clinical parameter of the respective subject.

According to some embodiments of the invention the method wherein the clinical parameter is selected from the group consisting of a sex, an age, a temperature, a time from symptoms onset and a weight.

According to some embodiments of the invention the analysis comprises machine learning.

According to some embodiments of the invention the machine learning comprises a supervised machine learning.

According to some embodiments of the invention the machine learning comprises at least one procedure selected from the group consisting of clustering, support vector machine, linear modeling, k-nearest neighbors analysis, decision tree learning, ensemble learning procedure, neural networks, probabilistic model, graphical model, Bayesian network, logistic regression and association rule learning.

According to some embodiments of the invention the method wherein the machine learning is selected from the group consisting of support vector machine, neural networks and logistic regression.

According to some embodiments of the invention the blood sample is whole blood.

According to some embodiments of the invention the blood sample is a fraction of whole blood.

According to some embodiments of the invention the blood fraction comprises serum or plasma.

According to some embodiments of the invention the expression value is determined electrophoretically or immunochemically.

According to some embodiments of the invention the immunochemical determination is effected by flow cytometry, radioimmunoassay, immunofluorescence or by an enzyme-linked immunosorbent assay.

According to an aspect of some embodiments of the present invention there is provided a method of predicting a prognosis for a disease. The method comprises measuring the TRAIL protein serum level in subject having the disease, wherein when the TRAIL level is below a predetermined level, the prognosis is poorer than for a subject having a disease having a TRAIL protein serum level above the predetermined level.

According to some embodiments of the invention the method wherein the disease is an infectious disease.

According to some embodiments of the invention the method wherein the disease is not an infectious disease.

According to an aspect of some embodiments of the present invention there is provided a method of determining a treatment course for a disease in a subject. The method comprises measuring the TRAIL protein serum level in the subject, wherein when the TRAIL level is below a predetermined level, the subject is treated with a treatment of last resort.

According to some embodiments of the invention the predetermined level is below 20 pg/ml.

According to an aspect of some embodiments of the present invention there is provided a method of determining an infection type in a female subject of fertility age.

The method comprises comparing the TRAIL protein serum level in the subject to a predetermined threshold, the predetermined threshold corresponding to the TRAIL protein serum level of a healthy female subject of fertility age, or a group of healthy female subjects of fertility age, wherein a difference between the TRAIL protein serum level and the predetermined threshold is indicative of an infection type.

According to an aspect of some embodiments of the present invention there is provided a method of determining an infection type in a male subject of fertility age.

The method comprises comparing the TRAIL protein serum level in the subject to a predetermined threshold, the predetermined threshold corresponding to the TRAIL protein serum level of a healthy male subject of fertility age, or a group of healthy male subjects of fertility age, wherein a difference between the TRAIL protein serum level and the predetermined threshold is indicative of an infection type.

According to some embodiments of the invention when the TRAIL protein serum level is above the predetermined threshold, the infection type is viral.

According to some embodiments of the invention when the TRAIL protein serum level is above the predetermined threshold, the infection type is not bacterial.

According to some embodiments of the invention when the TRAIL protein serum level is below the predetermined threshold, the infection type is bacterial.

According to some embodiments of the invention when the TRAIL protein serum level is below the predetermined threshold, the infection type is not viral.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1B. Study workflow. (A) An overview of the study workflow. n_{Bacterial}, n_{Viral} and n_{Control} represent the number of bacterial (including mixed bacterial plus viral co-infections), viral and control (with no apparent infectious disease) cases, respectively. (B) Proteins discovery and validation process.

FIGS. 2A-2C. The proteins TRAIL, IP-10 and CRP are differentially expressed in bacterial, viral and non-infectious patients. Box plots for TRAIL (A), IP-10 (B), and CRP (C), measured over the Majority cohort (n=765) are presented. Boxed line and circle correspond to group median and average respectively; t-test p-values between bacterial and viral groups and between infectious (bacterial and viral) vs. non-infectious (including healthy subjects) are depicted.

FIGS. 3A-3B. Comparison of the signature to lab parameters and protein biomarkers for diagnosing bacterial vs. viral patients. (A) Performance of clinical and lab parameters as well as the best performing pair (ANC and Lym %), triplet (ANC, Lym % and Pulse), and quadruplets (ANC, Lym %, Pulse, Mono %) of parameters, the values of which were combined using a logistic regression. Comparison was done on the Majority cohort (bacterial and viral patients, n=653), apart from pulse (recorded in 292 bacterial and 326 viral patients), and respiratory rate (recorded in 292 bacterial and 326 viral patients). The signature performed significantly better (P<10⁻¹⁵) than the optimal quadruplet. (B) The signature performed significantly better (P<10⁻⁸) than biomarkers with a well-established role in the host response to infections. For each of the select biomarkers, analysis was performed in a subgroup of the Majority cohort (43≤n≤154 for each analysis, a convenience sample, n depended on the strength of the signal). Error bars represent 95% CI.

FIG. 4. Signature performance is robust across different patient subgroups. Signature AUC in subgroups of the Majority cohort (bacterial and viral) are depicted. Square size is proportional to number of patients and error bars represent 95% CI. In the Pathogens analysis, each virus was compared to bacteria affecting the same physiological system, indicated in brackets. R-respiratory, S-systemic, C-central nervous system, G-gastrointestinal, U-urinary, K-skin. Only pathogens detected in more than 5 patients are presented. For subgroup definitions see Table 1 in Example 1.

FIG. 5. Calibration plot of the MLR model. In the top panel patients were grouped into 10 bins based on their predicted probabilities of a bacterial infection (x-axis), and compared to the observed fraction of bacterial infections within each bin (y-axis). Dashed line is a moving average (of size 5 bins). The bottom panel shows the distribution of predicted probabilities for bacterial (upper bars) and viral (lower bars).

FIGS. 6A-6B. Age distribution of the diagnosed patients. A. The entire study population (n=794); B. Pediatric patients only (n=445).

FIGS. 7A-7B. Distribution of detected pathogens in diagnosed patients (n=794). A. Distribution of detected pathogens by pathogenic subgroups; B. Distribution of detected pathogens by strain (strains detected from >1% of patients are presented). Distribution represents % of positive detections in patients with diagnosed infectious disease.

FIG. 8. Distribution of involved physiologic systems in patients diagnosed with an infectious disease (n=673).

FIGS. 9A-9B. Distribution of clinical syndromes (all diagnosed patients, n=794). A. Major clinical syndromes; B. Specific clinical syndromes.

FIG. 10. Distribution of maximal body temperatures (n=794).

FIG. 11. Distribution of time from initiation of symptoms (n=794). N/A-healthy controls or patients for which data was not obtained.

FIGS. 12A-12B. Comorbidities-related characterization of the patient population. A. Distribution of comorbidities (all chronically ill patients, n=305); B. Distribution of chronic medications (all chronically ill patients, n=305). Of note, some of the patients presented with several chronic diseases, and treated with several chronic medications.

FIG. 13. Distribution of recruitment sites (diagnosed patients, n=794).

FIGS. 14A-14B. Extrapolated PPV and NPV values for the signature as a function of the prevalence of bacterial infections, A. Unanimous (bacterial, viral) cohort (n=527), B. Majority (bacterial, viral) cohort (n=653).

FIGS. 15A-15E. Scatter plots of clinical parameters and laboratory measurements in bacterial, viral, and non-infectious patients (as indicated) in the Majority (bacterial, viral, non-infectious) cohort (n=765). Boxed line and circle correspond to group median and average respectively. T-test p-values between bacterial and viral groups and between infectious (bacterial and viral) vs. non-infectious (including healthy subjects) are depicted.

FIGS. 16A-16B. Comparison of the performance of the signature and PCT using different cutoffs. A. Performance measured in 76 patients from the Unanimous (bacterial, viral) cohort; B. Performance measured in 101 patients from the Majority (bacterial, viral) cohort. Error bars represent 95% CI. Signature sensitivity (left) and specificity (right) were calculated after filtering out 14% of the patients with a marginal immune response.

FIGS. 17A-17B. Comparison of the performance of the signature and CRP using different cutoffs. A. Performance measured in the Unanimous (bacterial, viral) cohort (n=527); B. Performance measured in the Majority (bacterial, viral) cohort (n=653). Error bars represent 95% CI. Signature sensitivity (left) and specificity (right) were calculated after filtering out 14% of the patients with a marginal immune response.

FIGS. 18A-18H. Scatter plots of levels of selected protein biomarkers (arbitrary units) in bacterial and viral patients. Boxed line and circle correspond to group median and average respectively. T-test p-values between bacterial and viral groups are depicted.

FIGS. 19A-19B. The clinical accuracy of the signature is robust to reduction in the technical accuracy of protein measurements. (A) The AUCs of the signature distinguishing bacterial from viral infection are estimated using a grayscale map as a function of CVs (std/mean) of TRAIL (y-axis) and CRP (x-axis) measurement. (B) AUC values on the diagonal of FIG. 19A a presented such that CV of TRAIL and CRP are equal.

FIG. 20 is a 3-dimensional visualization of bacterial (′+′), viral (‘∘’) and non-infectious (‘\hat{ }’) patients. Different patients types are mapped to distinct regions in the CRP (μg/ml), TRAIL and IP-10 (pg/ml) concentration map.

FIGS. 21A-21C. Probability of viral (A) bacterial or mixed (B) and non-infectious or healthy (C) as a function of TRAIL (y-axis), CRP (x-axis), and IP-10 concentrations, as obtained according to some embodiments of the present invention for IP-10 ranging from 0 to 100.

FIGS. 22A-22C. Probability of viral (A) bacterial or mixed (B) and non-infectious or healthy (C) as a function of TRAIL (y-axis), CRP (x-axis), and IP-10 concentrations, as obtained according to some embodiments of the present invention for IP-10 ranging from 100 to 200.

FIGS. 23A-23C. Probability of viral (A) bacterial or mixed (B) and non-infectious or healthy (C) as a function of TRAIL (y-axis), CRP (x-axis), and IP-10 concentrations, as obtained according to some embodiments of the present invention for IP-10 ranging from 200 to 300.

FIGS. 24A-24C. Probability of viral (A) bacterial or mixed (B) and non-infectious or healthy (C) as a function of TRAIL (y-axis), CRP (x-axis), and IP-10 concentrations, as obtained according to some embodiments of the present invention for IP-10 ranging from 300 to 400.

FIGS. 25A-25C. Probability of viral (A) bacterial or mixed (B) and non-infectious or healthy (C) as a function of TRAIL (y-axis), CRP (x-axis), and IP-10 concentrations, as obtained according to some embodiments of the present invention for IP-10 ranging from 400 to 500.

FIGS. 26A-26C. Probability of viral (A) bacterial or mixed (B) and non-infectious or healthy (C) as a function of TRAIL (y-axis), CRP (x-axis), and IP-10 concentrations, as obtained according to some embodiments of the present invention for IP-10 ranging from 500 to 1000.

FIGS. 27A-27C. Probability of viral (A) bacterial or mixed (B) and non-infectious or healthy (C) as a function of TRAIL (y-axis), CRP (x-axis), and IP-10 concentrations, as obtained according to some embodiments of the present invention for IP-10 ranging from 1000 to 2000.

FIGS. 28A-28C. Probability of viral (A) bacterial or mixed (B) and non-infectious or healthy (C) as a function of TRAIL (y-axis), CRP (x-axis), and IP-10 concentrations, as obtained according to some embodiments of the present invention for IP-10 which is 2000 or more.

FIGS. 29A-29F illustrate exemplary outputs of the method for distinguishing between bacterial and non-bacterial infection according to an embodiment of the present invention.

FIGS. 30A-30B are graphs illustrating the correlation between the rapid and slow protocol for measurement of TRAIL (FIG. 30A) and IP-10 (FIG. 30B).

FIG. 31 is a flowchart diagram of a method suitable for analyzing biological data obtained from a subject, according to various exemplary embodiments of the present invention.

FIGS. 32A-32B are schematic illustrations describing a procedure for calculating a distance of a surface from a plane according to some embodiments of the present invention.

FIGS. 33A-33D are schematic illustrations describing a procedure for obtaining the smooth version of a segment of a surface, according to some embodiments of the present invention.

FIG. 34 is a schematic illustration of a block diagram of a system for analyzing biological data, according to some embodiments of the present invention.

FIGS. 35A-35D are contour plots describing the probability of bacterial (FIG. 35A), viral (FIG. 35B), non-bacterial (FIG. 35C), and non-infectious (FIG. 35D) etiologies as a function of the coordinates δ₀ and δ₁. The probability values range between 0% (black) to 100% (white).

FIGS. 36A-36B. Low TRAIL levels are indicative or poor patient prognosis and outcome and high disease severity. (A) TRAIL concentrations in the serum of patients that were admitted to the ICU compared to all other patients (with infectious or non-infectious etiology). (B) TRAIL concentrations in the serum of pediatric patients that were admitted to the ICU or died compared to all other patients with infectious or non-infectious etiology.

FIGS. 37A-37B are graphs illustrating the difference in TRAIL concentrations in males and females of fertility age.

FIGS. 38A-38E are screenshots of a graphical user interface (GUI) suitable for receiving user input in a computer-implemented method for analyzing biological data according to some embodiments of the present invention.

FIGS. 39A and 39B are schematic illustrations of a block diagram of a system for analyzing biological data, in embodiments of the invention in which the system comprises a network interface (FIG. 39A) and a user interface (FIG. 39B).

### DESCRIPTION OF SPECIFIC EMBODIMENTS OF THE INVENTION

The present invention, in some embodiments thereof, relates to computational analysis, and, more particularly, but not exclusively, to computational analysis of biological data, e.g., for the purpose of distinguishing between bacterial infection and non-bacterial disease, and/or between a bacterial infection and viral infection, and/or between an infectious and non-infectious disease.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Different infectious agents have unique molecular patterns that can be identified and targeted by the immune system. Pathogen-associated molecular patterns (PAMPs) are an example of such molecules that are associated with different groups of pathogens and may be recognized by cells of the innate immune system using Toll-like receptors (TLRs) and other pattern recognition receptors (e.g. NOD proteins).

These patterns may vary considerably between different classes of pathogens and thus elicit different immune responses. For example, TLR-4 can recognize lipopolysaccharide, a constituent of gram negative bacteria, as well as lipoteichoic acids, constituent of gram positive bacteria, hence promoting an anti-microbial response of the immune system. TLR-3 can recognize single stranded RNA (often indicative of a viral infection) and thus prompt the appropriate anti-viral response. By distinguishing between different classes of pathogens (e.g bacterial versus viral) the immune system can mount the appropriate defense.

In the past few decades, several host markers have been identified that can be used for differential diagnosis of infection source in various indications. By measuring markers derived from the host rather than the pathogen, it is possible to minimize “false-positive” diagnoses due to non-pathogenic strains of bacteria that are part of the body's natural flora. One example is Procalcitonin (PCT), a precursor of the hormone calcitonin produced by the C-cells of the thyroid gland. PCT levels in the blood stream of healthy individuals is hardly detectable (in the pg/ml range) but it might increase dramatically, as a result of a severe infection with levels rising up to 100 ng/ml. PCT is heavily used to diagnose patients with systemic infection, sepsis, with sensitivity of 76% and specificity of 70%. However, studies that tested the diagnostic value of PCT in other non-systemic infection such as pneumonia or upper respiratory tract infections found it to be limited, especially when used in isolation.

The present inventors previously identified novel sets of biomarkers whose pattern of expression significantly correlates with infection type—as documented in International Patent Application WO2011132086 and WO2013/117746, both of which are incorporated herein by reference.

The present invention, in some embodiments thereof, is based on the use of signature of polypeptides for the diagnosis of bacterial infections, viral infections and non-bacterial, non-viral diseases. The methods of the present embodiments employ pattern recognition algorithms for the identification of the type of infection a subject is suffering from, which in turn allows for the selection of an appropriate treatment regimen. Various embodiments of the invention address limitations of current diagnostic solutions by: (i) allowing accurate diagnostics on a broad range of pathogens; (ii) enabling rapid diagnosis (within minutes); (iii) insensitivity to the presence of non-pathogenic bacteria and viruses (thus reducing the problem of false-positive); and (iv) eliminating the need for direct sampling of the pathogen, thus enabling diagnosis of inaccessible infections. Thus, some methods of the invention allow for the selection of subjects for whom antibiotic treatment is desired and prevent unnecessary antibiotic treatment of subjects having only a viral infection or a non-infectious disease. Some methods of the invention also allow for the selection of subjects for whom anti-viral treatment is advantageous.

To corroborate the findings in International Patent Application WO2013/117746, the present inventors have now increased the number of patients taking part in a multi-center clinical trial, enrolling 1002 hospital patients with different types of established infections as well as controls (patients with established non-viral/non-bacterial disease and healthy individuals).

Seeking to improve the level of accuracy and sensitivity of the previously described methods, the present inventors have now used a trinary classifier, which classifies patients (those having an established disease type) into one of three classes: bacterial infection, viral infection and non-bacterial, non-viral disease. Comparing the levels of a combination of polypeptides of a test subject with the expression patterns obtained in the study yielded superior results in terms of sensitivity and specificity compared to a binary classifier as summarized in Example 3 and Tables 9-12.

In the context of the present invention, the following abbreviations may be used: ANC=Absolute neutrophil count; ANN=Artificial neural networks; AUC=Area under the receiver operating curve; BP=Bordetella pertussis; CHF=Congestive heart failure; CI=Confidence interval; CID=Congenital immune deficiency; CLL=Chronic lymphocytic leukemia; CMV=Cytomegalovirus; CNS=Central nervous system; COPD=Chronic obstructive pulmonary disease; CP=Chlamydophila pneumonia; CRP=C-reactive protein; CSF=Cerebrospinal fluid; CV=Coefficient of variation; DOR=Diagnostic odds ratio; EBV=Epstein bar virus; eCRF=Electronic case report form; ED=Emergency department, ELISA=Enzyme-linked immunosorbent assay; FDR=False discovery rate; FMF=Familial Mediterranean fever; G-CSF=Granulocyte colony-stimulating factor; GM-CSF=Granulocyte-macrophage colony-stimulating factor; HBV=Hepatitis B virus; HCV=Hepatitis C virus; HI=Haemophilus influenza; HIV=Human immunodeficiency virus; IDE=Infectious disease experts; IL=Interleukin; IRB=institutional review board; IVIG=Intravenous immunoglobulin; KNN=K-nearest neighbors; LP=Legionella pneumophila; LR+=Positive likelihood ratio; LR-=Negative likelihood ratio; LRTI=Lower respiratory tract infections; mAb=Monoclonal antibodies; MDD=Minimum detectable dose; MDS=Myelodysplastic syndrome; MP=Mycoplasma pneumonia; MPD=Myeloproliferative disease; NPV=Negative predictive value; PCT=Procalcitonin; PED=Pediatric emergency department; PPV=Positive predictive value; QA=Quality assurance; RSV=Respiratory syncytial virus; RV=Rhinovirus; SIRS=systemic inflammatory syndrome; SP=Streptococcus pneumonia; STARD=Standards for Reporting of Diagnostic Accuracy; SVM=Support vector machine; TNF=Tumor necrosis factor; URTI=Upper respiratory tract infection; UTI=Urinary tract infection; WBC=White blood cell; WS=Wilcoxon rank-sum.

In the context of the present invention, the following statistical terms may be used:

“TP” is true positive, means positive test result that accurately reflects the tested-for activity. For example in the context of the present invention a TP, is for example but not limited to, truly classifying a bacterial infection as such.

“TN” is true negative, means negative test result that accurately reflects the tested-for activity. For example in the context of the present invention a TN, is for example but not limited to, truly classifying a viral infection as such.

“FN” is false negative, means a result that appears negative but fails to reveal a situation. For example in the context of the present invention a FN, is for example but not limited to, falsely classifying a bacterial infection as a viral infection.

“FP” is false positive, means test result that is erroneously classified in a positive category. For example in the context of the present invention a FP, is for example but not limited to, falsely classifying a viral infection as a bacterial infection.

“Sensitivity” is calculated by TP/(TP+FN) or the true positive fraction of disease subjects.

“Specificity” is calculated by TN/(TN+FP) or the true negative fraction of non-disease or normal subjects.

“Total accuracy” is calculated by (TN+TP)/(TN+FP+TP+FN).

“Positive predictive value” or “PPV” is calculated by TP/(TP+FP) or the true positive fraction of all positive test results. It is inherently impacted by the prevalence of the disease and pre-test probability of the population intended to be tested.

“Negative predictive value” or “NPV” is calculated by TN/(TN+FN) or the true negative fraction of all negative test results. It also is inherently impacted by the prevalence of the disease and pre-test probability of the population intended to be tested. See, e.g., O'Marcaigh A S, Jacobson R M, “Estimating The Predictive Value Of A Diagnostic Test, How To Prevent Misleading Or Confusing Results,” Clin. Ped. 1993, 32 (8): 485-491, which discusses specificity, sensitivity, and positive and negative predictive values of a test, e.g., a clinical diagnostic test.

“MCC” (Mathews Correlation coefficient) is calculated as follows: MCC=(TP*TN−FP*FN)/{(TP+FN)*(TP+FP)*(TN+FP)*(TN+FN)}\hat{ }0.5 where TP, FP, TN, FN are true-positives, false-positives, true-negatives, and false-negatives, respectively. Note that MCC values range between −1 to +1, indicating completely wrong and perfect classification, respectively. An MCC of 0 indicates random classification. MCC has been shown to be a useful for combining sensitivity and specificity into a single metric (Baldi, Brunak et al. 2000). It is also useful for measuring and optimizing classification accuracy in cases of unbalanced class sizes (Baldi, Brunak et al. 2000).

“Accuracy” refers to the degree of conformity of a measured or calculated quantity (a test reported value) to its actual (or true) value. Clinical accuracy relates to the proportion of true outcomes (true positives (TP) or true negatives (TN) versus misclassified outcomes (false positives (FP) or false negatives (FN)), and may be stated as a sensitivity, specificity, positive predictive values (PPV) or negative predictive values (NPV), Mathews correlation coefficient (MCC), or as a likelihood, odds ratio, Receiver Operating Characteristic (ROC) curve, Area Under the Curve (AUC) among other measures.

“Analytical accuracy” refers to the reproducibility and predictability of the measurement process itself, and may be summarized in such measurements as coefficients of variation (CV), Pearson correlation, and tests of concordance and calibration of the same samples or controls with different times, users, equipment and/or reagents. These and other considerations in evaluating new biomarkers are also summarized in Vasan, 2006.

“Performance” is a term that relates to the overall usefulness and quality of a diagnostic or prognostic test, including, among others, clinical and analytical accuracy, other analytical and process characteristics, such as use characteristics (e.g., stability, ease of use), health economic value, and relative costs of components of the test. Any of these factors may be the source of superior performance and thus usefulness of the test, and may be measured by appropriate “performance metrics,” such as AUC and MCC, time to result, shelf life, etc. as relevant.

By “statistically significant”, it is meant that the alteration is greater than what might be expected to happen by chance alone (which could be a “false positive”). Statistical significance can be determined by any method known in the art. Commonly used measures of significance include the p-value, which presents the probability of obtaining a result at least as extreme as a given data point, assuming the data point was the result of chance alone. A result is often considered highly significant at a p-value of 0.05 or less.

Aspects of the invention will now be described in detail.

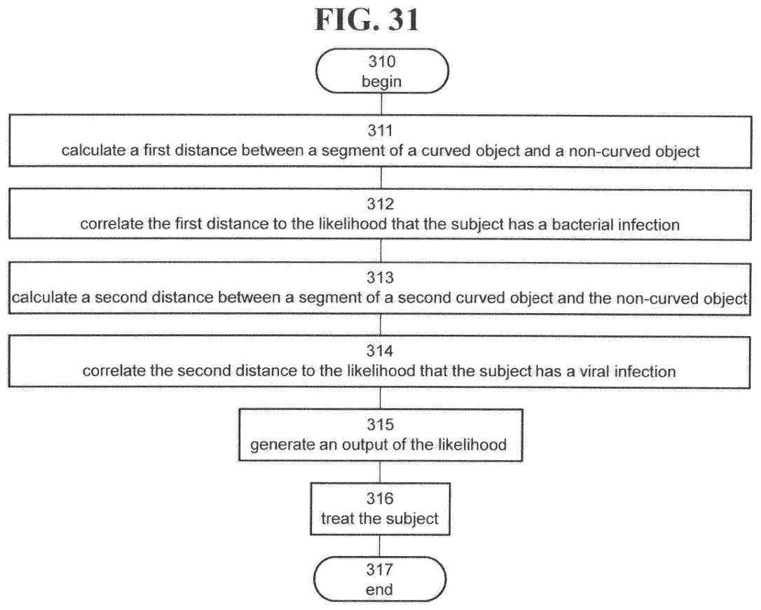

FIG. 31 is a flowchart diagram of a method suitable for analyzing biological data obtained from a subject, according to various exemplary embodiments of the present invention. It is to be understood that, unless otherwise defined, the operations described hereinbelow can be executed either contemporaneously or sequentially in many combinations or orders of execution. Specifically, the ordering of the flowchart diagrams is not to be considered as limiting. For example, two or more operations, appearing in the following description or in the flowchart diagrams in a particular order, can be executed in a different order (e.g., a reverse order) or substantially contemporaneously. Additionally, several operations described below are optional and may not be executed.

In some embodiments of the present invention the subject has been previously treated with an antibiotic, and in some embodiments of the present invention the subject has not been previously treated with an antibiotic.

Any of the methods described herein can be embodied in many forms. For example, it can be embodied in on a tangible medium such as a computer for performing the method operations. It can be embodied on a computer readable medium, comprising computer readable instructions for carrying out the method operations. It can also be embodied in electronic device having digital computer capabilities arranged to run the computer program on the tangible medium or execute the instruction on a computer readable medium.

Computer programs implementing the method of the present embodiments can commonly be distributed to users on a distribution medium such as, but not limited to, CD-ROMs or flash memory media. From the distribution medium, the computer programs can be copied to a hard disk or a similar intermediate storage medium. In some embodiments of the present invention, computer programs implementing the method of the present embodiments can be distributed to users by allowing the user to download the programs from a remote location, via a communication network, e.g., the internet. The computer programs can be run by loading the computer instructions either from their distribution medium or their intermediate storage medium into the execution memory of the computer, configuring the computer to act in accordance with the method of this invention. All these operations are well-known to those skilled in the art of computer systems.

The computational operations of the method of the present embodiments can be executed by a computer, either remote from the subject or near the subject. When the computer is remote from the subject, it can receive the data over a network, such as a telephone network or the Internet. To this end, a local computer can be used to transmit the data to the remote computer. This configuration allows performing the analysis while the subject is at a different location (e.g., at home), and also allows performing simultaneous analyses for multiple subjects in multiple different locations.

The computational operations of the method can also be executed by a cloud computing resource of a cloud computing facility. The cloud computing resource can include a computing server and optionally also a storage server, and can be operated by a cloud computing client as known in the art.

The method according to some embodiments may be used to “rule in” a bacterial infection. Alternatively, the method may be used to rule out a non-bacterial infection. The method according to some embodiments can be used to “rule out” a bacterial infection and “rule in” a non-bacterial disease.

The method according to some embodiments may be used to “rule in” a viral infection. Alternatively, the method may be used to rule out a non-viral infection.

The method according to some embodiments can be used to “rule out” a viral infection and “rule in” a non-viral disease.

The method according to some embodiments may be used to “rule in” an infectious disease. Alternatively, the method may be used to rule out a non-infectious disease. The method according to some embodiments can be used to “rule out” an infectious disease and “rule in” a non-infectious disease.

The biological data analyzed by the method contain expression values of a plurality of polypeptides in the blood of a subject. In some embodiments the biological data comprises expression values of only two polypeptides, in some embodiments the biological data comprises expression values of at least three polypeptides, in some embodiments biological data comprises expression values of only three polypeptides, in some embodiments biological data comprises expression values of at least four polypeptides, in some embodiments biological data comprises expression values of only four polypeptides, in some embodiments biological data comprises expression values of at least five polypeptides, and in some embodiments biological data comprises expression values of only five polypeptides.

The present Inventors contemplate many types of polypeptides. Representative examples include, without limitation, CRP, IP-10, TRAIL, IL1ra, PCT and SAA. In some embodiments the plurality of polypeptides comprises at least CRP and TRAIL, and in some embodiments the plurality of polypeptides comprises at least CRP, TRAIL and IP-10.

In some embodiments of the present invention, the biological data is provided in the form of a subject-specific dataset, as further detailed herein.

According to a particular embodiment, the levels of secreted (i.e. soluble) polypeptides (e.g., TRAIL, CRP and IP-10) are analyzed by the method.

The term “subject” as used herein is preferably a human. A subject can be male or female. The subject may be a newborn, baby, infant or adult. A subject can be one who has been previously diagnosed or identified as having an infection, and optionally has already undergone, or is undergoing, a therapeutic intervention for the infection. Alternatively, a subject can also be one who has not been previously diagnosed as having an infection. For example, a subject can be one who exhibits one or more risk factors for having an infection. A subject may also have an infection but show no symptoms of infection.

The subject whose disease is being diagnosed according to some embodiments of the present invention is referred to below as the “test subject”. The present Inventors have collected knowledge regarding the expression pattern of polypeptides, of a plurality of subjects whose disease has already been diagnosed, and have devised the analysis technique of the present embodiments based on the collected knowledge. This plurality of subjects is referred to below as “pre-diagnosed subjects” or “other subjects”.

As used herein, the phrase “bacterial infection” refers to a condition in which a subject is infected with a bacterium. The infection may be symptomatic or asymptomatic. In the context of this invention, the bacterial infection may also comprise a viral component (i.e. be a mixed infection being the result of both a bacteria and a virus).

The bacterial infection may be acute or chronic.

An acute infection is characterized by rapid onset of disease, a relatively brief period of symptoms, and resolution within days. A chronic infection is an infection that develops slowly and lasts a long time. One difference between acute and chronic infection is that during acute infection the immune system often produces IgM+ antibodies against the infectious agent, whereas the chronic phase of the infection is usually characteristic of IgM−/IgG+ antibodies. In addition, acute infections cause immune mediated necrotic processes while chronic infections often cause inflammatory mediated fibrotic processes and scaring. Thus, acute and chronic infections may elicit different underlying immunological mechanisms.

The bacterial infection may be the result of gram-positive, gram-negative bacteria or atypical bacteria.

The term “Gram-positive bacteria” as used herein refers to bacteria characterized by having as part of their cell wall structure peptidoglycan as well as polysaccharides and/or teichoic acids and are characterized by their blue-violet color reaction in the Gram-staining procedure. Representative Gram-positive bacteria include: Actinomyces spp., Bacillus anthracis, Bifidobacterium spp., Clostridium botulinum, Clostridium perfringens, Clostridium spp., Clostridium tetani, Corynebacterium diphtheriae, Corynebacterium jeikcium, Enterococcus faccalis, Enterococcus faccium, Erysipelothrix rhusiopathiae, Eubacterium spp., Gardnerella vaginalis, Gemella morbillorum, Leuconostoc spp., Mycobacterium abcessus, Mycobacterium avium complex, Mycobacterium chelonac, Mycobacterium fortuitum, Mycobacterium haemophilium, Mycobacterium kansasii, Mycobacterium leprac, Mycobacterium marinum, Mycobacterium scrofulaceum, Mycobacterium smegmatis, Mycobacterium terrac, Mycobacterium tuberculosis, Mycobacterium ulcerans, Nocardia spp., Peptococcus niger, Peptostreptococcus spp., Proprionibacterium spp., Staphylococcus aureus, Staphylococcus auricularis, Staphylococcus capitis, Staphylococcus cohnii, Staphylococcus epidermidis, Staphylococcus haemolyticus, Staphylococcus hominis, Staphylococcus lugdanensis, Staphylococcus saccharolyticus, Staphylococcus saprophyticus, Staphylococcus schleiferi, Staphylococcus similans, Staphylococcus warneri, Staphylococcus xylosus, Streptococcus agalactiae (group B streptococcus), Streptococcus anginosus, Streptococcus bovis, Streptococcus canis, Streptococcus equi, Streptococcus milleri, Streptococcus mitior, Streptococcus mutans, Streptococcus pneumoniae, Streptococcus pyogenes (group A streptococcus), Streptococcus salivarius, Streptococcus sanguis.

The term “Gram-negative bacteria” as used herein refer to bacteria characterized by the presence of a double membrane surrounding each bacterial cell.

Representative Gram-negative bacteria include Acinetobacter calcoaceticus, Actinobacillus actinomycetemcomitans, Aeromonas hydrophila, Alcaligenes xylosoxidans, Bacteroides, Bacteroides fragilis, Bartonella bacilliformis, Bordetella spp., Borrelia burgdorferi, Branhamella catarrhalis, Brucella spp., Campylobacter spp., Chalmydia pneumoniae, Chlamydia psittaci, Chlamydia trachomatis, Chromobacterium violaceum, Citrobacter spp., Eikenella corrodens, Enterobacter acrogenes, Escherichia coli, Flavobacterium meningosepticum, Fusobacterium spp., Haemophilus influenzae, Haemophilus spp., Helicobacter pylori, Klebsiella spp., Legionella spp., Leptospira spp., Moraxella catarrhalis, Morganella morganii, Mycoplasma pneumoniae, Neisseria gonorrhocac, Neisseria meningitidis, Pasteurella multocida, Plesiomonas shigelloides, Prevotella spp., Proteus spp., Providencia rettgeri, Pseudomonas aeruginosa, Pseudomonas spp., Rickettsia prowazekii, Rickettsia rickettsii, Rochalimaca spp., Salmonella spp., Salmonella typhi, Serratia marcescens, Shigella spp., Treponema carateum, Treponema pallidum, Treponema pallidum endemicum, Treponema pertenue, Veillonella spp., Vibrio cholerae, Vibrio vulnificus, Yersinia enterocolitica and Yersinia pestis.

The term “Atypical bacteria” refers to bacteria that do not fall into one of the classical “Gram” groups. Typically they are intracellular bacterial pathogens. They include, without limitations, Mycoplasmas spp., Legionella spp. Rickettsia spp., and Chlamydia spp.

The term “non-bacterial disease” as used herein, refers to any disease or condition that is not caused by infectious bacteria.

Referring to FIG. 31, the method begins at 310 and continues to 311 at which a first distance d between a segment S_{ROI} of a first curved object S and a non-curved object π is calculated. Generally, the first curved object S is a manifold in n dimensions, where n is a positive integer, and the non-curved object π is a hyperplane in an n+1 dimensional space.

The concept of n-dimensional manifolds and hyperplanes in n+1 dimensions are well known to those skilled in the art of geometry. For example, when n=1 the first curved object is a curved line, and the non-curved object π is a hyperplane in 2 dimensions, namely a straight line defining an axis. When n=2, the first curved object is a curved surface, and the non-curved object π is a hyperplane in 3 dimensions, namely a flat plane, referred to below as “a plane”.

The hyperplane π is defined by n directions. For example, when the non-curved object is an axis, it is defined by a single direction, and when the non-curved object is a plane it is defined by two directions, referred to as a first direction and a second direction.

The distance between the manifold S and hyperplane π is calculated at a point P over the hyperplane. P is defined by n coordinates. For example, when the hyperplane is an axis, P is defined by a single coordinate δ₁, along the single direction, and when the hyperplane is a plane, P is define by a pair of coordinates denoted (δ₀, δ₁), where do is referred to as “a first coordinate” and is defined along the first direction, and δ₁ is referred to as “a second coordinate” and is defined along the second direction. Unless explicitly stated otherwise, a reference to coordinate δ₀ describes an optional embodiment which is contemplated when S is a surface and π is a plane.

The directions are denoted using the same Greek letters as the respective coordinates, except that the directions are denoted by underlined Greek letters to indicate that these are vectors. Thus, the first direction is denoted δ₀, and the second direction is denoted δ₁.

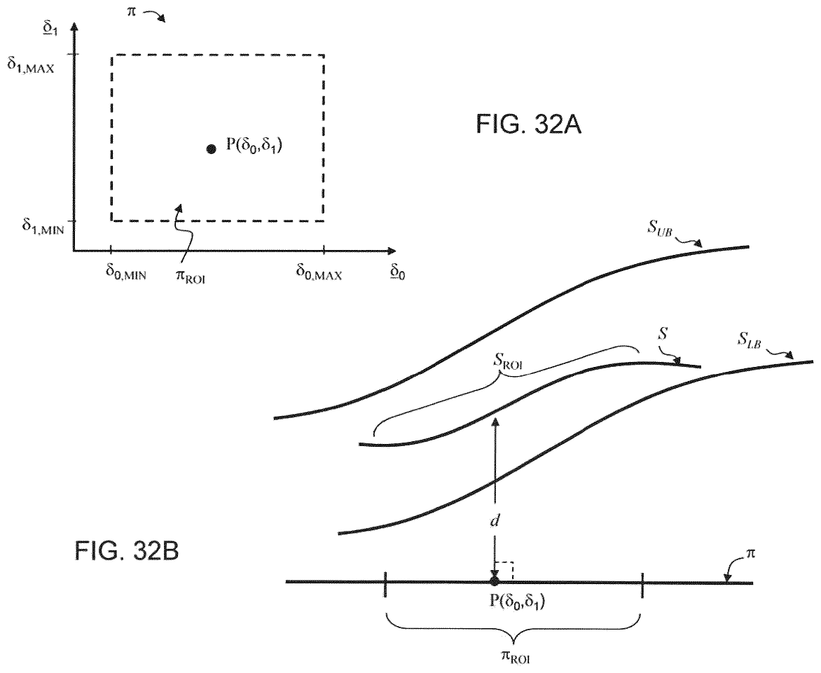

FIG. 32A illustrates the hyperplane π for the case of n=2. In these embodiments, π is a plane defined by directions δ₀ and δ₁. Also shown is a point P at (δ₀, δ₁). Directions δ₀ and δ₁, are shown orthogonal to each other, but this need not necessarily be the case, since the angle between δ₀ and δ₁ can be different from 90°. Within the plane π, there is a planar region-of-interest π_{ROI} spanning from a minimal first coordinate δ_{0,MIN} to a maximal first coordinate δ_{0,MAX} along direction δ₀, and from a minimal second coordinate δ_{1, MIN} to a maximal second coordinate δ_{1,MAX} along direction δ₁. The point P is within the region-of-interest π_{ROI}. When n=1 (not shown), π is an axis and the region-of-interest π_{ROI} is a linear segment of π spanning from δ_{1,MIN} to δ_{1,MAX} along direction δ₁.

The calculation of the first distance d is illustrated in FIG. 32B which illustrates the hyperplane π and manifold S. The distance d is measured from S to the point P, perpendicularly to π. It is to be understood that while each of objects π and S is illustrated as a one dimensional line, this need not necessarily be the case, since S and π are generally n-dimensional mathematical objects. For example, when S is a surface and π is a plane both π and S are two dimensional mathematical objects. The segment S_{ROI} of S is above a region-of-interest π_{ROI}. For example, when π is a plane π_{ROI} is a planar region-of-interest, and when π is an axis, π_{ROI} is a linear segment along the axis. Thus, π_{ROI} is the projection of S_{ROI} On T. For n=2, S_{ROI} is preferably a non-planar segment of (the surface) S, and for n=1, S_{ROI} is preferably a curved segment of (the curve) S.

Each of the n coordinates is defined by a combination of expression values of the polypeptides. For example, for n=1, the coordinate δ₁ is defined by a combination of expression values of the polypeptides, and for n=2 each of the coordinates δ₀ and δ₁ is defined by a different combination of expression values of the polypeptides.

For example, δ₁ and optionally also δ₀ are combinations of the polypeptides, according to the following equation:

$$\begin{matrix}{δ}_{0} = {a}_{0} + {a}_{1} {D}_{1} + {a}_{2} {D}_{2} + … + {ϕ}_{0} \\ {δ}_{1} = {b}_{0} + {b}_{1} {D}_{1} + {b}_{2} {D}_{2} + … + {ϕ}_{1} ,\end{matrix}$$

where a₀, a₁ . . . and b₀, b₁ . . . are constant and predetermined coefficients, and each of the variables D₁, D₂, . . . is an expression levels of one of the polypeptides, and ϕ₀ and ϕ₁ are functions that are nonlinear with respect to at least one of the expression levels.

Each of the functions ϕ₀ and ϕ₁ is optional and may, independently, be set to zero (or, equivalently, not included in the calculation of the respective coordinate). When ϕ₀=0 the coordinate do is a combination of the polypeptides, and when ϕ₁=0 the coordinate δ₁ is a combination of the polypeptides.

The nonlinear functions ϕ₀ and ϕ₁ can optionally and preferably be expressed as a sub of powers of expression levels, for example, according to the following equations:

ϕ₀=ΣᵢqᵢXᵢ^{γi}

ϕ₁=ΣᵢrᵢXᵢ^{λi},

where i is a summation index, qᵢ and rᵢ are sets of coefficients, Xᵢ∈{D₁, D₂, . . . }, and each of γi and λi is a numerical exponent. Note that the number of terms in each of the nonlinear functions ϕ₀ and ϕ₁ does not necessarily equals the number of the polypeptides, and that two or more terms in each sum may correspond to the same polypeptide, albeit with a different numerical exponent.

Representative examples of coefficients suitable for the present embodiments are provided in the Examples section that follows (see Tables 3, 13-17, 29 and 31-36).

When ϕ₀=0, ϕ₁=0 and the polypeptides include TRAIL, δ₀ is optionally and preferably an increasing function of an expression value of TRAIL, and δ₁ is a decreasing function of TRAIL. When ϕ₀=0, ϕ₁=0 and the polypeptides include CRP, δ₁ and optionally also δ₀ are optionally and preferably increasing functions of an expression value of CRP. When the polypeptides include IP-10, δ₁ and optionally also δ₀ are optionally and preferably are increasing functions of an expression value of IP-10.

In embodiments in which ϕ₀=0, ϕ₁=0 and the polypeptides include TRAIL, CRP and IP-10, each δ₀ and δ₁ can be a linear combination of TRAIL, CRP and IP-10, according to the following equation:

$$\begin{matrix}{δ}_{0} = {a}_{0} + {a}_{1} C + {a}_{2} I + {a}_{3} T \\ {δ}_{1} = {b}_{0} + {b}_{1} C + {b}_{2} I + {b}_{3} T ,\end{matrix}$$

where C, I and T are, respectively, the expression levels of CRP, IP-10 and TRAIL.

Preferably, both a₁ and b₁ are positive. Preferably both a₂ and b₂ are positive.

Preferably, a₃ is positive, and b₃ is negative. Representative examples of coefficients suitable for the embodiments in which the combination is linear combination and the polypeptides are CRP, IP-10 and TRAIL are provided in the Examples section that follows (see Tables 3, 13-17 and 33).

In embodiments in which ϕ₀≠0, ϕ₁≠0 and the polypeptides include TRAIL, CRP and IP-10, each δ₀ and δ₁ can be a combination of TRAIL, CRP and IP-10, according to the following equations:

$$\begin{matrix}{δ}_{0} = {a}_{0} + {a}_{1} C + {a}_{2} I + {a}_{3} T + {ϕ}_{0} \\ {δ}_{1} = {b}_{0} + {b}_{1} C + {b}_{2} I + {b}_{3} T + {ϕ}_{1} ,\end{matrix}$$

where each of ϕ₀ and ϕ₁ is a nonlinear function of at least one or at least two of C, I and T. As a representative example, ϕ₀ and ϕ₁ can be expressed as:

$$\begin{matrix}{ϕ}_{0} = {q}_{1} {C}^{{γ}^{1}} + {q}_{2} {C}^{{γ}^{2}} + {q}_{3} {T}^{{γ}^{3}} \\ {ϕ}_{1} = {r}_{1} {C}^{{γ}^{1}} + {r}_{2} {C}^{{γ}^{2}} + {r}_{3} {T}^{{γ}^{3}} .\end{matrix}$$

Representative examples of coefficients suitable for the embodiments in which the polypeptides are CRP, IP-10 and TRAIL and the nonlinear functions are not taken to be zero are provided in the Examples section that follows (see Table 36).

The boundaries δ_{0,MIN}, δ_{0,MAX}, δ_{1,MIN} and δ_{1, MAX} of π_{ROI} preferably correspond to the physiologically possible ranges of the expression values of the polypeptides.

When measured using the protocols described in Example 8, more preferably Example 9, below, the physiologically possible ranges are typically from 0 to about 400 pg/ml (CRP), from 0 to about 3000 pg/ml (IP-10), and from 0 to about 700 pg/ml (TRAIL). Some subjects may exhibit concentrations that lie outside these ranges. In various exemplary embodiments of the invention, when the expression values of TRAIL, CRP and IP-10 are measured according to the protocol described in Example 8, more preferably Example 9, below, the values of the coefficients a₀, . . . , a₃ and b₀, . . . , b₃ are taken from Table 3, below, and the boundaries of π_{ROI} are: δ_{0,MIN}=−1.3 δ_{0,MAX}=45 δ_{1,MIN}=−14.3 and δ_{1,MAX}=49.6.

When the expression values of TRAIL, CRP and IP-10 are measured by a protocol which is different from the protocol described in Example 8, more preferably Example 9, below, the values of the coefficients a₀, . . . , a₃ and b₀, . . . , b₃ are different from the values in Table 3 below, and therefore the boundaries of π_{ROI} are also different from the above values. In such cases, the values of the coefficients and boundaries are correlative to the aforementioned values wherein the correlation for each coefficient and boundary is derived from the correlation between the expression value of the respective protein as measured according to the protocol described in Example 8, more preferably Example 9, and the expression value of the respective protein as actually measured.

At least a major part of the segment S_{ROI} of curved object S is between two curved objects referred to below as a lower bound curved object S_{LB} and an upper bound curved object S_{UB}.

As used herein “major part of the segment S_{ROI}” refers to a part of a smoothed version S_{ROI} whose length (when n=1), surface area (when n=2) or volume (when n≥3) is 60% or 70% or 80% or 90% or 95% or 99% of a smoothed version of the length, surface area or volume of S_{ROI}, respectively.

As used herein, “a smooth version of the segment S_{ROI}” refers to the segment S_{ROI}, excluding regions of S_{ROI} at the vicinity of points at which the Gaussian curvature is above a curvature threshold, which is X times the median curvature of S_{ROI}, where X is 1.5 or 2 or 4 or 8.

The following procedure can be employed for the purpose of determining whether the major part of the segment S_{ROI} is between S_{LB} and S_{UB}. Firstly, a smoothed version of the segment S_{ROI} is obtained. Secondly, the length (when n=1), surface area (when n=2) or volume (when n≥3) A₁ of the smoothed version of the segment S_{ROI} is calculated. Thirdly, the length (when n=1) surface area (when n=2) or volume (when n≥3) A₂ of the part of the smoothed version of the segment S_{ROI} that is between S_{LB} and S_{UB} is calculated. Fourthly, the percentage of A₂ relative to A₁ is calculated.

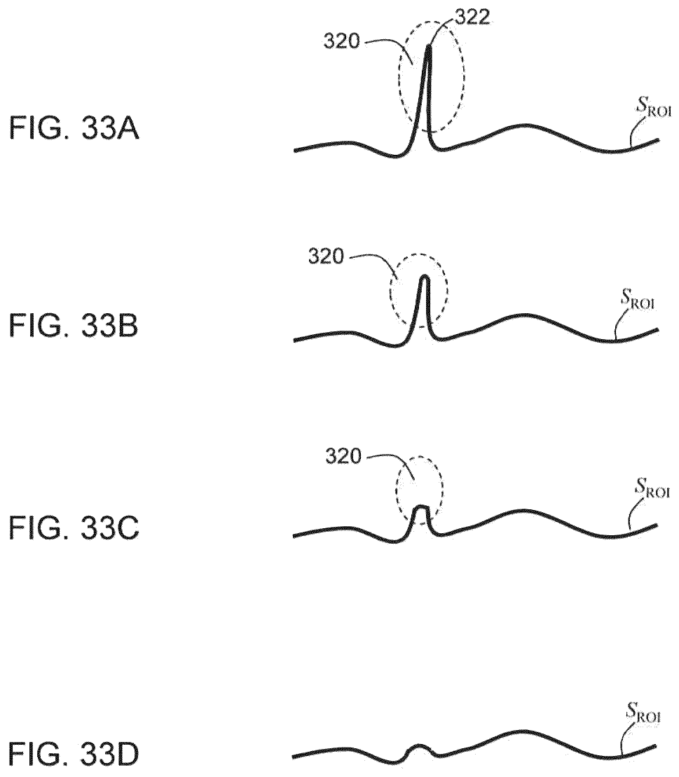

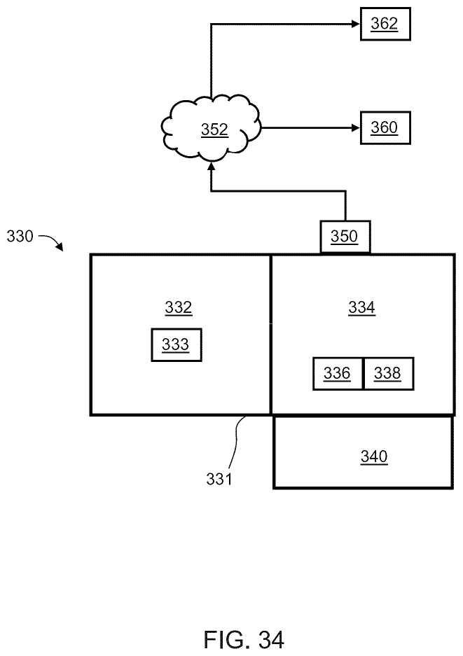

FIGS. 33A-33D illustrates a procedure for obtaining the smooth version of S_{ROI}.

For clarity of presentation, S_{ROI} is illustrated as a one dimensional segment, but the skilled person would understand that S_{ROI} is generally an n-dimensional mathematical object. The Gaussian curvature is calculated for a sufficient number of sampled points on S_{ROI}. For example, when the manifold is represented as point cloud, the Gaussian curvature can be calculated for the points in the point cloud. The median of the Gaussian curvature is then obtained, and the curvature threshold is calculated by multiplying the obtained median by the factor X. FIG. 33A illustrates S_{ROI} before the smoothing operation. Marked is a region 320 having one or more points 322 at which the Gaussian curvature is above the curvature threshold. The point or points at which with the Gaussian curvature is maximal within region 320 is removed and region 320 is smoothly interpolated, e.g., via polynomial interpolation, (FIG. 33B). The removal and interpolation is repeated iteratively (FIG. 33C) until the segment S_{ROI} does not contain regions at which the Gaussian curvature is above the curvature threshold (FIG. 33D).

When n=1 (namely when S is a curved line), S_{LB} is a lower bound curved line, and S_{UB} an upper bound curved line. In these embodiments, S_{LB} and S_{UB} can be written in the form:

$$\begin{matrix}{S}_{LB} = f ( {δ}_{1} ) - {ε}_{0} , \\ {S}_{UB} = f ( {δ}_{1} ) + {ε}_{1}\end{matrix}$$

where f(δ₁) is a probabilistic classification function of the coordinate δ₁ (along the direction δ₁) which represents the likelihood that the test subject has a bacterial infection. In some embodiments of the invention f(δ₁)=1/(1+exp (δ₁)). Both S_{LB} and S_{UB} are positive for any value of δ₁ within π_{ROI}. Also contemplated, are embodiments in which f(δ₁) is a probabilistic classification function which represents the likelihood that the test subject has a viral infection. Further contemplated, are embodiments in which f(δ₁) is a probabilistic classification function which represents the likelihood that the test subject has an infection.

When n=2 (namely when S is a curved surface), S_{LB} is a lower bound curved surface, and S_{UB} an upper bound curved surface. In these embodiments, S_{LB} and S_{UB} can be written in the form:

$$\begin{matrix}{δ}_{LB} = f ( {δ}_{0} , {δ}_{1} ) - {ε}_{0} , \\ {S}_{UB} = f ( {δ}_{0} , {δ}_{1} ) + {ε}_{1}\end{matrix}$$

where f(δ₀, δ₁) is a probabilistic classification function of the first and second coordinates (along the first and second directions) which represents the likelihood that the test subject has a bacterial infection. In some embodiments of the invention f(δ₀, δ₁)=exp (δ₁)/(1+exp (δ₀)+exp (δ₁)). Both S_{LB} and S_{UB} are positive for any value of δ₀ and δ₁ within π_{ROI}.

In any of the above embodiments each of the parameters ε₀ and ε₁ is less than 0.5 or less than 0.4 or less than 0.3 or less than 0.2 or less than 0.1 or less than 0.05.

Referring again to FIG. 31, the method proceeds to 312 at which the calculated distance d is correlated to the presence of, absence of, or likelihood that the subject has, a disease or condition corresponding to the type of the probabilistic function f. For example, when the probabilistic function f represents the likelihood that the test subject has a bacterial infection, the calculated distance d is correlated to the presence of, absence of, or likelihood that the subject has, a bacterial infection.

In various exemplary embodiments of the invention the correlation includes determining that the distance d is the likelihood that the subject has a bacterial infection. The likelihood is optionally and preferably compared to a predetermined threshold ω_{B}, wherein the method can determine that it is likely that the subject has a bacterial infection when the likelihood is above ω_{B}, and that it is unlikely that the subject has a bacterial infection otherwise. Typical values for ω_{B} include, without limitation, about 0.2, about 0.3, about 0.4, about 0.5, about 0.6 and about 0.7. Other likelihood thresholds are also contemplated.

In some embodiments of the present invention, when the method determines that it is likely that the subject has a bacterial infection, the subject is treated (316) for the bacterial infection, as further detailed herein.

The present inventors found a probabilistic classification function g(♦₀, δ₁) which represents the likelihood that the test subject has a viral infection. In various exemplary embodiments of the invention g(δ₀, δ₁) equals exp (δ₀)/(1+exp (δ₀)+exp (δ₁)).

The function g can, according to some embodiments of the present invention, be utilized also for estimating the presence of, absence of, or likelihood that the subject has, a viral infection. Thus, in some embodiments, the method proceeds to 313 at which a second distance between a segment of a second curved surface and the plane π is calculated, and 314 at which the second distance is correlated to the presence of, absence of, or likelihood that the subject has, a viral infection. The procedure and definitions corresponding to 313 and 314 are similar to the procedure and definitions corresponding to 311 and 312 described above, mutatis mutandis. Thus, for example, a major part of the segment of the second surface is between a second lower bound surface g(♦₀, δ₁)−ε₂ and a second upper bound surface g(δ₀, δ₁)+ε₃, wherein each of ε₂ and ε₃ is less than 0.5 or less than 0.4 or less than 0.3 or less than 0.2 or less than less than 0.1 or less than 0.05.

In some embodiments of the present invention, when the method determines that it is likely that the subject has a viral infection, the subject is treated (316) for the viral infection, as further detailed herein.

In various exemplary embodiments of the invention the correlation includes determining that the second distance is the likelihood that the subject has a viral infection. The likelihood is optionally and preferably compared to a predetermined threshold ωᵥ, wherein the method can determine that it is likely that the subject has a viral infection when the likelihood is above ωᵥ, that it is unlikely that the subject has a viral infection otherwise. Typical values for ωᵥ include, without limitation, about 0.5, about 0.6 about 0.7 and about 0.8. Other likelihood thresholds are also contemplated.

In embodiments in which operations 313 and 314 are executed, operations 311 and 312 can be either executed or not executed. For example, the present embodiments contemplate a procedure in which operations 311 and 312 are not executed, and the method determines the likelihood that the subject has a viral infection, without calculating the first distance and without correlating the first distance to the presence of, absence of, or likelihood that the subject has, a bacterial infection.

Alternatively, all operations 311-314 can be executed, wherein 311 and 312 are executed irrespectively of the outcome of 314, and 313 and 314 are executed irrespectively of the outcome of 312. In these embodiments, the method optionally and preferably determines both the likelihood that the subject has a bacterial infection, and the likelihood that the subject has a viral infection. Each of these likelihoods can be compared to the respective predetermined threshold (ω_{B} or ωᵥ). When each of the likelihoods is below the respective threshold, the method can determine that the patient is likely to have a non-bacterial and non-viral infectious disease. For example, the method can determine that it is likely that the subject has a non-infectious disease, a fungal disease or a parasitic disease.

Still alternatively, whether or not some operations are executed is dependent on the outcome of one or more other operations. For example, the method can execute 311 and 312, so as to determine the likelihood that the subject has a bacterial infection. Thereafter, the determined likelihood is compared to the threshold ω_{B}. The method skips the execution of 313 and 314 if the determined likelihood is above ω_{B}, and executes 313 and 314 otherwise. Another example of these embodiments is a procedure in which the method executes 313 and 314, so as to determine the likelihood that the subject has a viral infection. Thereafter, the determined likelihood is compared to the threshold ωᵥ. The method skips the execution of 311 and 312 if the determined likelihood is above ωᵥ, and executes 311 and 312 otherwise.

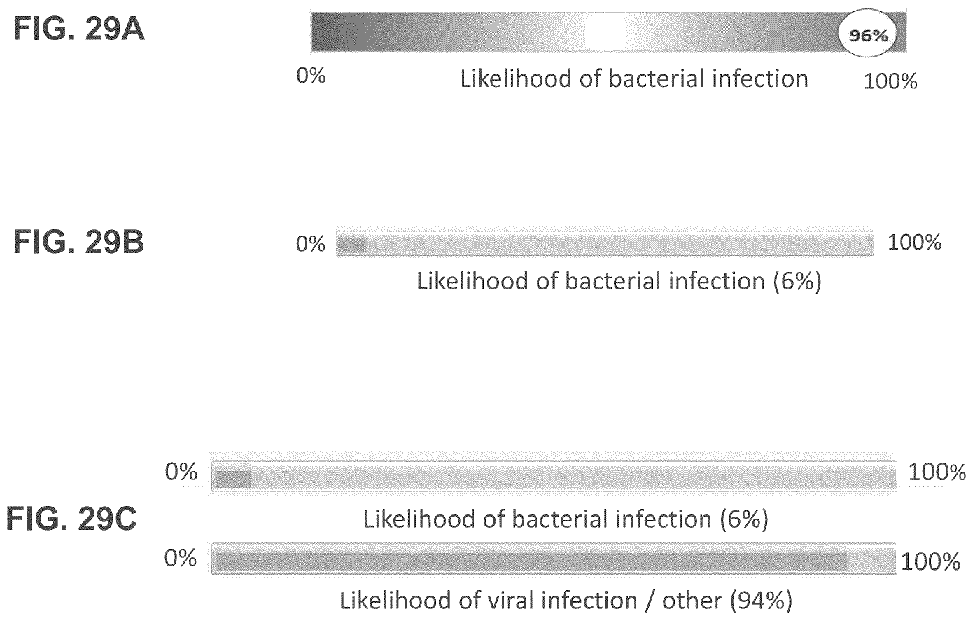

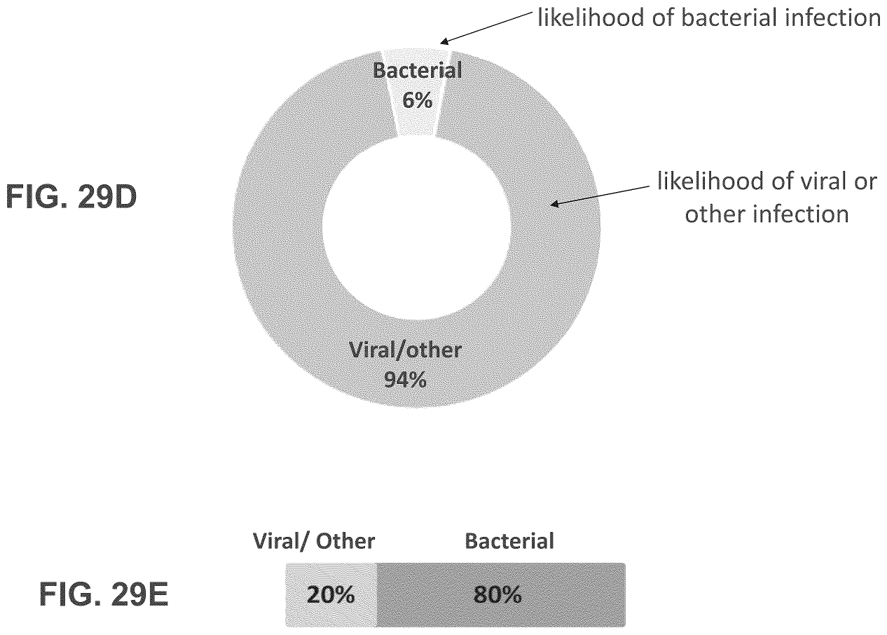

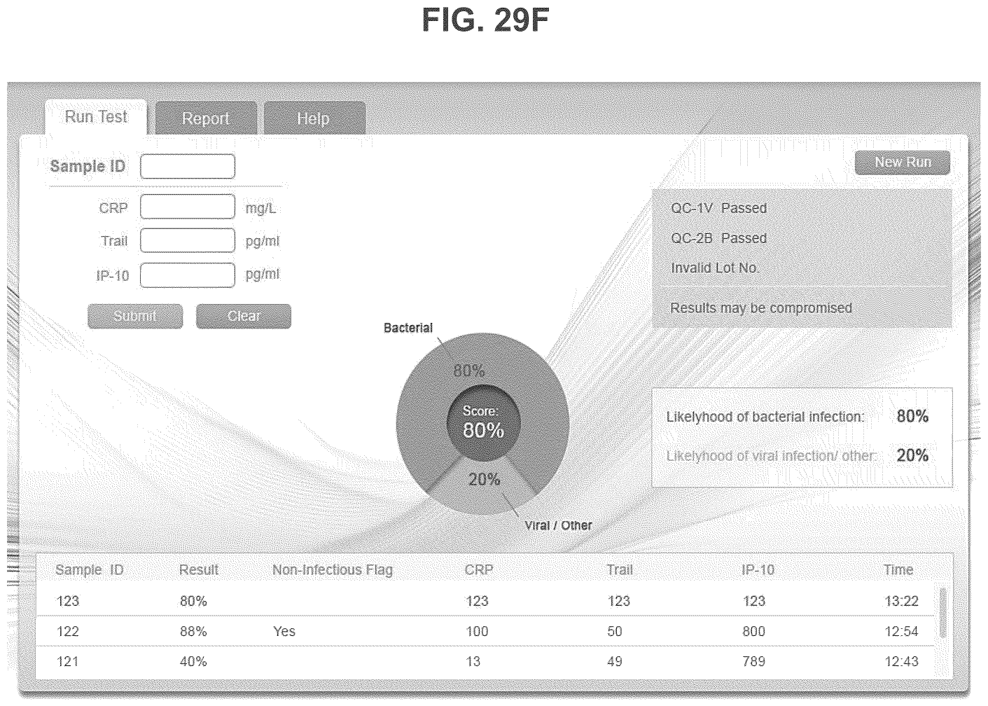

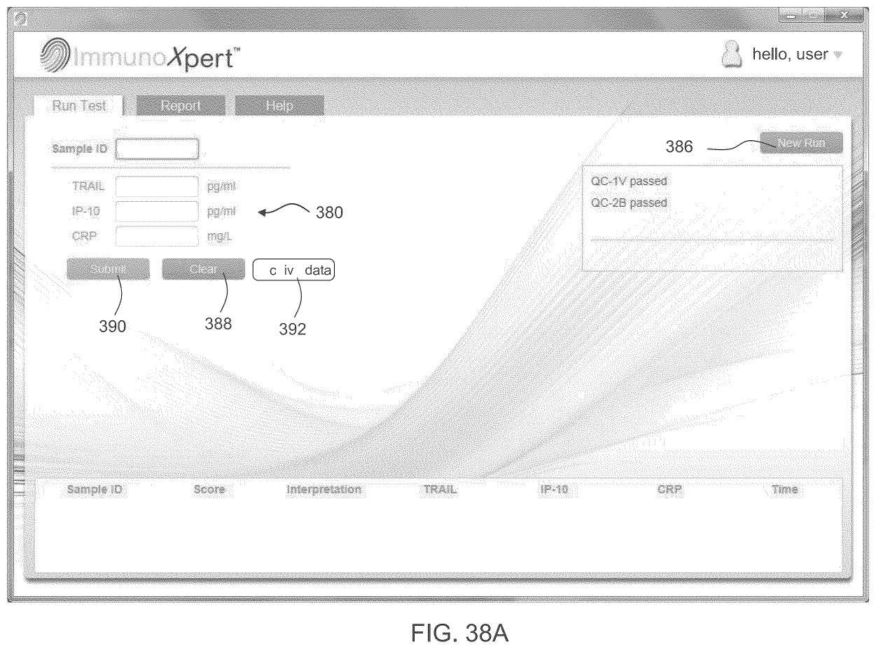

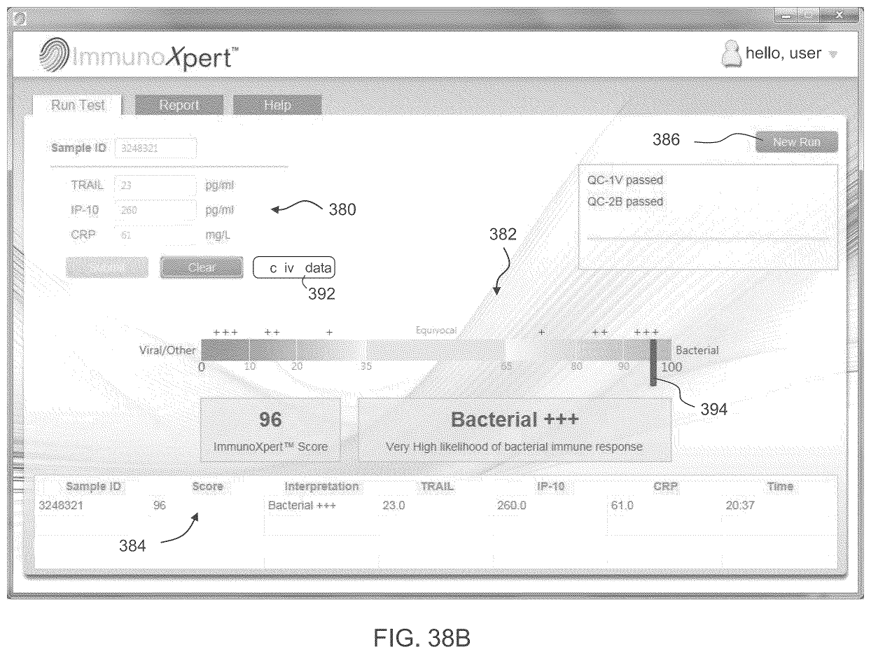

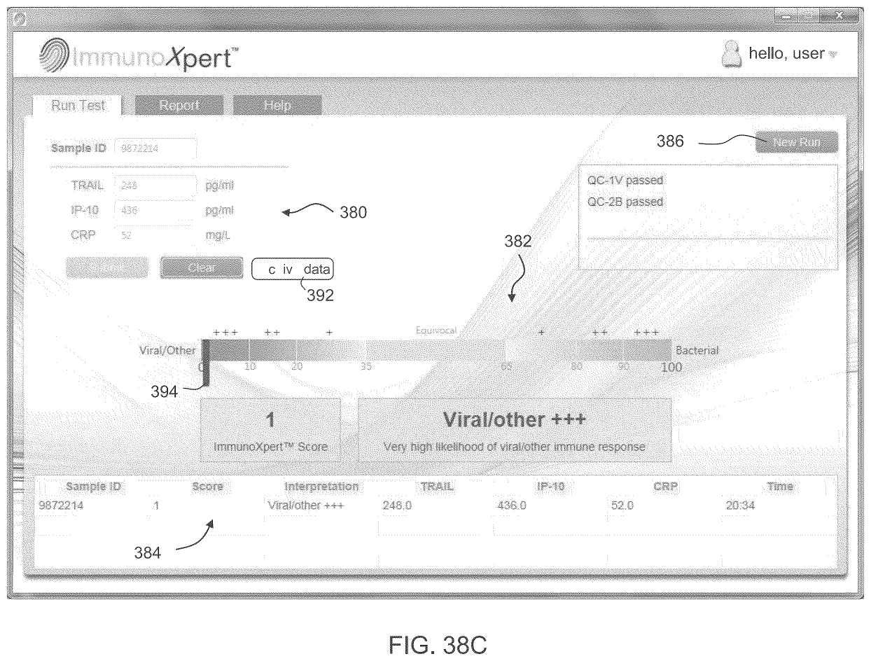

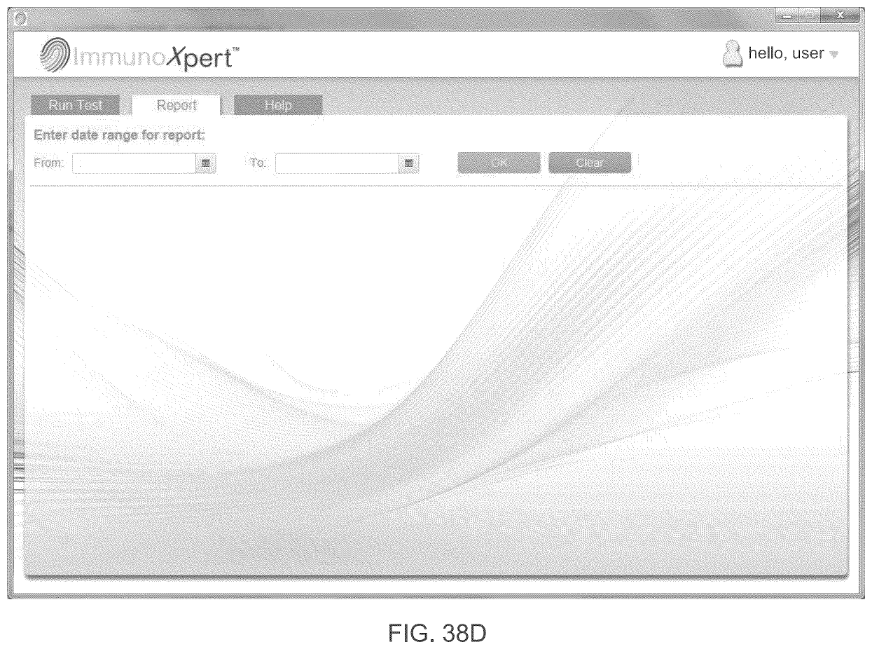

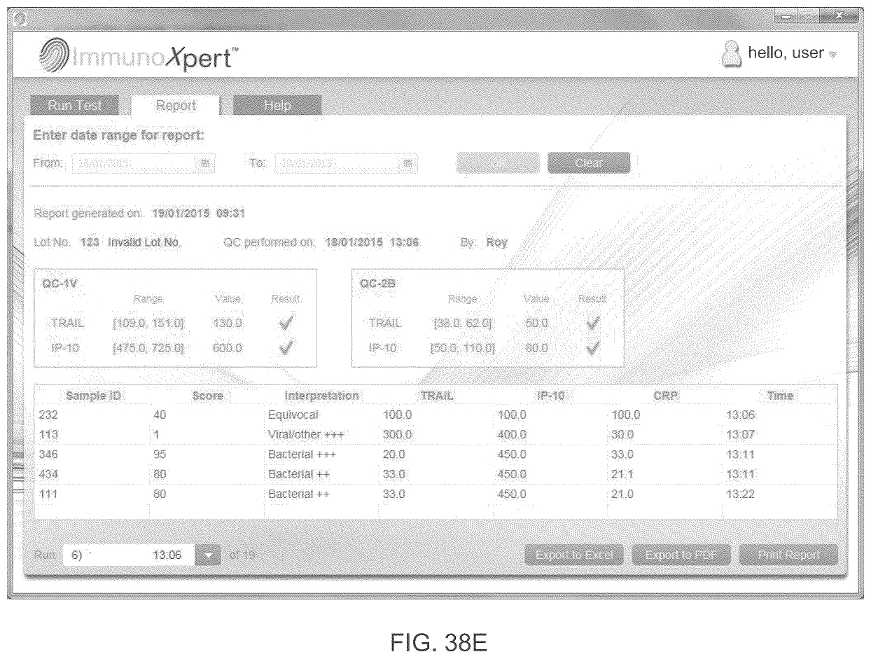

The method optionally and preferably continues to 315 at which an output of the likelihood(s) is generated. The output can be presented as text, and/or graphically and/or using a color index. The output can optionally include the results of the comparison to the threshold ω_{B}. FIGS. 29A-29F and 38A-38E illustrate exemplary outputs suitable for distinguishing between bacterial and non-bacterial infection according to an embodiment of the present invention.

The method ends at 317.

FIGS. 38A-38E are screenshots of a graphical user interface (GUI) suitable for receiving user input in a computer-implemented method for analyzing biological data according to some embodiments of the present invention.

The GUI comprises a calculation activation control 390, that may be in the form of a button control. The GUI may also comprise a plurality of expression value input fields 380, wherein each expression value input field is configured for receiving from a user an expression value of a polypeptide in the blood of a subject. The user feeds into the input fields the expression values of the polypeptides. Alternatively, the expression values are can be received by establishing a communication between the computer and an external machine (not shown) that measures the expression values. In these embodiments, it is not necessary for the user to manually feed the expression values into the input fields. In some embodiments, the GUI comprises a communication control 392, e.g., in the form of a button control, wherein the communication with the external machine is in response to an activation of the communication control by the user.

Responsively to an activation of control 390 by the user, the computer calculates a score based on the expression values as received automatically or via fields 380. The core can be the likelihood that the subject has a bacterial infection and/or a viral infection. The score can be calculated for example, by calculating a distance between a curved surface and a plane defined by the two directions as further detailed hereinabove.

A graphical scale 382 can be generated on the GUI. The graphical scale can include a first end, identified as corresponding to a viral infection, and a second end, identified as corresponding to a bacterial infection.

Once the score is calculated, a mark 394 can optionally and preferably be made on the graphical 382 at a location corresponding to the calculated likelihood. FIG. 38A shows the GUI before the values have been fed into the input fields, FIG. 38B shows mark 394 on scale 382 at a location that corresponds to a likelihood of 96% that the infection is bacterial, and FIG. 38C shows mark 394 on scale 382 at a location that corresponds to a likelihood of 1% that the infection is bacterial (or, equivalently, likelihood of 99% that the infection is viral). Optionally, the GUI also displays the calculated score numerically.

The GUI optionally and preferably includes one or more additional controls 386, 388 that may be in the form of button controls. For example, control 388 can instruct the computer to clear the input fields 380 when the user activates the control 388. This allows the user to feed values that correspond to a different sample. In some embodiments, the GUI also generates an output 384 that summarizes the results of the previous samples. Control 386 can instruct the computer to clear the input fields 380 as well as the output 384 when the user activates the control 386. This allows the user to begin a new run (optionally with multiple samples) without logging out of the GUI.

A representative example of a protocol suitable for the present embodiments is as follows.

The GUI presents an authenticated user with a dialog that allows the user to feed in quality control (QC) values of an experiment. The QC is validated, and the GUI in FIG. 38A is generated. The user feeds in the expression values in fields 380 and activate control 390 to receive the result (e.g., FIGS. 38B and 38C). To feed in expression values of another blood sample the user activates control 388. The result of each sample is added to output 384 which can be, for example, in the form of a table. To enter a new experiment without closing the software or logging out the user activates control 386 to clear output 384 and enter new QC values. Preferably, all the operations are logged in one or more log files.

In some embodiments of the present invention GUI also includes a report screen (FIGS. 38D and 38E) that displays the results of previous experiments, for example, in response to a date based request.

It will be appreciated that the polypeptide names presented herein are given by way of example. Many alternative names, aliases, modifications, isoforms and variations will be apparent to those skilled in the art. Accordingly, it is intended to embrace all the alternative protein names, aliases, modifications isoforms and variations.

Gene products, are identified based on the official letter abbreviation or gene symbol assigned by the international Human Genome Organization Naming Committee (HGNC) and listed at the date of this filing at the US National Center for Biotechnology Information (NCBI) web site also known as Entrez Gene.

TRAIL: The protein, TNF Related Apoptosis Inducing Ligand (TRAIL), encoded by this gene is a cytokine that belongs to the tumor necrosis factor (TNF) ligand family. Additional names of the gene include without limitations APO2L, TNF-related apoptosis-inducing ligand, TNFSF10 and CD253. TRAIL exists in a membrane bound form and a soluble form, both of which can induce apoptosis in different cells, such as transformed tumor cells. This protein binds to several members of the TNF receptor superfamily such as TNFRSF10A/TRAILR1, NFRSF10B/TRAILR2, NFRSF10C/TRAILR3, TNFRSF10D/TRAILR4, and possibly also to NFRSF11B/OPG. The activity of this protein may be modulated by binding to the decoy receptors such as NFRSF10C/TRAILR3, TNFRSF10D/TRAILR4, and NFRSF11B/OPG that cannot induce apoptosis. The binding of this protein to its receptors has been shown to trigger the activation of MAPK8/JNK, caspase 8, and caspase 3. Alternatively spliced transcript variants encoding different isoforms have been found for this gene. TRAIL can be proteolytically cleaved from the cell surface to produce a soluble form that has a homotrimeric structure.

According to a particular embodiment, the level of the soluble (i.e. secreted) form of TRAIL is measured.

According to another embodiment, the membrane form of TRAIL is measured.

According to still another embodiment, both the membrane form of TRAIL and the secreted form of TRAIL are measured.

According to another aspect of the present invention there is provided a method of determining an infection type in a subject comprising measuring the concentration of soluble TRAIL and insoluble TRAIL, wherein the concentration is indicative of the infection type.

In one embodiment, when the concentration of the soluble TRAIL is higher than a pre-determined threshold value, a bacterial infection is ruled out for the subject.

In another embodiment, when the concentration of the soluble TRAIL is higher than a pre-determined threshold value, a viral infection is ruled in for the subject.

Exemplary protein sequences for soluble TRAIL are set forth in SEQ ID NO: 37 and SEQ ID NO: 38.

An exemplary mRNA sequence of membrane human TRAIL is set forth in SEQ ID NO: 1.

An exemplary amino acid sequences of membrane human TRAIL is set forth in SEQ ID NOs: 4.

Other exemplary cDNA and amino acid sequences for TRAIL are set forth in SEQ ID NOs: 2, 3 and 5-8.

IP10: This gene encodes a chemokine of the CXC subfamily and ligand for the receptor CXCR3. Binding of this protein to CXCR3 results in pleiotropic effects, including stimulation of monocytes, natural killer and T-cell migration, and modulation of adhesion molecule expression. Additional names of the gene include without limitations: IP-10, CXCL10, Gamma-IP10, INP10 and chemokine (C—X—C motif) ligand 10.

Exemplary cDNA sequence of human IP10 is set forth in SEQ ID NOs: 9-12. An exemplary amino acid sequence of human IP10 is set forth in SEQ ID NO: 13.

CRP: C-reactive protein; additional aliases of CRP include without limitation RP11-419N10.4 and PTX1. The protein encoded by this gene belongs to the pentaxin family. It is involved in several host defense related functions based on its ability to recognize foreign pathogens and damaged cells of the host and to initiate their elimination by interacting with humoral and cellular effector systems in the blood. Consequently, the level of this protein in plasma increases greatly during acute phase response to tissue injury, infection, or other inflammatory stimuli. CRP displays several functions associated with host defense: it promotes agglutination, bacterial capsular swelling, phagocytosis and complement fixation through its calcium-dependent binding to phosphorylcholine.

Exemplary cDNA sequence of human CRP is set forth in SEQ ID NOs: 14-16.

An exemplary amino acid sequence of human CRP is set forth in SEQ ID NO: 17.

IL1RA: The protein encoded by this gene is a cytokine receptor that belongs to the interleukin 1 receptor family. This protein is a receptor for interleukin alpha (IL1A), interleukin beta (IL1B), and interleukin 1 receptor, type I (IL1R1/IL1RA). It is an important mediator involved in many cytokine induced immune and inflammatory responses. Additional names of the gene include without limitations: CD121A, IL-1RT1, p80, CD121a antigen, CD121A, IL1R and IL1ra.

Exemplary cDNA sequences of human IL1RA are set forth in SEQ ID NOs: 18, 19 and 20.

Exemplary amino acid sequences of human IL1RA are set forth in SEQ ID NOs: 21-24.

PCT: Procalcitonin (PCT) is a peptide precursor of the hormone calcitonin, the latter being involved with calcium homeostasis. Procalcitonin (“pCT”) is a protein consisting of 116 amino acids and having a molecular weight of about 13,000 dalton. It is the prohormone of calcitonin which under normal metabolic conditions is produced and secreted by the C cells of the thyroid. pCT and calcitonin synthesis is initiated by translation of preprocalcitonin (“pre-pCT”), a precursor peptide comprising 141 amino acids. The amino acid sequence of human pre-pCT was described by Moullec et al. in FEBS Letters, 167:93-97 in 1984. pCT is formed after cleavage of the signal peptide (first 25 amino acids of pre-pCT).

Exemplary cDNA sequences of human PCT are set forth in SEQ ID NOs: 31-32.

Exemplary amino acid sequences of human PCT are set forth in SEQ ID NOs: 33-36.

SAA: encodes a member of the serum amyloid A family of apolipoproteins. The encoded protein is a major acute phase protein that is highly expressed in response to inflammation and tissue injury. This protein also plays an important role in HDL metabolism and cholesterol homeostasis. High levels of this protein are associated with chronic inflammatory diseases including atherosclerosis, rheumatoid arthritis, Alzheimer's disease and Crohn's disease. This protein may also be a potential biomarker for certain tumors. Alternate splicing results in multiple transcript variants that encode the same protein.

Exemplary cDNA sequences of human SAA are set forth in SEQ ID NOs: 25-27.

Exemplary amino acid sequences of human SAA are set forth in SEQ ID NO:28-30.

It will be appreciated that since patient to patient DNA variations may give rise to SNPs which can cause differences in the amino acid sequence of the proteins, the present inventors also contemplate proteins having amino acid sequences at least 90%, 95% or 99% homologous to the sequences provided herein above.

Measuring the polypeptide (for example, TRAIL, IP-10 and CRP) levels is typically affected at the protein level as further described herein below.

Methods of Detecting Expression and/or Activity of Proteins

Expression and/or activity level of proteins expressed in the cells of the cultures of some embodiments of the invention can be determined using methods known in the arts and typically involve the use of antibodies. Such methods may be referred to as immunoassays. Immunoassays may be run in multiple steps with reagents being added and washed away or separated at different points in the assay. Multi-step assays are often called separation immunoassays or heterogeneous immunoassays. Some immunoassays can be carried out simply by mixing the reagents and sample and making a physical measurement. Such assays are called homogenous immunoassays or less frequently non-separation immunoassays. The use of a calibrator is often employed in immunoassays. Calibrators are solutions that are known to contain the analyte in question, and the concentration of that analyte is generally known. Comparison of an assay's response to a real sample against the assay's response produced by the calibrators makes it possible to interpret the signal strength in terms of the presence or concentration of analyte in the sample.

The antibody may be monoclonal, polyclonal, chimeric, or a fragment of the foregoing, and the step of detecting the reaction product may be carried out with any suitable immunoassay.

Suitable sources for antibodies for the detection of the polypeptides include commercially available sources such as, for example, Abazyme, Abnova, AssayPro, Affinity Biologicals, AntibodyShop, Aviva bioscience, Biogenesis, Biosense Laboratories, Calbiochem, Cell Sciences, Chemicon International, Chemokine, Clontech, Cytolab, DAKO, Diagnostic BioSystems, eBioscience, Endocrine Technologies, Enzo Biochem, Eurogentec, Fusion Antibodies, Genesis Biotech, GloboZymes, Haematologic Technologies, Immunodetect, Immunodiagnostik, Immunometrics, Immunostar, Immunovision, Biogenex, Invitrogen, Jackson ImmunoResearch Laboratory, KMI Diagnostics, Koma Biotech, LabFrontier Life Science Institute, Lee Laboratories, Lifescreen, Maine Biotechnology Services, Mediclone, MicroPharm Ltd., ModiQuest, Molecular Innovations, Molecular Probes, Neoclone, Neuromics, New England Biolabs, Novocastra, Novus Biologicals, Oncogene Research Products, Orbigen, Oxford Biotechnology, Panvera, PerkinElmer Life Sciences, Pharmingen, Phoenix Pharmaceuticals, Pierce Chemical Company, Polymun Scientific, Polysiences, Inc., Promega Corporation, Proteogenix, Protos Immunoresearch, QED Biosciences, Inc., R&D Systems, Repligen, Research Diagnostics, Roboscreen, Santa Cruz Biotechnology, Seikagaku America, Serological Corporation, Serotec, SigmaAldrich, StemCell Technologies, Synaptic Systems GmbH, Technopharm, Terra Nova Biotechnology, TiterMax, Trillium Diagnostics, Upstate Biotechnology, US Biological, Vector Laboratories, Wako Pure Chemical Industries, and Zeptometrix. However, the skilled artisan can routinely make antibodies, against any of the polypeptides described herein.

Polyclonal antibodies for measuring polypeptides include without limitation antibodies that were produced from sera by active immunization of one or more of the following: Rabbit, Goat, Sheep, Chicken, Duck, Guinea Pig, Mouse, Donkey, Camel, Rat and Horse.

Examples of additional detection agents, include without limitation: scFv, dsFv, Fab, sVH, F(ab′)₂, Cyclic peptides, Haptamers, A single-domain antibody, Fab fragments, Single-chain variable fragments, Affibody molecules, Affilins, Nanofitins, Anticalins, Avimers, DARPins, Kunitz domains, Fynomers and Monobody.

Enzyme linked immunosorbent assay (ELISA): Performing an ELISA involves at least one antibody with specificity for a particular antigen. The sample with an unknown amount of antigen is immobilized on a solid support (usually a polystyrene microtiter plate) either non-specifically (via adsorption to the surface) or specifically (via capture by another antibody specific to the same antigen, in a “_sandwich” ELISA). After the antigen is immobilized, the detection antibody is added, forming a complex with the antigen. The detection antibody can be covalently linked to an enzyme, or can itself be detected by a secondary antibody that is linked to an enzyme through bioconjugation. Between each step, the plate is typically washed with a mild detergent solution to remove any proteins or antibodies that are aspecifically bound. After the final wash step, the plate is developed by adding an enzymatic substrate to produce a visible signal, which indicates the quantity of antigen in the sample.

Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Automated Immunoassay: An automated analyzer applied to an immunoassay (often called “Automated Immunoassay”) is a medical laboratory instrument designed to measure different chemicals and other characteristics in a number of biological samples quickly, with minimal human assistance. These measured properties of blood and other fluids may be useful in the diagnosis of disease. Many methods of introducing samples into the analyzer have been invented. This can involve placing test tubes of sample into racks, which can be moved along a track, or inserting tubes into circular carousels that rotate to make the sample available. Some analyzers require samples to be transferred to sample cups. However, the effort to protect the health and safety of laboratory staff has prompted many manufacturers to develop analyzers that feature closed tube sampling, preventing workers from direct exposure to samples. Samples can be processed singly, in batches, or continuously. Examples of automated immunoassay machines include, without limitation, ARCHITECT ci4100, ci8200 (2003), ci16200 (2007), ARCHITECT i1000SR, ARCHITECT i2000, i2000SR, 14000SR, AxSYM/AxSYM Plus, 1994 U.S., DS2, AIMS, AtheNA, DSX, ChemWell, UniCel DxI 860i Synchron Access Clinical System, UniCel DxC 680i Synchron Access Clinical System, Access/Access 2 Immunoassay System, UniCel DxI 600 Access Immunoassay System, UniCel DxC 600i Synchron Access Clinical System, UniCel DxI 800 Access Immunoassay System, UniCel DxC 880i Synchron Access Clinical System, UniCel DxI 660i Synchron Access Clinical System, SPA PLUS (Specialist Protein Analyzer), VIDAS Immunoassay Analyzer, BioPlex 2200, PhD System EVOLIS PR 3100TSC Photometer, MAGO 4S/2011 Mago Plus Automated EIA Processor, LIAISON XL/2010 LIAISON, ETI-MAX 3000 Agility, Triturus, HYTEC 288 PLUSDSX, VITROS ECi Immunodiagnostic System, VITROS 3600 Immunodiagnostic System, Phadia Laboratory System 100E, Phadia Laboratory System 250, Phadia Laboratory System 1000, Phadia Laboratory System 2500, Phadia Laboratory System 5000, cobas e 602/2010, cobas c411, cobas c601, MODULAR ANALYTICS E170, Elecsys 2010, Dimension EXL 200/2011, Dimension Xpand Plus Integrated Chemistry System, Dimension RxL Max/Max Suite Integrated Chemistry System, Dimension RxL Integrated Chemistry System, Dimension EXL with LM Integrated Chemistry System, Stratus CS Acute Care Diagnostic System, IMMULITE 2000 XPi Immunoassay System, ADVIA Centaur CP Immunoassay System, IMMULITE 2000, IMMULITE 1000, Dimension Vista 500 Intelligent Lab System, Dimension Vista 1500 Intelligent Lab System, ADVIA Centaur XP, AIA-900, AIA-360, AIA-2000, AIA-600 II, AIA-1800. Measurements of CRP, IP-10 and TRAIL can also be performed on a Luminex machine.

Lateral Flow Immunoassays (LFIA): This is a technology which allows rapid measurement of analytes at the point of care (POC) and its underlying principles are described below. According to one embodiment, LFIA is used in the context of a hand-held device.

The technology is based on a series of capillary beds, such as pieces of porous paper or sintered polymer. Each of these elements has the capacity to transport fluid (e.g., urine) spontaneously. The first element (the sample pad) acts as a sponge and holds an excess of sample fluid. Once soaked, the fluid migrates to the second element (conjugate pad) in which the manufacturer has stored the so-called conjugate, a dried format of bio-active particles (see below) in a salt-sugar matrix that contains everything to guarantee an optimized chemical reaction between the target molecule (e.g., an antigen) and its chemical partner (e.g., antibody) that has been immobilized on the particle's surface. While the sample fluid dissolves the salt-sugar matrix, it also dissolves the particles and in one combined transport action the sample and conjugate mix while flowing through the porous structure. In this way, the analyte binds to the particles while migrating further through the third capillary bed. This material has one or more areas (often called stripes) where a third molecule has been immobilized by the manufacturer. By the time the sample-conjugate mix reaches these strips, analyte has been bound on the particle and the third ‘capture’ molecule binds the complex.

After a while, when more and more fluid has passed the stripes, particles accumulate and the stripe-area changes color. Typically there are at least two stripes: one (the control) that captures any particle and thereby shows that reaction conditions and technology worked fine, the second contains a specific capture molecule and only captures those particles onto which an analyte molecule has been immobilized. After passing these reaction zones the fluid enters the final porous material, the wick, that simply acts as a waste container. Lateral Flow Tests can operate as either competitive or sandwich assays.

Immunohistochemical analysis: Immunoassays carried out in accordance with some embodiments of the present invention may be homogeneous assays or heterogeneous assays. In a homogeneous assay the immunological reaction usually involves the specific antibody (e.g., anti-TRAIL, CRP and/or IP-10 antibody), a labeled analyte, and the sample of interest. The signal arising from the label is modified, directly or indirectly, upon the binding of the antibody to the labeled analyte. Both the immunological reaction and detection of the extent thereof can be carried out in a homogeneous solution. Immunochemical labels, which may be employed, include free radicals, radioisotopes, fluorescent dyes, enzymes, bacteriophages, or coenzymes.

In a heterogeneous assay approach, the reagents are usually the sample, the antibody, and means for producing a detectable signal. Samples as described above may be used. The antibody can be immobilized on a support, such as a bead (such as protein A and protein G agarose beads), plate or slide, and contacted with the specimen suspected of containing the antigen in a liquid phase.

According to a particular embodiment, the antibody is immobilized to a porous strip to form a detection site. The measurement or detection region of the porous strip may include a plurality of sites, one for TRAIL, one for CRP and one for IP-10. A test strip may also contain sites for negative and/or positive controls.

Alternatively, control sites can be located on a separate strip from the test strip. Optionally, the different detection sites may contain different amounts of antibodies, e.g., a higher amount in the first detection site and lesser amounts in subsequent sites. Upon the addition of test sample, the number of sites displaying a detectable signal provides a quantitative indication of the amount of polypeptides present in the sample. The detection sites may be configured in any suitably detectable shape and are typically in the shape of a bar or dot spanning the width of a test strip.

The support is then separated from the liquid phase and either the support phase or the liquid phase is examined for a detectable signal employing means for producing such signal. The signal is related to the presence of the analyte in the sample. Means for producing a detectable signal include the use of radioactive labels, fluorescent labels, or enzyme labels. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are oligonucleotides, immunoblotting, immunofluorescence methods, immunoprecipitation, chemiluminescence methods, electrochemiluminescence (ECL) or enzyme-linked immunoassays.

Those skilled in the art will be familiar with numerous specific immunoassay formats and variations thereof which may be useful for carrying out the method disclosed herein. See generally E. Maggio, Enzyme-Immunoassay, (1980) (CRC Press, Inc., Boca Raton, Fla.); see also U.S. Pat. No. 4,727,022 to Skold et al. titled “Methods for Modulating Ligand-Receptor Interactions and their Application,” U.S. Pat. No. 4,659,678 to Forrest et al. titled “Immunoassay of Antigens,” U.S. Pat. No. 4,376,110 to David et al., titled “Immunometric Assays Using Monoclonal Antibodies,” U.S. Pat. No. 4,275,149 to Litman et al., titled “Macromolecular Environment Control in Specific Receptor Assays,” U.S. Pat. No. 4,233,402 to Maggio et al., titled “Reagents and Method Employing Channeling,” and U.S. Pat. No. 4,230,767 to Boguslaski et al., titled “Heterogenous Specific Binding Assay Employing a Coenzyme as Label.”

Antibodies can be conjugated to a solid support suitable for a diagnostic assay (e.g., beads such as protein A or protein G agarose, microspheres, plates, slides or wells formed from materials such as latex or polystyrene) in accordance with known techniques, such as passive binding. Antibodies as described herein may likewise be conjugated to detectable labels or groups such as radiolabels (e.g., ³⁵S, ¹²⁵I, ¹³¹I), enzyme labels (e.g., horseradish peroxidase, alkaline phosphatase), and fluorescent labels (e.g., fluorescein, Alexa, green fluorescent protein, rhodamine) in accordance with known techniques.

Monoclonal antibodies for measuring TRAIL include without limitation: Mouse, Monoclonal (55B709-3) IgG; Mouse, Monoclonal (2E5) IgG1; Mouse, Monoclonal (2E05) IgG1; Mouse, Monoclonal (M912292) IgG1 kappa; Mouse, Monoclonal (IIIF6) IgG2b; Mouse, Monoclonal (2E1-1B9) IgG1; Mouse, Monoclonal (RIK-2) IgG1, kappa; Mouse, Monoclonal M181 IgG1; Mouse, Monoclonal VI10E IgG2b; Mouse, Monoclonal MAB375 IgG1; Mouse, Monoclonal MAB687 IgG1; Mouse, Monoclonal HS501 IgG1; Mouse, Monoclonal clone 75411.11 Mouse IgG1; Mouse, Monoclonal T8175-50 IgG; Mouse, Monoclonal 2B2.108 IgG1; Mouse, Monoclonal B-T24 IgG1; Mouse, Monoclonal 55B709.3 IgG1; Mouse, Monoclonal D3 IgG1; Goat, Monoclonal C19 IgG; Rabbit, Monoclonal H257 IgG; Mouse, Monoclonal 500-M49 IgG; Mouse, Monoclonal 05-607 IgG; Mouse, Monoclonal B-T24 IgG1; Rat, Monoclonal (N2B2), IgG2a, kappa; Mouse, Monoclonal (1A7-2B7), IgG1; Mouse, Monoclonal (55B709.3), IgG and Mouse, Monoclonal B-S23*IgG1, Human TRAIL/TNFSF10 MAb (Clone 75411), Mouse IgG1, Human TRAIL/TNFSF10 MAb (Clone 124723), Mouse IgG1, Human TRAIL/TNFSF10 MAb (Clone 75402), Mouse IgG1.

Antibodies for measuring TRAIL include monoclonal antibodies and polyclonal antibodies for measuring TRAIL. Antibodies for measuring TRAIL include antibodies that were developed to target epitopes from the list comprising of: Mouse myeloma cell line NSO-derived recombinant human TRAIL (Thr95-Gly281 Accession #P50591), Mouse myeloma cell line, NSO-derived recombinant human TRAIL (Thr95-Gly281, with an N-terminal Met and 6-His tag Accession #P50591), E. coli-derived, (Val114-Gly281, with and without an N-terminal Met Accession #: Q6IBA9), Human plasma derived TRAIL, Human serum derived TRAIL, recombinant human TRAIL where first amino acid is between position 85-151 and the last amino acid is at position 249-281.

Examples of monoclonal antibodies for measuring CRP include without limitation: Mouse, Monoclonal (108-2A2); Mouse, Monoclonal (108-7G41D2); Mouse, Monoclonal (12D-2C-36), IgG1; Mouse, Monoclonal (1G1), IgG1; Mouse, Monoclonal (5A9), IgG2a kappa; Mouse, Monoclonal (63F4), IgG1; Mouse, Monoclonal (67A1), IgG1; Mouse, Monoclonal (8B-5E), IgG1; Mouse, Monoclonal (B893M), IgG2b, lambda; Mouse, Monoclonal (C1), IgG2b; Mouse, Monoclonal (C11F2), IgG; Mouse, Monoclonal (C2), IgG1; Mouse, Monoclonal (C3), IgG1; Mouse, Monoclonal (C4), IgG1; Mouse, Monoclonal (C5), IgG2a; Mouse, Monoclonal (C6), IgG2a; Mouse, Monoclonal (C7), IgG1; Mouse, Monoclonal (CRP103), IgG2b; Mouse, Monoclonal (CRP11), IgG1; Mouse, Monoclonal (CRP135), IgG1; Mouse, Monoclonal (CRP169), IgG2a; Mouse, Monoclonal (CRP30), IgG1; Mouse, Monoclonal (CRP36), IgG2a; Rabbit, Monoclonal (EPR283Y), IgG; Mouse, Monoclonal (KT39), IgG2b; Mouse, Monoclonal (N-a), IgG1; Mouse, Monoclonal (NIG1), IgG1; Monoclonal (P5A9AT); Mouse, Monoclonal (S5G1), IgG1; Mouse, Monoclonal (SB78c), IgG1; Mouse, Monoclonal (SB78d), IgG1 and Rabbit, Monoclonal (Y284), IgG, Human C-Reactive Protein/CRP Biot MAb (Cl 232024), Mouse IgG2B, Human C-Reactive Protein/CRP MAb (Clone 232007), Mouse IgG2B, Human/Mouse/Porcine C-Reactive Protein/CRP MAb (Cl 232026), Mouse IgG2A.

Antibodies for measuring CRP include monoclonal antibodies for measuring CRP and polyclonal antibodies for measuring CRP.

Antibodies for measuring CRP also include antibodies that were developed to target epitopes from the list comprising of: Human plasma derived CRP, Human serum derived CRP, Mouse myeloma cell line NSO-derived recombinant human C-Reactive Protein/CRP (Phe17-Pro224 Accession #P02741).

Examples of monoclonal antibodies for measuring IP-10 include without limitation: IP-10/CXCL10 Mouse anti-Human Monoclonal (4D5) Antibody (LifeSpan BioSciences), IP-10/CXCL10 Mouse anti-Human Monoclonal (A00163.01) Antibody (LifeSpan BioSciences), MOUSE ANTI HUMAN IP-10 (AbD Serotec), RABBIT ANTI HUMAN IP-10 (AbD Serotec), IP-10 Human mAb 6D4 (Hycult Biotech), Mouse Anti-Human IP-10 Monoclonal Antibody Clone B-C50 (Diaclone), Mouse Anti-Human IP-10 Monoclonal Antibody Clone B-C55 (Diaclone), Human CXCL10/IP-10 MAb Clone 33036 (R&D Systems), CXCL10/INP10 Antibody 1E9 (Novus Biologicals), CXCL10/INP10 Antibody 2C1 (Novus Biologicals), CXCL10/INP10 Antibody 6D4 (Novus Biologicals), CXCL10 monoclonal antibody MOIA clone 2C1 (Abnova Corporation), CXCL10 monoclonal antibody (M05), clone 1E9 (Abnova Corporation), CXCL10 monoclonal antibody, clone 1 (Abnova Corporation), IP10 antibody 6D4 (Abcam), IP10 antibody EPR7849 (Abcam), IP10 antibody EPR7850 (Abcam).

Antibodies for measuring IP-10 include monoclonal antibodies for measuring IP-10 and polyclonal antibodies for measuring IP-10.

Antibodies for measuring IP-10 also include antibodies that were developed to target epitopes from the list comprising of: Recombinant human CXCL10/IP-10, non-glycosylated polypeptide chain containing 77 amino acids (aa 22-98) and an N-terminal His tag Interferon gamma inducible protein 10 (125 aa long), IP-10 His Tag Human Recombinant IP-10 produced in E. Coli containing 77 amino acids fragment (22-98) and having a total molecular mass of 8.5 kDa with an amino-terminal hexahistidine tag, E. coli-derived Human IP-10 (Val22-Pro98) with an N-terminal Met, Human plasma derived IP-10, Human serum derived IP-10, recombinant human IP-10 where first amino acid is between position 1-24 and the last amino acid is at position 71-98.

It will be appreciated that the expression level of the polypeptides described herein can be an absolute expression level, a normalized expression and/or a relative expression level.

In general scientific context, normalization is a process by which a measurement raw data is converted into data that may be directly compared with other so normalized data. In the context of the present invention, measurements of expression levels are prone to errors caused by, for example, unequal degradation of measured samples, different loaded quantities per assay, and other various errors. More specifically, any assayed sample may contain more or less biological material than is intended, due to human error and equipment failures. Thus, the same error or deviation applies to both the polypeptide of the invention and to the control reference, whose expression is essentially constant. Thus, division of TRAIL, IP-10 or CRP raw expression value by the control reference raw expression value yields a quotient which is essentially free from any technical failures or inaccuracies (except for major errors which destroy the sample for testing purposes) and constitutes a normalized expression value of the polypeptide. Since control reference expression values are equal in different samples, they constitute a common reference point that is valid for such normalization.

According to a particular embodiment, each of the polypeptide expression values are normalized using the same control reference.

It will further be appreciated that absolute expression values are dependent upon the exact protocol used, since each protocol typically leads to different signal to noise ratios, and consequentially to different concentrations being measured. More specifically, while the overall trend of the biomarkers will be preserved regardless of the protocol (e.g. TRAIL increases in viral infections and decreases in bacterial), the measurement scale is protocol dependent.

Such alterations in measured concentrations of proteins across different protocols can be compensated for by correlating the measurements of the two protocols and computing a transformation function, as illustrated in Example 5 herein below.

Typically, the samples which are analyzed are blood sample comprising whole blood, serum, plasma, leukocytes or blood cells. Preferably, the sample is whole blood, serum or plasma.

Of note, TRAIL and IP-10 and CRP are highly expressed in other tissues and samples including without limitation CSF, saliva and epithelial cells, bone marrow aspiration, urine, stool, alveolar lavage, sputum. Thus, some embodiments of the present invention can be used to measure TRAIL, CRP and IP-10 in such tissues and samples.

Preferably, the level of the polypeptides is measured within about 24 hours after the sample is obtained. Alternatively, the concentration of the polypeptides is measured in a sample that was stored at 12° C. or lower, when storage begins less than 24 hours after the sample is obtained.

Once the tests are carried out to determine the level of the polypeptides, a subject specific dataset is optionally generated which contains the results of the measurements.

The subject-specific dataset may be stored in a computer readable format on a non-volatile computer readable medium, and is optionally and preferably accessed by a hardware processor, such as a general purpose computer or dedicated circuitry.

As mentioned, the levels of the polypeptides in the test subjects blood are compared to the levels of the identical polypeptides in a plurality of subjects' blood, when the subjects have already been verified as having a bacterial infection, viral infection or non-bacterial/non-viral disease on the basis of parameters other than the blood level of the polypeptides. The levels of the polypeptides of the plurality of subjects together with their verified diagnosis can be stored in a second dataset, also referred to herein as the “group dataset” or “prediagnosed dataset”, as further described herein below.

The phrase “non-bacterial/non-viral disease” refers to disease that is not caused by a bacteria or virus. This includes diseases such as acute myocardial infarction, physical injury, epileptic attack, inflammatory disorders etc, fungal diseases, parasitic diseases etc.

The phrase “viral infection” as used herein refers to a disease that is caused by a virus and does not comprise a bacterial component.

Methods of analyzing a dataset, for example, for the purpose of calculating one or more probabilistic classification function representing the likelihood that a particular subject has a bacterial infection, or the likelihood that a particular subject has a viral infection or the likelihood that a particular subject has a non-bacterial non-viral disease, may be performed as described in Example 1 herein below. For example, diagnosis may be supported using PCR diagnostic assays such as (i) Seeplex® RV15 for detection of parainfluenza virus 1, 2, 3, and 4, coronavirus 229E/NL63, adenovirus A/B/C/D/E, bocavirus 1/2/3/4, influenza virus A and B, metapneumovirus, coronavirus OC43, rhinovirus A/B/C, respiratory syncytial virus A and B, and Enterovirus, or (ii) Seeplex® PB6 for detection of Streptococcus pneumoniae, Haemophilus influenzae, Chlamydophila pneumoniae, Legionella pneumophila, Bordetella pertussis, and Mycoplasma pneumoniae.

Blood cultures, urine cultures and stool cultures may be analyzed for Shigella spp., Campylobacter spp. and Salmonella spp.; serological testing (IgM and/or IgG) for cytomegalovirus (CMV), Epstein-Barr virus (EBV), Mycoplasma Pneumonia, and Coxiella burnetii (Q-Fever).

Radiological tests (e.g. chest X-ray for suspected lower respiratory tract infection [LRTI]) may be used to confirm chest infections.

Alternatively, or additionally at least one trained physician may be used to establish the diagnosis.

Methods of determining the expression level of the polypeptides in the pre-diagnosed subjects have been described herein above.

Preferably, the same method which is used for determining the expression level of the polypeptides in the pre-diagnosed subjects is used for determining the level of the polypeptides in the test subject. Thus, for example if an immunoassay type method is used for determining the expression level of the polypeptides in the pre-diagnosed subjects, then an immunoassay type method should be used for determining the level of the polypeptides in the test subject.

It will be appreciated that, the type of blood sample need not be identical in the test subject and the pre-diagnosed subjects. The present inventors were able to show that serum and plasma levels for TRAIL are very similar. Thus, for example, if a serum sample is used for determining the expression level of the polypeptides in the pre-diagnosed subjects, then a plasma sample may be used for determining the level of the polypeptides in the test subject.

The group dataset is preferably stored in a computer readable format on a non-volatile computer readable medium, and is optionally and preferably accessed by a hardware processor, such as a general purpose computer or dedicated circuitry. Both datasets can be stored on the same medium and are optionally and preferably accessed by the same hardware processor.

In the subject-specific dataset, each entry can optionally and preferably be described as a tuple (D, L) where D represents the polypeptide in the dataset and L represents the blood level of the polypeptide D. Thus, the dataset may be a two-dimensional dataset in which all the elements can be described by a vector in a two-dimensional space spanned by the polypeptide and respective response. In the group dataset, each entry can be described as a tuple (S, G, D, L) where S represents the particular subject, G represents the diagnosis of the subject S in the group dataset, D represents the polypeptide and L represents blood level of the polypeptide D. Thus, the exemplified illustration is of a four-dimensional dataset in which all the elements can be described by a vector in a four-dimensional space spanned by the subjects, diagnosis, polypeptide and respective responses. Some embodiments of the present invention contemplate use of datasets of higher dimensions. Such datasets are described hereinafter.

The group dataset may optionally and preferably also include one or more of, more preferably all, the entries of the subject-specific dataset. In embodiments in which group dataset includes all the entries of the subject-specific dataset, it is not necessary to use two separate datasets, since the entire dataset is contained in one inclusive dataset. Yet, such an inclusive dataset is optionally and preferably annotated in a manner that allows distinguishing between the portion of the inclusive dataset that is associated with the subject under analysis, and the portion of the inclusive dataset that is associated only with the other subjects. In the context of the present disclosure, the portion of the inclusive dataset that is associated with the subject under analysis is referred to as the subject-specific dataset even when it is not provided as a separate dataset. Similarly, the portion of the inclusive dataset that is associated only with the other subjects is referred to as the group dataset even when it is not provided as a separate dataset.

The group dataset preferably includes polypeptide levels of many subjects (e.g., at least 10 subjects being prediagnosed as having a viral infection, at least 10 subjects being prediagnosed as having a bacterial infection and at least 10 subjects being prediagnosed as having a non-bacterial/non-viral disease; or at least 20 subjects being prediagnosed as having a viral infection, at least 20 subjects being prediagnosed as having a bacterial infection and at least 20 subjects being prediagnosed as having a non-bacterial/non-viral disease; or at least 50 subjects being prediagnosed as having a viral infection, at least 50 subjects being prediagnosed as having a bacterial infection and at least 50 subjects being prediagnosed as having a non-bacterial/non-viral disease.

The group-specific dataset can include additional data that describes the subjects. Datasets that include additional data may be advantageous since they provide additional information regarding the similarities between the subject under analysis and the other subject, thereby increasing the accuracy of the predictability.

Representative examples of types of data other than the level of the polypeptides include, without limitation traditional laboratory risk factors and/or clinical parameters, as further described herein above.

The present embodiments contemplate subject-specific and group datasets that include additional data, aside from the polypeptides and respective levels. In some embodiments at least one of the datasets comprises one or more (e.g., a plurality of) multidimensional entries, each entry having at least three dimensions, in some embodiments at least one of the datasets comprises one or more (e.g., a plurality of) multidimensional entries, each entry having at least four dimensions, in some embodiments at least one of the datasets comprises one or more (e.g., a plurality of) multidimensional entries, each entry having at least five dimensions, and in some embodiments at least one of the datasets comprises one or more (e.g., a plurality of) multidimensional entries, each entry having more than five dimensions.

The additional dimensions of the datasets provides additional information pertaining to the subject under analysis, to the other subjects and/or to levels of polypeptides other than TRAIL, CRP and IP-10.

In some embodiments of the present invention the additional information pertains to at least one of traditional laboratory risk factors, clinical parameters, blood chemistry and/or a genetic profile.

“Traditional laboratory risk factors” encompass biomarkers isolated or derived from subject samples and which are currently evaluated in the clinical laboratory and used in traditional global risk assessment algorithms, such as absolute neutrophil count (abbreviated ANC), absolute lymphocyte count (abbreviated ALC), white blood count (abbreviated WBC), neutrophil % (defined as the fraction of white blood cells that are neutrophils and abbreviated Neu (%)), lymphocyte % (defined as the fraction of white blood cells that are lymphocytes and abbreviated Lym (%)), monocyte % (defined as the fraction of white blood cells that are monocytes and abbreviated Mon (%)), Sodium (abbreviated Na), Potassium (abbreviated K), Bilirubin (abbreviated Bili).

Preferably, at least one of the traditional laboratory risk factors of the subject under analysis is included in the subject specific dataset, and at least one of the traditional laboratory risk factors of one or more (more preferably all) of the other subjects is included in the group dataset. When the subject specific dataset includes at least one of the traditional laboratory risk factors, the risk factors can be included as a separate entry. When the group dataset includes the risk factors, the risk factors is optionally and preferably included per subject. Thus, for example, a group dataset entry can be described by the tuple (S, G, D, L {R}), where S, G, D and L have been introduced before and {R} is the at least one risk factor of subject S.

“Clinical parameters” encompass all non-sample or non-analyte biomarkers of subject health status or other characteristics, such as, without limitation, age (Age), ethnicity (RACE), gender (Sex), core body temperature (abbreviated “temperature”), maximal core body temperature since initial appearance of symptoms (abbreviated “maximal temperature”), time from initial appearance of symptoms (abbreviated “time from symptoms”), pregnancy, or family history (abbreviated FamHX).

Preferably, at least one of the clinical parameters of the subject under analysis is included in the subject specific dataset, and at least one of the clinical parameters of one or more (more preferably all) of the other subjects is included in the group dataset. When the subject specific dataset includes at least one of the clinical parameters, the clinical parameters can be included as a separate entry. When the group dataset includes the clinical parameters, the clinical parameters is optionally and preferably included per subject. Thus, for example, a group dataset entry can be described by the tuple (S, G, D, L {C}), where S, G, D and L have been introduced before and {C} is the clinical parameter of subject S.

As used herein “blood chemistry” refers to the concentration, or concentrations, of any and all substances dissolved in, or comprising, the blood. Representative examples of such substances, include, without limitation, albumin, amylase, alkaline phosphatase, bicarbonate, total bilirubin, BUN, C-reactive protein, calcium, chloride, LDL, HDL, total cholesterol, creatinine, CPK, γ-GT, glucose, LDH, inorganic phosphorus, lipase, potassium, total protein, AST, ALT, sodium, triglycerides, uric acid and VLDL.

According to one embodiment, the blood chemistry of the subject under analysis is included in the subject specific dataset, and the blood chemistry of one or more (more preferably all) of the other subjects is included in the group dataset. When the subject specific dataset includes the blood chemistry, the blood chemistry can be included as a separate entry. When the group dataset includes the blood chemistry, the blood chemistry is optionally and preferably included per subject. Thus, for example, a group dataset entry can be described by the tuple (S, G, D, L {C}), where S, G, D and L have been introduced before and {C} is the blood chemistry of subject S.

In some embodiments of the present invention the additional information pertains to a genetic profile of individual.

As used herein “genetic profile” refers to the analysis of a number of different genes. A genetic profile can encompass the genes in an entire genome of the individual, or it can encompass a specific subset of genes. The genetic profile may include genomic profile, a proteomic profile, an epigenomic profile and/or a transcriptomic profile.

Preferably, the genetic profile of the subject under analysis is included in the subject specific dataset, and the genetic profile of one or more (more preferably all) of the other subjects is included in the group dataset. When the subject specific dataset includes the genetic profile, the genetic profile can be included as a separate entry. When the group dataset includes the genetic profile, the genetic profile is optionally and preferably included per subject. Thus, for example, a group dataset entry can be described by the tuple (S, G, D, L {P}), where S, G, D and L have been introduced before and {P} is the genetic profile of subject S.

The method optionally and preferably continues to a step of storing the levels of the polypeptide, at least temporarily, on a non-volatile computer readable medium from which it can be extracted or displayed as desired.

Once the two datasets are accessed, the method continues to the analysis phase in order to diagnose the test subject.

The analysis is performed so as to compute one or more probabilistic classification functions f(δ₀, δ₁), g(♦₀, δ₁), h(δ₀, δ₁), representing the likelihoods that a particular subject has a bacterial infection, viral infection or non-viral, non-bacterial disease, respectively. Typically, f, g and h satisfy the relation f(δ₀, δ₁)+g(δ₀, δ₁)+h(δ₀, δ₁)=1. Each classification function is a function of the first coordinate δ₀ and the second coordinate δ₁, wherein each of the coordinates δ₀ and δ₁ is defined by a different combination of the expression values as further detailed hereinabove.

The analysis can be executed in more than one way.

According to one embodiment, the analysis uses a binary or, more preferably, trinary classifier to compute one or more of the probabilistic classification functions.

Preferably, the analysis sums the probability of the viral and the non-viral, non-bacterial disease in order to assign the likelihood of a non-bacterial infection. In another preferred embodiment, the analysis sums the probability of the viral and bacterial to assign the likelihood of an infectious disease. Yet in another preferred embodiment the analysis ignores the probability of the non-viral, non-bacterial disease, and performs a direct comparison of the bacterial and the viral probabilities. Exemplified interpretation functions suitable for analyzing the datasets according to some embodiments of the present invention are provided hereinunder.

The analysis of the datasets according to some embodiments of the present invention comprises executing a machine learning procedure.

As used herein the term “machine learning” refers to a procedure embodied as a computer program configured to induce patterns, regularities, or rules from previously collected data to develop an appropriate response to future data, or describe the data in some meaningful way.

Use of machine learning is particularly, but not exclusively, advantageous when the dataset includes multidimensional entries.

The group and subject datasets can be used as a training set from which the machine learning procedure can extract parameters that best describe the dataset. Once the parameters are extracted, they can be used to predict the type of infection.

In machine learning, information can be acquired via supervised learning or unsupervised learning. In some embodiments of the invention the machine learning procedure comprises, or is, a supervised learning procedure. In supervised learning, global or local goal functions are used to optimize the structure of the learning system. In other words, in supervised learning there is a desired response, which is used by the system to guide the learning.

In some embodiments of the invention the machine learning procedure comprises, or is, an unsupervised learning procedure. In unsupervised learning there are typically no goal functions. In particular, the learning system is not provided with a set of rules. One form of unsupervised learning according to some embodiments of the present invention is unsupervised clustering in which the data objects are not class labeled, a priori.

Representative examples of “machine learning” procedures suitable for the present embodiments, including, without limitation, clustering, association rule algorithms, feature evaluation algorithms, subset selection algorithms, support vector machines, classification rules, cost-sensitive classifiers, vote algorithms, stacking algorithms, Bayesian networks, decision trees, neural networks, instance-based algorithms, linear modeling algorithms, k-nearest neighbors analysis, ensemble learning algorithms, probabilistic models, graphical models, logistic regression methods (including multinomial logistic regression methods), gradient ascent methods, singular value decomposition methods and principle component analysis. Among neural network models, the self-organizing map and adaptive resonance theory are commonly used unsupervised learning algorithms. The adaptive resonance theory model allows the number of clusters to vary with problem size and lets the user control the degree of similarity between members of the same clusters by means of a user-defined constant called the vigilance parameter.

Following is an overview of some machine learning procedures suitable for the present embodiments.

Association rule algorithm is a technique for extracting meaningful association patterns among features.

The term “association”, in the context of machine learning, refers to any interrelation among features, not just ones that predict a particular class or numeric value. Association includes, but it is not limited to, finding association rules, finding patterns, performing feature evaluation, performing feature subset selection, developing predictive models, and understanding interactions between features.

The term “association rules” refers to elements that co-occur frequently within the datasets. It includes, but is not limited to association patterns, discriminative patterns, frequent patterns, closed patterns, and colossal patterns.

A usual primary step of association rule algorithm is to find a set of items or features that are most frequent among all the observations. Once the list is obtained, rules can be extracted from them.

The aforementioned self-organizing map is an unsupervised learning technique often used for visualization and analysis of high-dimensional data. Typical applications are focused on the visualization of the central dependencies within the data on the map. The map generated by the algorithm can be used to speed up the identification of association rules by other algorithms. The algorithm typically includes a grid of processing units, referred to as “neurons”. Each neuron is associated with a feature vector referred to as observation. The map attempts to represent all the available observations with optimal accuracy using a restricted set of models. At the same time the models become ordered on the grid so that similar models are close to each other and dissimilar models far from each other. This procedure enables the identification as well as the visualization of dependencies or associations between the features in the data.

Feature evaluation algorithms are directed to the ranking of features or to the ranking followed by the selection of features based on their impact.

The term “feature” in the context of machine learning refers to one or more raw input variables, to one or more processed variables, or to one or more mathematical combinations of other variables, including raw variables and processed variables.

Features may be continuous or discrete.

Information gain is one of the machine learning methods suitable for feature evaluation. The definition of information gain requires the definition of entropy, which is a measure of impurity in a collection of training instances. The reduction in entropy of the target feature that occurs by knowing the values of a certain feature is called information gain. Information gain may be used as a parameter to determine the effectiveness of a feature in explaining the type of infection. Symmetrical uncertainty is an algorithm that can be used by a feature selection algorithm, according to some embodiments of the present invention. Symmetrical uncertainty compensates for information gain's bias towards features with more values by normalizing features to a [0,1] range.

Subset selection algorithms rely on a combination of an evaluation algorithm and a search algorithm. Similarly to feature evaluation algorithms, subset selection algorithms rank subsets of features. Unlike feature evaluation algorithms, however, a subset selection algorithm suitable for the present embodiments aims at selecting the subset of features with the highest impact on the type of infection, while accounting for the degree of redundancy between the features included in the subset. The benefits from feature subset selection include facilitating data visualization and understanding, reducing measurement and storage requirements, reducing training and utilization times, and eliminating distracting features to improve classification.

Two basic approaches to subset selection algorithms are the process of adding features to a working subset (forward selection) and deleting from the current subset of features (backward elimination). In machine learning, forward selection is done differently than the statistical procedure with the same name. The feature to be added to the current subset in machine learning is found by evaluating the performance of the current subset augmented by one new feature using cross-validation. In forward selection, subsets are built up by adding each remaining feature in turn to the current subset while evaluating the expected performance of each new subset using cross-validation. The feature that leads to the best performance when added to the current subset is retained and the process continues. The search ends when none of the remaining available features improves the predictive ability of the current subset. This process finds a local optimum set of features.

Backward elimination is implemented in a similar fashion. With backward elimination, the search ends when further reduction in the feature set does not improve the predictive ability of the subset. The present embodiments contemplate search algorithms that search forward, backward or in both directions. Representative examples of search algorithms suitable for the present embodiments include, without limitation, exhaustive search, greedy hill-climbing, random perturbations of subsets, wrapper algorithms, probabilistic race search, schemata search, rank race search, and Bayesian classifier.

A decision tree is a decision support algorithm that forms a logical pathway of steps involved in considering the input to make a decision.

The term “decision tree” refers to any type of tree-based learning algorithms, including, but not limited to, model trees, classification trees, and regression trees.

A decision tree can be used to classify the datasets or their relation hierarchically. The decision tree has tree structure that includes branch nodes and leaf nodes. Each branch node specifies an attribute (splitting attribute) and a test (splitting test) to be carried out on the value of the splitting attribute, and branches out to other nodes for all possible outcomes of the splitting test. The branch node that is the root of the decision tree is called the root node. Each leaf node can represent a classification (e.g., whether a particular portion of the group dataset matches a particular portion of the subject-specific dataset) or a value. The leaf nodes can also contain additional information about the represented classification such as a confidence score that measures a confidence in the represented classification (i.e., the likelihood of the classification being accurate). For example, the confidence score can be a continuous value ranging from 0 to 1, which a score of 0 indicating a very low confidence (e.g., the indication value of the represented classification is very low) and a score of 1 indicating a very high confidence (e.g., the represented classification is almost certainly accurate).

Support vector machines are algorithms that are based on statistical learning theory. A support vector machine (SVM) according to some embodiments of the present invention can be used for classification purposes and/or for numeric prediction. A support vector machine for classification is referred to herein as “support vector classifier,” support vector machine for numeric prediction is referred to herein as “support vector regression”.

An SVM is typically characterized by a kernel function, the selection of which determines whether the resulting SVM provides classification, regression or other functions. Through application of the kernel function, the SVM maps input vectors into high dimensional feature space, in which a decision hyper-surface (also known as a separator) can be constructed to provide classification, regression or other decision functions. In the simplest case, the surface is a hyper-plane (also known as linear separator), but more complex separators are also contemplated and can be applied using kernel functions. The data points that define the hyper-surface are referred to as support vectors.

The support vector classifier selects a separator where the distance of the separator from the closest data points is as large as possible, thereby separating feature vector points associated with objects in a given class from feature vector points associated with objects outside the class. For support vector regression, a high-dimensional tube with a radius of acceptable error is constructed which minimizes the error of the data set while also maximizing the flatness of the associated curve or function. In other words, the tube is an envelope around the fit curve, defined by a collection of data points nearest the curve or surface.

An advantage of a support vector machine is that once the support vectors have been identified, the remaining observations can be removed from the calculations, thus greatly reducing the computational complexity of the problem. An SVM typically operates in two phases: a training phase and a testing phase. During the training phase, a set of support vectors is generated for use in executing the decision rule. During the testing phase, decisions are made using the decision rule. A support vector algorithm is a method for training an SVM. By execution of the algorithm, a training set of parameters is generated, including the support vectors that characterize the SVM. A representative example of a support vector algorithm suitable for the present embodiments includes, without limitation, sequential minimal optimization.

Regression techniques which may be used in accordance with the present invention include, but are not limited to linear Regression, Multiple Regression, logistic regression, probit regression, ordinal logistic regression ordinal Probit-Regression, Poisson Regression, negative binomial Regression, multinomial logistic Regression (MLR) and truncated regression.

A logistic regression or logit regression is a type of regression analysis used for predicting the outcome of a categorical dependent variable (a dependent variable that can take on a limited number of values, whose magnitudes are not meaningful but whose ordering of magnitudes may or may not be meaningful) based on one or more predictor variables. Logistic regressions also include a multinomial variant. The multinomial logistic regression model, is a regression model which generalizes logistic regression by allowing more than two discrete outcomes. That is, it is a model that is used to predict the probabilities of the different possible outcomes of a categorically distributed dependent variable, given a set of independent variables (which may be real-valued, binary-valued, categorical-valued, etc.).

The advantage of logistic regression is that it assigns an interpretable measure of prediction confidence—a probability. For example, patients predicted of having a bacterial infection with a probability of 75% and 99%, would both be assigned as bacterial when using an SVM interpretation function but the fact that the latter has a higher probability would be masked. Assigning the likelihood level of confidence adds valuable clinical information that may affect clinical judgment.

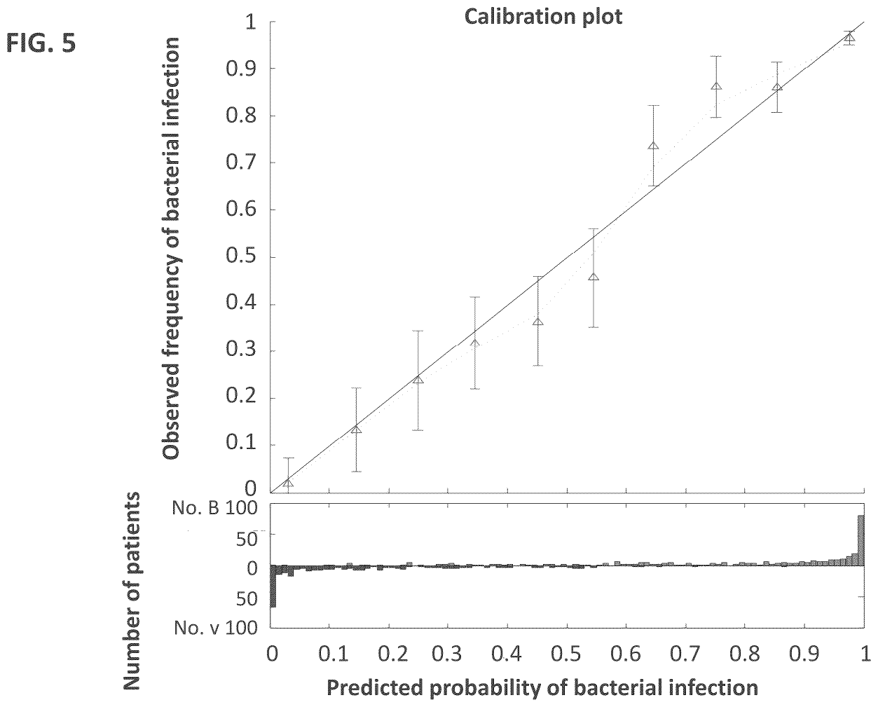

Importantly, calculating the likelihood of infection type for each patients, allows to rationally filter out patients for which the system knows that it cannot classify with high certainty. This is demonstrated in FIG. 5, herein. Thus, when the product assigns a likelihood of say 40% bacterial infection (40 out of 100 patients with the “40%” score will be bacterial).

Additionally, by using thresholds on the likelihood scores, one can assign non-binary classifications of the test-subject. By way of example a test-subject with a bacterial likelihood below 30% can be assigned a low probability of bacterial infection; between 30% and 70% an intermediate probability of bacterial infection and above 70% a high probability of a bacterial infections. Other thresholds may be used.

The Least Absolute Shrinkage and Selection Operator (LASSO) algorithm is a shrinkage and/or selection algorithm for linear regression. The LASSO algorithm may minimizes the usual sum of squared errors, with a regularization, that can be an L1 norm regularization (a bound on the sum of the absolute values of the coefficients), an L2 norm regularization (a bound on the sum of squares of the coefficients), and the like. The LASSO algorithm may be associated with soft-thresholding of wavelet coefficients, forward stagewise regression, and boosting methods. The LASSO algorithm is described in the paper: Tibshirani, R, Regression Shrinkage and Selection via the Lasso, J. Royal. Statist. Soc B., Vol. 58, No. 1, 1996, pages 267-288, the disclosure of which is incorporated herein by reference.

A Bayesian network is a model that represents variables and conditional interdependencies between variables. In a Bayesian network variables are represented as nodes, and nodes may be connected to one another by one or more links. A link indicates a relationship between two nodes. Nodes typically have corresponding conditional probability tables that are used to determine the probability of a state of a node given the state of other nodes to which the node is connected. In some embodiments, a Bayes optimal classifier algorithm is employed to apply the maximum a posteriori hypothesis to a new record in order to predict the probability of its classification, as well as to calculate the probabilities from each of the other hypotheses obtained from a training set and to use these probabilities as weighting factors for future predictions of the type of infection. An algorithm suitable for a search for the best Bayesian network, includes, without limitation, global score metric-based algorithm. In an alternative approach to building the network, Markov blanket can be employed. The Markov blanket isolates a node from being affected by any node outside its boundary, which is composed of the node's parents, its children, and the parents of its children.

Instance-based algorithms generate a new model for each instance, instead of basing predictions on trees or networks generated (once) from a training set.

The term “instance”, in the context of machine learning, refers to an example from a dataset.

Instance-based algorithms typically store the entire dataset in memory and build a model from a set of records similar to those being tested. This similarity can be evaluated, for example, through nearest-neighbor or locally weighted methods, e.g., using Euclidian distances. Once a set of records is selected, the final model may be built using several different algorithms, such as the naive Bayes.

The present invention can also be used to screen patient or subject populations in any number of settings. For example, a health maintenance organization, public health entity or school health program can screen a group of subjects to identify those requiring interventions, as described above, or for the collection of epidemiological data. Insurance companies (e.g., health, life or disability) may screen applicants in the process of determining coverage or pricing, or existing clients for possible intervention. Data collected in such population screens, particularly when tied to any clinical progression to conditions like infection, will be of value in the operations of, for example, health maintenance organizations, public health programs and insurance companies. Such data arrays or collections can be stored in machine-readable media and used in any number of health-related data management systems to provide improved healthcare services, cost effective healthcare, improved insurance operation, etc. See, for example, U.S. Patent Application No. 2002/0038227; U.S. Patent Application No. US 2004/0122296; U.S. Patent Application No. US 2004/0122297; and U.S. Pat. No. 5,018,067. Such systems can access the data directly from internal data storage or remotely from one or more data storage sites as further detailed herein.

A machine-readable storage medium can comprise a data storage material encoded with machine readable data or data arrays which, when using a machine programmed with instructions for using said data, is capable of use for a variety of purposes. Measurements of effective amounts of the biomarkers of the invention and/or the resulting evaluation of risk from those biomarkers can implemented in computer programs executing on programmable computers, comprising, inter alia, a processor, a data storage system (including volatile and non-volatile memory and/or storage elements), at least one input device, and at least one output device.

Program code can be applied to input data to perform the functions described above and generate output information. The output information can be applied to one or more output devices, according to methods known in the art. The computer may be, for example, a personal computer, microcomputer, or workstation of conventional design.

Each program can be implemented in a high level procedural or object oriented programming language to communicate with a computer system. However, the programs can be implemented in assembly or machine language, if desired. The language can be a compiled or interpreted language. Each such computer program can be stored on a storage media or device (e.g., ROM or magnetic diskette or others as defined elsewhere in this disclosure) readable by a general or special purpose programmable computer, for configuring and operating the computer when the storage media or device is read by the computer to perform the procedures described herein.

The health-related data management system of the invention may also be considered to be implemented as a computer-readable storage medium, configured with a computer program, where the storage medium so configured causes a computer to operate in a specific and predefined manner to perform various functions described herein.

The recorded output may include the assay results, findings, diagnoses, predictions and/or treatment recommendations. These may be communicated to technicians, physicians and/or patients, for example. In certain embodiments, computers will be used to communicate such information to interested parties, such as, patients and/or the attending physicians. Based on the output, the therapy administered to a subject can be modified.

In one embodiment, the output is presented graphically. In another embodiment, the output is presented numerically (e.g. as a probability). In another embodiment, the output is generated using a color index (for example in a bar display) where one color indicates bacterial infection and another color non-bacterial infection. The strength of the color correlates with the probability of bacterial infection/non-infection. Such a graphic display is presented in FIGS. 29A-29F.

In some embodiments, the output is communicated to the subject as soon as possible after the assay is completed and the diagnosis and/or prediction is generated. The results and/or related information may be communicated to the subject by the subject's treating physician. Alternatively, the results may be communicated directly to a test subject by any means of communication, including writing, such as by providing a written report, electronic forms of communication, such as email, or telephone. Communication may be facilitated by use of a computer, such as in case of email communications. In certain embodiments, the communication containing results of a diagnostic test and/or conclusions drawn from and/or treatment recommendations based on the test, may be generated and delivered automatically to the subject using a combination of computer hardware and software which will be familiar to artisans skilled in telecommunications. One example of a healthcare-oriented communications system is described in U.S. Pat. No. 6,283,761; however, the present disclosure is not limited to methods which utilize this particular communications system. In certain embodiments of the methods of the disclosure, all or some of the method steps, including the assaying of samples, diagnosing of diseases, and communicating of assay results or diagnoses, may be carried out in diverse (e.g., foreign) jurisdictions.

In some embodiments, the methods described herein are carried out using a system 330, which optionally and preferably, but not necessarily, comprises a hand-held device, which comprises at least two compartments the first which measures the amount of polypeptides in the blood (e.g. using an immunohistochemical method) and the second which computationally analyzes the results measured in the first compartment and provides an output relating to the diagnosis.

A block diagram of representative example of system 330 according to some embodiments of the present invention is illustrated in FIG. 34. System 330 can comprise a device 331 which can be, but is not necessarily a hand-held device.

Alternatively, device 331 which can be a desktop mountable or a desktop placeable device. System 330 can comprise a first compartment 332 having a measuring system 333 configured to measure the expression value of the polypeptides in the blood of a subject. Measuring system 333 can perform at least one automated assay selected from the group consisting of an automated ELISA, an automated immunoassay, and an automated functional assay. System 330 can also comprise a second compartment 334 comprising a hardware processor 336 having a computer-readable medium 338 for storing computer program instructions for executing the operations described herein (e.g., computer program instructions for defining the first and/or second coordinates, computer program instructions for defining the curved line and/or plane, computer program instructions for calculating the first and/or distances, computer program instructions for correlating the calculated distance(s) to the presence of, absence of, or likelihood that the subject has, a bacterial and/or viral infection). Hardware processor 336 is configured to receive expression value measurements from first compartment 332 and execute the program instructions responsively to the measurements. Optionally and preferably hardware processor 336 is also configured to output the processed data to a display device 340.

In some optional embodiments of the present invention, system 330 communicates with a communication network. In these embodiments, system 330 or hardware processor 336 comprises a network interface 350 that communicates with a communication network 352. In the representative illustration shown in FIG. 34, network 352 is used for transmitting the results of the analysis performed by hardware processor 336 (for example, the presence of, absence of, or likelihood that the subject has, a bacterial and/or viral infection) to one or more remote locations. For example, system 330 can transmit the analysis results to at least one of a laboratory information system 360, and/or a central server 362 that collects data from a plurality of systems like system 330.

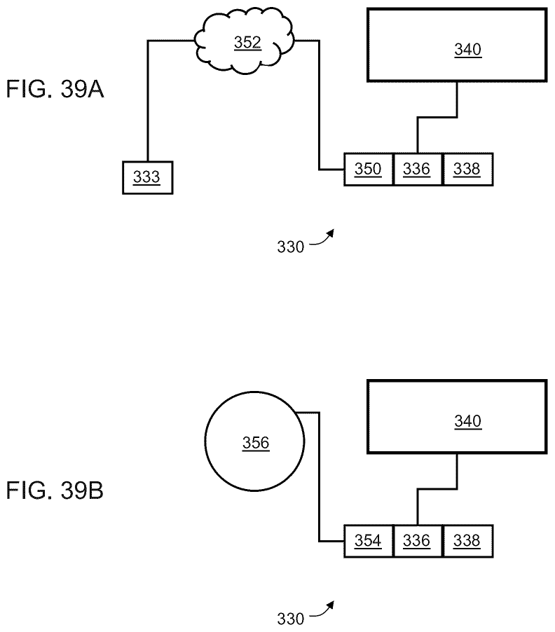

FIG. 39A is a schematic illustration showing a block diagram of system 330 in embodiments in which communication network 352 is used for receiving expression value measurements. In these embodiments, system 330 can comprise computer-readable medium 338, as further detailed hereinabove, and a hardware processor, such as, but not limited to, processor 336. Hardware processor 336 can comprise network interface 350. Via interface 350, hardware processor 336 receives expression value measurements from a measuring system, such as, but not limited to, measuring system 333, and executes the computer program instructions in computer-readable medium 338, responsively to the received measurements. Hardware processor 336 can then output the processed data to display device 340.

Combinations of the embodiments shown in FIGS. 34 and 39A are also contemplated. For example, interface 350 can be used both for receiving expression value measurements from network 352 and for transmitting the results of the analysis to network 352.

In some embodiments of the present invention system 330 communicates with a user, as schematically illustrated in the block diagram of FIG. 39B. In these embodiments, system 330 can comprise computer-readable medium 338, as further detailed hereinabove, and a hardware processor, such as, but not limited to, processor 336. Hardware processor 336 comprises a user interface 354 that communicates with a user 356. Via interface 350, hardware processor 336 receives expression value measurements from user 356. User 356 can obtain the expression value from an external source, or by executing at least one assay selected from the group consisting of an immunoassay and a functional assay, or by operating system 333 (not shown, see FIGS. 39A and 34). Hardware processor 336 executes the computer program instructions in computer-readable medium 338, responsively to the received measurements. Hardware processor 336 can then output the processed data to display device 340.

Once the diagnosis has been made, it will be appreciated that a number of actions may be taken.

Thus, for example, if a bacterial infection is ruled in, then the subject may be treated with an antibiotic agent.

Examples of antibiotic agents include, but are not limited to Daptomycin; Gemifloxacin; Telavancin; Ceftaroline; Fidaxomicin; Amoxicillin; Ampicillin; Bacampicillin; Carbenicillin; Cloxacillin; Dicloxacillin; Flucloxacillin; Mezlocillin; Nafcillin; Oxacillin; Penicillin G; Penicillin V; Piperacillin; Pivampicillin; Pivmecillinam; Ticarcillin; Aztreonam; Imipenem; Doripenem; Meropenem; Ertapenem; Clindamycin; Lincomycin; Pristinamycin; Quinupristin; Cefacetrile (cephacetrile); Cefadroxil (cefadroxyl); Cefalexin (cephalexin); Cefaloglycin (cephaloglycin); Cefalonium (cephalonium); Cefaloridine (cephaloradine); Cefalotin (cephalothin); Cefapirin (cephapirin); Cefatrizine; Cefazaflur; Cefazedone; Cefazolin (cephazolin); Cefradine (cephradine); Cefroxadine; Ceftezole; Cefaclor; Cefamandole; Cefmetazole; Cefonicid; Cefotetan; Cefoxitin; Cefprozil (cefproxil); Cefuroxime; Cefuzonam; Cefcapene; Cefdaloxime; Cefdinir; Cefditoren; Cefetamet; Cefixime; Cefmenoxime; Cefodizime; Cefotaxime; Cefpimizole; Cefpodoxime; Cefteram; Ceftibuten; Ceftiofur; Ceftiolene; Ceftizoxime; Ceftriaxone; Cefoperazone; Ceftazidime; Cefclidine; Cefepime; Cefluprenam; Cefoselis; Cefozopran; Cefpirome; Cefquinome; Fifth Generation; Ceftobiprole; Ceftaroline; Not Classified; Cefaclomezine; Cefaloram; Cefaparole; Cefcanel; Cefedrolor; Cefempidone; Cefetrizole; Cefivitril; Cefmatilen; Cefmepidium; Cefovecin; Cefoxazole; Cefrotil; Cefsumide; Cefuracetime; Ceftioxide; Azithromycin; Erythromycin; Clarithromycin; Dirithromycin; Roxithromycin; Telithromycin; Amikacin; Gentamicin; Kanamycin; Neomycin; Netilmicin; Paromomycin; Streptomycin; Tobramycin; Flumequine; Nalidixic acid; Oxolinic acid; Piromidic acid; Pipemidic acid; Rosoxacin; Ciprofloxacin; Enoxacin; Lomefloxacin; Nadifloxacin; Norfloxacin; Ofloxacin; Pefloxacin; Rufloxacin; Balofloxacin; Gatifloxacin; Grepafloxacin; Levofloxacin; Moxifloxacin; Pazufloxacin; Sparfloxacin; Temafloxacin; Tosufloxacin; Besifloxacin; Clinafloxacin; Gemifloxacin; Sitafloxacin; Trovafloxacin; Prulifloxacin; Sulfamethizole; Sulfamethoxazole; Sulfisoxazole; Trimethoprim-Sulfamethoxazole; Demeclocycline; Doxycycline; Minocycline; Oxytetracycline; Tetracycline; Tigecycline; Chloramphenicol; Metronidazole; Tinidazole; Nitrofurantoin; Vancomycin; Teicoplanin; Telavancin; Linczolid; Cycloserine 2; Rifampin; Rifabutin; Rifapentine; Bacitracin; Polymyxin B; Viomycin; Capreomycin.

If a viral infection is ruled in, the subject may be treated with an antiviral agent. Examples of antiviral agents include, but are not limited to Abacavir; Aciclovir; Acyclovir; Adefovir; Amantadine; Amprenavir; Ampligen; Arbidol; Atazanavir; Atripla; Balavir; Boceprevirertet; Cidofovir; Combivir; Dolutegravir; Darunavir; Delavirdine; Didanosine; Docosanol; Edoxudine; Efavirenz; Emtricitabine; Enfuvirtide; Entecavir; Ecoliever; Famciclovir; Fomivirsen; Fosamprenavir; Foscarnet; Fosfonet; Fusion inhibitor; Ganciclovir; Ibacitabine; Imunovir; Idoxuridine; Imiquimod; Indinavir; Inosine; Integrase inhibitor; Interferon type III; Interferon type II; Interferon type I; Interferon; Lamivudine; Lopinavir; Loviride; Maraviroc; Moroxydine; Methisazone; Nelfinavir; Nevirapine; Nexavir; Oseltamivir; Peginterferon alfa-2a; Penciclovir; Peramivir; Pleconaril; Podophyllotoxin; Raltegravir; Reverse transcriptase inhibitor; Ribavirin; Rimantadine; Ritonavir; Pyramidine; Saquinavir; Sofosbuvir; StavudineTelaprevir; Tenofovir; Tenofovir disoproxil; Tipranavir; Trifluridine; Trizivir; Tromantadine; Truvada; traporved; Valaciclovir; Valganciclovir; Vicriviroc; Vidarabine; Viramidine; Zalcitabine; Zanamivir; Zidovudine; RNAi antivirals; inhaled rhibovirons; monoclonal antibody respigams; neuriminidase blocking agents.

The information gleaned using the methods described herein may aid in additional patient management options. For example, the information may be used for determining whether a patient should or should not be admitted to hospital. It may also affect whether or not to prolong hospitalization duration. It may also affect the decision whether additional tests need to be performed or may save performing unnecessary tests such as CT and/or X-rays and/or MRI and/or culture and/or serology and/or PCR assay for specific bacteria and/or PCR assays for viruses and/or perform procedures such as lumbar puncture.

It is often clinically useful to assess patient prognosis, disease severity and outcome. The present inventors have now found that low levels of TRAIL (lower than about 20 pg/ml or about 15 pg/ml or about 10 pg/ml or about 5 pg/ml or about 2 pg/ml) are significantly correlated with poor patient prognosis and outcome, and high disease severity. For example, the present inventors showed that adult patients in the intensive care unit (ICU), which are generally severely ill, had significantly lower TRAIL levels compared to all other patients, which were less ill regardless of whether they had an infectious or non-infectious etiology.

Thus, according to another aspect of the present invention there is provided a method of predicting a prognosis for a disease comprising measuring the TRAIL protein serum level in subject having the disease, wherein when the TRAIL level is below a predetermined level, the prognosis is poorer than for a subject having a disease having a TRAIL protein serum level above the predetermined level.

Methods of measuring TRAIL protein serum levels are described herein above.

The disease may be an infectious disease or a non-infectious disease. The subject may have a disease which has been diagnosed or non-diagnosed.

Particular examples of diseases include without limitation bacterial infections (e.g. bacteremia, meningitis, respiratory tract infections, urinal tract infections etc.), sepsis, physical injury and trauma, cardiovascular diseases, multi-organ failure associated diseases, drug-induced nephrotoxicity, acute kidney disease, renal injury, advanced cirrhosis and liver failure, acute or chronic left heart failure, pulmonary hypertension with/without right heart failure, and various types of malignancies.

According to another embodiment, additional polypeptides are measured which aid in increasing the accuracy of the prediction. Thus, for example, other polypeptide which may be measured include IP-10, CRP, IL1RA, PCT and SAA.

According to a particular embodiment, IP-10, CRP and TRAIL are measured.

According to another embodiment, only TRAIL is measured.

The present inventors have found that patients having very low levels of TRAIL (as classified herein above) have lower chance of recovery, and higher chance of complications. Accordingly, the present inventors propose that when it is found that a subject has very low levels of TRAIL they should be treated with agents that are only used as a last resort.

Such agents for example may be for example experimental agents that have not been given full FDA approval. Other last resort agents are those that are known to be associated with severe side effects. Another exemplary last resort agent is an antibiotic such as vancomycin (which is typically not provided so as to prevent the spread of antibiotic resistance).

It will be appreciated that agents that are not typically considered as last resort agents can also be provided, but in doses which exceed the clinically acceptable dose.

According to this aspect of the present invention, if the TRAIL level is above a predetermined level, then the patient should typically not be treated with a last resort agent.

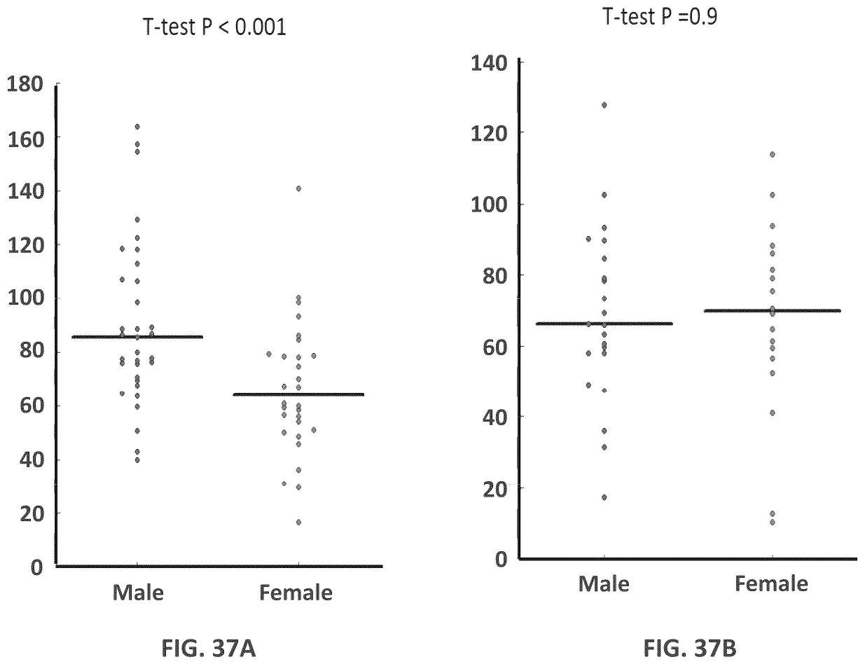

The present inventors have now found that basal levels of TRAIL in healthy individuals or patients with a non-infectious disease are lower in females compared to males during fertility age (t-test P<0.001) (see FIG. 37A), but is invariant in pre- or post-fertility age (t-test P=0.9, FIG. 37A). This trend was not observed in patients with an infectious disease.

This age dependent dynamics can be used to improve models distinguishing between bacterial, viral and non-infectious or healthy individuals, as would be evident to one skilled in the art.

For example, the model can include age and gender parameters. If the subject's age is within a certain range indicative of fertility (e.g. about 13 to 45 years) and the subject is male, then TRAIL model coefficients of males at fertility age can be used. If the subject's age is within the range indicative of fertility and the subject is female then TRAIL model coefficients of females at fertility age can be used. If the subject's age is outside the range indicative of fertility then TRAIL model coefficients that are gender invariant can be used.

Thus, according to another aspect of the invention there is provided a method of determining an infection type in a female subject of fertility age, the method comprising comparing the TRAIL protein serum level in the subject to a predetermined threshold, said predetermined threshold corresponding to the TRAIL protein serum level of a healthy female subject of fertility age, or a group of healthy female subjects of fertility age, wherein a difference between said TRAIL protein serum level and said predetermined threshold is indicative of an infection type.

Thus, according to another aspect of the invention there is provided a method of determining an infection type in a male subject of fertility age, the method comprising comparing the TRAIL protein serum level in the subject to a predetermined threshold, said predetermined threshold corresponding to the TRAIL protein serum level of a healthy male subject of fertility age, or a group of healthy male subjects of fertility age, wherein a difference between said TRAIL protein serum level and said predetermined threshold is indicative of an infection type.

It will be appreciated that predetermined thresholds can be used to either rule in or rule out an infection type.

Thus, for example if the TRAIL protein serum level is above a first predetermined threshold, the infection type is viral.

If, for example the TRAIL protein serum level is above a second predetermined threshold, the infection type is not bacterial.

If for example, the TRAIL protein serum level is below a third predetermined threshold, the infection type is bacterial.

If for example the TRAIL protein serum level is below a fourth predetermined threshold, the infection type is not viral.

Typically, the healthy male or female subject, referred to herein has no known disease. According to a particular embodiment, the control subject has no infectious disease.

Typically, the difference between the TRAIL protein serum level of the subject and the predetermined threshold is a statistically significant difference, as further described herein above.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization-A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### A Host-Proteome Signature for Distinguishing Between Bacterial and Viral Infections: A Prospective Multi-Center Observational Study

### Methods

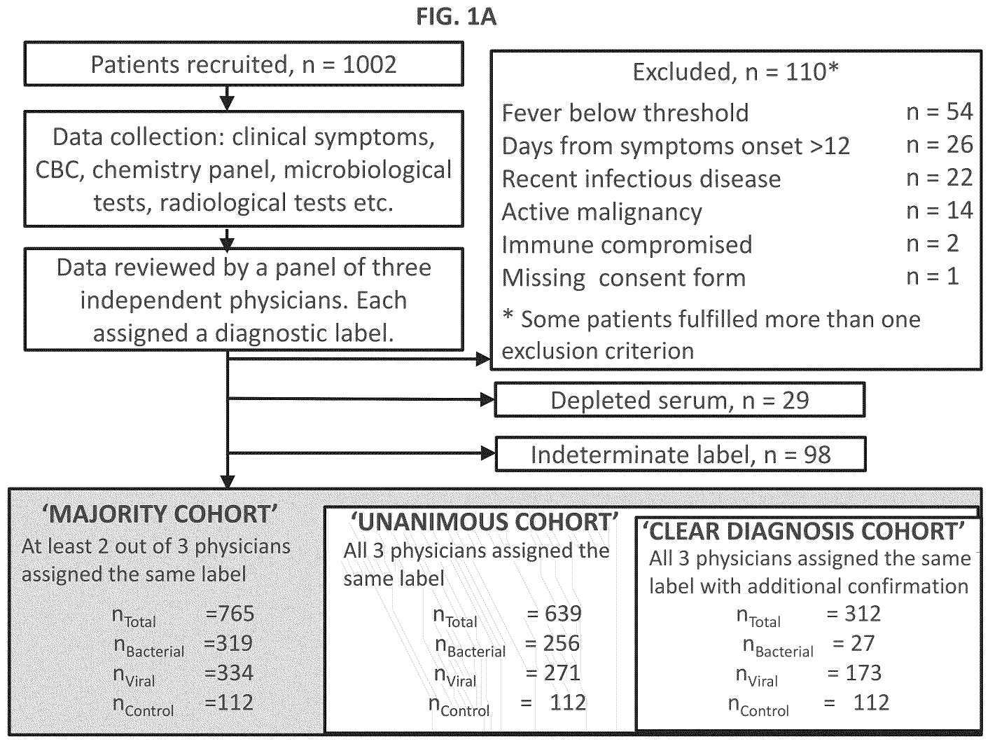

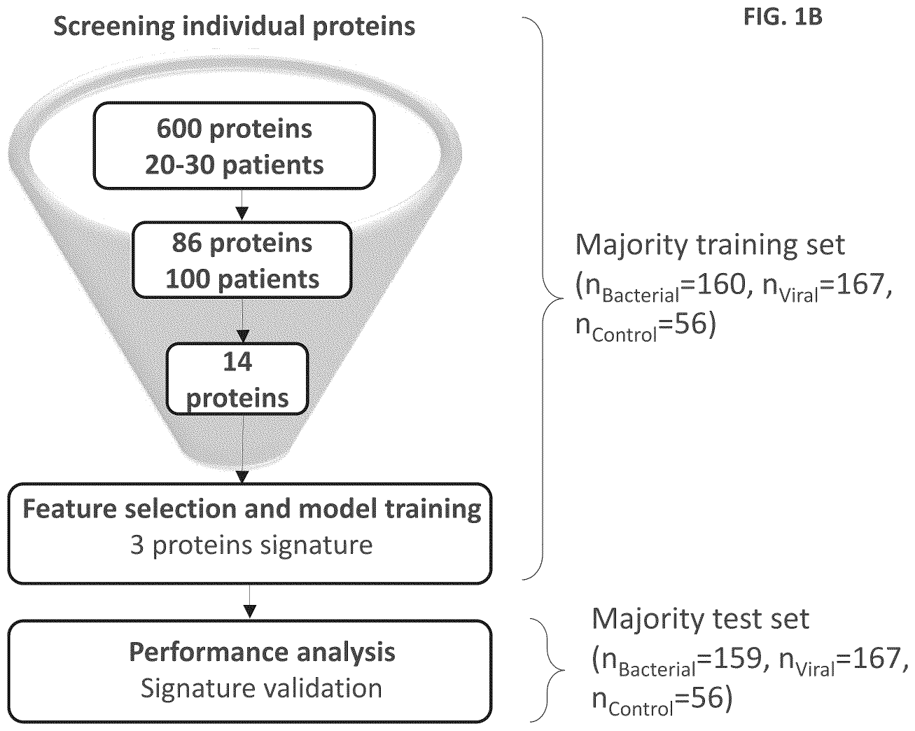

Study population: A total of 1002 patients took part in the study. Pediatric patients (≤18 years) were recruited from pediatric emergency departments (PED), pediatric wards and surgical departments, and adults (>18 years) from emergency departments (ED), internal medicine departments and surgical departments. Informed consent was obtained from each participant or legal guardian, as applicable. Inclusion criteria for the infectious disease cohort included: clinical suspicion of an acute infectious disease, peak fever>37.5° C. since symptoms onset, and duration of symptoms≤12 days. Inclusion criteria for the control group included: clinical impression of a non-infectious disease (e.g. trauma, stroke and myocardial infarction), or healthy subjects. Exclusion criteria included: evidence of any episode of acute infectious disease in the two weeks preceding enrollment; diagnosed congenital immune deficiency; current treatment with immunosuppressive or immunomodulatory therapy; active malignancy, proven or suspected human immunodeficiency virus (HIV)-1, hepatitis B virus (HBV), or hepatitis C virus (HCV) infection (FIG. 1A). Importantly, in order to enable broad generalization, antibiotic treatment at enrollment did not cause exclusion from the study.

Enrollment process and data collection: For each patient, the following baseline variables were recorded: demographics, physical examination, medical history (e.g. main complaints, underlying diseases, chronically-administered medications, comorbidities, time of symptom onset, and peak temperature), complete blood count (CBC) obtained at enrollment, and chemistry panel (e.g. creatinine, urea, electrolytes, and liver enzymes). A nasal swab was obtained from each patient for further microbiological investigation, and a blood sample was obtained for protein screening and validation. Additional samples were obtained as deemed appropriate by the physician (e.g. urine and stool samples in cases of suspected urinary tract infection [UTI], and gastroenteritis [GI] respectively). Radiological tests were obtained at the discretion of the physician (e.g. chest X-ray for suspected lower respiratory tract infection [LRTI]). Thirty days after enrollment, disease course and response to treatment were recorded. All information was recorded in a custom electronic case report form (eCRF).

Microbiological investigation: Patients underwent two multiplex-PCR diagnostic assays from nasal swab samples: (i) Seeplex® RV15 (n=713), for detection of parainfluenza virus 1, 2, 3, and 4, coronavirus 229E/NL63, adenovirus A/B/C/D/E, bocavirus 1/2/3/4, influenza virus A and B, metapneumovirus, coronavirus OC43, rhinovirus A/B/C, respiratory syncytial virus A and B, and Enterovirus, and (ii) Seeplex® PB6 (n=633) for detection of Streptococcus pneumoniae, Haemophilus influenzae, Chlamydophila pneumoniae, Legionella pneumophila, Bordetella pertussis, and Mycoplasma pneumoniae. Multiplex-PCR assays were performed by a certified service laboratory. Patients were also tested for additional pathogens according to their suspected clinical syndrome, including: blood culture (n=420), urine culture (n=188) and stool culture for Shigella spp., Campylobacter spp. and Salmonella spp. (n=66); serological testing (IgM and/or IgG) for cytomegalovirus (CMV), Epstein-Barr virus (EBV), Mycoplasma Pneumonia, and Coxiella burnetii (Q-Fever) (n=167, n=130, n=206 and n=41 respectively).

Establishing the reference standard: The Clear Diagnosis, Unanimous and Majority cohorts: A rigorous composite reference standard was created following recommendations of the Standards for Reporting of Diagnostic Accuracy (STARD).³⁸ First, a thorough clinical and microbiological investigation was performed for each patient as described above. Then, all the data collected throughout the disease course was reviewed by a panel of three physicians. For adult patients (>18 years) the panel included the attending physician and two infectious disease specialists, while for children and adolescents (≤18 years) it included the attending pediatrician, an infectious disease expert and a senior attending pediatrician. Each panel member assigned one of the following diagnostic labels to each patient: (i) bacterial; (ii) viral; (iii) no apparent infectious disease or healthy (controls); and (iv) indeterminate. Patients with mixed infections (bacteria plus virus) were labeled as bacterial because they are managed similarly (e.g. treated with antibiotics). Importantly, the panel members were blinded to the labeling of their peers and to the results of the signature.

This process was used to create three cohorts with an increasing level of diagnostic certainty (FIG. 1A):
(i) Majority cohort: Patients were assigned the same label by at least two of the three panel members;
(ii) Unanimous cohort (a subgroup of the Majority cohort): Patients were assigned the same label by all three panel members (the terms “unanimous cohort” and “consensus cohort” are used herein interchangeably); and
(iii) Clear Diagnosis cohort (a subgroup of the Unanimous cohort): Bacterial labeled patients were unanimously diagnosed by all three panel members, had WBC>15,000/μl (a cutoff indicative of increased bacterial infection risk¹¹) and one of the following microbiological confirmations: bacteremia (with positive blood culture), bacterial meningitis (with positive CSF culture), pyelonephritis (with positive urine culture and ultrasound demonstration of renal involvement), UTI (with positive urine culture), septic shock (with positive blood culture), or peritonsillar abscess (proven by surgical exploration or computerized tomography). Viral labeled patients were unanimously diagnosed by panel members and had and a positive test result of a virus.

Additionally, control labeled patients were unanimously diagnosed by all three panel members.

Samples, procedures and protein measurements: Venous blood samples were stored at 4° C. for up to 5 hours on site and subsequently fractionated into plasma, serum and total leukocytes and stored at −80° C. Nasal swabs and stool samples were stored at 4° C. for up to 72 hours and subsequently transported to a certified service laboratory for multiplex PCR-based assay. In the screening phase, host-proteins were measured in serum and leukocytes using enzyme linked immunosorbent assay (ELISA), Luminex technology, protein arrays and Flow cytometry (on freshly isolated leukocytes). After screening and signature construction (see Host-proteome screening section), three proteins were selected and measured as follows: CRP was measured via either Cobas 6000, Cobas Integra 400, Cobas Integra 800, or Modular Analytics P800 (Roche). TRAIL and IP-10 were measured using commercial ELISA kits (MeMed Diagnostics).

Statistical analysis: The primary analysis was based on area under the receiver operating characteristics curve (AUC), Sensitivity (TP/P), Specificity (TN/N), Positive likelihood ratio (LR+=Sensitivity/[1−Specificity]), Negative likelihood ratio (LR−=[1−Sensitivity]/Specificity) and Diagnostic odds ratio (DOR=LR+/LR−), where P, N, TP and TN correspond to positives (bacterial patients), negatives (viral patients), true positives (correctly diagnosed bacterial patients), and true negatives (correctly diagnosed viral patients), respectively. Statistical analysis was performed with MATLAB. Sample size calculations are presented in Example 2 herein below.

Host-proteome screening: A general overview of the process for developing, training and testing the multivariate logistic model is depicted in FIG. 1B. Briefly, a systematic literature screen and bioinformatics analysis was performed that identified 600 protein candidates that were likely to be differentially expressed in peripheral blood samples of bacterial versus viral patients, some of which have a known role in the host immune response to infection and others with no direct link to the immune system. Next, each protein candidate was measured on 20-30 patients from the training set (50% viral and 50% bacterial) and a Wilcoxon rank-sum (WS) P-value<0.01 was used to screen proteins with statistically significant differential measurements. This resulted in a set of 86 proteins (false discovery rate [FDR] of 0.07). Each of these proteins was then evaluated in 100 additional patients (50% viral and 50% bacterial) and further screened using a t-test cutoff of P<10⁻⁴, resulting in 14 proteins that were significantly differentially expressed in viral versus bacterial patients (FDR<0.001).

Signature development and validation: A feature selection process was applied to identify the optimal combination of proteins. Two feature selection schemes were used: mutual-information min-max³⁹ and forward greedy wrapper⁴⁰, which use a series of iterations to add or remove features. The process was terminated when the increase in performance on the training set was no longer statistically significant (P>0.05). Both processes converged to the same final set of three proteins. To integrate the protein levels into a single score, multiple computational models were examined. Their performances were not significantly different (P>0.1 as further detailed in Example 2 herein below). A Multinomial Logistic Regression (MLR) model was chosen because provides a probabilistic interpretation by assigning a likelihood score to a patient's diagnosis. The signature uses this property to filter out patients whose probability of bacterial infection is intermediate: between 0.35 and 0.55. The term ‘marginal immune response’ is used to describe these patients because their profile borders between bacterial and viral host-responses. The patients in the Majority cohort were divided into training and test sets, each comprising 50% of the patients (FIG. 1B). The training set included the 120 patients who participated in the screening process and additional patients that were randomly assigned. The test set included the remaining patients and was used for independent assessment of the signature performance. Importantly, none of the test set patients were used to train the algorithms, or to select the proteins. A leave-10%-out cross-validation was used to estimate model performance. More details on the model construction are provided in Example 2 herein below).

### Results

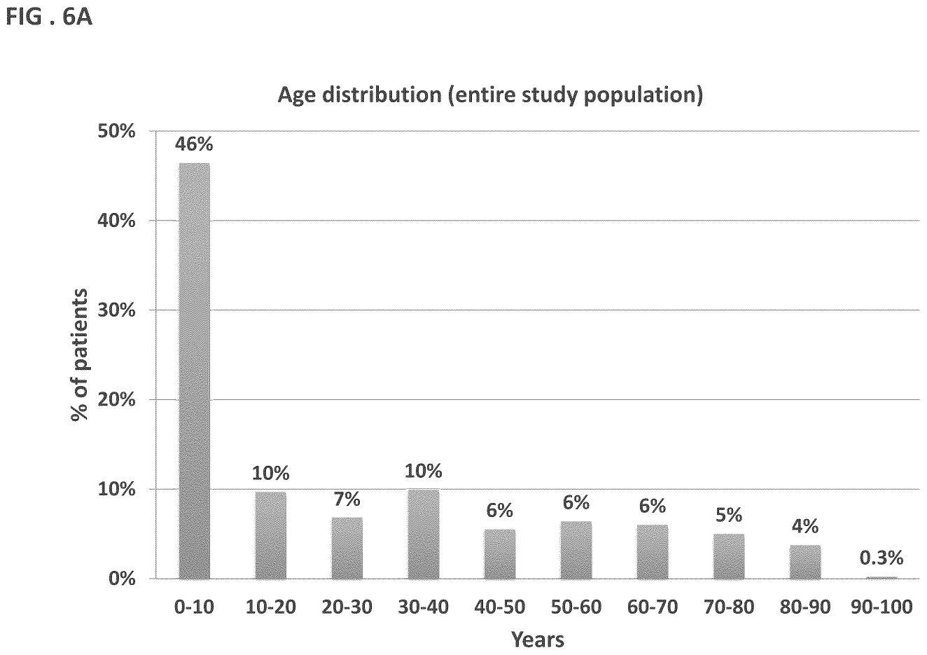

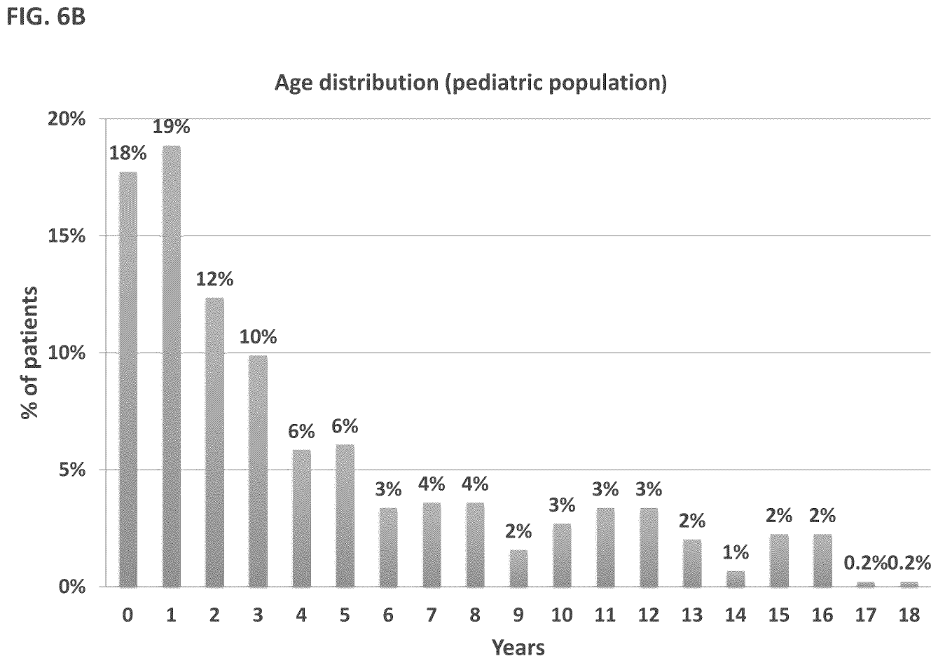

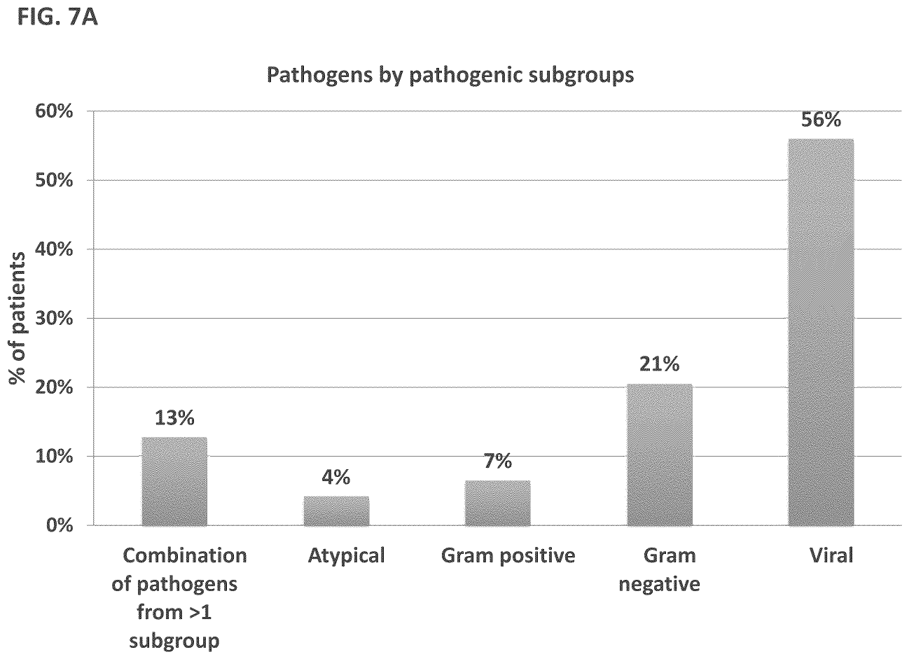

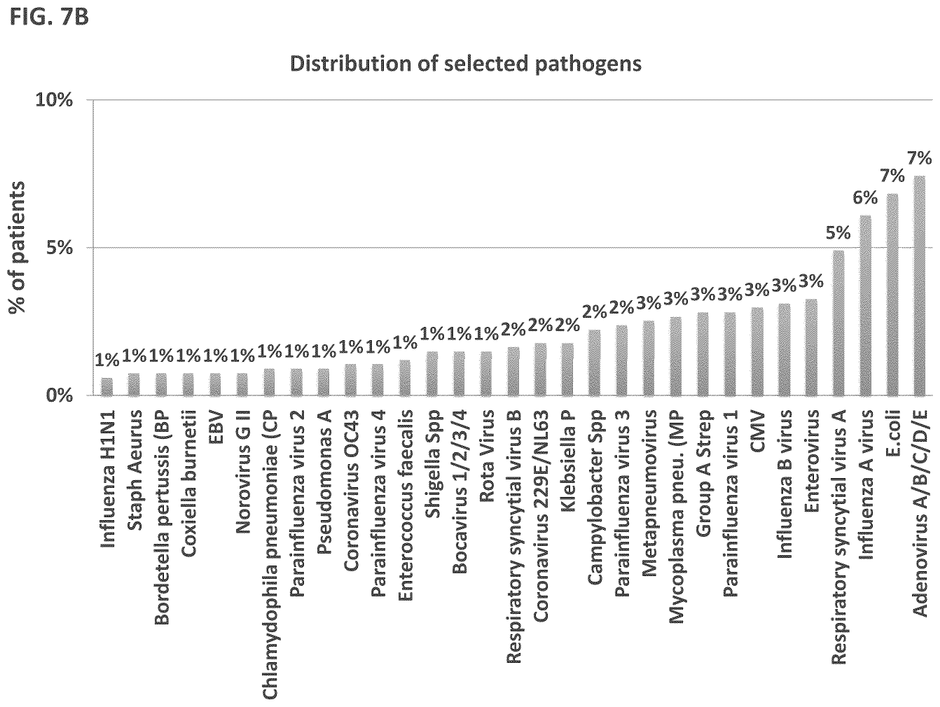

Patient characteristics: Three physicians independently assigned a label to each patient (either bacterial, viral, controls, or indeterminate). The labels were used to create three cohorts with increasing level of diagnostic certainty: Majority (n=765), Unanimous (n=639) and Clear Diagnosis (n=312) cohorts (FIG. 1A). Additionally, 98 patients were labeled as indeterminate, because the physicians could not establish disease etiology or there was no majority labeling. A detailed characterization of the Majority cohort is depicted in Table 1. Briefly, the cohort was balanced with respect to gender (47% females, 53% males) and included 56% pediatric patients (≤18 years) and 44% adults (>18 years). Patients presented with a wide range of clinical syndromes (e.g. RTI, UTI, and systemic infections), maximal temperatures (36-41.5° C.), time from symptoms onset (0-12 days), comorbidities, and medications (Table 1 and FIGS. 6A-12B). Altogether, 56 pathogen species were detected that are responsible for the vast majority of acute infectious diseases in the Western world (FIGS. 7A-7B).

TABLE 1
Baseline characteristics of the majority cohort patients.

| | | | Children | Adults |
|---|---|---|---|---|
| | | Total | (≤18 years) | (>18 years) |
| Criteria | | n = 765 | n = 432 | n = 333 |
| Age (years) | <3 | 211 (28) | | |
| | 3-6 | 93 (12) | | |
| | 6-9 | 46 (6) | | |
| | 9-18 | 82 (11) | | |
| | 18-30 | 55 (7) | | |
| | 30-60 | 161 (21) | | |
| | >60 | 117 (15) | | |
| Gender | Female | 363 (47) | 205 (47) | 158 (47) |
| Maximal | <37.5 | 106 (14) | 28 (6) | 78 (23) |
| Temperature | 37.5-38.4 | 154 (20) | 68 (16) | 86 (26) |
| (° C.) | 38.5-39.4 | 294 (38) | 164 (38) | 130 (39) |
| | 39 5-40 4 | 196 (26) | 157 (36) | 39 (12) |
| | >40.5 | 15 (2) | 15 (3) | 0 (0) |
| Time from | 0-1 | 175 (24) | 118 (27) | 57 (17) |
| symptoms | 2-3 | 265 (36) | 161 (37) | 104 (31) |
| onset (days) | 4-5 | 161 (22) | 89 (21) | 72 (22) |
| | 6-7 | 109 (15) | 52 (12) | 57 (17) |
| | 8-9 | 10 (1) | 2 (0.5) | 8 (2) |
| | 10-12 | 14 (2) | 2 (0.5) | 12 (4) |
| | N/A | 31 (4) | 8 (2) | 23 (7) |
| Clinical | Cellofills | 28 (4) | 7 (2) | 21 (6) |
| syndrome | CNS | 14 (2) | 9 (2) | 5 (2) |
| | GI | 89 (11.5) | 66 (15) | 23 (7) |
| | LRTI | 158 (21) | 84 (19) | 74 (22) |
| | Non-infectious | 112 (14.5) | 29 (7) | 83 (25) |
| | Other | 12 (1.5) | 4 (1) | 8 (2.5) |
| | Systemic | 150 (19.5) | 110 (26) | 40 (12) |
| | URTI | 145 (19) | 104 (24) | 41 (12) |
| | UTI | 57 (7) | 19 (4) | 38 (11) |
| Recruiting site | Pediatrics & Internal | 293 (38) | 137 (32) | 156 (47) |
| | PED & ED | 472 (62) | 295 (68) | 177 (53) |
| Hospitalization | Not hospitalized | 272 (36) | 174 (40) | 98 (29) |
| duration (days) | 1-2 | 206 (28) | 126 (29) | 80 (24) |
| | 3-4 | 170 (22) | 94 (22) | 76 (23) |
| | 5-6 | 53 (7) | 24 (6) | 29 (9) |
| | 7-8 | 31 (4) | 7 (1.5) | 24 (7) |
| | >8 | 33 (4) | 7 (1.5) | 26 (8) |
| Season | Autumn | 181 (24) | 111 (26) | 70 (21) |
| | Spring | 208 (27) | 124 (29) | 84 (25) |
| | Summer | 170 (22) | 98 (23) | 72 (22) |
| | Winter | 206 (27) | 99 (23) | 107 (32) |
| Smoking | Yes | 74 (10) | 0 (0) | 74 (22) |
| | No | 691 (90) | 432 (100) | 259 (78) |
| Antibiotic | Yes | 432 (56) | 207 (48) | 225 (68) |
| prescription | No | 333 (44) | 225 (52) | 108 (32) |
| Detected | Not detected | 219 (29) | 79 (18) | 140 (42) |
| microorganisms | | | | |
| Viruses | Adenovirus A/B/C/D/E | 50 (7) | 47 (11) | 3 (1) |
| | Bocavtrus 1/2/3/4 | 9 (1) | 9 (2) | 0 (0) |
| | CMV & EBV | 25 (3) | 23 (5) | 2 (0.6) |
| | Coronavirus 229E/NL63/OC43 | 19 (2) | 14 (3) | 5 (2) |
| | Enteric viruses | 19 (2) | 16 (4) | 3 (1) |
| | Enterovirus | 21 (3) | 20 (5) | 1 (0.3) |
| | Influenza A virus | 45 (6) | 24 (6) | 21 (6) |
| | Influenza B virus | 19 (2) | 14 (3) | 5 (2) |
| | Metapneumovirus | 17 (2) | 13 (3) | 4 (1) |
| | Parainfluenza 1/2/3/4 | 48 (6) | 41 (9) | 7 (2) |
| | Respiratory syncytial virus A/B | 40 (5) | 38 (9) | 2 (0.6) |
| | Rhinovirus A/B/C | 87 (11) | 73 (17) | 14 (4) |
| Bacteria | Atypical bacteria | 27 (4) | 7 (2) | 20 (6) |
| | E. coli | 44 (6) | 17 (4) | 27 (8) |
| | Enterococcus faecalis | 10 (1) | 0 (0) | 10 (3) |
| | Group A Step | 19 (2) | 16 (4) | 3 (1) |
| | Haemophilus influenzae | 179 (23) | 148 (34) | 31 (9) |
| | Streptococcus pneumoniae | 306 (40) | 207 (48) | 99 (30) |

Values are numbers (percentages). Only microorganisms that were detected in more than 5 patients are presented.
CNS—central nervous system, GI—gastroenteritis, LRTI—lower respiratory tract infection, UTRI—upper respiratory tract infection, UTI—urinary tract infection, N/A—healthy controls or patients in which data was not obtained.
Influenza A subgroup included H1N1 strains. The atypical bacteria subgroup included Chlamydophila pneumoniae, Mycoplasma pneumonia and Legionella pneumophila. The Enteric viruses subgroup included Rota virus, Astrovirus, Enteric Adenovirus and Norovirus G I/II. In the clinical syndrome analysis the LRTI group included pneumonia, bronchiolitis, acute bronchitis, and laryngitis; URTI group included pharyngitis, acute otitis media, acute sinusitis and acute tonsillitis.

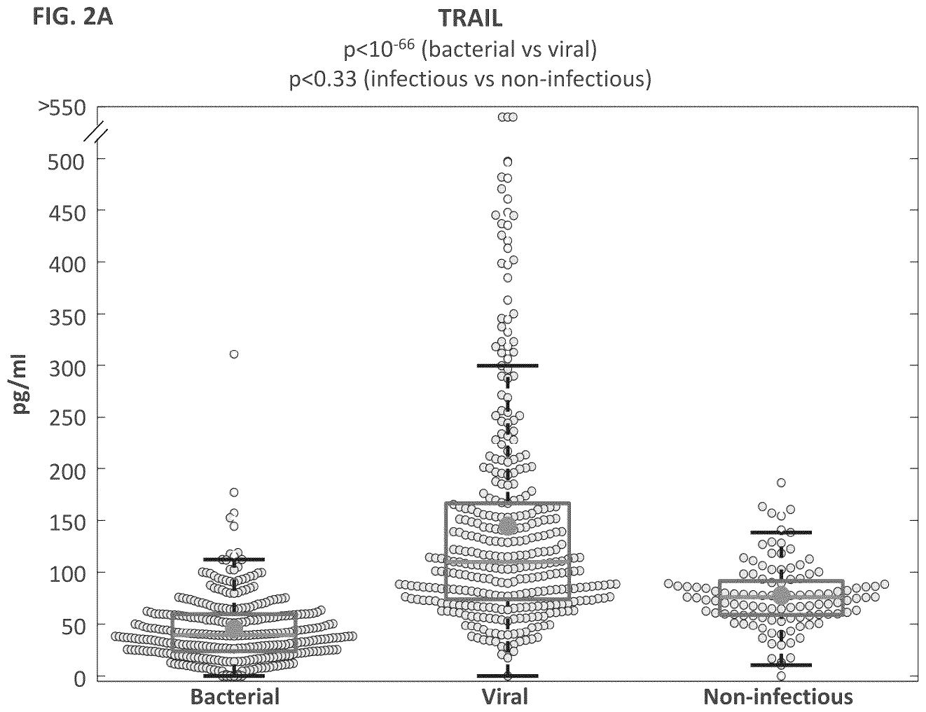

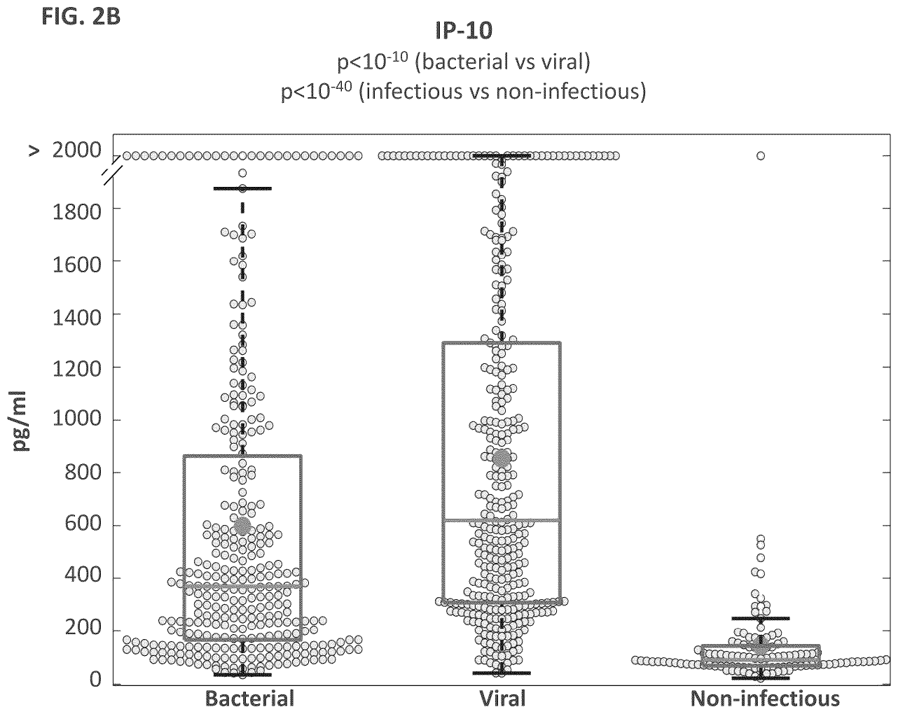

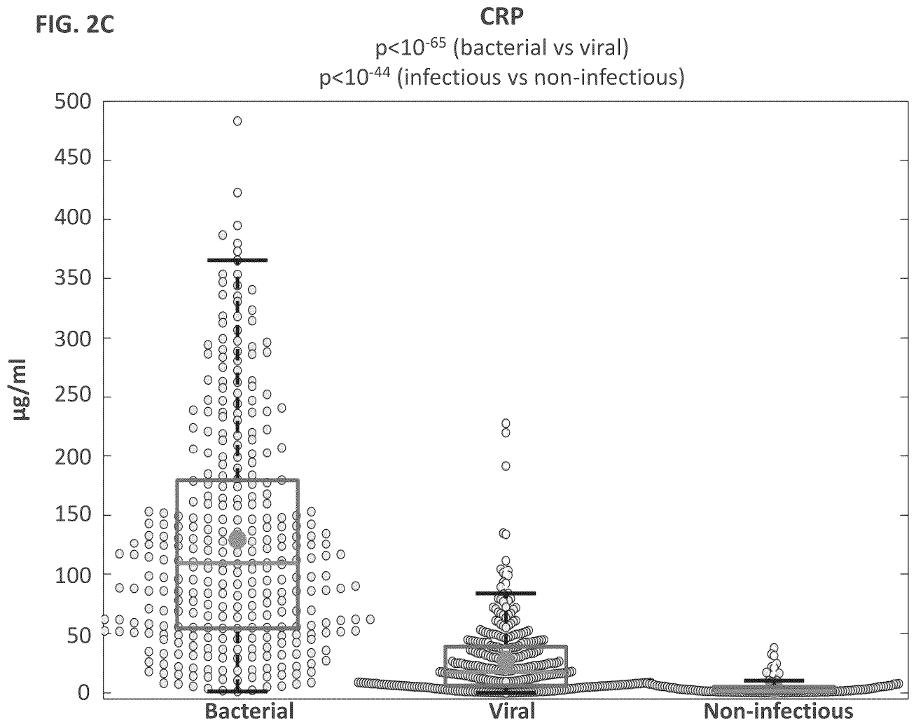

Signature performance on the Clear Diagnosis, Unanimous and Majority cohorts: Of the 600 screened host-proteins and their combinations, the best signature for discriminating bacterial, viral and control patients in the Majority cohort training set included three soluble proteins: TNF-related apoptosis-inducing ligand (TRAIL), Interferon gamma-induced protein 10 (IP-10), and C-reactive protein (CRP) (FIGS. 2A-2C). Signature AUC for distinguishing between bacterial and viral infections on the test set of the Majority cohort was 0.94±0.04. Similar results were obtained using leave-10%-out cross-validation on the entire Majority cohort (AUC-0.94±0.02). The signature significantly outperformed all the individual proteins evaluated in the screening phase (P<10⁻⁶). The training and testing procedures were repeated on the Unanimous and Clear Diagnosis cohorts, yielding AUCs of 0.96±0.02 and 0.99±0.01, respectively. This stepwise increase in performance is aligned with the increased certainty of reference standard assignment in the three cohorts (Table 2, herein below).

TABLE 2
Signature measures of accuracy for diagnosing bacterial vs. viral infections

| B. Marginal immune response | | | | | | |
|---|---|---|---|---|---|---|
| filter | | | A. All patients | | | |
| | | Clear | | | Clear | |
| Majority | Unanimous | diagnosis | Majority | Unanimous | diagnosis | Accuracy |
| cohort | cohort | cohort | cohort | cohort | cohort | measure |
| 0.94 | 0.97 | 0.99 | 0.94 | 0.96 | 0.99 | AUC |
| (0.92, 0.96) | (0.95, 0.99) | (0.98, 1.00) | (0.92, 0.96) | (0.94, 0.98) | (0.98, 1.00) | |
| 0.91 | 0.93 | 0.96 | 0.88 | 0.90 | 0.94 | Total |
| (0.88, 0.94) | (0.9, 0.96) | (0.93, 0.99) | (0.85, 0.90) | (0.87, 0.92) | (0.91, 0.97) | accuracy |
| 0.92 | 0.94 | 0.96 | 0.87 | 0.88 | 0.96 | Sensitivity |
| (0.88, 0.96) | (0.9, 0.98) | (0.88, 1.00) | (0.83, 0.91) | (0.84, 0.91) | (0.88, 1.00) | |
| 0.89 | 0.93 | 0.97 | 0.90 | 0.92 | 0.93 | Specificity |
| (0.86, 0.89) | (0.9, 0.96) | (0.89, 0.97) | (0.86, 0.93) | (0.89, 0.96) | (0.89, 0.97) | |
| 8.4 | 13.4 | 32.0 | 8.7 | 11.0 | 13.7 | LR+ |
| (6, 12) | (8, 21) | (13, 78) | (6, 12) | (7, 16) | (8, 24) | |
| 0.09 | 0.07 | 0.04 | 0.14 | 0.13 | 0.04 | LR− |
| (0.06, 0.13) | (0.04, 0.11) | (0.01, 0.26) | (0.11, 0.19) | (0.09, 0.18) | (0.01, 0.27) | |
| 93 | 208 | 776 | 60 | 84 | 319 | DOR |
| (53, 164) | (99, 436) | (92, 6528) | (37, 98) | (47, 150) | (43, 2383) | |

A. Performance estimates and their 95% CIs were obtained using a leave-10%- out cross-validation on all patients in the Clear Diagnosis cohort (n_{Bacterial} = 27, n_{Viral} = 173), Unanimous (n_{Bacterial} = 256, n_{Viral} = 271), and Majority (n_{Bacterial} = 319, n_{Viral} = 334) cohorts.
B. The analysis was repeated after filtering out patients with a marginal immune response (Clear Diagnosis [n_{Bacterial} = 27, n_{Viral} = 159, n_{marginal} = 14], Unanimous [n_{Bacterial} = 233, n_{Viral} = 232, n_{marginal} = 62], and Majority [n_{Bacterial} = 290, n_{Viral} = 277, n_{marginal} = 88]), which resembles the way clinicians are likely to use the signature.

Next, the present inventors used the signature to distinguish between infectious (bacterial or viral) and non-infectious controls on the Majority cohort test set, yielding an AUC of 0.96±0.02. Further evaluation using leave-10%-out cross-validation gave similar results (AUC=0.96±0.01). The signature outperformed any of the individual proteins (P<10⁻⁸). Again, evaluation on the Unanimous and Clear Diagnosis cohorts showed improved AUCs of 0.97±0.02, and 0.97±0.03, respectively. To obtain conservative estimations of signature performance, the analysis that follows focuses on the Majority cohort.

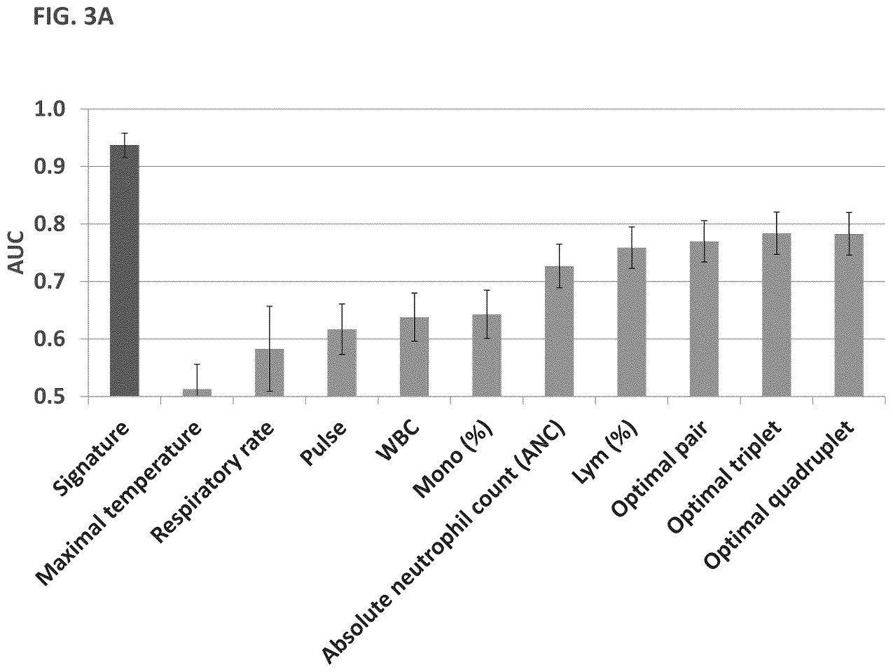

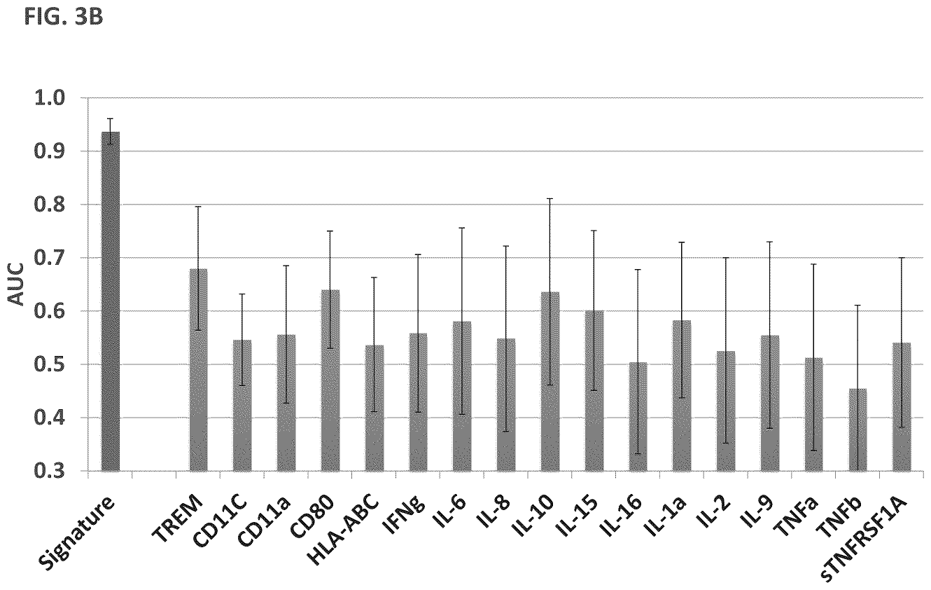

Comparison with laboratory measurements, clinical parameters, and well-established biomarkers: The signature was compared with well-established clinical parameters and laboratory measurements, including white blood count (WBC), absolute neutrophil count (ANC), percentage neutrophils, maximal temperature, pulse, and respiratory rate (FIG. 3A and Example 2). The signature surpassed all individual parameters (P<10-18). Next, the signature was compared to a combination of several clinical parameters. To this end, multinomial logistic models were generated for all combinations of up to four clinical parameters. The best performing pair, triplet and quadruplet are depicted in FIG. 3A (adding a fifth parameter did not improve performance). The signature was significantly better than the best performing clinical parameters combination (P<10⁻¹⁵), which consisted of ANC, pulse, % lymphocytes and % monocytes, (AUC=0.94±0.02 vs. 0.77±0.04). Next, the signature performance was compared to PCT and CRP, two proteins routinely used in clinical practice to diagnose sepsis and bacterial infections (Example 2). The signature performed significantly better than both proteins (P<10⁻⁸ and P<10⁻⁶, respectively). The signature also performed better than a wide range of host-proteins with an established role in the immune response to infection, including sepsis and bacterial-related (e.g. TREM, IL-6 and IL-8), virus-related (e.g. IFN-γ and IL-2), and inflammation-related (e.g. IL-1a and TNF-α) proteins (P<10⁻⁸) (FIG. 3B and Example 2, herein below).

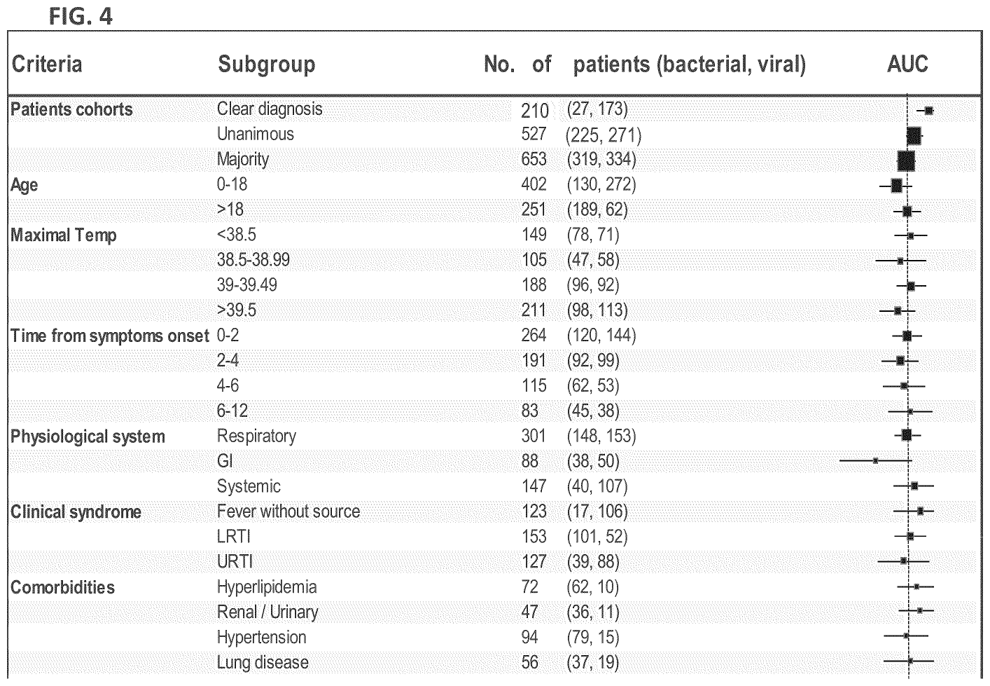

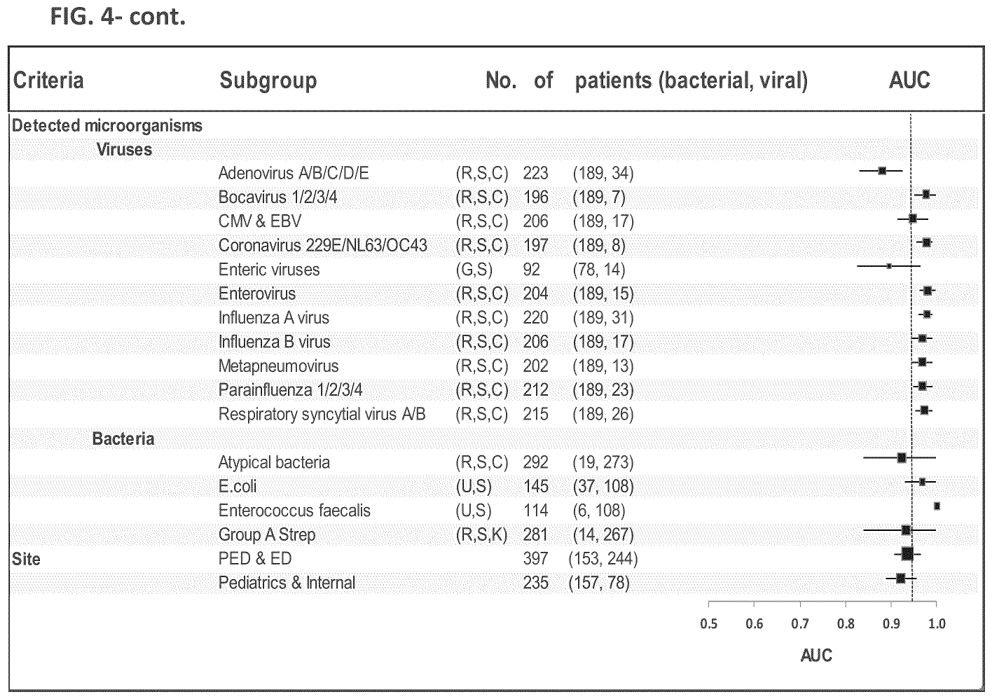

Signature performance is robust across different patient subgroups: Patient and pathogen heterogeneity, which are inherent in real-life clinical settings, might negatively affect the diagnostic utility of any individual host-biomarker. To examine whether the signature, a combination of multiple biomarkers, can maintain steady performance despite patient-to-patient variability, subgroup analyses were performed. The signature was robust (AUCs between 0.87 and 1.0) across a wide range of patient characteristics, including age, clinical syndrome, time from symptom onset, maximal temperature, pathogen species, comorbidities, treatment with medications for chronic diseases, and clinical site (FIG. 4 and Example 2, herein below). The signature was also tested on the subgroup of patients who were technically excluded, but had unanimous labeling by the expert panel, which yielded an AUC of 0.96±0.06 (n_{Bacterial}=27, n_{Viral}=14). This might suggest that the signature is applicable more broadly to conditions that were initially excluded (e.g. sub-febrile patients).

Signature performance remains unaffected by the presence of potential colonizers: Many disease-causing bacteria are also part of the natural flora, and are frequently found in asymptomatic subjects.^{12,42-44} Such bacteria pose a considerable diagnostic challenge, because merely detecting them does not necessarily imply a causative role in the disease; therefore, appropriate treatment may remain unclear. The present inventors asked whether the signature performance is affected by their presence.

Streptococcus pneumoniae (SP) and Haemophilus influenzae (HI), detected by PCR on nasal swabs, were the two most common bacteria in the Majority group (Table 1, herein above). High rates of SP and HI were found amongst both bacterial and viral patients (SP: 36% and 47%; HI: 20% and 32%), substantiating the understanding that their mere presence does not necessarily cause a disease.¹² The patients were stratified based on whether or not they had SP (SP+: n_{Bacterial}=116, n_{Viral}=157; SP−: n_{Bacterial}=203, n_{Viral}=177) and AUC performance of the two groups was compared. A significant difference was not observed (0.93±0.03 vs. 0.94±0.02, P=0.31). The presence or absence of HI did not affect signature performance either (0.94±0.04 vs. 0.93±0.02; HI+: n_{Bacterial}=63, n_{Viral}=106; HI−: n_{Bacterial}=256, n_{Viral}=228, P=0.34). This indicates that the signature remains unaffected by carriage of SP and HI.

### Discussion

A rigorous composite reference standard strategy was constructed that included the collection of clinical data, a chemistry panel, and a wide array of microbiological tests, followed by labeling by three independent physicians. This process generated a hierarchy of three sub-cohorts with decreasing size and increasing reference standard certainty: Majority, Unanimous and Clear Diagnosis. The respective signature AUCs were 0.94±0.02, 0.96±0.02, and 0.99±0.01. This stepwise increase in performance may be attributed to the increase in reference standard certainty. However, the increased accuracy, particularly in the Clear Diagnosis cohort, may also be partially due to a selection bias of patients with severe illness or straightforward diagnosis. Therefore, the primary analysis presented herein focused on the Majority cohort, which captures a wider spectrum of illness severity and difficult-to-diagnose cases. This cohort potentially includes some erroneous labeling, thereby leading to conservative estimations of the signature accuracy.

The signature addresses several challenges of current microbiological tests. (i) The difficulty of diagnosing inaccessible or unknown infection sites. The signature accurately diagnosed such cases, including lower respiratory tract infections (AUC 0.95±0.03, n=153) and fever without source (AUC=0.97±0.03, n=123). (ii) Prolonged time to results (hours to days). The signature measures soluble proteins, which are readily amenable to rapid measurement (within minutes) on hospital-deployed automated immunoassay machines and point-of-care devices. (iii) Mixed infections may lead to diagnostic uncertainty, because detection of a virus does not preclude bacterial co-infection.^{14,15} The signature addresses this by classifying mixed infections together with pure bacterial infections, thus prompting physicians to manage both groups similarly with regard to antibiotics treatment. The fact that mixed co-infections elicited a proteome host-response that is similar to pure bacterial, rather than a mixture of responses, may indicate pathway dominance of bacterial over viral. (iv) A significant drawback of microbiological tests, PCRs in particular, is detection of potential colonizers in subjects with non-bacterial diseases.^{12,13} The signature performance was unaffected by the presence or absence of potential colonizers.

Host-proteins, such as PCT, CRP and IL-6, are routinely used to assist in the diagnosis of bacterial infections because they convey additional information over clinical symptoms, blood counts and microbiology.¹¹ However, inter-patient and pathogen variability limit their usefullness.²¹⁻²⁷ Combinations of host-proteins have the potential to overcome this, but have thus far yielded insignificant-to-moderate diagnostic improvement over individual proteins.¹¹ This modest improvement may be due to the reliance on combinations of bacterial-induced proteins that are sensitive to the same factors, and are therefore less capable of compensating for one another. Accordingly, a larger improvement was observed in combinations that included host-proteins, clinical parameters and other tests.^{11,35-37} Obtaining these multiple parameters in real-time, however, is often not feasible.

To address this, a combination of proteins with complementary behaviors was identified. Specifically, it was found that TRAIL was induced in response to viruses and suppressed by bacteria, IP-10 was higher in viral than bacterial infections, and CRP was higher in bacterial than viral infections. While the utility of elevated CRP to suggest bacterial infections is well established^{31,45}, the inclusion of novel viral-induced proteins, to complement routinely used bacterial-induced proteins, substantially contributed to the signature's robustness across a wide range of subgroups, including time from symptom onset, pathogen species and comorbidities among others. For example, adenoviruses, an important subgroup of viruses that cause 5%-15% of acute infections in children are particularly challenging to diagnose because they induce clinical symptoms that mimic a bacterial infection.⁴⁶ Routine laboratory parameters perform poorly on this subgroup compared to the signature (AUCs=0.60±0.10 [WBC], 0.58±0.10 [ANC], 0.88±0.05 [signature]; n=223).

Despite advances in infectious disease diagnosis, timely identification of bacterial infections remains challenging, leading to antibiotic misuse with its profound health and economic consequences. To address the need for better treatment guidance, the present inventors have developed and validated a signature that combines novel and traditional host-proteins for differentiating between bacterial and viral infections. The present finding in a large sample size of patients is promising, suggesting that this host-signature has the potential to help clinicians manage patients with acute infectious disease and reduce antibiotic misuse.

### Example 2

### A Host-Proteome Signature for Distinguishing Between Bacterial and Viral Infections: A Prospective Multi-Center Observational Study—Supplementary Material

Measures of accuracy: The signature integrates the levels of three protein biomarkers measured in a subject, and computes a numerical score that reflects the probability of a bacterial vs. viral infection. To quantify the diagnostic accuracy of the signature a cutoff on the score was used and the following measures were applied: Sensitivity, specificity, positive predictive value (PPV), negative predictive value (NPV), total accuracy, positive likelihood ratio (LR+), negative likelihood ratio (LR−), and diagnostic odds ratio (DOR). These measures are defined as follows:

$$\begin{matrix}Sensitivity = \frac{TP}{TP + FN} \\ Specificity = \frac{TN}{TN + FP} \\ total accuracy = \frac{TP + TN}{TP + FN + TN + FP} \\ PPV = \frac{TP}{TP + FP} = \frac{sensitivity \cdot prevalence}{sensitivity \cdot prevalence + ( 1 - specificity ) \cdot ( 1 - prevalence )} \\ NPV = \frac{TN}{TN + FN} = \frac{specificity \cdot ( 1 - prevalence )}{specificity \cdot ( 1 - prevalence ) + ( 1 - sensitivity ) \cdot ( prevalence )} \\ LR += \frac{Sensitivity}{1 - Specificity} \\ LR -= \frac{1 - Sensitivity}{Specificity} \\ DOR = \frac{LR +}{LR -}\end{matrix}$$

P, N, TP, FP, TN, FN are positives, negatives, true-positives, false-positives, true-negatives, and false-negatives, respectively. Prevalence is the relative frequency of the positive class (i.e., prevalence=P/(P+N)). Unless mentioned otherwise, positives and negatives refer to patients with bacterial and viral infections, respectively.

The area under the receiver operating curve (AUC) was also used to perform cutoff independent comparisons of different diagnostic methods. For details on formulation and confidence interval (CI) computation of the AUC see Hanley and McNeil.¹ 95% CIs of the accuracy measures throughout this document are reported. Sample size: The primary study objective was to obtain the performance of the signature for classifying patients with viral and bacterial etiologies. It was estimated that the sample size required to reject the null hypothesis that the sensitivity and specificity over the entire population, P, are lower than P0=75% with significance level of 1%, power of 90% for a difference of 15% (P1−P0≥15%), which yielded 394 patients (197 viral and 197 bacterial). Additionally it was anticipated that roughly 15% of the patients will have an indeterminate source of infection, 10% would be excluded for technical reasons and 10% will be healthy or non-infectious controls. Taken together, the study required the recruitment of at least 607 patients. This requirement was fulfilled because 1002 patients were recruited.

Constructing a computation model logistic model: To integrate the protein levels into a single predictive score, multiple computational models were examined including Artificial Neural Networks (ANN), Support Vector Machines (SVM), Bayesian Networks (BN), K-Nearest Neighbor (KNN) and Multinomial Logistic Regression (MLR).^{2,3} The AUCs for distinguishing between bacterial and viral infections obtained on the Majority cohort using a leave-10%-out cross validation were 0.93±0.02 (ANN), 0.93±0.02 (SVM [linear]), 0.94±0.02 [SVM (radial basis function)], 0.92±0.02 (BN), 0.91±0.02 (KNN) and 0.94±0.02 (MLR). Significant difference in the performances of ANN, SVM and MLR models (P>0.1 when comparing their AUCs) were not observed. The present inventors chose to use MLR because it provides a probabilistic interpretation by assigning a likelihood score to a patient's diagnosis.

The present inventors trained and tested the MLR signature for distinguishing between bacterial and non-bacterial etiologies. Since the prevalence of underlying etiologies varies across different clinical settings, the model priors were adjusted to reflect equal baseline prevalence (50% bacterial and 50% non-bacterial). Within the non-bacterial group the priors were adjusted to 45% viral and 5% non-infectious, to reflect the anticipated higher prevalence of viral versus non-infectious patients among subjects with suspicious for acute infection. The MLR weights and their respective 95% confidence intervals, as well as the p-values associated with each coefficient are summarized in Tables 3-4 herein below. In the bacterial versus viral infection analysis the probabilities were adjusted to sum up to 1 (P_{b_adjusted}=[P_{b}+Pᵥ] and P_{b_adjusted}=[P_{b}+Pᵥ], where P_{b} and Pᵥ correspond to the probability of bacterial and viral infections respectively).

TABLE 3
MLR coefficients and their respective standard error

| Second Coordinate | First Coordinate | |
|---|---|---|
| δ₁ (bacterial) | δ₀ (viral) | |
| b₀ = −0.378 ± 0.732 | a₀ = −1.299 ± 0.651 | Constant |
| b₁ = −0.020 ± 0.0084 | a₁ = 0.0088 ± 0.0064 | TRAIL |
| b₂ = 0.0875 ± 0.015 | a₂ = 0.0605 ± 0.0145 | CRP |
| b₃ = 0.0050 ± 0.0014 | a₃ = 0.0053 ± 0.0014 | IP-10 |

TABLE 4
The p-values associated with each MLR coefficient.

| Class (bacterial) | Class (viral) | |
|---|---|---|
| <0.001 | <0.001 | Constant |
| <0.001 | 0.008 | TRAIL |
| <0.001 | <0.001 | CRP |
| <0.001 | <0.001 | IP-10 |

Logistic calibration curves: In order to assess the validity of the MLR model, the calculated prediction probabilities were compared with the actually observed outcomes (FIG. 5). The predicted probabilities are highly compatible with the observed ones, further demonstrating the model validity.

Summary of the patient cohorts used in this study: A total of 1002 patients were recruited and 892 were enrolled (110 were excluded based on pre-determined exclusion criteria). Based on the reference standard process described in the ‘Methods’ section of Example 1, patients were assigned to four different diagnosis groups: (i) bacterial; (ii) viral; (iii) no apparent infectious disease or healthy (controls); and (iv) indeterminate. Patients with mixed infections (bacteria plus virus) were labeled as bacterial because they are managed similarly (e.g. treated with antibiotics) (FIG. 1A). In total, 89% of all enrolled patients were assigned a diagnosis, a rate which approaches the literature-documented limit.⁴⁻⁶ The following sections provide a detailed description of patient characteristics, which includes all the patients with a final diagnosis (n=794): 765 patients of the Majority cohort and 29 patients for which the serum samples were depleted during the screening phase (FIGS. 1A-1B).

Age and gender distribution: Patients of all ages were recruited to the study. The patients with agreed diagnosis (diagnosed patients; n=794) included more pediatric (≤18 years) than adult (>18 years) patients (445 patients [56%] vs. 349 [44%]). The age distribution was relatively uniform for patients aged 20-80 years and peaked at <4 years of age for pediatric patients (FIGS. 6A-6B). The observed age distribution for pediatric patients is consistent with that expected and represents the background distribution in the inpatient setting⁷ (e.g., the emergency department [ED], pediatrics departments, and internal departments). Patients of both genders were recruited to the study. The patient population was balanced in respect to gender distribution (47% females, 53% males).

Detected pathogens: A wide panel of microbiological tools were used in order to maximize pathogen detection rate. At least one pathogen was detected in 65% of patients with an acute infectious disease (56% of all 794 diagnosed patients). A total of 36 different pathogens were actively detected using multiplex PCR, antigen detection, and serological investigation. Additional 20 pathogens were detected using standard culture techniques or in-house PCR. Altogether, 56 different pathogens from all major pathogenic subgroups were detected (FIG. 7A). This rate of pathogen identification is similar to that reported in previously published studies and included pathogens from all major pathogenic subgroups (Gram-negative bacteria, Gram-positive bacteria, atypical bacteria, RNA viruses, and DNA viruses). In 13% of the patients, pathogens from more than one of the aforementioned pathogenic subgroups were detected (FIG. 7A).

The pathogenic strains found in this study are responsible for the vast majority of acute infectious diseases in the Western world and included key pathogens such as influenza A/B, respiratory syncytial virus (RSV), parainfluenza, E. Coli, Group A Streptococcus, etc. Notably, analysis of the detected pathogens revealed that none of the pathogens is dominant (FIG. 7B).

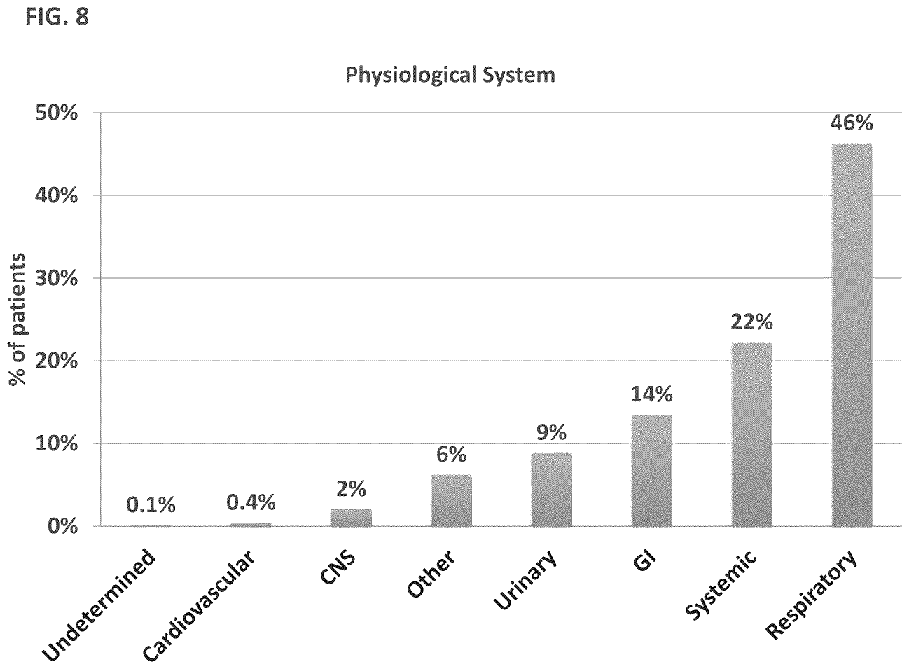

Involved physiologic systems and clinical syndromes: The infectious disease patients (all diagnosed patients [n=794], excluding those with non-infectious diseases or healthy subjects, n=673) presented with infections in a variety of physiologic systems (FIG. 8). The most frequently involved physiologic system was the respiratory system (46%), followed by systemic infections (22%). All infections that did not involve the aforementioned systems and were not gastrointestinal, urinary, cardiovascular, or central nervous system (CNS) infections were categorized as ‘Other’ (e.g., cellulitis, abscess). The observed distribution of physiologic system involvement represents the natural distribution and is consistent with that reported for large cohorts of patients sampled year-round.

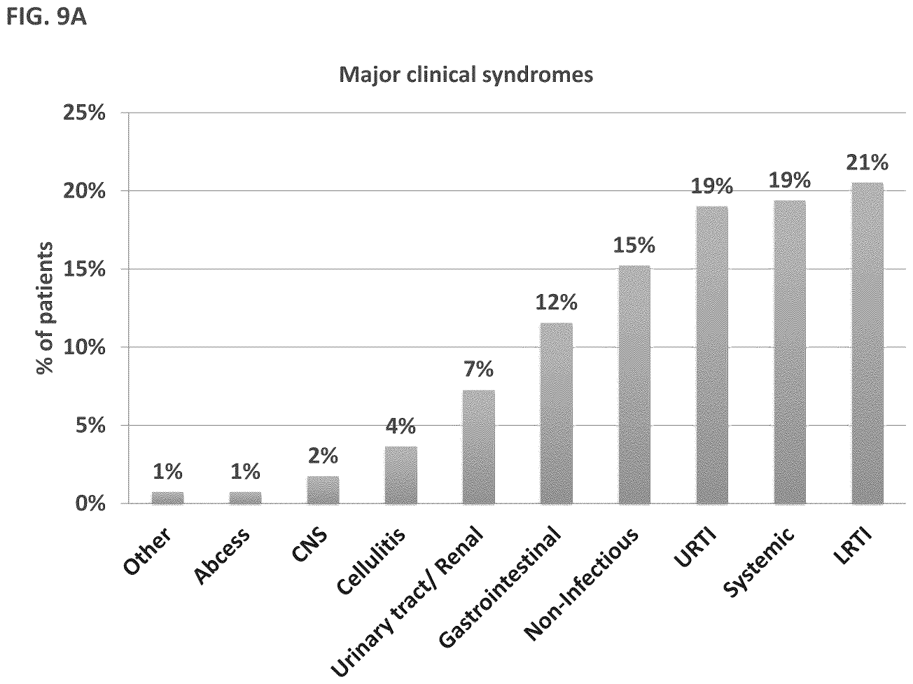

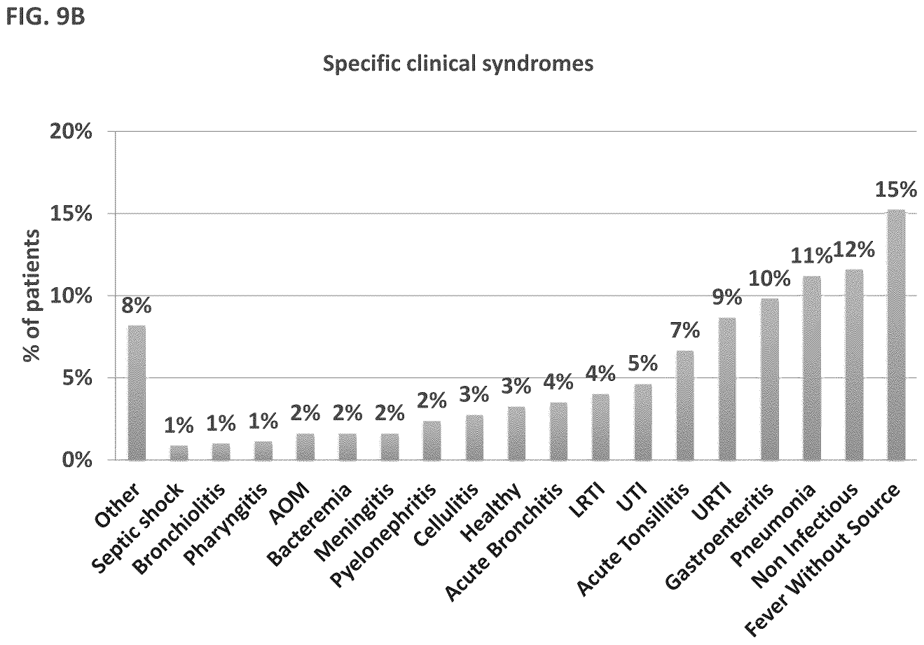

The diagnosed patients in the present study (n=794) presented with a variety of clinical syndromes (FIGS. 9A-9B) that reflects the expected clinical heterogeneity in a cohort of pediatric and adult patients collected year-round. The most frequent clinical syndrome was LRTI (21%) including mainly pneumonia, bronchitis, bronchiolitis, chronic obstructive pulmonary disease (COPD) exacerbation, and non-specific LRTI. The second most frequent syndrome was systemic infection (19%) including mainly fever without a source and occult bacteremia cases. Systemic infections were primarily detected in children <3 years of age but were also detected in a few adult patients. Systemic infections constitute a real clinical challenge as balancing between patient risk and the costs of testing/treatment is unclear. The third most frequent clinical syndrome was URTI (19%) including mainly acute tonsillitis, acute pharyngitis, non-specific URTI, acute sinusitis, and acute otitis media. The next most frequent syndromes were gastroenteritis (12%), UTI (7%), and cellulitis (4%). CNS infections (2%) included septic and aseptic meningitis. Additional clinical syndromes (1%) were classified as ‘Other’ and included less common infections (e.g., otitis externa, epididymitis, etc.). The observed pattern of clinical syndrome distribution represents most of the frequent and clinically relevant syndromes and is consistent with previously published large studies.

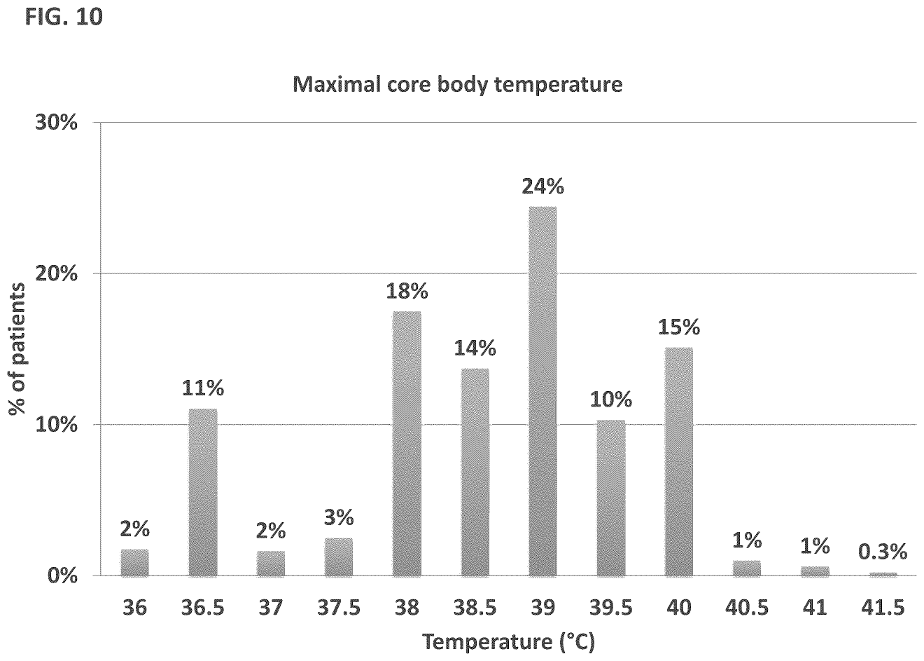

Core body temperature: Core body temperature is an important parameter in evaluating infectious disease severity. The distribution of maximal body temperatures was examined in all of the diagnosed patients (n=794) using the highest measured body temperature (per-os or per-rectum). The distribution of the maximal body temperatures was relatively uniform between 38° C. and 40° C. with a peak of at 39° C. (FIG. 10). Body temperature<37.5° C. was reported for 15% of patients (the subgroup of patients with non-infectious diseases or healthy subjects). Body temperature≥40.5° C. was rare (<3% of patients). Altogether, the observed distribution represents the normal range of temperatures in the clinical setting.

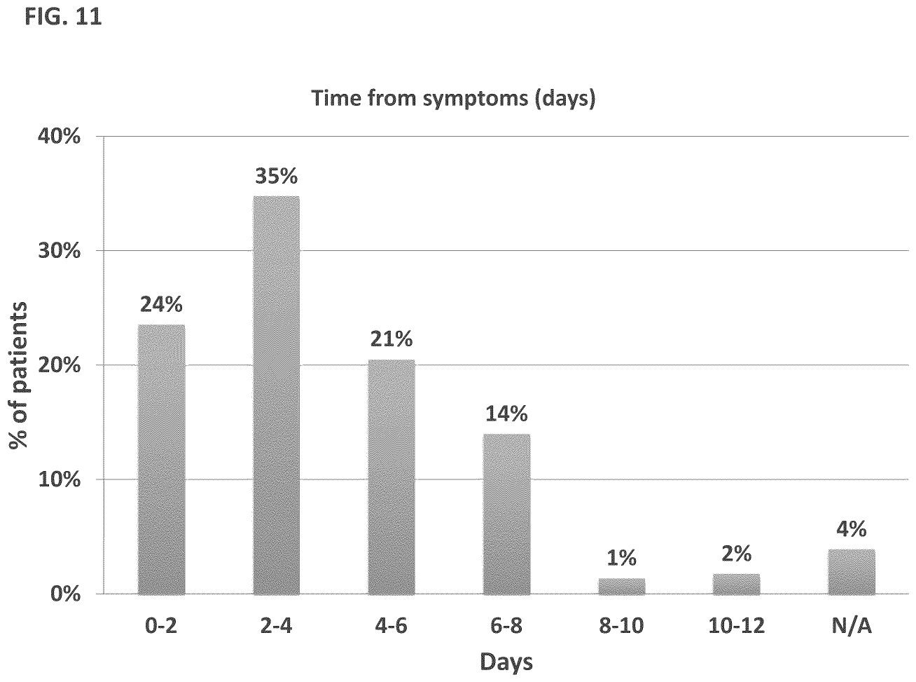

Time from symptoms onset: ‘Time from symptoms’ was defined as the duration (days) from the appearance of the first presenting symptom (the first presenting symptom could be fever but could also be another symptom such as nausea or headache preceding the fever). The distribution of ‘time from symptoms’ in our cohort (all diagnosed patients, n=794) peaked at 2-4 days after the initiation of symptoms (35% of patients) with substantial proportions of patients turning to medical assistance either sooner or later (FIG. 11).

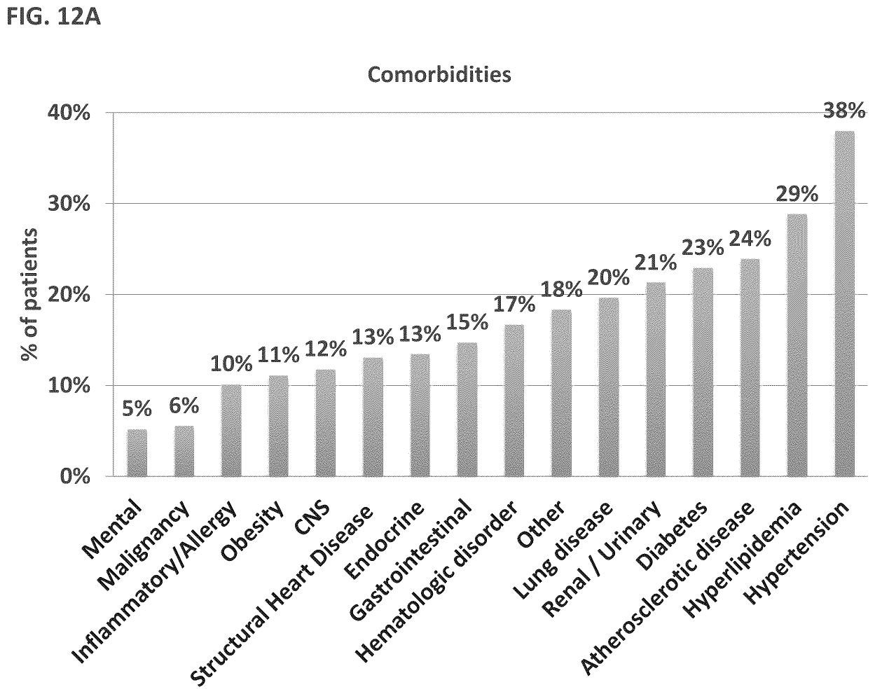

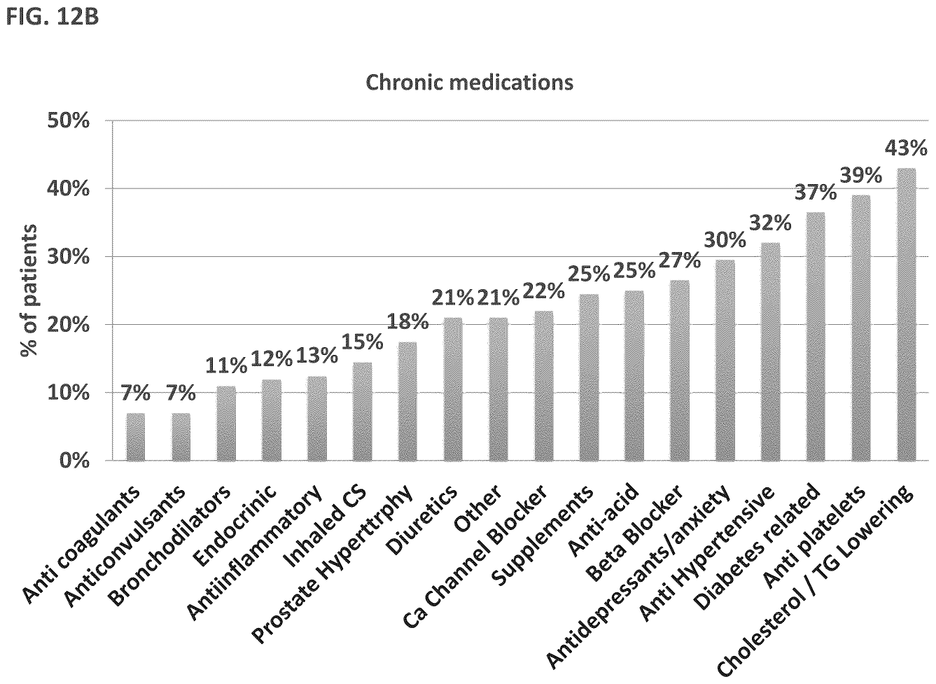

Comorbidities and chronic drug regimens: Comorbidities and chronic drug regimens may, theoretically, affect a diagnostic test. Out of the diagnosed patients 62% had no comorbidities whereas 38% had ≥1 chronic disease. In addition, 75% of patients were not treated with chronic medications and 25% were treated with ≥1 chronic medication. The most frequent chronic diseases in our patient population were hypertension, hyperlipidemia, lung diseases (e.g., COPD, asthma, etc.), diabetes mellitus (mostly type 2), and ischemic heart disease, mirroring the most common chronic diseases in the Western world (FIG. 12A). The distribution of chronic drugs used by our patient population strongly correlated with the range of reported chronic diseases (e.g., 29% of the patients with comorbidities had hyperlipidemia and lipid lowering agents were the most frequently used drugs). Other frequently used drugs included aspirin, blood glucose control drugs, and beta blockers (FIG. 12B).

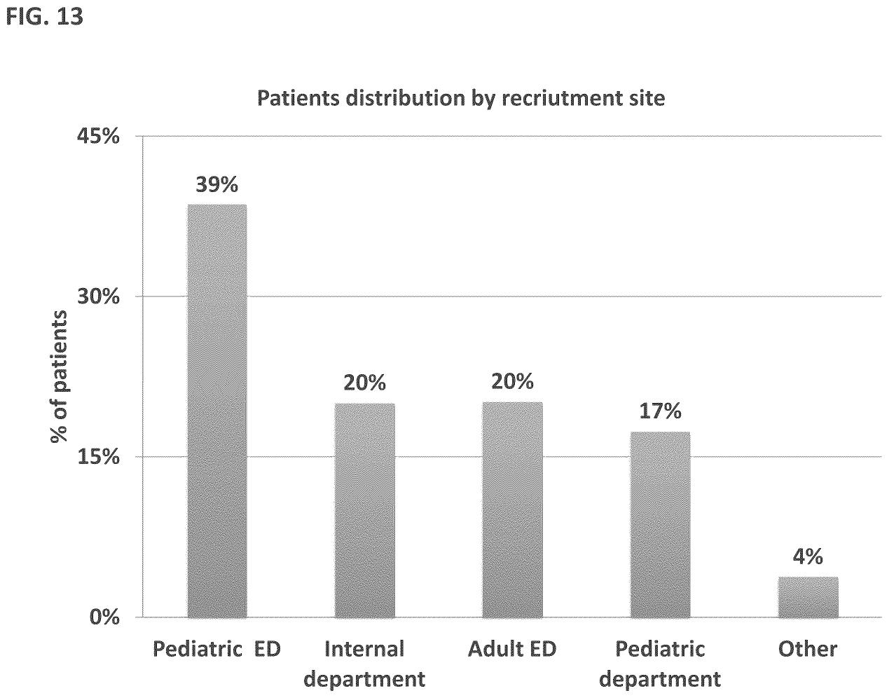

Patient recruitment sites: Pediatric patients (≤18 years) were recruited from pediatric emergency departments (PED), pediatric wards and surgical departments, and adults (>18 years) from emergency departments (ED), internal medicine departments and surgical departments. The pediatric ED was the most common recruitment site (39%) and the other sites were comparable (17-20%) reflecting a relatively balanced recruitment process. The ratio between ED patients and hospitalized patients was ˜1:1 for adults and ˜2:1 for children (FIG. 13).

Characteristics of excluded patients: Of the 1002 patients recruited for the study, 110 patients (11%) were excluded (some patients fulfilled more than one exclusion criterion). The most frequent reason for exclusion was having a fever below the study threshold of 37.5° C. (n=54), followed by time from symptom initiation of >12 days (n=26) and having a recent (in the preceding 14 days) infectious disease (n=22). Other reasons for exclusion included having an active malignancy (n=14), and being immunocompromised (e.g., due to treatment with an immunosuppressive drug; n=2).

Characteristics of indeterminate patients: A total of 98 patients were defined as indeterminate based on the inability of the expert panel to reliably establish a composite reference standard, despite the rigorous collection of laboratory and clinical information. While it is not possible to directly examine the signature performance in these patients in the absence of a reference standard, it is possible to analyze their host-protein response in order to assess whether they differ from patients with a reference standard. We compared the distribution of TRAIL, IP-10 and CRP in acute infection patients with a reference standard (n=653) to those without a reference standard (n=98). No statistically significant difference was observed (Kolmogorov Smirnov test P=0.20, 0.25, 0.46 for TRAIL, IP-10 and CRP, respectively). The similarity in the host-protein response between patients with and without a reference standard implies that the present approach may be useful for diagnosing indeterminate patients in the clinical setting.

The signature performance remains robust across different patient subgroups: In Example 1, the present inventors demonstrated that the signature remained robust across a wide range of patient characteristics including age, clinical syndrome, time from symptom onset, maximal temperature, pathogen species, comorbidities, and the clinical site with AUCs ranging from 0.87 to 1.0 (FIG. 4). In this Example, a review of the performance of the signature across additional patient subgroups is provided.

Stratification by chronic drug regimens: In real-world clinical practice, patients are often under various chronic drug regimens, which could, potentially, affect the level of proteins comprising the signature. The present inventors therefore examined whether the most used drugs (by categories) in our cohort impact the signature's performance. None of the evaluated drug groups were associated with significant alterations in the signature's accuracy (Table 5).

TABLE 5
Evaluation of the signature's sensitivity to various types of chronic
drug regimens.

| Viral | Bacterial | Total | | | |
|---|---|---|---|---|---|
| patients, | patients, | patients, | | | |
| n | n | n | AUC [95% CI] | | Drug category |
| 7 | 43 | 50 | [0.90, 1.00] | 0.95 | Anti Hypertensive |
| 6 | 48 | 54 | [0.96, 1.00] | 0.99 | Anti platelets |
| 7 | 35 | 42 | [0.80, 1.00] | 0.90 | Anti-acid |
| 4 | 25 | 29 | [0.93, 1.00] | 0.98 | Antidepressants |
| 5 | 35 | 40 | [0.88, 1.00] | 0.95 | Beta Blocker |
| 5 | 34 | 39 | [0.86, 1.00] | 0.94 | Ca Channel |
| | | | | | Blocker |
| 11 | 53 | 64 | [0.89, 1.00] | 0.94 | Cholesterol/TG |
| | | | | | Lowering |
| 5 | 35 | 40 | [0.74, 1.00] | 0.87 | Diabetic |
| 5 | 25 | 30 | [0.83, 1.00] | 0.93 | Diuretics |
| 4 | 14 | 18 | [0.93, 1.00] | 0.98 | Hormonal |
| 8 | 18 | 26 | [0.87, 0.99] | 0.95 | Inhaled CS |
| 4 | 21 | 25 | [0.84, 1.00] | 0.94 | Prostate |
| | | | | | Hypertrophy |

Sepsis based stratification: Sepsis is a potentially fatal medical condition characterized by a whole-body inflammatory state (called systemic inflammatory response syndrome [SIRS]) and the presence of a known or suspected infection. Patients with a bacterial sepsis benefit from early antibiotic therapy; delayed or misdiagnosis can have serious or even fatal consequences. The present inventors focused on adult patients for whom the definition of SIRS is clear and examined the ability of the signature to distinguish between adult patients with bacterial sepsis and those with viral infections as well as between adult patients with bacterial sepsis and those with viral sepsis.

Adult patients with bacterial sepsis were defined according to the American College of Chest Physicians and the Society of Critical Care Medicine. SIRS was defined by the presence of at least two of the following findings: (i) body temperature<36° C. or >38° C., (ii) heart rate>90 beats per minute, (iii) respiratory rate>20 breaths per minute or, on blood gas, a PaCO₂<32 mm Hg (4.3 kPa), and (iv) WBC<4,000 cells/mm³ or >12,000 cells/mm³ or >10% band forms. It was found that the signature achieved very high levels of accuracy in distinguishing between adult patients with bacterial sepsis and those with viral sepsis (AUC of 0.97 and 0.93 for the Unanimous [adult bacterial sepsis, adult viral sepsis] and the Majority [adult bacterial sepsis, adult viral sepsis] cohorts, respectively). These results demonstrate the utility of the signature in differentiating adult patients with bacterial sepsis from adult patients with viral infections.

TABLE 6
Signature accuracy in diagnosing bacterial sepsis vs. viral sepsis in
adult patients

| Viral | Bacterial | Total | | | |
|---|---|---|---|---|---|
| patients, n | patients, n | patients, n | AUC [95% CI] | | |
| 21 | 93 | 114 | [0.94, 1.00] | 0.97 | Unanimous |
| 35 | 112 | 147 | [0.89, 0.97] | 0.93 | Majority |

Bacterial vs. non-bacterial patients stratification: Antibiotic misuse typically stems from the use of these drugs to treat non-bacterial (viral or non-infectious) patients or due to delayed or missed diagnosis of bacterial infections.

Therefore, the present inventors further examined the signature performance for distinguishing between bacterial and non-bacterial patients. The entire Majority cohort was evaluated using leave-10%-out cross-validation, yielding an AUC of 0.94±0.02. Improved performances were shown when evaluating the Unanimous cohort (AUC of 0.96±0.02), and after filtering out patients with a marginal immune response (Table 7).

TABLE 7
Signature measures of accuracy for diagnosing bacterial vs. non-
bacterial (viral and non-infectious) patients.

| B. Marginal | | | | |
|---|---|---|---|---|
| immune response filter | | A. All patients | | |
| Majority | Unanimous | Majority | Unanimous | Accuracy |
| cohort | cohort | cohort | cohort | measure |
| 0.95 | 0.96 | 0.94 | 0.96 | AUC |
| (0.93, 0.97) | (0.94, 0.98) | (0.92, 0.96) | (0.94, 0.98) | |
| 0.91 | 0.93 | 0.88 | 0.91 | Total accuracy |
| (0.89, 0.93) | (0.91, 0.95) | (0.85, 0.91) | (0.89, 0.93) | |
| 0.91 | 0.92 | 0.87 | 0.88 | Sensitivity |
| (0.88, 0.95) | (0.88, 0.95) | (0.83, 0.91) | (0.85, 0.91) | |
| 0.92 | 0.94 | 0.90 | 0.93 | Specificity |
| (0.89, 0.95) | (0.91, 0.96) | (0.87, 0.93) | (0.91, 0.95) | |
| 11.4 | 15.3 | 8.7 | 12.6 | LR+ |
| (8, 16) | (10, 23) | (6, 12) | (9, 18) | |
| 0.1 | 0.08 | 0.14 | 0.13 | LR− |
| (0.07, 0.14) | (0.05, 0.13) | (0.11, 0.19) | (0.09, 0.18) | |
| 116 | 180 | 60 | 97 | DOR |
| (67, 200) | (94, 344) | (38, 94) | (56, 168) | |

A. Performance estimates and their 95% CIs were obtained using a leave-10%-out cross-validation on all patients in the Unanimous (n_{Bacterial} = 256, n_{Non-bacterial} = 383), and Majority (n_{Bacterial} = 319, n_{Non-bacterial} = 446) cohorts. B. The analysis was repeated after filtering out patients with a marginal immune response (Unanimous [n_{Bacterial} = 237, n_{Non-bacterial} = 343, n_{Marginal} = 59], and Majority [n_{Bacterial} = 292, n_{Non-bacterial} = 387, n_{Marginal} = 86]), which resembles the way clinicians are likely to use the signature.

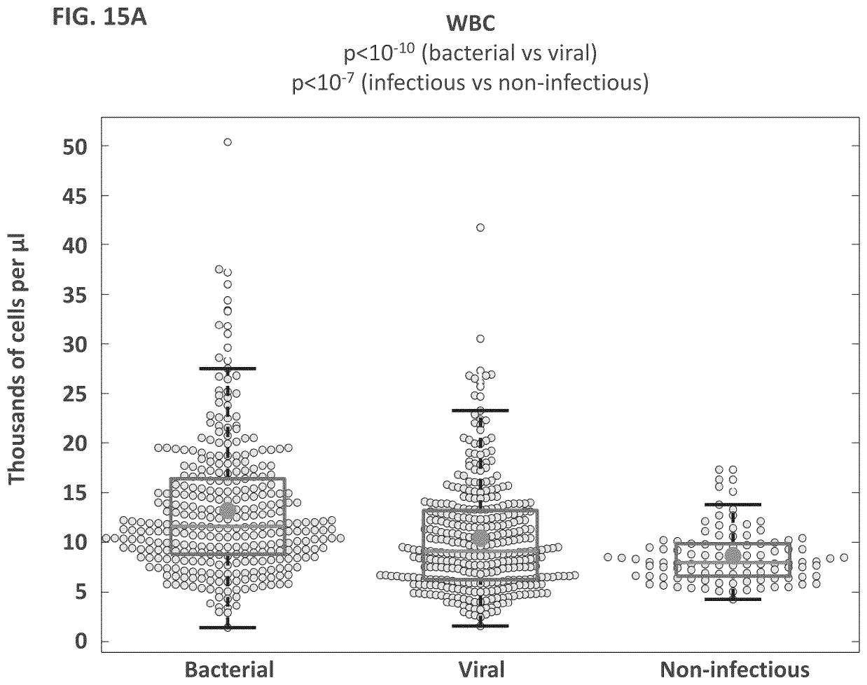

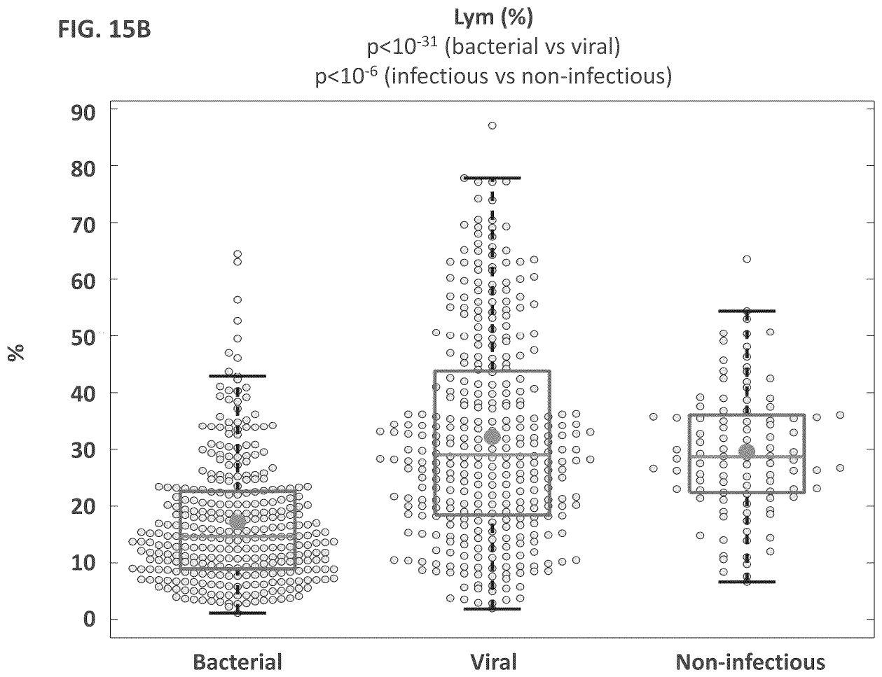

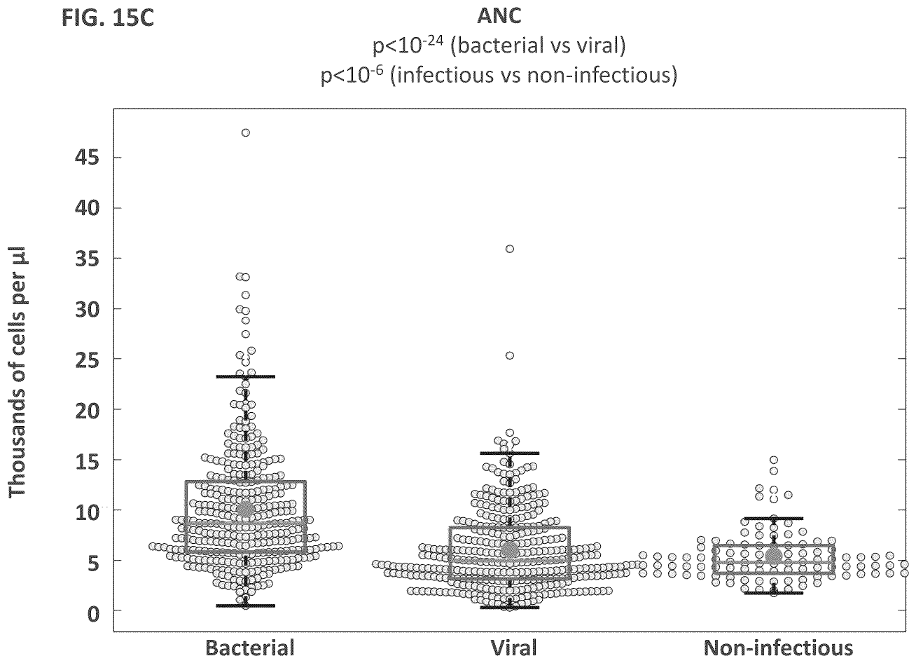

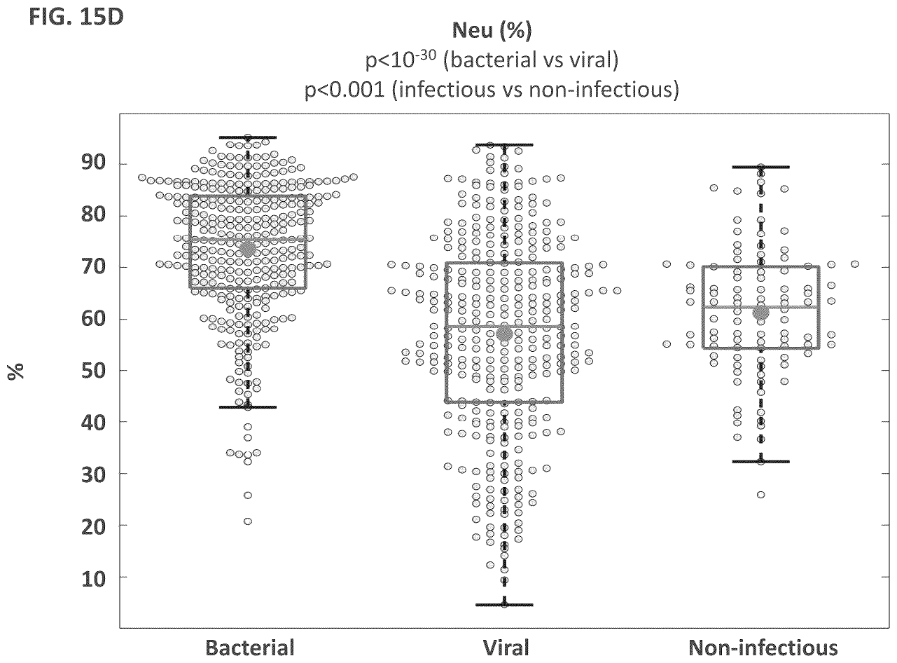

Protein stability at different temperatures can affect the assay performance: The utility of a biomarker depends on its stability in real-life clinical settings (e.g., its decay rate when the sample is stored at room temperature prior to analyte measurement). To address this, we examined the stability of TRAIL, CRP and IP-10 in serum samples from four independent individuals during 24 hours at 4° C. and 25° C. Aliquots of 100 μL from each plasma sample were pipetted into 0.2 mL tubes and kept at 4° C. or 25° C. from 0 to 24 hours. Subsequently, the levels of the analytes were measured (different time-points of the same analytes were measured using the same plate and reagents). The analyte half-lives at 4° and 25° C. were greater than 72 hours for TRAIL, CRP and IP-10 (FIGS. 15A-15C). Of note, in the real clinical setting, if the samples are stored at room temperature, the concentrations of TRAIL, IP-10 and CRP should be measured within about 24 after the sample is obtained. Preferably they should be measured within 5 hours, 4 hours, 3 hours, 2 hours, 1 hour, or even immediately after the sample was obtained. Alternatively, the sample should be stored at a temperature lower than 10° C., and then TRAIL can be measured more than 24 after obtaining the sample.

The three protein combination outperforms any individual and pairs of proteins: The combination of the three proteins outperforms that of the individual and pairs of proteins for distinguishing bacterial vs. viral and infectious vs. non-infectious patients.

TABLE 8
Bacterial vs. viral

| | Proteins | Protein | Protein |
|---|---|---|---|
| AUC | #3 | #2 | #1 |
| 0.89 | — | — | TRAIL |
| 0.88 | — | — | CRP |
| 0.66 | — | — | IP-10 |
| 0.95 | — | CRP | TRAIL |
| 0.93 | — | IP-10 | CRP |
| 0.90 | — | IP-10 | TRAIL |
| 0.96 | IP-10 | CRP | TRAIL |

TABLE 9
Infectious vs. Noninfectious

| | Proteins | Protein | Protein |
|---|---|---|---|
| AUC | #3 | #2 | #1 |
| 0.60 | — | — | TRAIL |
| 0.87 | — | — | CRP |
| 0.89 | — | — | IP-10 |
| 0.90 | — | CRP | TRAIL |
| 0.95 | — | IP-10 | CRP |
| 0.89 | — | IP10 | TRAIL |
| 0.96 | IP-10 | CRP | TRAIL |

Performance analysis as a function of the prevalence of bacterial infections: The prevalence of bacterial and viral infections is setting dependent. For example, in the winter, a pediatrician in the outpatient setting is expected to encounter substantially more viral infections than a physician in the hospital internal department during the summer. Notably, some measures of diagnostic accuracy such as AUC, sensitivity, and specificity are invariant to the underlying prevalence, whereas other measures of accuracy, such as PPV and NPV are prevalence dependent. In this section, the expected signature performance in terms of PPV and NPV in clinical settings with different prevalence of bacterial and viral infections is reviewed.

As the basis for this analysis the signature accuracy measures were used that were obtained using the Unanimous (bacterial, viral) and Majority (bacterial, viral) cohorts. The prevalence of bacterial infections in the Unanimous cohort was 51.7% yielding a PPV of 93%±3% and NPV of 93%±3%. The prevalence of bacterial infections in the Majority cohort was 48.7% yielding a PPV of 89%±3% and NPV of 92%±3%.

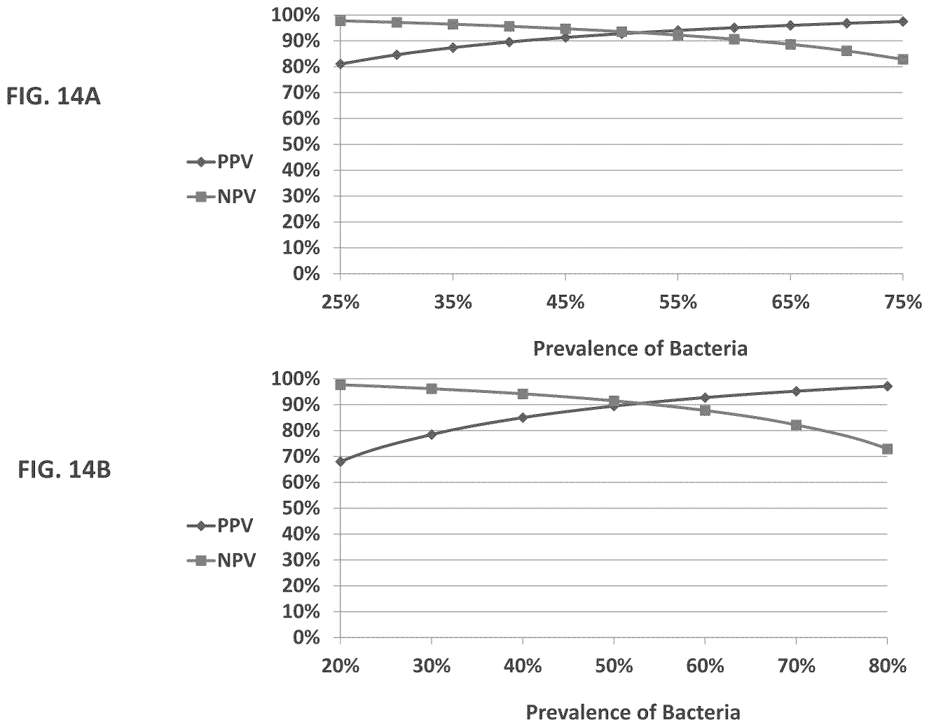

The measured sensitivity and specificity was used to compute the expected changes in the signature PPV and NPV as a function of the prevalence of bacterial infections (FIGS. 14A-14B).

Examples of different clinical settings and the extrapolated signature PPV and NPV for each of them are presented in Table 10A.

TABLE 10A
Extrapolated signature PPV and NPV in different
clinical settings, based on the Unanimous cohort.

| | | Prevalence | | |
|---|---|---|---|---|
| | | of Bacterial | | |
| NPV | PPV | infections* | Age | Setting |
| 98% | 76% | 20% | Children | Outpatient |
| 97% | 85% | 35% | Adults | Outpatient |
| 94% | 93% | 50% | Children | Inpatient |
| 78% | 98% | 80% | Adults | Inpatient |

*An average annual prevalence. Estimates of bacterial infection prevalence are based on data reported in the Bacterial etiology chapter, Part 7 of Harrison's Internal Medicine 17^{th} Edition.

The signature outperforms standard laboratory and clinical parameters for diagnosing bacterial vs. viral infections: Standard laboratory and clinical parameters, some of which are routinely used in clinical practice to aid in the differential diagnosis of an infection source, were evaluated in the Majority cohort (bacterial, viral, non-infectious, n=765). The evaluated parameters included ANC, % neutrophils, % lymphocytes, WBC, and maximal temperature. In accordance with the well-established clinical role of these parameters, we observed a statistically significant difference in their levels between bacterial and viral patients (FIGS. 15A-15E). For example, bacterial patients had increased levels of ANC (P<10⁻²⁴), and WBC (P<10⁻¹⁰), whereas viral patients had a higher % lymphocytes (P<10⁻³¹). The signature was significantly more accurate than any of the individual features (P<10⁻¹⁸) and their combinations (P<10⁻¹⁵), see FIG. 3A).

The signature outperforms protein biomarkers with a well-established immunological role: The signature outperformed all clinical parameters and the 600 proteins that were evaluated during the screening phase (see FIGS. 3A-3B). The following section further compares the signature to selected proteins that are routinely used in the clinical setting or that have an immunological role.

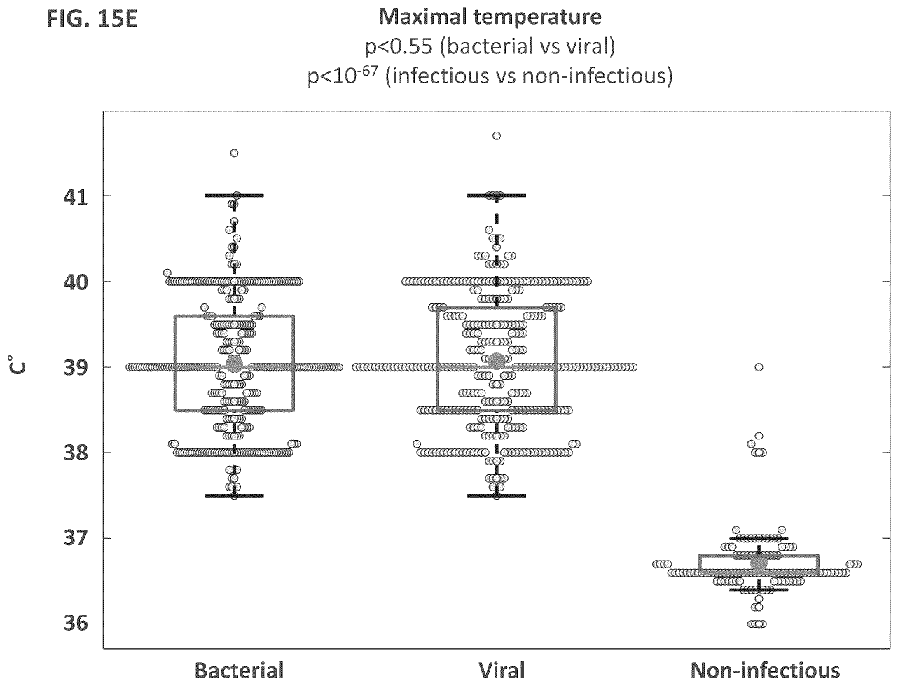

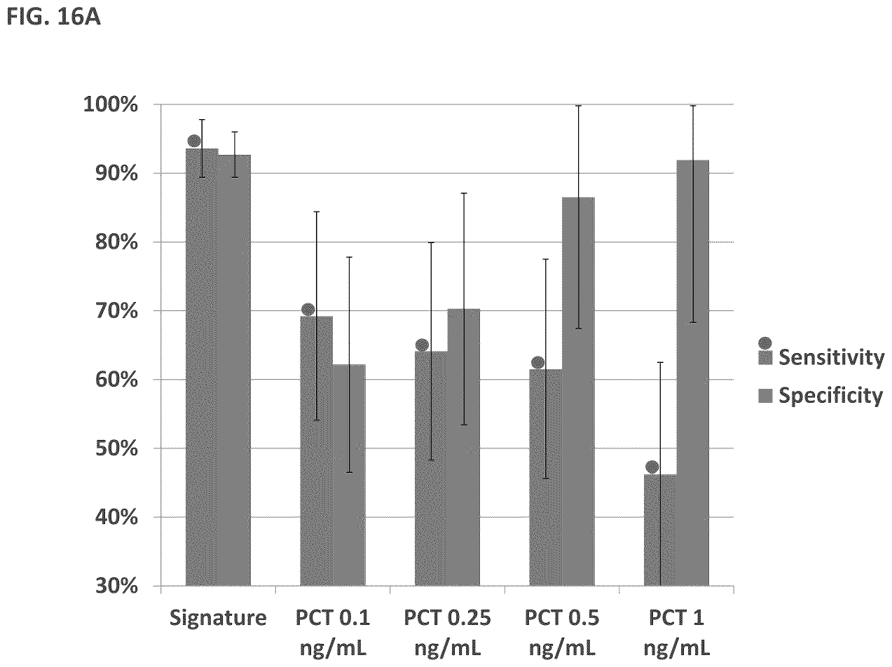

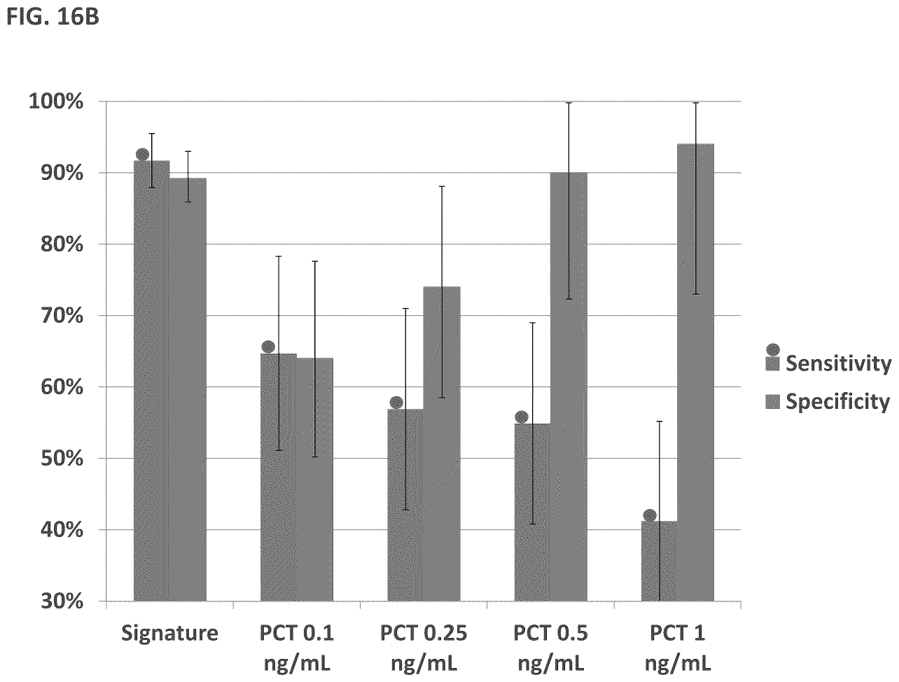

One of the most widely used and useful protein biomarkers for differentiating sepsis from other non-infectious causes of SIRS in critically ill patients is procalcitonin (PCT). Whether PCT can be used to distinguish between local bacterial and viral infections is less clear. To test this, we measured PCT concentrations in 76 randomly selected patients from the Unanimous (bacterial, viral) cohort (n_{Bacterial}=39, n_{Viral}=37) and 101 randomly selected patients from the Majority (bacterial, viral) cohort (n_{Bacterial}=51, n_{Viral}=50) and compared the diagnostic accuracy based on PCT levels to that of the signature. PCT accuracy was calculated using the standard cutoffs routinely applied in the clinical setting (0.1 ng/ml, 0.25 ng/ml, 0.5 ng/ml, and 1 ng/mL.¹⁹⁻²³ Maximal PCT sensitivity of 69% was attained at a cutoff of 0.1 mg/mL and resulted in a specificity of 62% (for the Unanimous [bacterial, viral] cohort). For the same cohort, the signature showed significantly higher sensitivity of 94% (P<0.001) and specificity of 93% (P<0.001) (FIG. 16A). A comparison using the patients from the Majority (bacterial, viral) cohort showed similar results (FIG. 16B).

Overall, despite its high diagnostic and prognostic value for sepsis detection in critically ill patients, our results indicate that PCT is less accurate in distinguishing between patients with local infections (bacterial vs. viral).

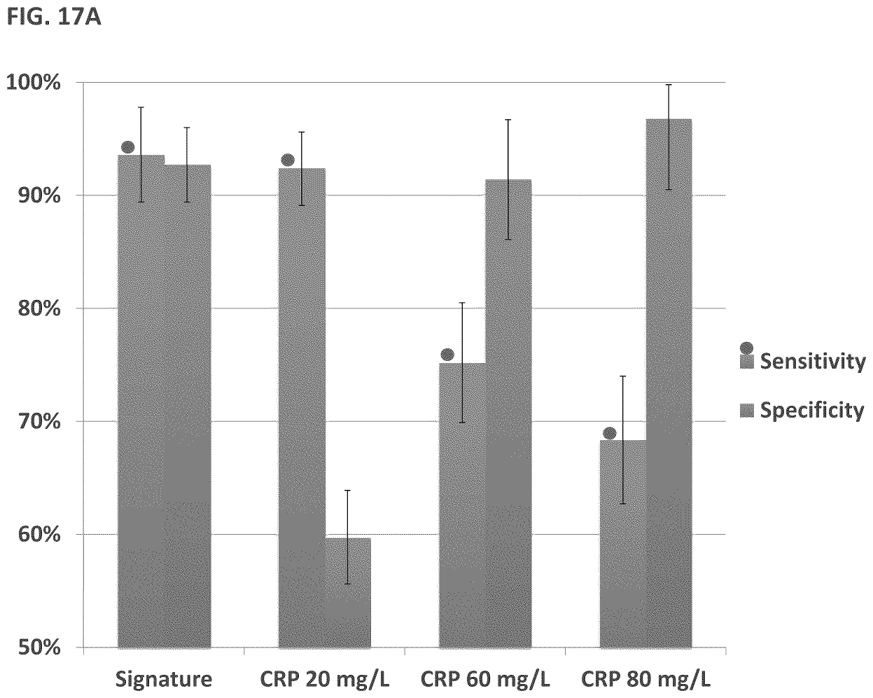

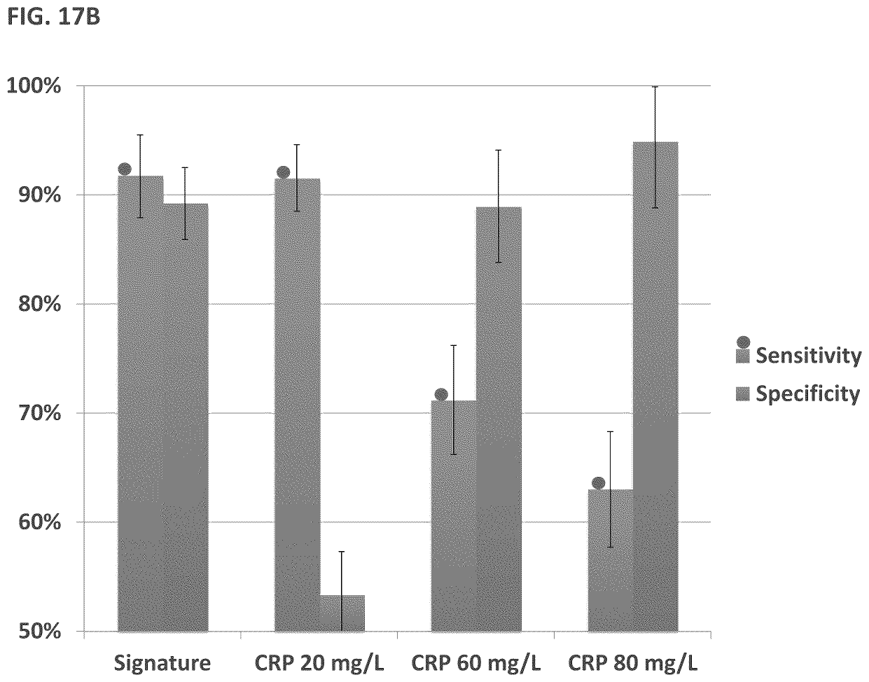

Another protein biomarker used in the clinical setting is the C-reactive protein (CRP), an acute phase response protein that is up-regulated in infections and other inflammatory conditions. The performance of CRP was compared to that of the signature using the entire Unanimous (bacterial, viral) and Majority (bacterial, viral) cohorts. CRP accuracy was determined using several standard cutoffs applied in the clinical setting.²⁴⁻²⁶ Maximal CRP sensitivity of 92% was attained at 20 mg/mL cutoff resulting in a specificity of 60% (for the Unanimous [bacterial, viral] cohort) (FIG. 17A). The signature had a similar sensitivity (94%) and a significantly higher specificity (93%, P<10⁻⁹) in the same cohort. Similar results were observed using the Majority (bacterial, viral) cohort (FIG. 17B). Overall, the signature has a similar sensitivity to CRP with a 20 mg/L cutoff but a considerably higher specificity for distinguishing bacterial from viral patients.

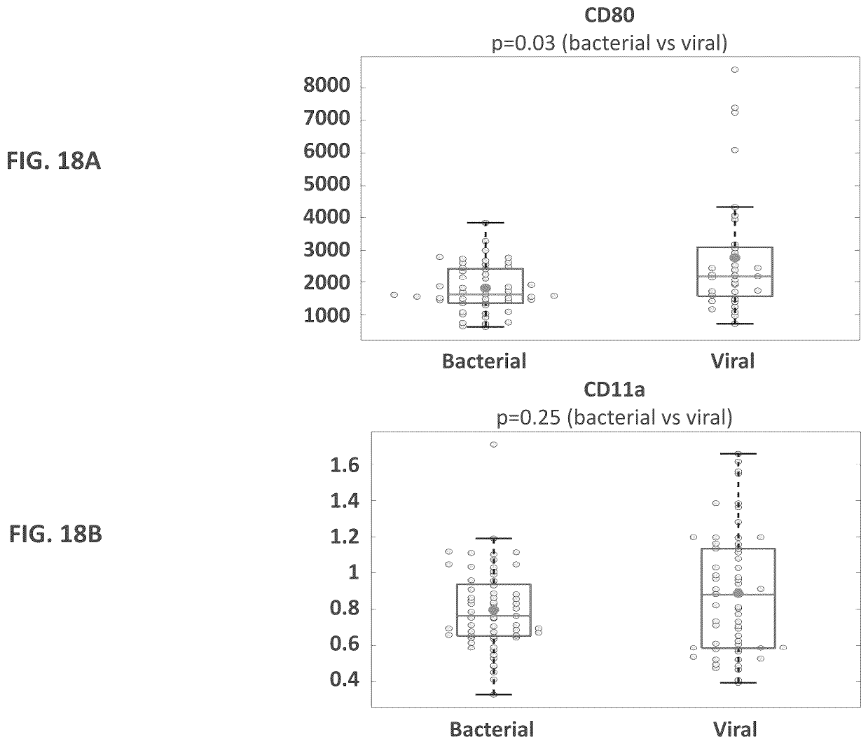

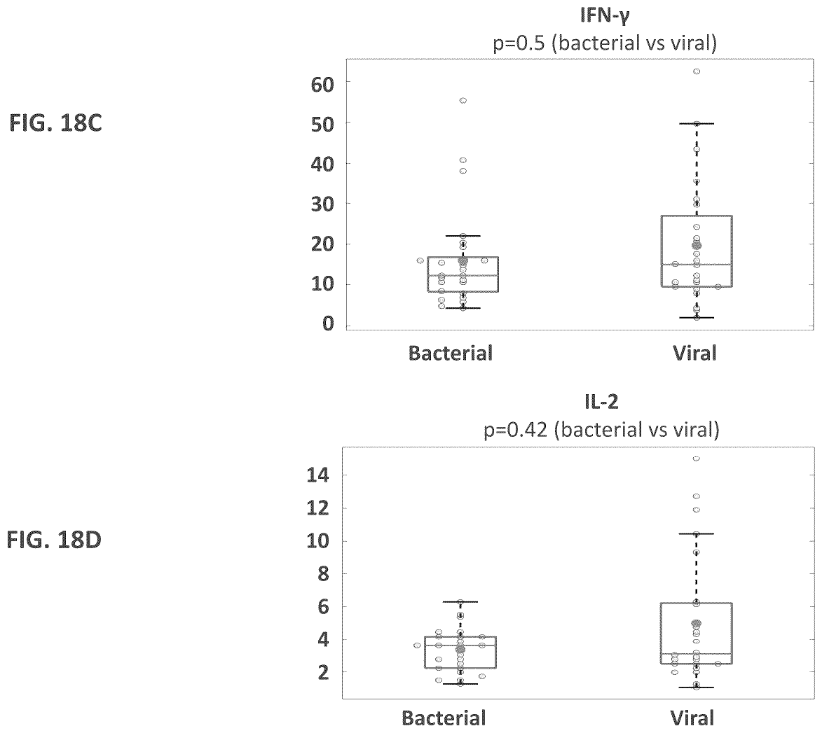

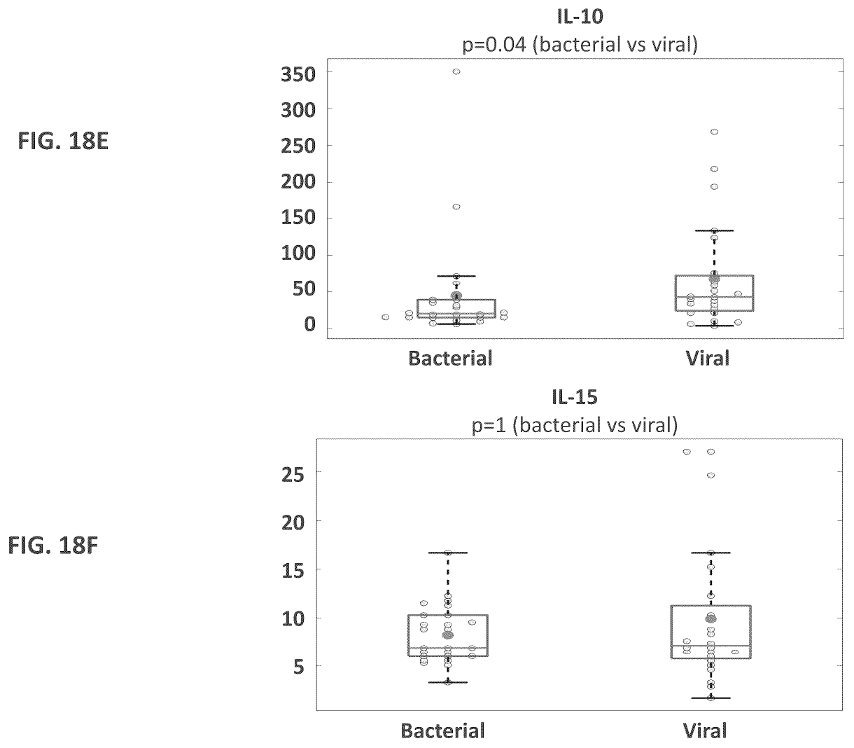

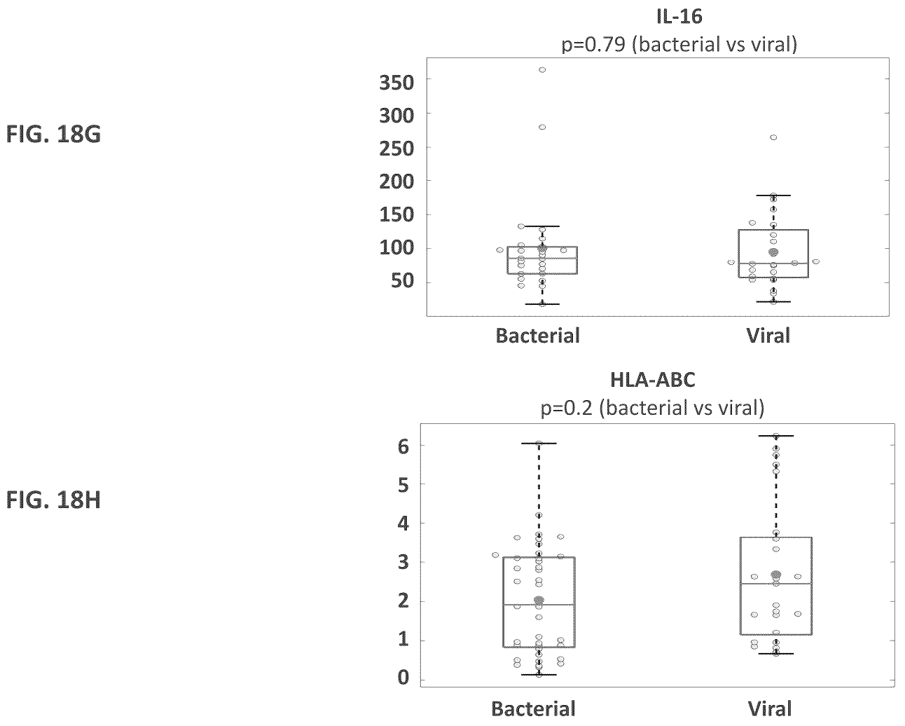

Next, the differential response of protein biomarkers with a well-established role in the host response to infections was examined (Table 10B and FIGS. 18A-18H). Each biomarker was tested on at least 43 patients (about half bacterial and half viral), and if it showed promising results, it was further tested on additional patients (up to 150).

TABLE 10B
A list of protein biomarkers with a well-established role
in the host response against infections, and the number of
patients used to test each biomarker (for each analysis the analyzed
patients included approximately half bacterial and half viral patients).

| No. of | | Protein |
|---|---|---|
| patients | Short description | biomarker |
| 120 | CD11a is expressed by all leukocytes as part of the integrin | CD11a |
| | lymphocyte function-associated antigen-1 (LFA-1). LFA-1 | |
| | plays a central role in leukocyte intercellular adhesion through | |
| | interactions with its ligands, ICAMs 1-3 (intercellular adhesion | |
| | molecules 1 through 3). CD11a also functions in lymphocyte | |
| | co-stimulatory signaling. | |
| 79 | CD11C is an integrin α X chain protein and mediates cell-cell | CD11C |
| | interactions during inflammatory responses. | |
| 82 | CD80 is a membrane receptor involved in the co-stimulatory signal | CD80 |
| | essential for T-lymphocyte activation. The binding of CD28 or | |
| | CTLA-4 to CD80 induces T-cell proliferation and cytokine | |
| | production. | |
| 65 | These are MHC class I antigens associated with β2-microglobulin | HLA-A, B, C |
| | and are expressed by all human nucleated cells. HLA-A, B, C are | |
| | central in cell-mediated immune response and tumor surveillance. | |
| 49 | IFN-γ is a soluble cytokine. IFN-γ participates in innate and adaptive | IFN-γ |
| | immunity against viral and intracellular bacterial infections and in | |
| | tumor control. | |
| 43 | IL-1a is a member of the IL-1 cytokine family. IL-1a is a pleiotropic | IL-1a |
| | cytokine involved in various immune responses, inflammatory | |
| | processes, and hematopoiesis. IL-1a is produced by monocytes and | |
| | macrophages as a proprotein, which is proteolytically processed and | |
| | released in response to cell injury, thereby inducing apoptosis. | |
| 49 | IL-2 is produced by T-cells in response to antigenic or mitogenic | IL-2 |
| | stimulation. IL-2 is required for T-cell proliferation and other | |
| | activities crucial for regulation of the immune response. | |
| 43 | IL-6 is a cytokine that functions in inflammation and maturation of B | IL-6 |
| | cells. IL-6 is an endogenous pyrogen capable of inducing fever in | |
| | people with autoimmune diseases or infections. | |
| 43 | IL-8 is a member of the CXC chemokine family and functions as one | IL-8 |
| | of the major mediators of the inflammatory response. | |
| 43 | IL-9 is a cytokine that acts as a regulator of a variety of | IL-9 |
| | hematopoietic cells. | |
| | IL-9 supports IL-2 independent and IL-4 independent growth of | |
| | helper T-cells. | |
| 48 | IL-10 is a cytokine produced primarily by monocytes and to a lesser | IL-10 |
| | extent by lymphocytes. IL-10 has pleiotropic effects in | |
| | immunoregulation and inflammation. | |
| 49 | IL-15 is a cytokine that stimulates the proliferation of T-lymphocytes. | IL-15 |
| 49 | IL-16 functions as a chemo-attractant, a modulator of T cell | IL-16 |
| | activation, and an inhibitor of HIV replication. | |
| 54 | sTNFRSF1A is a receptor for TNFSF2/TNF-α and homo-trimeric | sTNFRSF1A |
| | TNFSF1/lymphotoxin-α that contributes to the induction of non- | |
| | cytocidal TNF effects including anti-viral state and activation of the | |
| | acid sphingomyelinase. | |
| 43 | TNF-α is a cytokine secreted mainly by macrophages. TNF-α can | TNF-α |
| | induce cell death of certain tumor cell lines. It is a potent pyrogen | |
| | causing fever directly or by stimulation of IL-1 secretion. | |
| 43 | TNF-β is a potent mediator of inflammatory and immune responses. | TNF-β |
| | It is produced by activated T and B lymphocytes and is involved in | |
| | the regulation of various biological processes including cell | |
| | proliferation, differentiation, apoptosis, lipid metabolism, | |
| | coagulation, and neurotransmission. | |
| 150 | TREM is a pro-inflammatory amplifier present on neutrophils and | TREM |
| | monocytes. | |

Since these biomarkers do not have a well-established cutoff in the clinical setting, we used their AUCs as a basis for comparison (FIG. 3B) The most informative biomarker was TREM (AUC of 0.68±0.09). The accuracy of TREM was significantly lower than that of the signature (P<10⁻⁹ when comparing the two AUCs; FIG. 3B). These results demonstrate that mere participation of a protein in the host response to an infection does not necessarily imply diagnostic utility. For example, although IFN-γ has a well-established role in the immune response to viruses and intracellular bacteria, its short half-life (<20 h)²⁷ limits its diagnostic utility (as its concentration in the blood is highly dependent on the time from infection onset).

### Example 3

### Trinary Classifier Outperforms a Binary Classifier

In the binary model the classifier is trained by classifying all samples as either ‘Bacterial’ or ‘Non-bacterial’ (‘Viral’ and ‘Non-infectious’ are grouped). In the trinary model, the classifier learns to distinguish between three classes ‘Bacterial’, ‘Viral’ and ‘Non-infectious’. The probability of the viral and the non-infectious are then grouped together to give the probability of ‘non-bacterial’. This was demonstrated on the present data.

Both of the above classifiers were evaluated using a leave 10%-out cross-validation on both the Majority and Unanimous cohorts.

### Results

Running the binary classifier on the majority cohort yields the results as summarized in Table 10C, herein below:

TABLE 10C

| Reference class | | |
|---|---|---|
| | Viral and | |
| | non- | |
| Bacterial | infectious | |
| (B) | (V + NI) | |
| 63 | 411 | V + NI |
| 256 | 35 | B |

The sensitivity of the classifier on the Majority cohort is 80.3% and the specificity is 92.2%.

Running the multinomial based classifier on the same dataset yields the following results summarized in Table 10D.

TABLE 10D

| Reference class | | |
|---|---|---|
| (B) | (V + NI) | |
| 54 | 417 | V + NI |
| 265 | 29 | B |

It can be seen that this classifier outperforms the previous one both in terms of sensitivity and in terms of specificity. The sensitivity was improved to 83.1% and the specificity to 93.5%.

Running the binary classifier on the Unanimous cohort yields the results summarized in Table 11.

TABLE II

| Reference class | | |
|---|---|---|
| (B) | (V + NI) | |
| 39 | 358 | V + NI |
| 217 | 25 | B |

The sensitivity of the classifier on the Unanimous cohort is 84.8% and the specificity is 93.5%.

Running the multinomial based classifier on the same dataset yields the results summarized in Table 12.

TABLE 12

| Reference class | | |
|---|---|---|
| (B) | (V + NI) | |
| 38 | 364 | V + NI |
| 218 | 19 | B |

This classifier outperforms the previous one both in terms of sensitivity and in terms of specificity. The sensitivity was improved to 85.2% and the specificity to 95.0%.

In summary, the trinary classifier outperforms the binary based classifier both in terms of sensitivity and in terms of specificity on both datasets tested.

### Example 4

The Clinical Accuracy of the Signature Remains Robust Even when Analytical Accuracy is Reduced

It is important to assess how clinical accuracy is affected by the increase in the CV (std/mean) of the proteins measurements, because often different measurement devices, particularly those that are useful at the point-of-care, show increased CVs (i.e. reduced analytical accuracy).

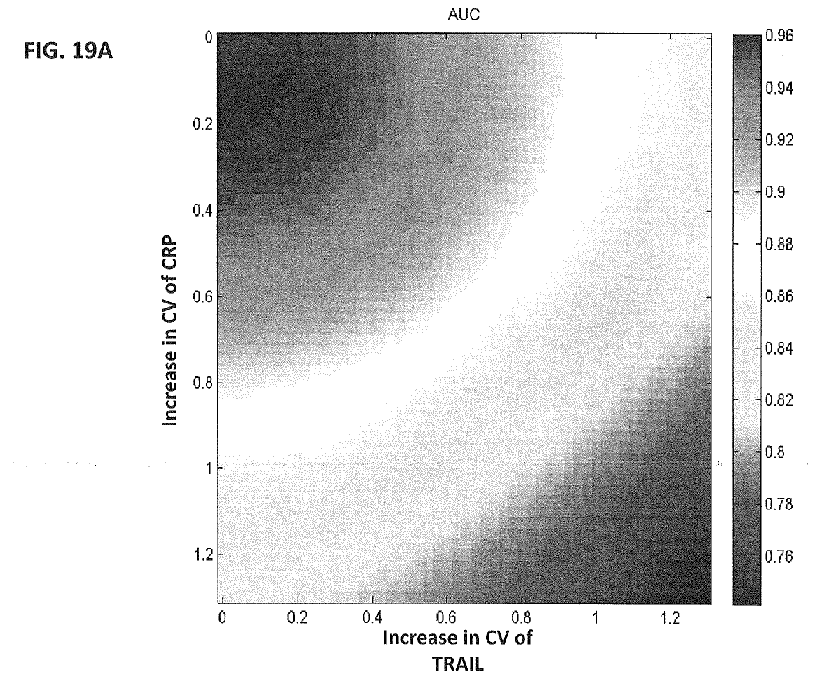

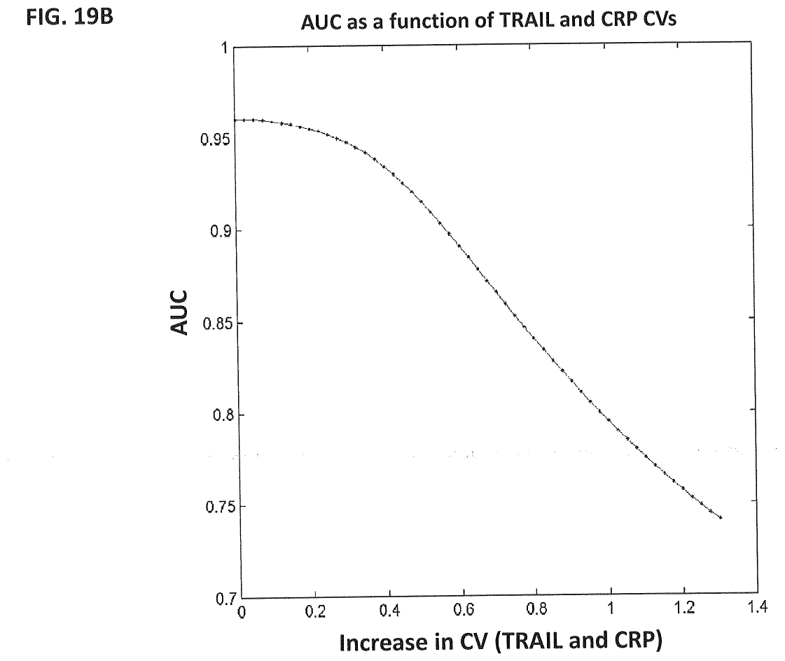

The present inventors examined the change in AUC of the signature for distinguishing bacterial from viral infection as a function of the increase in CV of both TRAIL and CRP. This was done by taking the original patient data of the Unanimous cohort and simulating an increase in CV using monte-carlo simulations (FIGS. 19A-19B). Specifically, for each combination of TRAIL and CRP CVs, 100 simulated measurements were assigned to each of the patients and the AUC in each case was recomputed. The average AUC per CV combination is depicted. It can be seen that the signature clinical accuracy (in terms of AUC) is robust to the increases in technical CV. For example, increasing the ELISA CV by 0, 0.24 and 0.4 leads to a reduction in AUCs of 0.96, 0.95 and 0.94 respectively. Similar results are obtained when increasing the CV of IP-10, and when repeating the simulations on the Majority cohort.

This result may be explained by the usage of multiple biomarkers that compensate for one another. This surprising finding is useful because it opens the way to perform measurements of the proteins on cheap and rapid technologies (such as POC technologies), which often show reduced analytical sensitivity (compared for example to automated immunoassays or ELISA), without losing clinical accuracy.

### Example 5

Different ELISA protocols can be applied for measuring TRAIL and IP-10, which would lead to different signal to noise ratios, and consequentially to different concentrations being measured. More specifically, while the overall trend of the biomarkers will be preserved regardless of the protocol (e.g. TRAIL increases in viral infections and decreases in bacterial), the measurement scale is protocol dependent. In the following subsections, examples of protocols are described that lead to different measured concentrations of IP-10 and TRAIL.

Measurements of soluble IP-10 and TRAIL using ELISA—Protocol no. 1: To determine the concentrations of soluble IP-10 and TRAIL in human plasma and serum samples, a standard Sandwich ELISA (Enzyme-linked immunosorbent assay) was used. Briefly, the wells of 96-well plate were coated with capture-antibody specific to TRAIL and IP-10 and diluted in coating buffer (e.g. 1×PBS) followed by overnight incubation at 4° C. The wells were washed twice with washing buffer (e.g. 1×PBS with 0.2% Tween-20) and subsequently blocked with blocking buffer containing proteins (e.g. 1×PBS with 0.2% Tween-20 and 5% non-fat milk) for at least 2 hours at room temperature or overnight at 4° C. Wells were then washed twice with washing buffer. Protein standards and plasma or serum samples were incubated for two hour at room temperature. Then, the wells were washed three times with a washing buffer and subsequently incubated with an HRP conjugated detection-antibody specific to TRAIL and IP-10, diluted in blocking buffer for two hours at room temperature.

The wells were washed four times with a washing buffer and incubated with a reaction solution that contained an HRP substrate (e.g. TMB; 3, 3′, 5,5′-Tetramethylbenzidine). After adequate color development, a stop solution was added to each well. The absorbance of the HRP reaction product in 450 nm was determined using standard spectrophotometer. This protocol took 5 (TRAIL) and 4.75 (IP10) hours respectively and is referred to herein as the slow protocol.

### Measurements of Soluble IP-10 and TRAIL Using ELISA—Protocol No. 2:

Reducing assay time allows for increased clinical utility. To further reduce the protocol run time, the protocol was optimized for measuring TRAIL and IP10 and reduced to less than 100 minutes. The rapid protocol was performed as follows:

50 μl of assay diluent and 50 μl of Standards was added to samples or controls per well. The reaction was incubated for 30 minutes at room temperature on a horizontal orbital microplate shaker (3 mm orbit) set at 550 rpm. Each well was then aspirated and washed four times by using a wash buffer. Next, 200 μl of Conjugate was added to each well and the reactions were incubated for 45 minutes at room temperature on the shaker. The wells were washed four times with a washing buffer and incubated with a reaction solution that contained an HRP substrate (e.g. TMB; 3, 3′, 5,5′-Tetramethylbenzidine). After 10-25 minutes, a stop solution was added to each well. The absorbance of the HRP reaction product in 450 nm was determined using a standard spectrophotometer. This protocol took 99 (TRAIL) and 85 (IP-10) minutes respectively and is referred to herein as the rapid protocol.

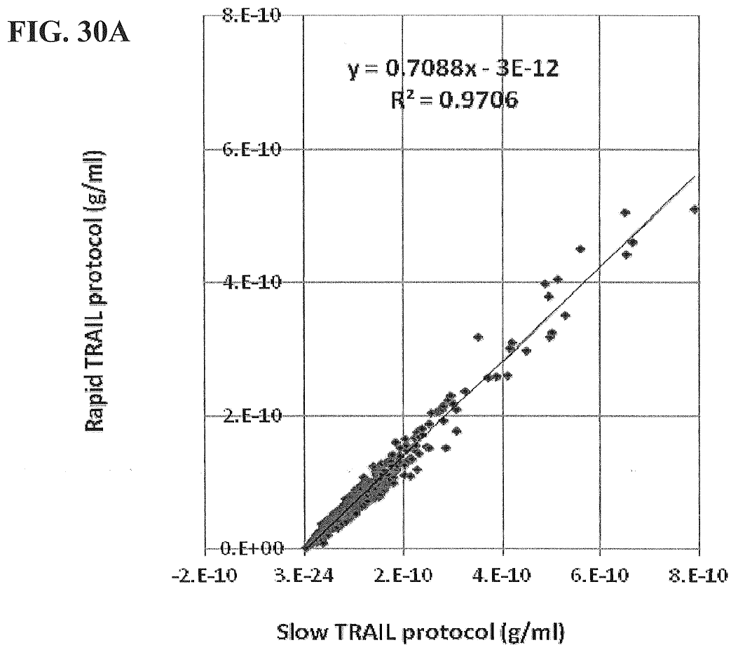

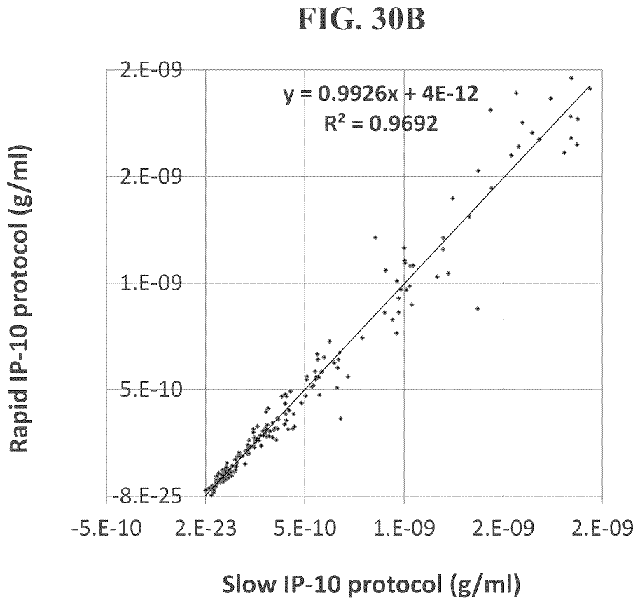

The slow and the rapid protocol measurements were compared using 357 samples for TRAIL and 189 samples for IP-10, and showed highly correlated results (FIGS. 30A-30B).

Of note, the average TRAIL concentration obtained using the rapid protocol was roughly 70 percent less than that obtained using the slow protocol concentration. Such alterations in measured concentrations of proteins across different protocols often occur and can be compensated for by correlating the measurements of the two protocols and computing a transformation function. For example, the transformation function y_slow=0.709×y_rapid−3e−12 may be used to translate the concentrations of the rapid protocol and the slow protocol. This translation preserves TRAIL's accuracy. Other, translation functions and protocols can be developed by one skilled in the art that also preserve the accuracy. In summary, the behavior of TRAIL remains the same across the two protocols (i.e. highest in viral, lower in non-infectious and lowest in bacterial), despite a shift in the calculated concentrations.

### Different Protocols and Cohorts Lead to Different Model Coefficients:

An example of the multinomial logistic model coefficients generated on the majority patients cohort when measuring IP-10 and TRAIL with the slow protocol is shown in Table 13:

TABLE 13

| Second Coordinate | First Coordinate | |
|---|---|---|
| δ₁ (bacterial) | δ₀ (viral) | |
| b₀ = −1.5389 ± 0.75676 | a₀ = −1.7331 ± 0.62936 | Const |
| b₁ = 0.0851 ± 0.015288 | a₁ = 0.0514 ± 0.014896 | CRP (mg/ml) |
| b₂ = 0.0046 ± 0.001372 | a₂ = 0.0049 ± 0.001372 | IP10 (pg/ml) |
| b₃ = −0.0155 ± 0.007056 | a₃ = 0.0048 ± 0.005096 | TRAIL (pg/ml) |

An example of the multinomial logistic model coefficients generated on the consensus patients cohort when measuring IP-10 and TRAIL with the slow protocol is shown in Table 14.

TABLE 14

| Second Coordinate | First Coordinate | |
|---|---|---|
| δ₁ (bacterial) | δ₀ (viral) | |
| b₀ = 2.6091 ± 0.9357 | a₀ = −2.6866 ± 0.75048 | Const |
| b₁ = 0.0866 ± 0.016856 | a₁ = 0.0499 ± 0.016464 | CRP (mg/ml) |
| b₂ = 0.0052 ± 0.001568 | a₂ = 0.0059 ± 0.001568 | IP10 (pg/ml) |
| b₃ = −0.0115 ± 0.008232 | a₃ = 0.0084 ± 0.005684 | TRAIL (pg/ml) |

Since the frequency of the subgroups in the patient cohort deviates from the anticipated frequency in the general population, one can further adjust the model coefficients to reflect a predetermined prior probability using standard techniques for coefficient adjustment (for example see G. King and L Zeng, Statistics in Medicine 2002). For example, the following examples show multinomial logistic model coefficients generated on the majority patients cohort when measuring IP-10 and TRAIL with the slow protocol, reflecting prior probability of 45% bacterial, 45% viral and 10% non-infectious.

Model coefficients (trained on majority cohort) after prior adjustment are summarized in Table 15:

TABLE 15

| Second Coordinate | First Coordinate | |
|---|---|---|
| δ₁ (bacterial) | δ₀ (viral) | |
| b₀ = −1.1302 ± 0.75676 | a₀ = −1.4151 ± 0.62936 | Const |
| b₁ = 0.0851 ± 0.015288 | a₁ = 0.0514 ± 0.014896 | CRP (mg/ml) |
| b₂ = 0.0046 ± 0.001372 | a₂ = 0.0049 ± 0.001372 | IP10 (pg/ml) |
| b₃ = −0.0155 ± 0.007056 | a₃ = 0.0048 ± 0.005096 | TRAIL (pg/ml) |

Model coefficients (trained on consensus cohort) after prior adjustment are summarized in Table 16.

TABLE 16

| Second Coordinate | First Coordinate | |
|---|---|---|
| δ₁ (bacterial) | δ₀ (viral) | |
| b₀ = −1.7833 ± 0.9357 | a₀ = −2.083 ± 0.75048 | Const |
| b₁ = 0.0866 ± 0.016856 | a₁ = 0.0499 ± 0.016464 | CRP (mg/ml) |
| b₂ = 0.0052 ± 0.001568 | a₂ = 0.0059 ± 0.001568 | IP10 (pg/ml) |
| b₃ = −0.0115 ± 0.008232 | a₃ = 0.0084 ± 0.005684 | TRAIL (pg/ml) |

Of note, other combinations of coefficients can be chosen to produce similar results, as would be evident to one skilled in the art. Other protocols for measuring proteins that affect the measured protein concentrations would yield different model coefficients. For example, the rapid protocol for measuring TRAIL reduces the computed concentrations to roughly 70% of the concentrations computed in the slow protocol. Thus, one way to adjust for this is to alter the model coefficients of TRAIL to account for this change. Another way is to divide the rapid protocol measurements of TRAIL by 70% and plug in to the above mentioned models that were developed for the slow protocol.

It is often preferable to use a log transformation on the protein measurements in order to improve model accuracy and calibration (i.e. better fit between the predicted risk of a certain infection and the observed risk).

An example of a model with log transformation of TRAIL and IP-10 is depicted in Table 17 (model was trained on the consensus cohort):

TABLE 17

| Second Coordinate | First Coordinate | |
|---|---|---|
| δ₁ (bacterial) | δ₀ (viral) | |
| b₀ = −5.9471 ± 3.3391 | a₀ = −14.8487 ± 3.3839 | Const |
| b₁ = 0.0833 ± 0.016856 | a₁ = 0.0437 ± 0.017052 | CRP (mg/ml) |
| b₂ = 1.3868 ± 0.48608 | a₂ = 2.0148 ± 0.4408 | IP10 (pg/ml) |
| b₃ = −0.788 ± 0.60505 | a₃ = 0.8946 ± 0.61348 | TRAIL (pg/ml) |

### Example 6

### Hypersurface Parameterization

Given the concentrations of CRP [C], TRAIL [T] and IP-10 [P] we define:

$$\begin{matrix}{δ}_{0} = - 1.299 + 0.0605 \times [ C ] + 0.0053 \times [ P ] + 0.0088 \times [ T ] \\ {δ}_{1} = - 0.378 + 0.0875 \times [ C ] + 0.005 \times [ P ] - 0.0201 \times [ T ]\end{matrix}$$

The probabilities can then be calculated by:

$$\begin{matrix}P ( Viral ) = \frac{{e}^{{δ}_{0}}}{1 + {e}^{{δ}_{0}} + {e}^{{δ}_{1}}} \\ P ( Bacterial ) = \frac{{e}^{{δ}_{1}}}{1 + {e}^{{δ}_{0}} + {e}^{{δ}_{1}}} \\ P ( Non - infectious ) = \frac{1}{1 + {e}^{{δ}_{0}} + {e}^{{δ}_{1}}}\end{matrix}$$

We define the hyper surface in the [C], [T], [P] space:

$$\frac{{e}^{{δ}_{1}}}{1 + {e}^{{δ}_{0}} + {e}^{{δ}_{1}}} = ω$$

that is used to distinguish between bacterial and non-bacterial patients. In one preferred embodiment. In other preferred embodiments. Given a patient's [C], [T], [P] values that patient is classified as bacterial if

$$\frac{{e}^{{δ}_{1}}}{1 + {e}^{{δ}_{0}} + {e}^{{δ}_{1}}} > ω ,$$

else he/she are classified as non-bacterial.

We define the set all hyper plains that can be used to distinguish between bacterial and non-bacterial infections as those that reside within the following two hyper surfaces:

$$\begin{matrix}\frac{{e}^{{δ}_{1}}}{1 + {e}^{{δ}_{0}} + {e}^{{δ}_{1}}} = ω + {ϵ}_{1} \\ \frac{{e}^{{δ}_{1}}}{1 + {e}^{{δ}_{0}} + {e}^{{δ}_{1}}} = ω - {ϵ}_{0}\end{matrix}$$

ϵ₁ can be any number between 0 and 1−. In some preferred embodiments ϵ₁ is smaller then 0.5, 0.4, 0.3, 0.2 or 0.1.
ϵ₀ can be any number between 0 and ω. In some preferred embodiments ϵ₀, is smaller then 0.5, 0.4, 0.3, 0.2 or 0.1.

Illustrated examples of surfaces are provided in Example 7.

### Example 7

### Graphical Representation of Classification

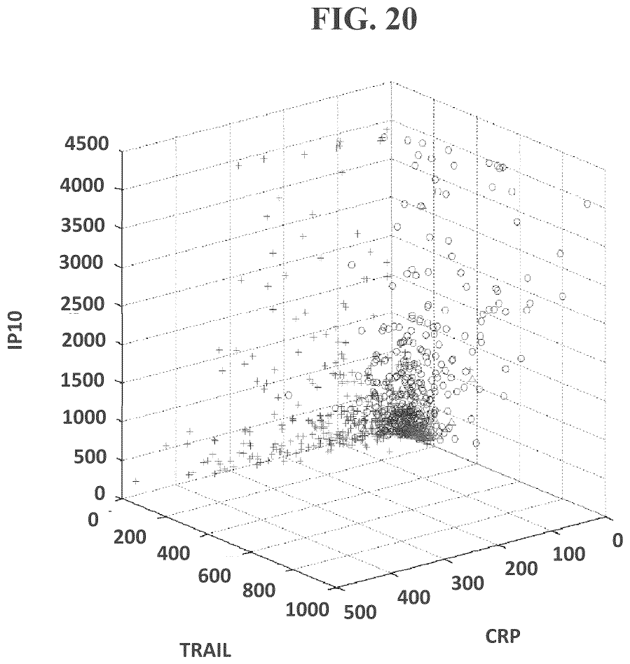

FIG. 20 is a 3-dimensional visualization of bacterial (‘+’), viral (‘o’) and non-infectious (‘\hat{ }’) patients. Different patients types are mapped to distinct regions in the CRP (μg/ml), TRAIL and IP-10 (pg/ml) concentration map.

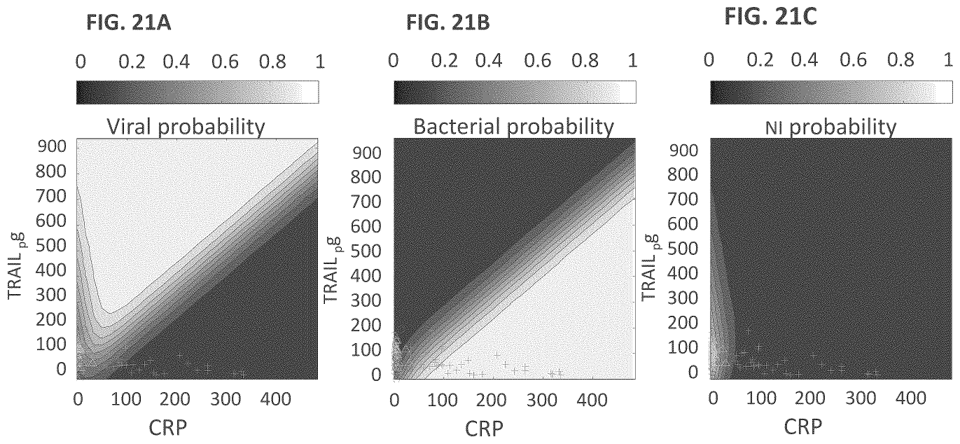

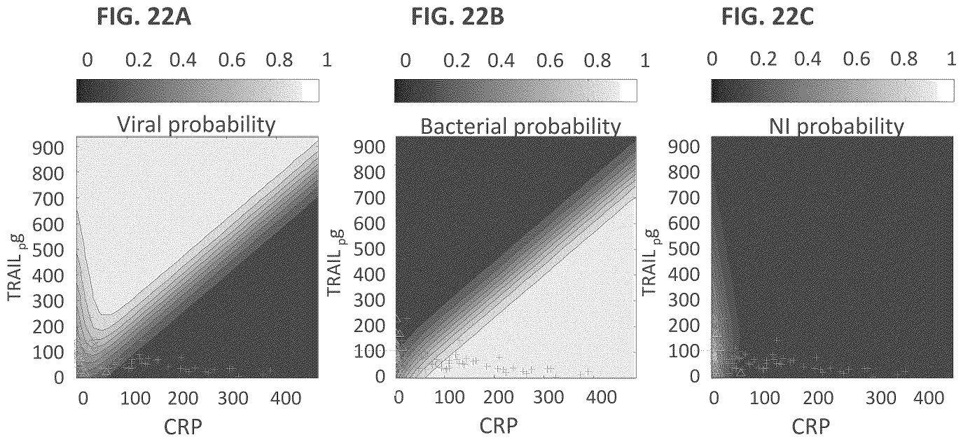

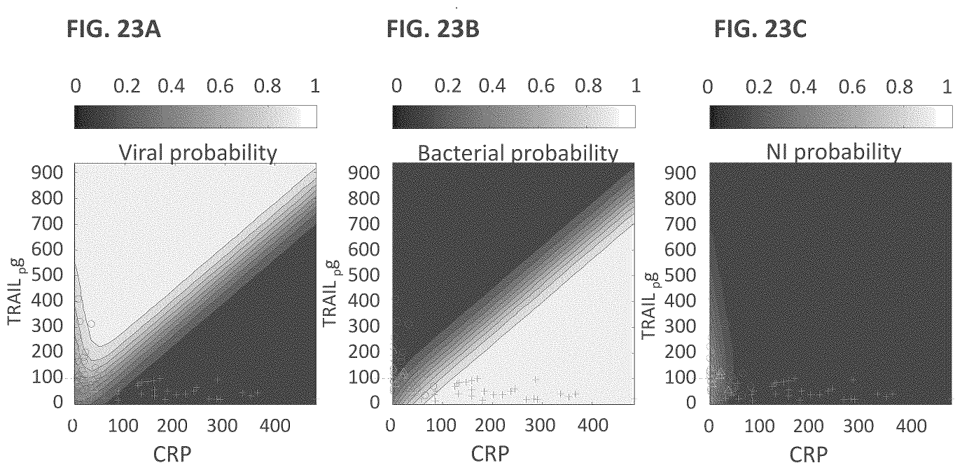

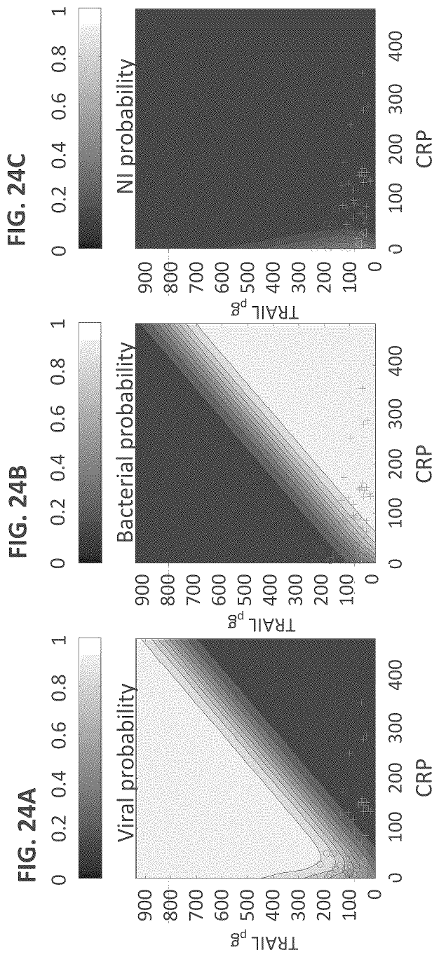

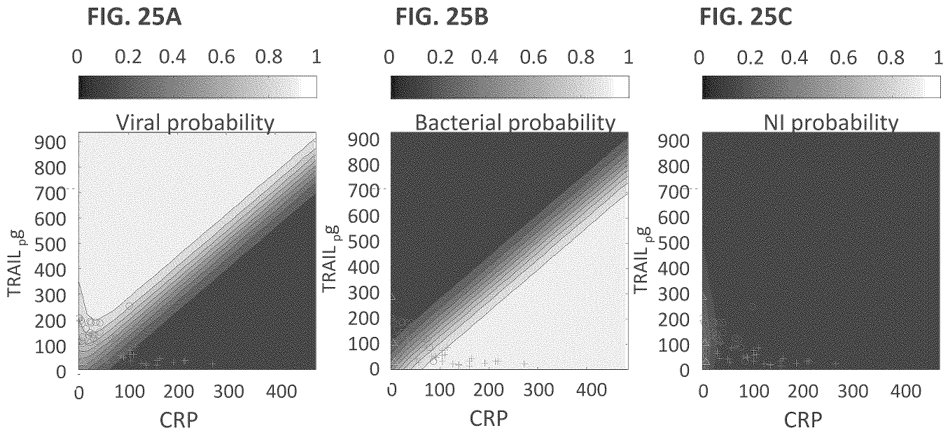

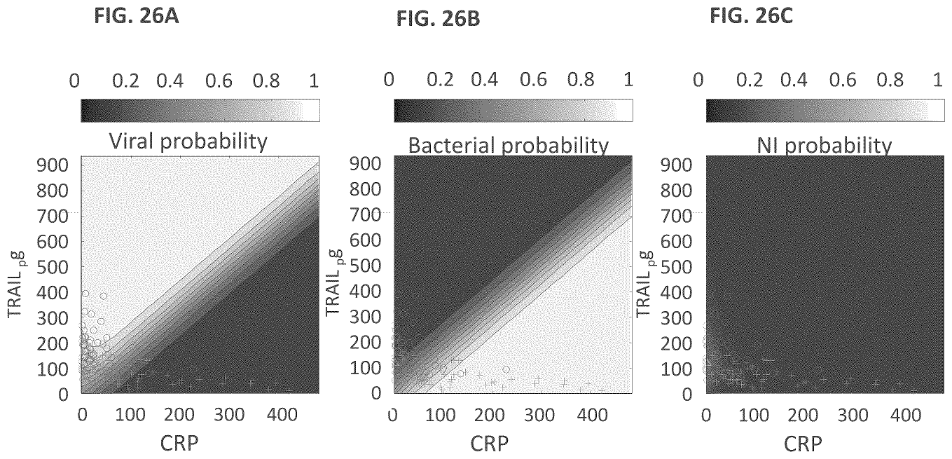

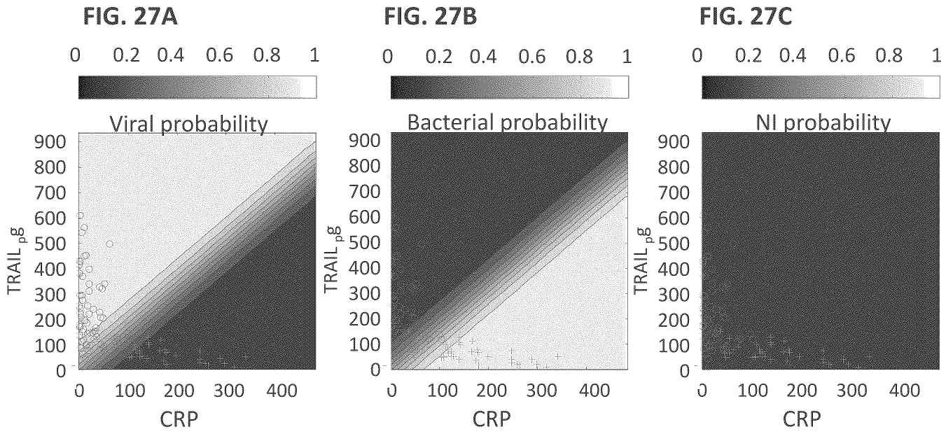

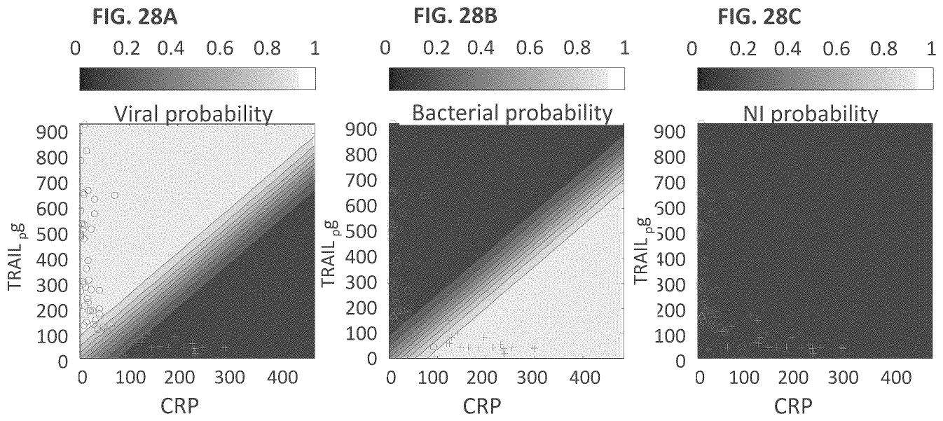

By way of example probability surfaces were generated using a multinomial logistic regression. Contour plots of the surfaces are shown in FIGS. 21A-28C, as a function of TRAIL (y-axis), CRP (x-axis), and IP-10 concentrations. FIGS. 21A, 22A, 23A, 24A, 25A, 26A, 27A, 28A, show probabilities of viral infectious, FIGS. 21B, 22B, 23B, 24B, 25B, 26B, 27B, 28B, show probabilities of bacterial or mixed infectious, and FIGS. 21C, 22C, 23C, 24C, 25C, 26C, 27C, 28C, show probabilities of non-infectious or healthy. FIGS. 21A-21C correspond to IP10ₚg ranging from 0 to 100, FIGS. 22A-22C correspond to IP10ₚg ranging from 100 to 200, FIGS. 23A-23C correspond to IP10ₚg ranging from 200 to 300, FIGS. 24A-24C correspond to IP10ₚg ranging from 300 to 400, FIGS. 25A-25C correspond to IP10ₚg ranging from 400 to 500, FIGS. 26A-26C correspond to IP10ₚg ranging from 500 to 1000, FIGS. 27A-27C correspond to IP10ₚg ranging from 1000 to 2000 and FIGS. 28A-28C correspond to IP10ₚg which is 2000 or above.

Patients with bacterial or mixed are marked with a ‘+’; viral with a ‘o’ and non-infectious or healthy with a ‘\hat{ }’. It can be seen in that low levels of IP-10 are associated with non-infectious disease, higher levels with bacterial and highest with viral. Low levels of TRAIL are associated with bacterial infections, higher with non-infectious and healthy, and highest with viral. Low levels of CRP are associated with non-infectious disease and healthy subjects, higher with viral infection and highest with bacterial. The combination of the three proteins generates a probability function whose diagnostic performance outperforms any of the individual or pairs of proteins.

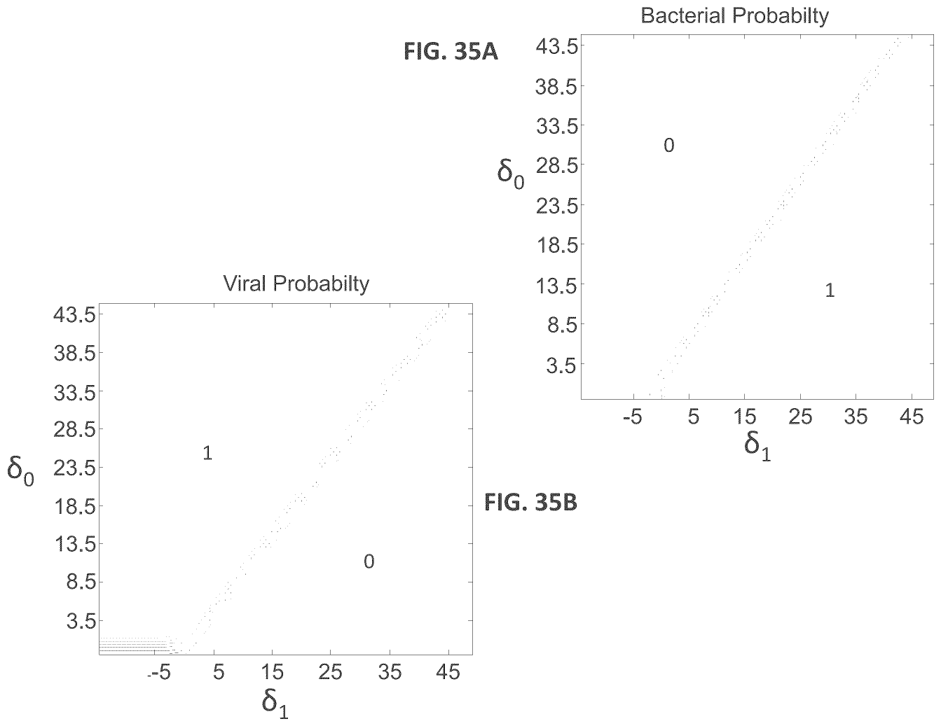

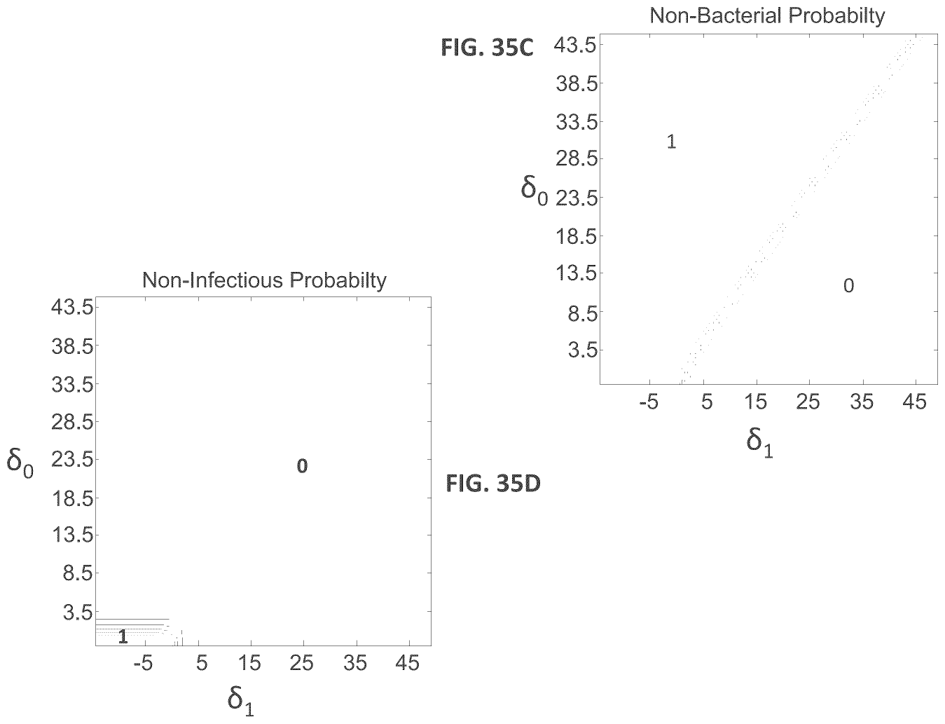

FIGS. 35A-35D are contour plots describing the probability of bacterial (FIG. 35A), viral (FIG. 35B), non-bacterial (FIG. 35C), and non-infectious (FIG. 35D) etiologies as a function of the coordinates δ₀ and δ₁. The probability values range between 0% (black) to 100% (white).

### Example 8

### Exemplified Protocols for Measuring Expression Levels

In general, without limitation expression value of TRAIL can be measured using an ELISA or automated immunoassay; expression value of IP-10 can be measured using an ELISA assay; and expression value of CRP can be measured using an ELISA or automated immunoassay. The expression value of CRP can also be measured using a functional assay based on its calcium-dependent binding to phosphorylcholine.

### Protocol A:

### Suitable Protocol for Measuring an Expression Value of TRAIL

(a) immobilize TRAIL present in a sample using an antibody to a solid support;
(b) contact immobilized TRAIL with a second antibody that specifically binds to TRAIL; and
(c) quantify the amount of antibody that binds to the immobilized TRAIL.

### Suitable Protocol for Measuring an Expression Value of IP-10

(a) immobilize IP-10 present in a sample using a capture antibody to a solid support;
(b) contact immobilized IP-10 with a second antibody that specifically binds to IP-10; and
(c) quantify the amount of antibody that binds to the immobilized IP-10.

### Suitable Protocol for Measuring an Expression Value of CRP

(a) immobilize CRP present in a sample using a capture antibody to a solid support;
(b) contact immobilized CRP with a second antibody that specifically binds to I CRP; and
(c) quantify the amount of antibody that binds to the immobilized CRP.

### Protocol B:

### Suitable Protocol for Measuring an Expression Value of TRAIL

(a) Incubate a sample with a first antibody that specifically binds to TRAIL, wherein the said first antibody is immobilized to a solid phase;
(b) Wash;
(c) Add second antibody that specifically binds to TRAIL, wherein the second antibody is conjugated to an enzyme; wash
(d) Add enzyme substrate and quantify the amount of antibody that binds to the immobilized sample.

### Suitable Protocol for Measuring an Expression Value of IP-10

(a) Incubate a sample with a first antibody that specifically binds to IP-10, wherein the said first antibody is immobilized to a solid phase;
(b) Wash;
(c) Add second antibody that specifically binds to IP-10, wherein the second antibody is conjugated to an enzyme; wash
(d) Add enzyme substrate and quantify the amount of antibody that binds to the immobilized sample.

### Suitable Protocol for Measuring an Expression Value of CRP

(a) Incubate a sample with a first antibody that specifically binds to CRP, wherein the said first antibody is immobilized to a solid phase;
(b) Wash;
(c) Add second antibody that specifically binds to CRP, wherein the second antibody is conjugated to an enzyme; wash
(d) Add enzyme substrate and quantify the amount of antibody that binds to the immobilized sample.

### Protocol C:

### Suitable Protocol for Measuring an Expression Value of CRP

(a) measure the turbidity of a mixture of lipids;
(b) contact sample with a known amount of the lipids (preferably phosphorylcholine) in the presence of Calcium; and
(c) measure the turbidity of the solution, wherein increase in turbidity correlates with the amount of CRP.

### Example 9

### Detailed Description of ELISA for Analyzing the Amount of TRAIL and IP-10

Sample collection and storage: Exposure of samples to room temperature should be minimized (less than 6 hours). A serum separator tube (SST) is used and the samples are allowed to clot for at least 30 minutes before centrifugation (5 minutes at 1200×g). Serum may be assayed immediately, or aliquoted and stored at 4-8° C. for up to 24 hours or at ≤−20° C. for up to 3 months. Repeated freeze-thaw cycles should be avoided.

Reagent preparation: All reagents should be brought to room temperature before use.

Substrate solution: Color Reagents A and B should be mixed together in equal volumes within 10 minutes of use. Protect from light.

QC-1V, QC-2B and Standards: Thaw all QC and Standards and remove 150 uL from each vial to a separate marked Polypropylene test tube. Move back to −20° C. immediately after use.

### Trail Measurements:

The materials used for analyzing TRAIL are provided in Table 18, herein below.

TABLE 18

| Storage | | |
|---|---|---|
| conditions | Description | Part |
| Store at | 96 well microplate (12 strips of 8 wells) | TRAIL |
| 2-8° C. | coated with anti-TRAIL antibody | Microplate |
| | 21 ml of anti-TRAIL specific antibody | TRAIL |
| | conjugated to horseradish peroxidase | Conjugate |
| | with preservatives | |
| | 11 ml of a buffered protein base with | Assay |
| | preservatives | diluent |
| | | MM1S |
| | 21 mL of a 25-fold concentrated solution | Wash Buffer |
| | of buffered surfactant with preservative | Concentrate |
| | 12 mL of stabilized hydrogen peroxide | Color |
| | | reagent A |
| | 12 mL of tetramethylbenzidine (TMB) | Color |
| | | reagent B |
| | 6 mL of 2N sulfuric acid | Stop |
| | | solution |
| | 4 adhesive strips | Plate sealer |
| Store | 6 vials containing 0.7 ml of recombinant | 6 TRAIL |
| at −20° C. | human TRAIL in buffered protein base | Standards |
| immediately | with preservatives at the following con- | |
| after | centrations 500, 250, 125, 62.5, 31.2 and | |
| receiving. | 0 [pg/mL] | |
| | 1 ml | QC-1V |
| | 1 ml | QC-2B |

### TRAIL ELISA Procedure

a) Prepare samples, reagents and standards as indicated above.
b) Remove excess microplate strips from the plate frame, return them to the foil pouch containing the desiccant pack, and reseal.
c) Add 50 μL of Assay Diluent MM1S to each well.
d) Add 50 μL of Standard, samples, or QC per well. Cover with the adhesive strip provided.
e) Incubate for 30 minutes at room temperature on a microplate shaker (3 mm orbit) set at 550 rpm.
f) Aspirate each well and wash, repeating the process 4 times. Wash by filling each well with Wash Buffer (300 μL). After the last wash, remove any remaining Wash Buffer by aspirating or decanting. Invert the plate and blot it against clean paper towels.
g) Add 200 μL of TRAIL Conjugate to each well. Cover with a new adhesive strip. Incubate for 45 minutes at room temperature on a microplate shaker (3 mm orbit) set at 550 rpm.
h) Repeat the aspiration/wash as in step (g).
i) Add 200 μL of Substrate solution to each well. Incubate for 24 to 30 minutes at room temperature. Protect from light.
j) Add 50 μL of Stop solution to each well. The color in the wells should change from blue to yellow. If the color in the wells is green or the color change does not appear uniform, gently tap the plate to ensure thorough mixing.
k) Determine the optical density of each well immediately, using a microplate reader set to 450 nm. Set wavelength correction to 570 nm, which will correct for optical imperfections in the plate.

TRAIL calculation of concentrations: Average the duplicate readings for each sample and subtract the average zero standard optical density (O.D.). Create a standard curve by plotting the mean absorbance for each standard (y-axis) against the concentration (x-axis) and draw a best-fit linear curve. The minimal r² should not fall below 0.96. In case lower r² values are present, repeat the experiment to get reliable results.

Precision: Precision was evaluated based on the CLSI (formerly NCCLS) EP05-A₂ guidelines. Three samples with concentrations at the low (11.4 pg/ml), intermediate (58.8 pg/ml), and high (539.0 pg/ml) physiological concentrations were used to assess precision. Results are summarized in Table 19, where Sᵣ is within-run precision and S_{T} is within-device precision:

TABLE 19

| High | Medium | Low | |
|---|---|---|---|
| (539.0 pg/ml) | (58.8 pg/ml) | (11.4 pg/ml) | |
| 18 | 18 | 18 | # of runs |
| 36 | 36 | 36 | # of duplicates |
| 13.2 | 2.45 | 0.84 | Sᵣ pg/mL |
| 2.5% | 4.2% | 7.3% | Sᵣ CV (%) |
| 29.7 | 3.6 | 1.3 | S_{T} pg/mL |
| 5.5% | 6.1% | 11.5% | S_{T} CV (%) |

Recovery: Recovery was evaluated by spiking three levels of human recombinant TRAIL (250, 125 and 62.5 pg/mL) into 5 human serum samples with no detectable levels of TRAIL. The spiked values and the average recovery was then measured and calculated, as shown in Table 20 below.

TABLE 20

| Range | Average % Recovery | Sample |
|---|---|---|
| 75-78% | 77% | Serum (n = 5) |

Linearity: To assess the linearity of the assay, five clinical samples containing high concentrations of TRAIL were serially diluted using a serum substitute to produce samples with values within the physiological range of the assay. Linearity was, on average, 97%, 100% and 105% for 1:2, 1:4 and 1:8 dilutions, respectively, as summarized in Table 21 below.

TABLE 21

| Serum (n = 5) | | |
|---|---|---|
| 97% | Average % of | 1:2 |
| | expected | |
| 90-104% | Range % | |
| 100% | Average % of | 1:4 |
| | expected | |
| 90-108% | Range % | |
| 105% | Average % of | 1:8 |
| | expected | |
| 90-121% | Range % | |

Sensitivity: To estimate the Limitation of Blank (LOB), we tested 72 blank samples of serum substitute. The mean of the blank samples was 0.78 pg/ml and the standard deviation was 1.39 pg/ml. Therefore, the calculated LOB is 3.07 pg/ml. To estimate the Limitation of Detection (LOD), the CLSI EP17-A guidelines were followed. Briefly, the measurement distribution around seven predetermined concentrations were characterized, each with 30 independent measurements (210 measurements) yielding an LOD of 10 pg/ml.

Calibration: This immunoassay is calibrated against a purified NSO-expressed recombinant human TRAIL.

Expected values: Samples from apparently healthy adult (>18 years) were measured for the presence of TRAIL. The range and mean values are summarized in Table 22.

TABLE 22

| Range pg/ml | Mean pg/ml | Sample Type |
|---|---|---|
| 17-157 | 90 | Serum (n = 34) |

Cross reactivity and interference: This assay recognizes natural and recombinant human TRAIL. The factors 4-1BB Ligand, APRIL, BAFF/BLyS, CD27 Ligand, CD30 Ligand, CD40 Ligand, Fas Ligand, GITR Ligand, LIGHT, LT a1/B2, LT a2/B1, OPG, OX40 Ligand, TNF-α, TNF-β, TRAIL R3, TRAIL R4, TRANCE and TWEAK were prepared at 50 ng/mL in serum substitution and assayed for cross-reactivity. Additionally, preparations of these factors at 50 pg/mL in a mid-range recombinant human TRAIL control were tested for interference. No significant cross-reactivity or interference was observed.

IP-10 measurements: The materials used for analyzing IP-10 are provided in Table 23, herein below.

TABLE 23

| Storage | | |
|---|---|---|
| conditions | Description | Part |
| Store at | 96 well microplate (12 strips of 8 wells) | IP-10 |
| 2-8° C. | coated with anti-IP-10 antibody | Microplate |
| | 21 ml of anti-IP-10 specific antibody | IP-10 |
| | conjugated to horseradish peroxidase | Conjugate |
| | with preservatives | |
| | 11 ml of a buffered protein base with | Assay |
| | preservatives | diluent |
| | | MM56 |
| | 21 mL of a 25-fold concentrated solution | Wash Buffer |
| | of buffered surfactant with preservative | Concentrate |
| | 12 mL of stabilized hydrogen peroxide | Color |
| | | reagent A |
| | 12 mL of tetramethylbenzidine (TMB) | Color |
| | | reagent B |
| | 6 mL of 2N sulfuric acid | Stop |
| | | solution |
| | 4 adhesive strips | Plate sealer |
| Store at −20° C. | 6 vials containing 0.7 ml of recombinant | 6 IP-10 |
| immediately | human IP-10 in buffered protein base with | Standards |
| after | preservatives at the following concentrations | |
| receiving | 1000, 500, 250, 125, 62.5 and 0 [pg/mL] | |
| | 1 ml | QC-1V |
| | 1 ml | QC-2B |

### IP-10 ELISA Procedure

a) Prepare samples, reagents and standards as indicated herein above.
b) Remove excess microplate strips from the plate frame, return them to the foil pouch containing the desiccant pack, and reseal.
c) Add 50 μL of Assay Diluent MM56 to each well.
d) Add 50 μL of Standard, sample or QC per well. Cover with the adhesive strip provided.
e) Incubate for 30 minutes at room temperature on a microplate shaker (3 mm orbit) set at 550 rpm.
f) Aspirate each well and wash, repeating the process 4 times. Wash by filling each well with Wash Buffer (300 μL). After the last wash, remove any remaining Wash Buffer by aspirating or decanting. Invert the plate and blot it against clean paper towels.
g) Add 200 μL of IP-10 Conjugate to each well. Cover with a new adhesive strip. Incubate for 45 minutes at room temperature on a microplate shaker (3 mm orbit) set at 550 rpm.
h) Repeat the aspiration/wash as in step (g).
i) Add 200 μL of Substrate solution to each well. Incubate for 10 minutes at room temperature. Protect from light.
j) Add 50 μL of Stop solution to each well. The color in the wells should change from blue to yellow. If the color in the wells is green or the color change does not appear uniform, gently tap the plate to ensure thorough mixing.
k) Determine the optical density of each well immediately, using a microplate reader set to 450 nm. Set wavelength correction to 570 nm, which will correct for optical imperfections in the plate.

IP-10 calculation of concentrations: Average the duplicate readings for each sample and subtract the average zero standard optical density (O.D.). Create a standard curve by plotting the mean absorbance for each standard (y-axis) against the concentration (x-axis) and draw a best-fit linear curve. The minimal r² should not fall below 0.96. In case lower r² values are present, repeat the experiment to get reliable results.

Precision: Precision was evaluated based on the CLSI (formerly NCCLS) EP05-A2 guidelines. Three samples with concentrations at the low (69.4 pg/ml), intermediate (228.2 pg/ml), and high (641.5 pg/ml) physiological concentrations were used to assess precision. Results are summarized in Table 24 where Sᵣ is within-run precision and S_{T} is within-device precision:

TABLE 24

| High | Medium | Low | |
|---|---|---|---|
| (641.5 pg/ml) | (228.2 pg/ml) | (69.4 pg/ml) | |
| 18 | 18 | 18 | # of runs |
| 36 | 36 | 36 | # of duplicates |
| 21.1 | 5.6 | 4.0 | Sᵣ pg/mL |
| 3.3% | 2.4% | 5.8% | Sᵣ CV (%) |
| 37.2 | 12.9 | 4.9 | S_{T} pg/mL |
| 5.8% | 5.7% | 7.1% | S_{T} CV (%) |

Recovery: Recovery was evaluated by spiking three levels of human IP-10, 500, 250 and 125 pg/mL into 5 human serum samples with no detectable levels of IP-10. The spiked values and the average recovery was than measured and calculated as illustrated in Table 25 below.

TABLE 25

| Range | Average % Recovery | Sample |
|---|---|---|
| 72-80% | 77 | Serum/plasma (n = 5) |

Linearity: To assess the linearity of the IP-10 assay, 5 clinical samples containing high concentrations of IP-10 ranging between 873.7 to 1110.4 pg/mL were serially diluted with a serum substitute to produce samples with values within the physiological range of the assay. Linearity was, on average, 98%, 102% and 104% in 1:2, 1:4 and 1:8 dilutions, respectively, as summarized in Table 26 herein below.

TABLE 26

| Serum (n = 5) | | |
|---|---|---|
| 98% | Average % of | 1:2 |
| | expected | |
| 93-102% | Range % | |
| 102% | Average % of | 1:4 |
| | expected | |
| 97-107% | Range % | |
| 104% | Average % of | 1:8 |
| | expected | |
| 96-111% | Range % | |

Sensitivity: To estimate the Limitation of Blank (LOB), we tested 72 blank samples of serum substitute. The mean of the blank samples was 0.23 pg/ml and the standard deviation was 1.26 pg/ml, yielding an LOB of 2.29 pg/ml.

To estimate the Limitation of Detection (LOD), the CLSI EP17-A guidelines were applied. Briefly, the measurement distribution around seven predetermined concentrations were characterized, each with 30 independent measurements (210 measurements) yielding an LOD of 10 pg/ml.

Calibration: This immunoassay is calibrated against a highly purified E.-coli-expressed recombinant human IP-10.

Expected values: Samples from apparently healthy adult volunteers were measured for the presence of IP-10. The range and mean values are shown in Table 27 below.

TABLE 27

| Range pg/ml | Mean pg/ml | Sample Type |
|---|---|---|
| 29-525 | 119 | Serum (n = 34) |

Cross reactivity and interference: This assay recognizes natural and recombinant human IP-10. The factors BLC/BCA-1, ENA-78, GCP-2, GROα, GRO γ, IFN-γ, IL-8, I-TAC, MIG, NAP-2, SDF-1α and SDF-1β were prepared at 50 ng/ml in serum substitution and assayed for cross-reactivity. Additionally, preparations of these factors at 50 μg/mL in a mid-range recombinant human IP-10 control were tested for interference. No significant cross-reactivity or interference was observed.

### Example 10

### Trail and Disease Prognosis

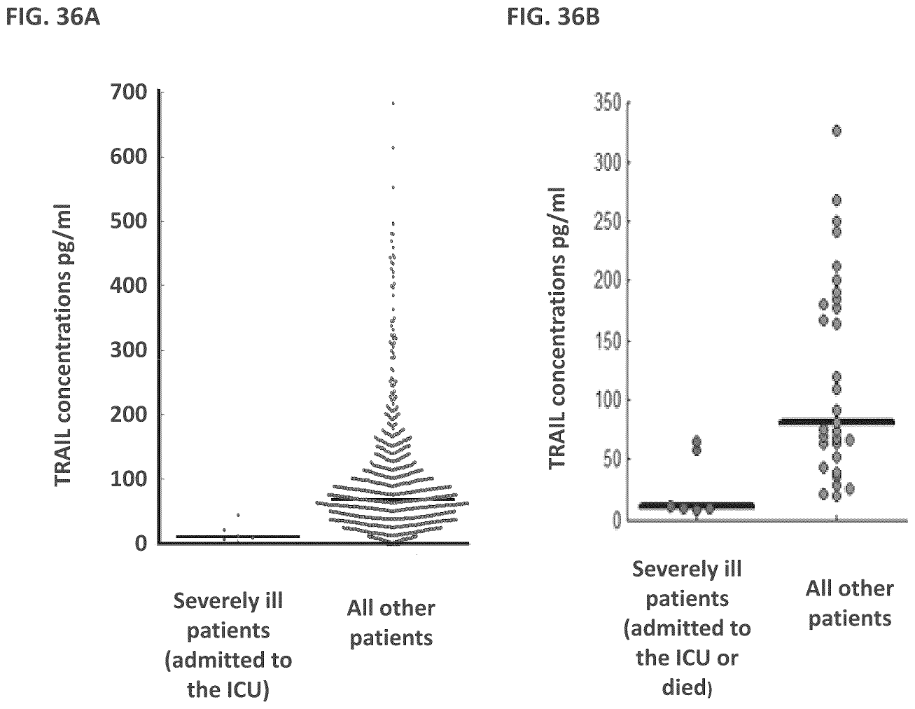

It is often clinically useful to assess patient prognosis, disease severity and outcome. The present inventors found that low levels of TRAIL are significantly correlated with poor patient prognosis and outcome, and high disease severity. For example, adult patients in the intensive care unit (ICU) had significantly lower TRAIL levels compared to all other patients, which were less ill regardless of whether they had an infectious or non-infectious etiology. Median serum concentrations were 9 pg/ml vs. 80 pg respectively, (ranksum P<0.001, FIG. 36A), for severely ill and all other patients respectively.

40 Dutch pediatric patients, 3 months to 5 years of age. The TRAIL serum level was measured in 40 Dutch pediatric patients, 3 months to 5 years of age. It was found that those patients that were eventually admitted to the ICU (an indication of disease complication and poor prognosis) or even died had significantly lower TRAIL serum concentrations compared to the rest of the patients (median of 11 vs. 85, respectively; ranksum P<0.001) as depicted in FIG. 36B. Strikingly, the lowest TRAIL levels (<5 pgml) were measured in the only two children that died in the entire cohort. These results indicate that TRAIL could be used as a prognostic marker for predicting disease severity and outcome.

### Example 11

### Trail Age and Gender Parameters

Basal levels of TRAIL in healthy individuals or patients with a non-infectious disease are lower in females compared to males during fertility age (t-test P<0.001) (FIG. 37A), but is invariant in pre- or post-fertility age (t-test P=0.9, FIG. 37B). This trend was not observed in patients with an infectious disease.

### Example 12

### Exemplified Manifolds, Hyperplanes and Coordinates

### One-Dimensional Manifold

When n=1, the manifold S is a curved line and the hyperplane π is an axis defining a single direction δ₁. The coordinate δ₁ in this Example is optionally and preferably a linear combination b₀+b₁D₁+b₂D₂+ . . . , of the polypeptides D₁, D₂, etc.

Table 28 below lists diagnostic performance (in AUCs) attained for n=1. The performance were computed using a leave-10%-out cross validation on the cohort specified in each row. In rows 1-4, the analyzed subjects had either bacterial or viral infections and the coordinate δ₁ was calculated so that the probabilistic classification function f(δ₁) represented the likelihood that the test subject had a bacterial infection. In rows 5-8, the analyzed subjects were infectious or non-infections and the coordinate δ₁ was calculated so that the probabilistic classification function f(δ₁) represented the likelihood that the test subject had an infection. In rows 10-12, the analyzed subjects had either bacterial or non-bacterial infection and the coordinate δ₁ was calculated so that the probabilistic classification function f(δ₁) represented the likelihood that the test subject had a bacterial infection. In rows 1-4, the columns P and N correspond to the number of Bacterial and Viral patients respectively, in rows 5-8, the columns P and N correspond to the number of infectious and non-infectious patients, respectively, and in rows 9-12, the columns P and N correspond to the number Bacterial and non-Bacterial patients respectively. Majority and Consensus indicate the type of cohort on which the model was validated.

TABLE 28

| N | P | AUC | Polypeptides | | | Cohort | No. |
|---|---|---|---|---|---|---|---|
| 334 | 319 | 0.93 | | TRAIL | CRP | Majority | 1 |
| 334 | 319 | 0.94 | TRAIL | IP-10 | CRP | Majority | 2 |
| 271 | 256 | 0.95 | | TRAIL | CRP | Consensus | 3 |
| 271 | 256 | 0.96 | TRAIL | IP-10 | CRP | Consensus | 4 |
| 112 | 653 | 0.93 | | TRAIL | CRP | Majority | 5 |
| 112 | 653 | 0.96 | TRAIL | IP-10 | CRP | Majority | 6 |
| 112 | 527 | 0.93 | | TRAIL | CRP | Consensus | 7 |
| 112 | 527 | 0.97 | TRAIL | IP-10 | CRP | Consensus | 8 |
| 446 | 319 | 0.94 | | TRAIL | CRP | Majority | 9 |
| 446 | 319 | 0.94 | TRAIL | IP-10 | CRP | Majority | 10 |
| 383 | 256 | 0.95 | | TRAIL | CRP | Consensus | 11 |
| 383 | 256 | 0.96 | TRAIL | IP-10 | CRP | Consensus | 12 |

Table 29 below lists the coefficients b₀, b₁, b₂, etc that were used to define the coordinate δ₁, for each of the 12 cases listed in Table 28, respectively. The first coefficient on the left is b₀, and then from left to right, the coefficients correspond to the order of the polypeptides in each row of Table 28. The coefficients correspond to the following concentration scales for each polypeptide: TRAIL (pg/ml), IP-10 (pg/ml) and CRP (ug/ml).

For a given set of polypeptides, the obtained coefficients have small variations among the different cohorts. Nevertheless, the coefficients for the probabilistic classification functions and coordinates of the present embodiments preferably correspond to those obtained for the Majority Cohort.

TABLE 29

| Coefficients | | | | No. |
|---|---|---|---|---|
| | −0.029953 | 0.027472 | 0.64814 | 1 |
| −0.029013 | −0.00028168 | 0.028119 | 0.71542 | 2 |
| | −0.033669 | 0.034565 | 0.636 | 3 |
| −0.03195 | −0.00058691 | 0.035748 | 0.79543 | 4 |
| | 0.016837 | 0.17237 | −2.0549 | 5 |
| 0.005213 | 0.00592 | 0.1263 | −2.3344 | 6 |
| | 0.018624 | 0.16625 | −2.3469 | 7 |
| 0.0079169 | 0.0061124 | 0.12261 | −2.7949 | 8 |
| | −0.027839 | 0.034954 | −0.08503 | 9 |
| −0.027916 | 2.2524e−05 | 0.034878 | −0.088207 | 10 |
| | −0.030997 | 0.044289 | −0.26606 | 11 |
| −0.03042 | −0.00018635 | 0.044938 | −0.23907 | 12 |

Table 30 below lists diagnostic performance (in AUCs) attained for one-dimensional manifold. The performance were computed using a leave-10%-out cross validation on the Majority cohort. In rows 1-55, the analyzed subjects had either bacterial or viral infections and the probabilistic classification function f(δ₁) represented the likelihood that the test subject had a bacterial infection. In rows 56-110, the analyzed subjects were infectious or non-infections and the probabilistic classification function f(δ₁) represented the likelihood that the test subject had an infection. In rows 1-55, the columns P and N correspond to the number of Bacterial and Viral patients respectively, and in rows 56-110, the columns P and N correspond to the number of infectious and noninfectious patients, respectively.

TABLE 30

| N | P | AUC | Polypeptides | | | | | | No. |
|---|---|---|---|---|---|---|---|---|---|
| 141 | 142 | 0.88 | | | | | IL1ra | CRP | 1 |
| 299 | 295 | 0.90 | | | | | IP-10 | CRP | 2 |
| 50 | 51 | 0.87 | | | | | PCT | CRP | 3 |
| 241 | 255 | 0.90 | | | | | SAA | CRP | 4 |
| 142 | 142 | 0.64 | | | | | IP-10 | IL1ra | 5 |
| 14 | 19 | 0.62 | | | | | PCT | IL1ra | 6 |
| 122 | 124 | 0.83 | | | | | SAA | IL1ra | 7 |
| 142 | 142 | 0.88 | | | | | TRAIL | IL1ra | 8 |
| 49 | 51 | 0.74 | | | | | PCT | IP-10 | 9 |
| 242 | 251 | 0.85 | | | | | SAA | IP-10 | 10 |
| 297 | 295 | 0.88 | | | | | TRAIL | IP-10 | 11 |
| 40 | 45 | 0.78 | | | | | SAA | PCT | 12 |
| 50 | 51 | 0.87 | | | | | TRAIL | PCT | 13 |
| 244 | 255 | 0.90 | | | | | TRAIL | SAA | 14 |
| 141 | 142 | 0.90 | | | | IP-10 | IL1ra | CRP | 15 |
| 14 | 19 | 0.82 | | | | PCT | IL1ra | CRP | 16 |
| 121 | 124 | 0.89 | | | | SAA | IL1ra | CRP | 17 |
| 141 | 142 | 0.94 | | | | TRAIL | IL1ra | CRP | 18 |
| 49 | 51 | 0.89 | | | | PCT | IP-10 | CRP | 19 |
| 239 | 251 | 0.91 | | | | SAA | IP-10 | CRP | 20 |
| 40 | 45 | 0.88 | | | | SAA | PCT | CRP | 21 |
| 50 | 51 | 0.93 | | | | TRAIL | PCT | CRP | 22 |
| 241 | 255 | 0.94 | | | | TRAIL | SAA | CRP | 23 |
| 14 | 19 | 0.62 | | | | PCT | IP-10 | IL1ra | 24 |
| 122 | 124 | 0.85 | | | | SAA | IP-10 | IL1ra | 25 |
| 142 | 142 | 0.88 | | | | TRAIL | IP-10 | IL1ra | 26 |
| 13 | 17 | 0.76 | | | | SAA | PCT | IL1ra | 27 |
| 14 | 19 | 0.71 | | | | TRAIL | PCT | IL1ra | 28 |
| 122 | 124 | 0.92 | | | | TRAIL | SAA | IL1ra | 29 |
| 39 | 45 | 0.81 | | | | SAA | PCT | IP-10 | 30 |
| 49 | 51 | 0.86 | | | | TRAIL | PCT | IP-10 | 31 |
| 242 | 251 | 0.91 | | | | TRAIL | SAA | IP-10 | 32 |
| 40 | 45 | 0.86 | | | | TRAIL | SAA | PCT | 33 |
| 14 | 19 | 0.83 | | | PCT | IP-10 | IL1ra | CRP | 34 |
| 121 | 124 | 0.92 | | | SAA | IP-10 | IL1ra | CRP | 35 |
| 141 | 142 | 0.94 | | | TRAIL | IP-10 | IL1ra | CRP | 36 |
| 13 | 17 | 0.74 | | | SAA | PCT | IL1ra | CRP | 37 |
| 14 | 19 | 0.90 | | | TRAIL | PCT | IL1ra | CRP | 38 |
| 121 | 124 | 0.94 | | | TRAIL | SAA | IL1ra | CRP | 39 |
| 39 | 45 | 0.88 | | | SAA | PCT | IP-10 | CRP | 40 |
| 49 | 51 | 0.92 | | | TRAIL | PCT | IP-10 | CRP | 41 |
| 239 | 251 | 0.94 | | | TRAIL | SAA | IP-10 | CRP | 42 |
| 40 | 45 | 0.92 | | | TRAIL | SAA | PCT | CRP | 43 |
| 13 | 17 | 0.70 | | | SAA | PCT | IP-10 | IL1ra | 44 |
| 14 | 19 | 0.70 | | | TRAIL | PCT | IP-10 | IL1ra | 45 |
| 122 | 124 | 0.91 | | | TRAIL | SAA | IP-10 | IL1ra | 46 |
| 13 | 17 | 0.82 | | | TRAIL | SAA | PCT | IL1ra | 47 |
| 39 | 45 | 0.85 | | | TRAIL | SAA | PCT | IP-10 | 48 |
| 13 | 17 | 0.82 | | SAA | PCT | IP-10 | IL1ra | CRP | 49 |
| 14 | 19 | 0.75 | | TRAIL | PCT | IP-10 | IL1ra | CRP | 50 |
| 121 | 124 | 0.94 | | TRAIL | SAA | IP-10 | IL1ra | CRP | 51 |
| 13 | 17 | 0.78 | | TRAIL | SAA | PCT | IL1ra | CRP | 52 |
| 39 | 45 | 0.92 | | TRAIL | SAA | PCT | IP-10 | CRP | 53 |
| 13 | 17 | 0.62 | | TRAIL | SAA | PCT | IP-10 | IL1ra | 54 |
| 13 | 17 | 0.74 | TRAIL | SAA | PCT | IP-10 | IL1ra | CRP | 55 |
| 87 | 283 | 0.91 | | | | | IL1ra | CRP | 56 |
| 102 | 594 | 0.96 | | | | | IP-10 | CRP | 57 |
| 6 | 101 | 0.85 | | | | | PCT | CRP | 58 |
| 78 | 496 | 0.91 | | | | | SAA | CRP | 59 |
| 87 | 284 | 0.89 | | | | | IP-10 | IL1ra | 60 |
| 6 | 33 | 0.79 | | | | | PCT | IL1ra | 61 |
| 64 | 246 | 0.91 | | | | | SAA | IL1ra | 62 |
| 87 | 284 | 0.86 | | | | | TRAIL | IL1ra | 63 |
| 6 | 100 | 0.73 | | | | | PCT | IP-10 | 64 |
| 81 | 493 | 0.96 | | | | | SAA | IP-10 | 65 |
| 107 | 592 | 0.91 | | | | | TRAIL | IP-10 | 66 |
| 3 | 85 | 0.89 | | | | | SAA | PCT | 67 |
| 7 | 101 | 0.60 | | | | | TRAIL | PCT | 68 |
| 81 | 499 | 0.93 | | | | | TRAIL | SAA | 69 |
| 87 | 283 | 0.95 | | | | IP-10 | IL1ra | CRP | 70 |
| 6 | 33 | 0.76 | | | | PCT | IL1ra | CRP | 71 |
| 64 | 245 | 0.92 | | | | SAA | IL1ra | CRP | 72 |
| 87 | 283 | 0.93 | | | | TRAIL | IL1ra | CRP | 73 |
| 6 | 100 | 0.81 | | | | PCT | IP-10 | CRP | 74 |
| 78 | 490 | 0.97 | | | | SAA | IP-10 | CRP | 75 |
| 3 | 85 | 0.88 | | | | SAA | PCT | CRP | 76 |
| 6 | 101 | 0.87 | | | | TRAIL | PCT | CRP | 77 |
| 78 | 496 | 0.95 | | | | TRAIL | SAA | CRP | 78 |
| 6 | 33 | 0.77 | | | | PCT | IP-10 | IL1ra | 79 |
| 64 | 246 | 0.94 | | | | SAA | IP-10 | IL1ra | 80 |
| 87 | 284 | 0.90 | | | | TRAIL | IP-10 | IL1ra | 81 |
| 3 | 30 | 0.72 | | | | SAA | PCT | IL1ra | 82 |
| 6 | 33 | 0.67 | | | | TRAIL | PCT | IL1ra | 83 |
| 64 | 246 | 0.90 | | | | TRAIL | SAA | IL1ra | 84 |
| 3 | 84 | 0.98 | | | | SAA | PCT | IP-10 | 85 |
| 6 | 100 | 0.68 | | | | TRAIL | PCT | IP-10 | 86 |
| 81 | 493 | 0.96 | | | | TRAIL | SAA | IP-10 | 87 |
| 3 | 85 | 0.98 | | | | TRAIL | SAA | PCT | 88 |
| 6 | 33 | 0.77 | | | PCT | IP-10 | IL1ra | CRP | 89 |
| 64 | 245 | 0.95 | | | SAA | IP-10 | IL1ra | CRP | 90 |
| 87 | 283 | 0.95 | | | TRAIL | IP-10 | IL1ra | CRP | 91 |
| 3 | 30 | 0.73 | | | SAA | PCT | IL1ra | CRP | 92 |
| 6 | 33 | 0.74 | | | TRAIL | PCT | IL1ra | CRP | 93 |
| 64 | 245 | 0.92 | | | TRAIL | SAA | IL1ra | CRP | 94 |
| 3 | 84 | 0.98 | | | SAA | PCT | IP-10 | CRP | 95 |
| 6 | 100 | 0.77 | | | TRAIL | PCT | IP-10 | CRP | 96 |
| 78 | 490 | 0.97 | | | TRAIL | SAA | IP-10 | CRP | 97 |
| 3 | 85 | 0.80 | | | TRAIL | SAA | PCT | CRP | 98 |
| 3 | 30 | 0.91 | | | SAA | PCT | IP-10 | IL1ra | 99 |
| 6 | 33 | 0.67 | | | TRAIL | PCT | IP-10 | IL1ra | 100 |
| 64 | 246 | 0.94 | | | TRAIL | SAA | IP-10 | IL1ra | 101 |
| 3 | 30 | 0.78 | | | TRAIL | SAA | PCT | IL1ra | 102 |
| 3 | 84 | 0.65 | | | TRAIL | SAA | PCT | IP-10 | 103 |
| 3 | 30 | 0.91 | | SAA | PCT | IP-10 | IL1ra | CRP | 104 |
| 6 | 33 | 0.66 | | TRAIL | PCT | IP-10 | IL1ra | CRP | 105 |
| 64 | 245 | 0.95 | | TRAIL | SAA | IP-10 | IL1ra | CRP | 106 |
| 3 | 30 | 0.73 | | TRAIL | SAA | PCT | IL1ra | CRP | 107 |
| 3 | 84 | 0.97 | | TRAIL | SAA | PCT | IP-10 | CRP | 108 |
| 3 | 30 | 0.78 | | TRAIL | SAA | PCT | IP-10 | IL1ra | 109 |
| 3 | 30 | 0.73 | TRAIL | SAA | PCT | IP-10 | IL1ra | CRP | 110 |

Table 31 below list the coefficients b₀, b₁, b₂, etc that were used to define the coordinate δ₁, for each of the 110 cases listed in Table 30, respectively. The first coefficient on the left is b₀, and then from left to right, the coefficients correspond to the order of the polypeptides in each row of Table 30. The coefficients correspond to the following concentration scales for each polypeptide: TRAIL (pg/ml). IP-10 (pg/ml). CRP (ug/ml), PCT (ng/ml), SAA (g/ml) and IL1ra (g/ml).

TABLE 31

| Coefficients | | | | | | | No. |
|---|---|---|---|---|---|---|---|
| | | | | −9849178.8 | 0.0363 | −1.997 | 1 |
| | | | | −0.0009 | 0.039722 | −1.6069 | 2 |
| | | | | 0.6405 | 0.054137 | −2.9681 | 3 |
| | | | | 1098.3777 | 0.034353 | −2.33196 | 4 |
| | | | | −0.00089 | 47954608.09 | 0.4715979 | 5 |
| | | | | 4.5607 | −69280395.624 | −0.74822 | 6 |
| | | | | 5283.68 | −33345728.8342 | −1.706206 | 7 |
| | | | | −0.03151 | 43833567.7377 | 3.0601663 | 8 |
| | | | | 0.86013 | −0.00060898 | −0.13268 | 9 |
| | | | | 4677.8311 | −0.0009684361 | −1.01872 | 10 |
| | | | | −0.0288 | 0.00031349 | 2.5632 | 11 |
| | | | | 2349.8702 | 1.1895403 | −1.35195 | 12 |
| | | | | −0.019169 | 0.4382 | 1.4742 | 13 |
| | | | | −0.02176 | 2962.7685 | 1.08972 | 14 |
| | | | −0.00165 | 6.264E+7 | 0.039986 | −1.27532 | 15 |
| | | | 1.07655 | −8.42E+7 | 0.0475326 | −2.3376 | 16 |
| | | | 2098.4 | −2.22E+7 | 0.027867 | −2.23709 | 17 |
| | | | −0.0266 | 2.0497E+7 | 0.030146 | 0.9001561 | 18 |
| | | | 0.65349 | −0.0005 | 0.051698 | −2.5383 | 19 |
| | | | 1378.2 | −0.00109 | 0.034481544 | −1.6940577 | 20 |
| | | | −1243.01 | 1.4735726 | 0.054245413 | −2.7487888 | 21 |
| | | | −0.010529 | 0.42793 | 0.04535 | −1.421 | 22 |
| | | | −0.01891 | 183.3117 | 0.0312776 | 0.1044034 | 23 |
| | | | 4.8755 | −0.001241 | −4107077 | −0.0013248 | 24 |
| | | | 5777 | −0.001377 | 21179055 | −1.054077 | 25 |
| | | | −0.03151 | −1.118−06 | 43882108 | 3.0605 | 26 |
| | | | 4823 | 2.91 | −68741718 | −1.9806377 | 27 |
| | | | −0.0342 | 1.941 | 113905139.6 | 2.844483 | 28 |
| | | | −0.0264 | 3745.49 | −7296968.1 | 1.4399 | 29 |
| | | | 2427.6 | 1.3263344 | −0.000765 | −0.8562752 | 30 |
| | | | −0.020588 | 0.38993 | 0.00045394 | 1.357 | 31 |
| | | | −0.021174 | 3048.4182 | −0.000163 | 1.0917705 | 32 |
| | | | −0.013629 | 1431.011 | 0.89320046 | 0.48274 | 33 |
| | | 1.5 | −0.003888 | 75533424 | 0.07214 | −0.6620 | 34 |
| | | 2425.771 | −0.002 | 59894763 | 0.034006 | −1.433018 | 35 |
| | | −0.0251 | −0.00084 | 50294164 | 0.03259 | 1.074937 | 36 |
| | | 893.395 | 1.1316 | −70994467 | 0.038 | −2.302 | 37 |
| | | −0.0477 | −0.084 | −81575254 | 0.061632 | 1.903272 | 38 |
| | | −0.02483 | 1236 | 10145313 | 0.025 | 0.65146 | 39 |
| | | −949.2 | 1.528887 | −5.5688E−4 | 0.04984696 | −2.32016 | 40 |
| | | −0.011113 | 0.40033 | 0.00021523 | 0.045264 | −1.4662 | 41 |
| | | −0.0177 | 329.7448 | −0.0003975 | 0.03169 | 0.14333 | 42 |
| | | −0.011 | −1930.2 | 1.24 | 0.050385 | −1.109923 | 43 |
| | | 6082.17 | 4.286 | −0.002014 | 2715886 | −1.087150 | 44 |
| | | −0.0397 | 2.126 | 0.00092636 | −1508120 | 2.9154 | 45 |
| | | −0.0252 | 4082.939 | −0.00062 | 17100158 | 1.55114 | 46 |
| | | −0.0560 | 7639.7 | 0.68134 | −27909258 | 2.85226 | 47 |
| | | −0.0134 | 1423.99 | 0.87764371 | 6.13e−05 | 0.446 | 48 |
| | 4736.86 | 1.250 | −0.00652 | 172681901 | 0.07676 | −0.3021 | 49 |
| | −0.044 | −0.121 | −0.000873 | −4.62E+7 | 0.0671 | 1.937 | 50 |
| | −0.0219 | 1576.6 | −0.00134 | 54069432 | 0.029267 | 0.78878 | 51 |
| | −0.055 | 3598 | −0.098620 | −74159142 | 0.041577 | 2.309 | 52 |
| | −0.0116 | −2055 | 1.188 | 0.00023 | 0.0512 | −1.1542 | 53 |
| | −0.055 | 8903.82 | 1.03 | −0.0012627 | 14035678 | 3.2 | 54 |
| −0.078 | 14133 | −0.687 | −0.009695 | 1.062E+8 | 0.10 | 5.59 | 55 |
| | | | | 3.996E+8 | 0.11089 | −1.021759 | 56 |
| | | | | 0.0063336 | 0.11347 | −1.9467 | 57 |
| | | | | 860.3249 | 0.0639025 | −84.98948 | 58 |
| | | | | 9898.8177 | 0.091563631 | −0.3299621 | 59 |
| | | | | 0.00721 | 107920251.6624 | −1.0006445 | 60 |
| | | | | 419.2 | 596535240 | −41.585735 | 61 |
| | | | | 14320 | 234257296.8937 | −0.4789050 | 62 |
| | | | | 0.00066 | 812307573.5455 | 0.09918792 | 63 |
| | | | | 1089.4251 | 0.00069423293 | −107.18015 | 64 |
| | | | | 12590.5 | 0.00967490979 | −2.05501 | 65 |
| | | | | −0.00905 | 0.0092076 | 0.19189 | 66 |
| | | | | 165893.71 | 122.7205081 | −11.30895 | 67 |
| | | | | 0.0041105 | 6.5788 | 0.98581 | 68 |
| | | | | 0.010541 | 19453.2163 | −1.366750 | 69 |
| | | | 0.0062 | −77782071 | 0.10876 | −2.301980 | 70 |
| | | | 393.7 | 559628637 | 0.048935 | −39.915 | 71 |
| | | | 8656.83 | 244256710 | 0.0663 | −0.885780 | 72 |
| | | | 0.0129 | 157875482 | 0.142003 | −2.694252 | 73 |
| | | | 846.608 | 0.0014 | 0.07831107 | −84.66684 | 74 |
| | | | 5900.1661 | 0.00927 | 0.081369191 | −2.5885198 | 75 |
| | | | 131629 | 108.84 | 0.06793071342 | −10.12169 | 76 |
| | | | 0.011421 | 822.6365 | 0.08303337 | −82.88872 | 77 |
| | | | 0.013257 | 10662.5415 | 0.106214424 | −2.33978 | 78 |
| | | | 417.43 | −0.000381 | 744190123.3893 | −41.369532 | 79 |
| | | | 12128 | 0.0091619 | −130390666 | −2.266204 | 80 |
| | | | −0.005459 | 0.007583 | 82287681 | −0.50010 | 81 |
| | | | 377360 | −8.1908 | 6837963488 | −2.47028 | 82 |
| | | | 0.00099 | 418.212 | 560182293 | −41.5502 | 83 |
| | | | 0.011194 | 17111.2 | 29398797 | −1.8577 | 84 |
| | | | 21649017 | 28.96307 | 0.4328 | −156.16 | 85 |
| | | | 0.00330 | 1086.1672 | 0.00029753173 | −107.01823 | 86 |
| | | | −9.3941e−05 | 12572.6828 | 0.00969 | −2.0464 | 87 |
| | | | 24.2 | 80696477 | 471.6 | −2614.99 | 88 |
| | | 392.929 | −0.0001 | 611767730 | 0.0491 | −39.82 | 89 |
| | | 6854 | 0.00937 | −157521601 | 0.070555 | −2.81351 | 90 |
| | | 0.005871 | 0.00552 | −61236289 | 0.118 | −2.9416 | 91 |
| | | 403954 | −8.6576 | 7107285383 | −0.07356 | −2.349 | 92 |
| | | 0.00857 | 373.75 | 383823513 | 0.05763 | −38.781 | 93 |
| | | 0.013998 | 9692.125 | −4665192.1 | 0.0965657 | −2.782 | 94 |
| | | 4998296 | −132.70 | 0.3202 | 10.567847038 | −132.7427 | 95 |
| | | 0.00927 | 827.6066 | 0.000498 | 0.08349426 | −83.41464 | 96 |
| | | 0.00369 | 6461.9905 | 0.008696 | 0.084631596 | −2.9639303 | 97 |
| | | 2.32E+12 | 4.83248e+18 | −1.05E+14 | 9037614498892 | 1.185E+14 | 98 |
| | | 9471186 | −296 | 0.196688 | 116933544267 | −99.64 | 99 |
| | | 0.002761 | 413.88 | −0.00058 | 713679677.7954 | −41.177966 | 100 |
| | | 0.00349 | 12684.8 | 0.0088176 | −124943185 | −2.6391378 | 101 |
| | | 1.3718 | 8853215 | −272.0191 | 68076716508 | −163.16785 | 102 |
| | | 0.9352 | 11007611 | 24.21772 | 0.09197 | −134.8402 | 103 |
| | 5448434 | −195 | 0.1975318 | 32157214873 | 5.367 | −82.7 | 104 |
| | 0.024158 | 327.2 | −0.002344 | 823767988 | 0.0803 | −35.325 | 105 |
| | 0.0065 | 7390.9 | 0.008791 | −151905670 | 0.080040 | −3.579 | 106 |
| | 2.78 | −1129873 | −106.418 | 43593035460 | 29.2 | −338.972 | 107 |
| | 1.563 | −96788.08 | −22.217 | 0.4843 | 8.2370 | −237.8248 | 108 |
| | 4.06E+12 | 1.757e+18 | 2.798E+13 | 3.97E+12 | −5.96133e+22 | −8.51E+14 | 109 |
| 1.839 | −9.83E+5 | −16.687 | 0.58062 | −4575512593 | 9.549 | −276.3 | 110 |

### Two-Dimensional Manifold

When n=2, the manifold S is a curved surface and the hyperplane π is a flat plane defined by the first direction δ₀ and the second direction δ₁. The coordinate δ₀ in this Example is optionally and preferably a linear combination a₀+a₁D₁+a₂D₂+ . . . , of the polypeptides D₁, D₂, etc; and the coordinate δ₁ in this Example is optionally and preferably a linear combination b₀+b₁D₁+b₂D₂+ . . . , of the polypeptides D₁, D₂, etc.

Tables 32-35 below list diagnostic performance (in AUCs) attained for n=2. The performance were computed using a leave-10%-out cross validation on a subset of the majority cohort that had sufficient serum to measure all the proteins. The coordinates δ₀ and δ₁ were calculated so that the probabilistic classification function f(δ₀, δ₁) represented the likelihood that the test subject had a bacterial infection. The AUC values correspond to classifications according to Bacterial versus Viral (second column from right—B vs. V) and infectious vs. non-infectious (rightmost column—I vs. NI). Shown are results for the embodiments in which the plurality of polypeptides includes two polypeptides (Table 32), three polypeptides (Table 33), four polypeptides (Table 34) and five polypeptides (Table 35). The coefficients for the coordinates δ₀ and δ₁ are presented for each polypeptide, wherein “const” correspond to do when applied to the coordinate δ₀ and b₀ when applied to the coordinate δ₁. The coefficients correspond to the following concentration scales for each protein: TRAIL (pg/ml), IP-10 (pg/ml), CRP (ug/ml), PCT (ng/ml), SAA (g/ml) and IL1ra (g/ml).

TABLE 32

| AUC | AUC | | | | |
|---|---|---|---|---|---|
| (I vs. NI) | (B vs. V) | | | | |
| 0.91 | 0.88 | TRAIL | IP-10 | Const | |
| | | 0.0006 | 0.0086 | −0.3333 | δ0 |
| | | −0.0294 | 0.0089 | 2.4481 | δ1 |
| 0.95 | 0.89 | IP-10 | CRP | Const | |
| | | 0.0055 | 0.0517 | −0.474 | δ0 |
| | | 0.0046 | 0.0902 | −1.9201 | δ1 |
| 0.96324 | 0.85647 | SAA | IP-10 | Const | |
| | | 9623.7195 | 0.0089 | −1.0634 | δ0 |
| | | 14280.3897 | 0.0079 | −2.0098 | δ1 |
| 0.89408 | 0.63901 | IP-10 | IL1ra | Const | |
| | | 0.0077 | 77589304.64 | −0.2347 | δ0 |
| | | 0.0069 | 122880671.4 | 0.3245 | δ1 |
| 0.735 | 0.70468 | PCT | IP-10 | Const | |
| | | 0.1778 | 0.0012 | 1.3717 | δ0 |
| | | 0.9426 | 0.0007 | 1.3073 | δ1 |
| 0.93 | 0.94 | TRAIL | CRP | Const | |
| | | 0.0129 | 0.0647 | −0.551 | δ0 |
| | | −0.0077 | 0.0953 | −0.1177 | δ1 |
| 0.92719 | 0.90714 | TRAIL | SAA | Const | |
| | | 0.0146 | 15457.6689 | −1.0101 | δ0 |
| | | −0.0081 | 18311.8735 | 0.2736 | δ1 |
| 0.85523 | 0.88673 | TRAIL | IL1ra | Const | |
| | | 0.0118 | 660539652.3 | −0.1638 | δ0 |
| | | −0.0224 | 691029794.9 | 3.3011 | δ1 |
| 0.69731 | 0.86706 | TRAIL | PCT | Const | |
| | | 0.0095 | 0.6699 | 0.7941 | δ0 |
| | | −0.0105 | 1.0871 | 2.4419 | δ1 |
| 0.92 | 0.89 | SAA | CRP | Const | |
| | | 7927.9578 | 0.0371 | 0.9937 | δ0 |
| | | 9043.9184 | 0.0704 | −1.2549 | δ1 |
| 0.93 | 0.87 | IL1ra | CRP | Const | |
| | | 357544464 | 0.0549 | 0.9321 | δ0 |
| | | 345095895 | 0.0895 | −0.8849 | δ1 |
| 0.85 | 0.88 | PCT | CRP | Const | |
| | | 0.1493 | 0.0543 | 1.225 | δ0 |
| | | 0.71 | 0.1052 | −1.48 | δ1 |
| 0.9154 | 0.82529 | SAA | IL1ra | Const | |
| | | 11965 | 233885248 | 0.9453 | δ0 |
| | | 17194.2625 | 201037678 | −0.6599 | δ1 |
| 0.84314 | 0.78722 | SAA | PCT | Const | |
| | | 6627 | −0.6192 | 1.4185 | δ0 |
| | | 8964 | 0.2744 | 0.1417 | δ1 |
| 0.82323 | 0.58647 | PCT | IL1ra | Const | |
| | | −1.0932 | 601268546 | 1.3547 | δ0 |
| | | 0.7431 | 600085479 | 0.7175 | δ1 |

TABLE 33

| AUC | AUC | | | | | |
|---|---|---|---|---|---|---|
| (I vs. NI) | (B vs. V) | TRAIL | IP-10 | CRP | Const | |
| 0.96 | 0.94 | 0.005 | 0.0053 | 0.0555 | −1.0317 | δ0 |
| | | −0.0143 | 0.005 | 0.0884 | −0.6693 | δ1 |
| | | TRAIL | SAA | IP-10 | Const | |
| 0.96 | 0.91 | 0.0047 | 9804.469 | 0.0087 | −1.636 | δ0 |
| | | −0.0167 | 12810.9197 | 0.0085 | −0.435 | δ1 |
| | | TRAIL | IP-10 | IL1ra | Const | |
| 0.90 | 0.89 | 0.0056 | 0.0072 | 24233992.13 | −0.7474 | δ0 |
| | | −0.0282 | 0.0073 | 57162308.55 | 2.6252 | δ1 |
| | | TRAIL | PCT | IP-10 | Const | |
| 0.66 | 0.85 | 0.008 | 0.7463 | 0.0005 | 0.71 | δ0 |
| | | −0.0136 | 1.1103 | 0.001 | 2.1832 | δ1 |
| | | SAA | IP-10 | CRP | Const | |
| 0.97318 | 0.91325 | 4964.9078 | 0.0079 | 0.0389 | −1.1506 | δ0 |
| | | 6345.7097 | 0.0069 | 0.0729 | −2.7684 | δ1 |
| | | IP-10 | IL1ra | CRP | Const | |
| 0.95695 | 0.90645 | 0.0062 | −72572842.54 | 0.0635 | −0.5109 | δ0 |
| | | 0.0046 | −16278785.64 | 0.1025 | −1.6901 | δ1 |
| | | PCT | IP-10 | CRP | Const | |
| 0.8 | 0.88475 | 0.1083 | 0.0016 | 0.0598 | 0.1233 | δ0 |
| | | 0.6599 | 0.0011 | 0.1081 | −2.1504 | δ1 |
| | | SAA | IP-10 | IL1ra | Const | |
| 0.94944 | 0.85722 | 9571.3145 | 0.0094 | −141670519.4 | −0.97 | δ0 |
| | | 15309.775 | 0.008 | −119518794.5 | −1.932 | δ1 |
| | | SAA | PCT | IP-10 | Const | |
| 0.95635 | 0.79658 | 6137.1652 | −0.6596 | 0.0047 | −0.5085 | δ0 |
| | | 8580.4524 | 0.2775 | 0.004 | −1.3306 | δ1 |
| | | PCT | IP-10 | IL1ra | Const | |
| 0.73737 | 0.69549 | −1.1448 | 0.0005 | 540518195.3 | 1.0752 | δ0 |
| | | 0.7431 | −0.0003 | 578154355.6 | 0.9893 | δ1 |
| | | TRAIL | SAA | CRP | Const | |
| 0.94489 | 0.93838 | 0.0147 | 8741.563 | 0.0419 | −1.1898 | δ0 |
| | | −0.0045 | 8922.431 | 0.0715 | −0.9205 | δ1 |
| | | TRAIL | IL1ra | CRP | Const | |
| 0.92941 | 0.94316 | 0.0158 | 142723684.3 | 0.0735 | −1.1214 | δ0 |
| | | −0.0124 | 142922206.2 | 0.1005 | 0.254 | δ1 |
| | | TRAIL | PCT | CRP | Const | |
| 0.85644 | 0.91373 | 0.0132 | 0.3236 | 0.066 | −0.695 | δ0 |
| | | 0.0019 | 0.6114 | 0.1084 | −1.7666 | δ1 |
| | | TRAIL | SAA | IL1ra | Const | |
| 0.91298 | 0.91698 | 0.0165 | 13897.6693 | 19314215.49 | −1.1796 | δ0 |
| | | −0.0114 | 17471.1789 | −373899.4284 | 0.5955 | δ1 |
| | | TRAIL | SAA | PCT | Const | |
| 0.9451 | 0.85 | 0.0281 | 13902.8636 | −0.0348 | −2.1844 | δ0 |
| | | 0.0141 | 15302.3132 | 0.7361 | −1.6348 | δ1 |
| | | TRAIL | PCT | IL1ra | Const | |
| 0.73737 | 0.8797 | 0.0126 | 1.6517 | 445497461.8 | −0.3418 | δ0 |
| | | −0.0203 | 2.4203 | 638669048.4 | 2.2766 | δ1 |
| | | SAA | IL1ra | CRP | Const | |
| 0.91932 | 0.88856 | 7641.7563 | 224710899.2 | 0.0265 | 0.8638 | δ0 |
| | | 9730.7248 | 201425116.6 | 0.0536 | −1.256 | δ1 |
| | | SAA | PCT | CRP | Const | |
| 0.90588 | 0.88556 | 8520.704 | −1.4792 | 0.0207 | 1.1579 | δ0 |
| | | 7599.3621 | −0.2234 | 0.0695 | −1.3994 | δ1 |
| | | PCT | IL1ra | CRP | Const | |
| 0.84343 | 0.86842 | −0.6599 | 547844063.4 | 0.0388 | 0.8368 | δ0 |
| | | −0.1506 | 473174484.1 | 0.0873 | −1.6604 | δ1 |
| | | SAA | PCT | IL1ra | Const | |
| 0.9 | 0.81448 | 10349.4815 | −2.3088 | 565967860.9 | 1.0109 | δ0 |
| | | 15172.8663 | −0.2687 | 515166286.4 | −1.0283 | δ1 |

TABLE 34

| AUC | AUC | | | | | | |
|---|---|---|---|---|---|---|---|
| (I vs. | (B vs. | | | | | | |
| NI) | V) | TRAIL | SAA | IP-10 | CRP | Const | |
| 0.97 | 0.94 | 0.0058 | 5383.841 | 0.0075 | 0.0394 | −1.7981 | δ0 |
| | | −0.012 | 5731.9467 | 0.007 | 0.0702 | −1.5541 | δ1 |
| | | TRAIL | IP-10 | IL1ra | CRP | Const | |
| 0.96 | 0.94 | 0.0091 | 0.0053 | −6.995E+7 | 0.0703 | −1.5229 | δ0 |
| | | −0.0166 | 0.0046 | −3.228E+7 | 0.101 | −0.2128 | δ1 |
| | | TRAIL | PCT | IP-10 | CRP | Const | |
| 0.78667 | 0.903 | 0.0101 | 0.2921 | 0.0007 | 0.0651 | −0.6733 | δ0 |
| | | −0.0021 | 0.5293 | 0.001 | 0.1077 | −1.8383 | δ1 |
| | | TRAIL | SAA | IP-10 | IL1ra | Const | |
| 0.94957 | 0.91777 | 0.0091 | 10289.5699 | 0.0088 | 153195983.2 | −2.036 | δ0 |
| | | −0.0169 | 14282.9357 | 0.0082 | 138993063.2 | −0.2825 | δ1 |
| | | TRAIL | SAA | PCT | IP-10 | Const | |
| 0.93254 | 0.8433 | 0.0218 | 12161.0003 | −0.2264 | 0.0068 | −3.3387 | δ0 |
| | | 0.0083 | 13578.3133 | 0.5366 | 0.0068 | −2.9001 | δ1 |
| | | TRAIL | PCT | IP-10 | IL1ra | Const | |
| 0.65657 | 0.86842 | 0.0147 | 1.6805 | −0.0004 | 481673333.4 | −0.356 | δ0 |
| | | −0.0268 | 2.4993 | 0.001 | 491494579.8 | 2.4805 | δ1 |
| | | SAA | IP-10 | IL1ra | CRP | Const | |
| 0.95829 | 0.92002 | 6131.1692 | 0.0088 | −1.5446E+8 | 0.028 | −1.0249 | δ0 |
| | | 8579.4749 | 0.0067 | −9.6352E+7 | 0.0614 | −2.3655 | δ1 |
| | | SAA | PCT | IP-10 | CRP | Const | |
| 0.9881 | 0.8735 | 4377.1407 | −1.4641 | 0.0064 | 0.0419 | −1.4913 | δ0 |
| | | 3810.7522 | −0.1982 | 0.0059 | 0.0857 | −3.62 | δ1 |
| | | PCT | IP-10 | IL1ra | CRP | Const | |
| 0.74242 | 0.89098 | −0.4843 | 0.0004 | 4.54739E+8 | 0.0378 | 0.6379 | δ0 |
| | | −0.2044 | −0.0018 | 4.84865E+8 | 0.0969 | −0.7642 | δ1 |
| | | SAA | PCT | IP-10 | IL1ra | Const | |
| 0.94444 | 0.77828 | 4951.1109 | −2.8236 | 0.0095 | 212692846.6 | −0.802 | δ0 |
| | | 10430.5725 | −0.1446 | 0.008 | 210027138.1 | −2.0339 | δ1 |
| | | TRAIL | SAA | IL1ra | CRP | Const | |
| 0.92564 | 0.93742 | 0.0163 | 8701.5399 | 2.10729E+7 | 0.0386 | −1.3076 | δ0 |
| | | −0.0099 | 9890.6956 | 1.31614E+7 | 0.062 | −0.2694 | δ1 |
| | | TRAIL | SAA | PCT | CRP | Const | |
| 0.95294 | 0.91111 | 0.0253 | 11551.5028 | −1.3285 | 0.0278 | −1.8221 | δ0 |
| | | 0.0141 | 9802.9581 | −0.2648 | 0.0748 | −2.7829 | δ1 |
| | | TRAIL | PCT | IL1ra | CRP | Const | |
| 0.79798 | 0.89474 | 0.0137 | −0.1689 | 2.756E+8 | 0.0476 | −0.6344 | δ0 |
| | | −0.0264 | −0.236 | 2.7563E+8 | 0.0994 | 0.5587 | δ1 |
| | | TRAIL | SAA | PCT | IL1ra | Const | |
| 0.85556 | 0.92308 | 0.0343 | 12347.4916 | −0.5098 | 432026875.9 | −2.2741 | δ0 |
| | | −0.0152 | 19586.5686 | −0.4124 | 426850211.8 | 0.0383 | δ1 |
| | | SAA | PCT | IL1ra | CRP | Const | |
| 0.9 | 0.85068 | 2665.2949 | −0.5099 | 6.42961E+8 | 0.0552 | 0.5611 | δ0 |
| | | 3734.4091 | −0.3614 | 5.88426E+8 | 0.0941 | −1.8313 | δ1 |

TABLE 35

| AUC | AUC | | | | | | | |
|---|---|---|---|---|---|---|---|---|
| (I vs. | (B vs. | | | | | | | |
| NI) | V) | TRAIL | SAA | IP-10 | IL1ra | CRP | Const | |
| 0.95963 | 0.94381 | 0.0092 | 6688.18 | 0.0082 | −1.6265E+8 | 0.0336 | −2.1333 | δ0 |
| | | −0.0136 | 8261.93 | 0.0069 | −1.17187E+8 | 0.0619 | −1.1202 | δ1 |
| | | TRAIL | SAA | PCT | IP-10 | CRP | Const | |
| 0.95635 | 0.89972 | 0.0178 | 6302.89 | −1.297 | 0.0074 | 0.0517 | −3.4117 | δ0 |
| | | 0.0063 | 4437.96 | −0.249 | 0.0076 | 0.1 | −4.4957 | δ1 |
| | | TRAIL | PCT | IP-10 | IL1ra | CRP | Const | |
| 0.71717 | 0.88346 | 0.0246 | −0.2302 | −0.0017 | 5.4749E+8 | 0.0616 | −1.3864 | δ0 |
| | | −0.017 | −0.2819 | −0.0012 | 5.0261E+8 | 0.1096 | −0.1627 | δ1 |
| | | TRAIL | SAA | PCT | IP-10 | IL1ra | Const | |
| 0.85556 | 0.87783 | 0.0529 | 5922.72 | −0.7334 | 0.0149 | 2530173.292 | −6.1686 | δ0 |
| | | 0.0043 | 14225.92 | −0.282 | 0.0139 | 32115407.24 | −3.7073 | δ1 |
| | | SAA | PCT | IP-10 | IL1ra | CRP | Const | |
| 0.91111 | 0.819 | −22863.96 | −0.2611 | 0.0141 | −8.7081E+8 | 0.1586 | −2.8588 | δ0 |
| | | −18573.7 | −0.3918 | 0.008 | 7.27742E+8 | 0.2362 | −3.2596 | δ1 |
| | | TRAIL | SAA | PCT | IL1ra | CRP | Const | |
| 0.87778 | 0.90045 | 0.0397 | −7661.57 | −0.4075 | 6.98426E+8 | 0.1355 | −3.522 | δ0 |
| | | −0.008 | −4178.89 | −0.4915 | 6.53495E+8 | 0.1689 | −1.7514 | δ1 |

### Example 13

### Exemplified Coordinates That Include Nonlinear Functions

It was unexpectedly found by the present Inventor that incorporation of the nonlinear functions ϕ₀ and ϕ₁ in the calculation of the coordinates δ₁ and δ2 captures more subtle trends in the data, while retaining a probabilistic framework that allows meaningful interpretation of the results. In this Example, the coordinates δ0 and δ1 were calculated according to the following equations:

$$\begin{matrix}{δ}_{0} = {a}_{0} + {a}_{1} C + {a}_{2} I + {a}_{3} T + {ϕ}_{0} \\ {δ}_{1} = {b}_{0} + {b}_{1} C + {b}_{2} I + {b}_{3} T + {ϕ}_{1} ,\end{matrix}$$

and the nonlinear functions were defined as:

$$\begin{matrix}{ϕ}_{0} = {q}_{1} {C}^{{γ}^{1}} + {q}_{2} {C}^{{γ}^{2}} + {q}_{3} {T}^{{γ}^{3}} \\ {ϕ}_{1} = {r}_{1} {C}^{{γ}^{1}} + {r}_{2} {C}^{{γ}^{2}} + {r}_{3} {T}^{{γ}^{3}} .\end{matrix}$$

where γ1=0.5, γ2=2 and γ3=0.5.

Table 36 details the coefficients and constants used in this Example.

TABLE 36

| First Coordinate | Second Coordinate | |
|---|---|---|
| δ₀ (viral) | δ₁ (bacterial) | |
| a₀ = −0.8388 | b₀ = 5.5123 | Const |
| a₁ = −0.0487 | b₁ = −0.0636 | CRP (mg/ml) |
| q₁ = 1.1367 | r₁ = 1.4877 | CRP^{0.5} (mg/ml)^{0.5} |
| q₂ = −5.14 × 10⁻⁰⁵ | r₂ = 3.50 × 10⁻⁰⁵ | CRP² (mg/ml)² |
| a₂ = 0.0089 | b₂ = 0.0085 | IP10 (pg/ml) |
| a₃ = 0.0408 | b₃ = 0.0646 | TRAIL (pg/ml) |
| q₃ = -0.6064 | r₃ = −1.8039 | TRAIL^{0.5} (pg/ml)^{0.5} |

The performance of the model presented in Table 36 was examined on the Microbiologically Confirmed Cohort (AUC of 0.95±0.03), Unanimous Cohort (AUC of 0.95±0.02) and the Study cohort (AUC of 0.93±0.02). The signature performance improved as the size of the equivocal region increases.

Tables 37A-C below detail signature measures of accuracy for diagnosing bacterial versus viral infections when using the nonlinear model of the present Example. Performance estimates and their 95% CIs were obtained on the Microbiologically Confirmed sub-cohort (Table 37A; n=241), Unanimous sub-cohort (Table 37B; n=527), and Study Cohort (Table 37C; n=653), using different sizes of equivocal regions as indicated. Tables 37D-F below detail percentage of patients who had equivocal immune response in the Study Cohort when applying different thresholds, and Tables 37G-H below detail signature sensitivity and specificity when applying different equivocal immune response thresholds obtained on the Study Cohort. In Tables 37D-H the leftmost columns represents a minimal equivocal immune response threshold and the uppermost row represents a maximal equivocal immune response threshold.

TABLE 37A

| Equivocal | Equivocal | Equivocal | Equivocal | | |
|---|---|---|---|---|---|
| immune | immune | immune | immune | | |
| response | response | response | response | | |
| filter | filter | filter | filter | All | Accuracy |
| (10-90) | (20-80) | (30-70) | (35-65) | patients | measure |
| 0.98, | 0.96, | 0.94, | 0.93, | 0.89, | Total |
| (0.96, | (0.93, | (0.91, | (0.90, | (0.85, | accuracy |
| 1.00) | 0.99) | 0.97) | 0.97) | 0.93) | |
| 0.96, | 0.96, | 0.95, | 0.93, | 0.88, | Sensitivity |
| (0.90, | (0.91, | (0.89, | (0.87, | (0.80, | |
| 1.00) | 1.00) | 1.00) | 1.00) | 0.96) | |
| 0.99, | 0.96, | 0.94, | 0.94, | 0.90, | Specificity |
| (0.97, | (0.93, | (0.90, | (0.90, | (0.87, | |
| 1.00) | 0.99) | 0.98) | 0.97) | 0.94) | |
| 65% | 78% | 87% | 90% | 100% | % of |
| | | | | | patients |
| | | | | | included |

TABLE 37B

| Equivocal | Equivocal | Equivocal | Equivocal | | |
|---|---|---|---|---|---|
| immune | immune | immune | immune | | |
| response | response | response | response | | |
| filter | filter | filter | filter | All | Accuracy |
| (10-90) | (20-80) | (30-70) | (35-65) | patients | measure |
| 0.97, | 0.95, | 0.93, | 0.92, | 0.88, | Total |
| (0.95, | (0.93, | (0.90, | (0.89, | (0.85, | accuracy |
| 0.99) | 0.97) | 0.95) | 0.94) | 0.91) | |
| 0.96, | 0.93, | 0.91, | 0.90, | 0.85, | Sensitivity |
| (0.93, | (0.90, | (0.87, | (0.86, | (0.81, | |
| 0.99) | 0.97) | 0.95) | 0.94) | 0.89) | |
| 0.98, | 0.96, | 0.94, | 0.93, | 0.91, | Specificity |
| (0.96, | (0.93, | (0.91, | (0.90, | (0.87, | |
| 1.00) | 0.99) | 0.97) | 0.97) | 0.94) | |
| 63% | 76% | 86% | 90% | 100% | % of |
| | | | | | patients |
| | | | | | included |

TABLE 37C

| Equivocal | Equivocal | Equivocal | Equivocal | | |
|---|---|---|---|---|---|
| immune | immune | immune | immune | | |
| response | response | response | response | | |
| filter | filter | filter | filter | All | Accuracy |
| (10-90) | (20-80) | (30-70) | (35-65) | patients | measure |
| 0.95, | 0.92, | 0.90, | 0.89, | 0.85, | Total |
| (0.93, | (0.90, | (0.87, | (0.86, | (0.83, | accuracy |
| 0.98) | 0.95) | 0.92) | 0.91) | 0.88) | |
| 0.95, | 0.92, | 0.89, | 0.87, | 0.83, | Sensitivity |
| (0.91, | (0.88, | (0.85, | (0.83, | (0.79, | |
| 0.98) | 0.95) | 0.92) | 0.91) | 0.87) | |
| 0.95, | 0.93, | 0.91, | 0.90, | 0.87, | Specificity |
| (0.92, | (0.89, | (0.88, | (0.87, | (0.84, | |
| 0.98) | 0.96) | 0.95) | 0.94) | 0.91) | |
| 58% | 72% | 84% | 88% | 100% | % of |
| | | | | | patients |
| | | | | | included |

TABLE 37D

| | | | | | | | | | | | | | | | | | | |
|---|---|---|---|---|---|---|---|---|---|---|---|---|---|---|---|---|---|---|
| 0.95 | 0.9 | 0.85 | 0.8 | 0.75 | 0.7 | 0.65 | 0.6 | 0.55 | 0.5 | 0.45 | 0.4 | 0.35 | 0.3 | 0.25 | 0.2 | 0.15 | 0.1 | |
| 52.8 | 47.2 | 44.0 | 40.9 | 38.6 | 36.3 | 34.8 | 33.2 | 31.2 | 29.1 | 26.3 | 24.0 | 22.7 | 20.5 | 17.6 | 13.9 | 10.4 | 6.6 | 0.05 |
| 46.2 | 40.6 | 37.4 | 34.3 | 32.0 | 29.7 | 28.2 | 26.6 | 24.7 | 22.5 | 19.8 | 17.5 | 16.1 | 13.9 | 11.0 | 7.4 | 3.8 | | 0.1 |
| 42.4 | 36.8 | 33.5 | 30.5 | 28.2 | 25.9 | 24.3 | 22.8 | 20.8 | 18.7 | 15.9 | 13.6 | 12.3 | 10.1 | 7.2 | 3.5 | | | 0.15 |
| 38.9 | 33.2 | 30.0 | 27.0 | 24.7 | 22.4 | 20.8 | 19.3 | 17.3 | 15.2 | 12.4 | 10.1 | 8.7 | 6.6 | 3.7 | | | | 0.2 |
| 35.2 | 29.6 | 26.3 | 23.3 | 21.0 | 18.7 | 17.2 | 15.6 | 13.6 | 11.5 | 8.7 | 6.4 | 5.1 | 2.9 | | | | | 0.25 |
| 32.3 | 26.6 | 23.4 | 20.4 | 18.1 | 15.8 | 14.2 | 12.7 | 10.7 | 8.6 | 5.8 | 3.5 | 2.1 | | | | | | 0.3 |
| 30.2 | 24.5 | 21.3 | 18.2 | 15.9 | 13.6 | 12.1 | 10.6 | 8.6 | 6.4 | 3.7 | 1.4 | | | | | | | 0.35 |
| 28.8 | 23.1 | 19.9 | 16.8 | 14.5 | 12.3 | 10.7 | 9.2 | 7.2 | 5.1 | 2.3 | | | | | | | | 0.4 |
| 26.5 | 20.8 | 17.6 | 14.5 | 12.3 | 10.0 | 8.4 | 6.9 | 4.9 | 2.8 | | | | | | | | | 0.45 |
| 23.7 | 18.1 | 14.9 | 11.8 | 9.5 | 7.2 | 5.7 | 4.1 | 2.1 | | | | | | | | | | 0.5 |
| 21.6 | 15.9 | 12.7 | 9.6 | 7.4 | 5.1 | 3.5 | 2.0 | | | | | | | | | | | 0.55 |
| 19.6 | 13.9 | 10.7 | 7.7 | 5.4 | 3.1 | 1.5 | | | | | | | | | | | | 0.6 |
| 18.1 | 12.4 | 9.2 | 6.1 | 3.8 | 1.5 | | | | | | | | | | | | | 0.65 |
| 16.5 | 10.9 | 7.7 | 4.6 | 2.3 | | | | | | | | | | | | | | 0.7 |
| 14.2 | 8.6 | 5.4 | 2.3 | | | | | | | | | | | | | | | 0.75 |
| 11.9 | 6.3 | 3.1 | | | | | | | | | | | | | | | | 0.8 |
| 8.9 | 3.2 | | | | | | | | | | | | | | | | | 0.85 |
| 5.7 | | | | | | | | | | | | | | | | | | 0.9 |

TABLE 37E

| | | | | | | | | | | | | | | | | | | |
|---|---|---|---|---|---|---|---|---|---|---|---|---|---|---|---|---|---|---|
| 0.95 | 0.9 | 0.85 | 0.8 | 0.75 | 0.7 | 0.65 | 0.6 | 0.55 | 0.5 | 0.45 | 0.4 | 0.35 | 0.3 | 0.25 | 0.2 | 0.15 | 0.1 | |
| 53.6 | 43.6 | 38.2 | 33.5 | 29.5 | 26.0 | 23.5 | 21.6 | 18.8 | 16.6 | 13.8 | 11.3 | 10.3 | 9.1 | 7.5 | 5.3 | 3.4 | 2.5 | 0.05 |
| 51.1 | 41.1 | 35.7 | 31.0 | 27.0 | 23.5 | 21.0 | 19.1 | 16.3 | 14.1 | 11.3 | 8.8 | 7.8 | 6.6 | 5.0 | 2.8 | 0.9 | | 0.1 |
| 50.2 | 40.1 | 34.8 | 30.1 | 26.0 | 22.6 | 20.1 | 18.2 | 15.4 | 13.2 | 10.3 | 7.8 | 6.9 | 5.6 | 4.1 | 1.9 | | | 0.15 |
| 48.3 | 38.2 | 32.9 | 28.2 | 24.1 | 20.7 | 18.2 | 16.3 | 13.5 | 11.3 | 8.5 | 6.0 | 5.0 | 3.8 | 2.2 | | | | 0.2 |
| 46.1 | 36.1 | 30.7 | 26.0 | 21.9 | 18.5 | 16.0 | 14.1 | 11.3 | 9.1 | 6.3 | 3.8 | 2.8 | 1.6 | | | | | 0.25 |
| 44.5 | 34.5 | 29.2 | 24.5 | 20.4 | 16.9 | 14.4 | 12.5 | 9.7 | 7.5 | 4.7 | 2.2 | 1.3 | | | | | | 0.3 |
| 43.3 | 33.2 | 27.9 | 23.2 | 19.1 | 15.7 | 13.2 | 11.3 | 8.5 | 6.3 | 3.4 | 0.9 | | | | | | | 0.35 |
| 42.3 | 32.3 | 27.0 | 22.3 | 18.2 | 14.7 | 12.2 | 10.3 | 7.5 | 5.3 | 2.5 | | | | | | | | 0.4 |
| 39.8 | 29.8 | 24.5 | 19.7 | 15.7 | 12.2 | 9.7 | 7.8 | 5.0 | 2.8 | | | | | | | | | 0.45 |
| 37.0 | 27.0 | 21.6 | 16.9 | 12.9 | 9.4 | 6.9 | 5.0 | 2.2 | | | | | | | | | | 0.5 |
| 34.8 | 24.8 | 19.4 | 14.7 | 10.7 | 7.2 | 4.7 | 2.8 | | | | | | | | | | | 0.55 |
| 32.0 | 21.9 | 16.6 | 11.9 | 7.8 | 4.4 | 1.9 | | | | | | | | | | | | 0.6 |
| 30.1 | 20.1 | 14.7 | 10.0 | 6.0 | 2.5 | | | | | | | | | | | | | 0.65 |
| 27.6 | 17.6 | 12.2 | 7.5 | 3.4 | | | | | | | | | | | | | | 0.7 |
| 24.1 | 14.1 | 8.8 | 4.1 | | | | | | | | | | | | | | | 0.75 |
| 20.1 | 10.0 | 4.7 | | | | | | | | | | | | | | | | 0.8 |
| 15.4 | 5.3 | | | | | | | | | | | | | | | | | 0.85 |
| 10.0 | | | | | | | | | | | | | | | | | | 0.9 |

TABLE 37F

| | | | | | | | | | | | | | | | | | | |
|---|---|---|---|---|---|---|---|---|---|---|---|---|---|---|---|---|---|---|
| 0.95 | 0.9 | 0.85 | 0.8 | 0.75 | 0.7 | 0.65 | 0.6 | 0.55 | 0.5 | 0.45 | 0.4 | 0.35 | 0.3 | 0.25 | 0.2 | 0.15 | 0.1 | |
| 52.1 | 50.6 | 49.4 | 47.9 | 47.3 | 46.1 | 45.5 | 44.3 | 43.1 | 41.0 | 38.3 | 36.2 | 34.4 | 31.4 | 27.2 | 22.2 | 17.1 | 10.5 | 0.05 |
| 41.6 | 40.1 | 38.9 | 37.4 | 36.8 | 35.6 | 35.0 | 33.8 | 32.6 | 30.5 | 27.8 | 25.7 | 24.0 | 21.0 | 16.8 | 11.7 | 6.6 | | 0.1 |
| 35.0 | 33.5 | 32.3 | 30.8 | 30.2 | 29.0 | 28.4 | 27.2 | 26.0 | 24.0 | 21.3 | 19.2 | 17.4 | 14.4 | 10.2 | 5.1 | | | 0.15 |
| 29.9 | 28.4 | 27.2 | 25.7 | 25.1 | 24.0 | 23.4 | 22.2 | 21.0 | 18.9 | 16.2 | 14.1 | 12.3 | 9.3 | 5.1 | | | | 0.2 |
| 24.9 | 23.4 | 22.2 | 20.7 | 20.1 | 18.9 | 18.3 | 17.1 | 15.9 | 13.8 | 11.1 | 9.0 | 7.2 | 4.2 | | | | | 0.25 |
| 20.7 | 19.2 | 18.0 | 16.5 | 15.9 | 14.7 | 14.1 | 12.9 | 11.7 | 9.6 | 6.9 | 4.8 | 3.0 | | | | | | 0.3 |
| 17.7 | 16.2 | 15.0 | 13.5 | 12.9 | 11.7 | 11.1 | 9.9 | 8.7 | 6.6 | 3.9 | 1.8 | | | | | | | 0.35 |
| 15.9 | 14.4 | 13.2 | 11.7 | 11.1 | 9.9 | 9.3 | 8.1 | 6.9 | 4.8 | 2.1 | | | | | | | | 0.4 |
| 13.8 | 12.3 | 11.1 | 9.6 | 9.0 | 7.8 | 7.2 | 6.0 | 4.8 | 2.7 | | | | | | | | | 0.45 |
| 11.1 | 9.6 | 8.4 | 6.9 | 6.3 | 5.1 | 4.5 | 3.3 | 2.1 | | | | | | | | | | 0.5 |
| 9.0 | 7.5 | 6.3 | 4.8 | 4.2 | 3.0 | 2.4 | 1.2 | | | | | | | | | | | 0.55 |
| 7.8 | 6.3 | 5.1 | 3.6 | 3.0 | 1.8 | 1.2 | | | | | | | | | | | | 0.6 |
| 6.6 | 5.1 | 3.9 | 2.4 | 1.8 | 0.6 | | | | | | | | | | | | | 0.65 |
| 6.0 | 4.5 | 3.3 | 1.8 | 1.2 | | | | | | | | | | | | | | 0.7 |
| 4.8 | 3.3 | 2.1 | 0.6 | | | | | | | | | | | | | | | 0.75 |
| 4.2 | 2.7 | 1.5 | | | | | | | | | | | | | | | | 0.8 |
| 2.7 | 1.2 | | | | | | | | | | | | | | | | | 0.85 |
| 1.5 | | | | | | | | | | | | | | | | | | 0.9 |

TABLE 37G

| | | | | | | | | | | | | | | | | | | |
|---|---|---|---|---|---|---|---|---|---|---|---|---|---|---|---|---|---|---|
| 0.95 | 0.9 | 0.85 | 0.8 | 0.75 | 0.7 | 0.65 | 0.6 | 0.55 | 0.5 | 0.45 | 0.4 | 0.35 | 0.3 | 0.25 | 0.2 | 0.15 | 0.1 | |
| 98.0 | 98.3 | 98.5 | 98.6 | 98.7 | 98.7 | 98.8 | 98.8 | 98.8 | 98.9 | 95.6 | 92.9 | 92.0 | 90.7 | 89.2 | 87.1 | 85.4 | 84.6 | 0.05 |
| 92.9 | 94.1 | 94.6 | 95.0 | 95.3 | 95.5 | 95.6 | 95.7 | 95.9 | 96.0 | 92.9 | 90.4 | 89.5 | 88.3 | 86.8 | 84.8 | 83.2 | | 0.1 |
| 91.2 | 92.7 | 93.3 | 93.7 | 94.1 | 94.3 | 94.5 | 94.6 | 94.8 | 94.9 | 92.0 | 89.5 | 88.6 | 87.4 | 85.9 | 84.0 | | | 0.15 |
| 87.9 | 89.8 | 90.7 | 91.3 | 91.7 | 92.1 | 92.3 | 92.5 | 92.8 | 92.9 | 90.1 | 87.7 | 86.8 | 85.7 | 84.3 | | | | 0.2 |
| 84.3 | 86.8 | 87.8 | 88.6 | 89.2 | 89.6 | 89.9 | 90.1 | 90.5 | 90.7 | 88.0 | 85.7 | 84.8 | 83.8 | | | | | 0.25 |
| 81.9 | 84.7 | 85.8 | 86.7 | 87.4 | 87.9 | 88.3 | 88.5 | 88.9 | 89.2 | 86.5 | 84.3 | 83.5 | | | | | | 0.3 |
| 80.1 | 83.1 | 84.3 | 85.3 | 86.0 | 86.6 | 87.0 | 87.3 | 87.7 | 88.0 | 85.4 | 83.2 | | | | | | | 0.35 |
| 78.8 | 81.9 | 83.3 | 84.3 | 85.1 | 85.7 | 86.1 | 86.4 | 86.8 | 87.1 | 84.6 | | | | | | | | 0.4 |
| 75.5 | 79.0 | 80.5 | 81.6 | 82.5 | 83.2 | 83.7 | 84.0 | 84.5 | 84.8 | | | | | | | | | 0.45 |
| 72.1 | 76.0 | 77.6 | 78.9 | 79.9 | 80.6 | 81.1 | 81.5 | 82.1 | | | | | | | | | | 0.5 |
| 73.1 | 76.7 | 78.2 | 79.4 | 80.4 | 81.1 | 81.6 | 81.9 | | | | | | | | | | | 0.55 |
| 74.2 | 77.5 | 78.9 | 80.1 | 81.0 | 81.6 | 82.1 | | | | | | | | | | | | 0.6 |
| 74.9 | 78.0 | 79.4 | 80.5 | 81.3 | 82.0 | | | | | | | | | | | | | 0.65 |
| 75.8 | 78.7 | 80.0 | 81.0 | 81.8 | | | | | | | | | | | | | | 0.7 |
| 76.9 | 79.6 | 80.8 | 81.7 | | | | | | | | | | | | | | | 0.75 |
| 78.0 | 80.5 | 81.6 | | | | | | | | | | | | | | | | 0.8 |
| 79.3 | 81.5 | | | | | | | | | | | | | | | | | 0.85 |
| 80.5 | | | | | | | | | | | | | | | | | | 0.9 |

TABLE 37H

| | | | | | | | | | | | | | | | | | | |
|---|---|---|---|---|---|---|---|---|---|---|---|---|---|---|---|---|---|---|
| 0.95 | 0.9 | 0.85 | 0.8 | 0.75 | 0.7 | 0.65 | 0.6 | 0.55 | 0.5 | 0.45 | 0.4 | 0.35 | 0.3 | 0.25 | 0.2 | 0.15 | 0.1 | |
| 97.5 | 94.5 | 92.3 | 89.7 | 88.6 | 86.7 | 85.7 | 83.9 | 82.1 | 79.2 | 80.1 | 80.8 | 81.3 | 82.1 | 83.1 | 84.2 | 85.2 | 86.3 | 0.05 |
| 97.9 | 95.5 | 93.6 | 91.4 | 90.5 | 88.8 | 88.0 | 86.4 | 84.9 | 82.3 | 83.0 | 83.5 | 83.9 | 84.5 | 85.3 | 86.1 | 86.9 | | 0.1 |
| 98.2 | 95.9 | 94.2 | 92.2 | 91.4 | 89.9 | 89.1 | 87.7 | 86.2 | 83.9 | 84.4 | 84.8 | 85.1 | 85.7 | 86.3 | 87.1 | | | 0.15 |
| 98.3 | 96.2 | 94.7 | 92.7 | 92.0 | 90.6 | 89.8 | 88.5 | 87.1 | 84.9 | 85.4 | 85.7 | 86.0 | 86.5 | 87.1 | | | | 0.2 |
| 98.4 | 96.5 | 95.0 | 93.2 | 92.5 | 91.1 | 90.5 | 89.2 | 87.9 | 85.8 | 86.2 | 86.5 | 86.8 | 87.2 | | | | | 0.25 |
| 98.5 | 96.7 | 95.3 | 93.5 | 92.9 | 91.6 | 90.9 | 89.7 | 88.5 | 86.4 | 86.8 | 87.1 | 87.3 | | | | | | 0.3 |
| 98.5 | 96.8 | 95.4 | 93.8 | 93.1 | 91.9 | 91.2 | 90.0 | 88.9 | 86.9 | 87.2 | 87.5 | | | | | | | 0.35 |
| 98.6 | 96.9 | 95.5 | 93.9 | 93.3 | 92.0 | 91.4 | 90.2 | 89.1 | 87.1 | 87.5 | | | | | | | | 0.4 |
| 98.6 | 96.9 | 95.6 | 94.0 | 93.4 | 92.2 | 91.6 | 90.4 | 89.3 | 87.4 | | | | | | | | | 0.45 |
| 98.7 | 97.0 | 95.8 | 94.2 | 93.6 | 92.4 | 91.8 | 90.7 | 89.6 | | | | | | | | | | 0.5 |
| 96.4 | 94.8 | 93.6 | 92.1 | 91.6 | 90.4 | 89.9 | 88.8 | | | | | | | | | | | 0.55 |
| 95.1 | 93.6 | 92.4 | 91.0 | 90.4 | 89.3 | 88.8 | | | | | | | | | | | | 0.6 |
| 93.9 | 92.4 | 91.3 | 89.9 | 89.3 | 88.3 | | | | | | | | | | | | | 0.65 |
| 93.3 | 91.8 | 90.7 | 89.3 | 88.8 | | | | | | | | | | | | | | 0.7 |
| 92.1 | 90.7 | 89.6 | 88.3 | | | | | | | | | | | | | | | 0.75 |
| 91.6 | 90.2 | 89.1 | | | | | | | | | | | | | | | | 0.8 |
| 90.2 | 88.8 | | | | | | | | | | | | | | | | | 0.85 |
| 89.1 | | | | | | | | | | | | | | | | | | 0.9 |

The signature performance was further examined on the Study Cohort when 5 excluding the following two subgroups: (i) patients whose blood sample was taken after more than 3 days of antibiotic treatment in the hospital and (ii) patients with a suspected gastroenteritis. Details of the model performance on the Microbiologically Confirmed Cohort (AUC of 0.96±0.04), Unanimous Cohort (AUC of 0.96±0.02) and the Study cohort (AUC of 0.95±0.02) is further depicted in Table 38A-C.

Tables 38A-C detail signature measures of accuracy for diagnosing bacterial vs. viral infections using the non-linear MLR model. Performance estimates and their 95% CIs were obtained on the Microbiologically Confirmed sub-cohort (Table 38A; n=200), Unanimous sub-cohort (Table 38B; n=402), and Study Cohort (Table 38C; n=491), when excluding patients with over 3 days of antibiotics treatment at the hospital and/or suspicion of gastroenteritis.

TABLE 38A

| Equivocal | Equivocal | Equivocal | Equivocal | | |
|---|---|---|---|---|---|
| immune | immune | immune | immune | | |
| response | response | response | response | | |
| filter | filter | filter | filter | All | Accuracy |
| (10-90) | (20-80) | (30-70) | (35-65) | patients | measure |
| 0.98, | 0.96, | 0.95, | 0.95, | 0.91, | Total |
| (0.96, 1) | (0.93, | (0.92, | (0.92, 1) | (0.86, | accuracy |
| | 0.99) | 0.99) | | 0.95) | |
| 0.94, | 0.95, | 0.96, | 0.96, | 0.90, | Sensitivity |
| (87, 1) | (0.89, 1) | (0.89, 1) | (0.89, 1) | (0.82, | |
| | | | | 0.99) | |
| 1, | 0.97, | 0.95, | 0.95, | 0.91, | Specificity |
| (1, 1) | (0.93, 1) | (0.92, | (0.91, | (0.86, | |
| | | 0.99) | 0.99) | 0.95) | |
| 65% | 80% | 88% | 90% | 100% | % of |
| | | | | | patients |
| | | | | | included |

TABLE 38B

| Equivocal | Equivocal | Equivocal | Equivocal | | |
|---|---|---|---|---|---|
| immune | immune | immune | immune | | |
| response | response | response | response | | |
| filter | filter | filter | filter | All | Accuracy |
| (10-90) | (20-80) | (30-70) | (35-65) | patients | measure |
| 0.98, | 0.96, | 0.95, | 0.94, | 0.91, | Total |
| (0.96, 1) | (0.94, | (0.93, | (0.92, | (0.88, | accuracy |
| | 0.98) | 0.97) | 0.97) | 0.94) | |
| 0.98, | 0.95, | 0.94, | 0.93, | 0.89, | Sensitivity |
| (0.95, 1) | (0.92, | (0.90, | (0.89, | (0.85, | |
| | 0.99) | 0.98) | 0.97) | 0.94) | |
| 0.99, | 0.97, | 0.95, | 0.95, | 0.92, | Specificity |
| (0.97, 1) | (0.94, | (0.93, | (0.92, | (0.88, | |
| | 0.99) | 0.98) | 0.98) | 0.96) | |
| 65% | 79% | 88% | 91% | 100% | % of |
| | | | | | patients |
| | | | | | included |

TABLE 38C

| Equivocal | Equivocal | Equivocal | Equivocal | | |
|---|---|---|---|---|---|
| immune | immune | immune | immune | | |
| response | response | response | response | | |
| filter | filter | filter | filter | All | Accuracy |
| (10-90) | (20-80) | (30-70) | (35-65) | patients | measure |
| 0.97, | 0.94, | 0.93, | 0.91, | 0.88, | Total |
| (0.95, | (0.92, | (0.90, | (0.89, | (0.85, | accuracy |
| 0.99) | 0.97) | 0.95) | 0.94) | 0.91) | |
| 0.97, | 0.95, | 0.92, | 0.91, | 0.87, | Sensitivity |
| (0.94, | (0.91, | (0.88, | (0.87, | (0.83, | |
| 1) | 0.98) | 0.96) | 0.95) | 0.92) | |
| 0.97, | 0.94, | 0.93, | 0.92, | 0.89, | Specificity |
| (0.94, | (0.91, | (0.90, | (0.89, | (0.85, | |
| 1) | 0.97) | 0.96) | 0.96) | 0.92) | |
| 59% | 74% | 85% | 88% | 100% | % of |
| | | | | | patients |
| | | | | | included |

### Example 14

### Antibiotics Based Stratification

Of the 653 patients with suspicion of acute infection, 427 received antibiotics (299 had bacterial diagnosis and 128 had viral diagnosis). The AUC of the signature for distinguishing between the bacterial and viral infected patients in the antibiotics treated patients sub-cohort was 0.93±0.02. No statistically significant difference was observed between the performance on the antibiotics treated patients and the general cohort (0.94±0.02 versus 0.93±0.02; P=0.5).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A computer-implemented method for analyzing biological data, the method comprising:
displaying on a display device a graphical user interface (GUI);
receiving values of biomarkers in the blood of a subject;
responsively to an activation of a calculation activation control by a user, automatically calculating at least one score based on said values, said score being indicative of likelihood for an infection; and
displaying said likelihood over said GUI.

2. The method of claim 1, wherein said receiving said values comprises communicating with a machine that measures said values.

3. The method of claim 2, wherein said GUI further comprises a communication control, wherein said communicating with said machine is in response to an activation of said communication control by the user.

4. The method of claim 1, wherein said GUI comprising a plurality of value input fields, wherein said receiving said values is via said input fields.

5. The method of claim 4, wherein said GUI comprises a clearing control, and the method comprises clearing said value input fields responsively to an activation of said clearing control by said user.

6. The method of claim 4, comprising clearing said value input automatically.

7. The method of claim 1, comprising displaying said at least one score numerically on said GUI.

8. The method according to claim 1, wherein said at least one score comprises a score indicative a bacterial infection.

9. The method according to claim 1, wherein said at least one score comprises a score indicative a viral infection.

10. The method according to claim 1, wherein said at least one score comprises a score indicative a non-infection disease.

11. The method according to claim 1, wherein said at least one score comprises a score indicative a severity of said infection, and the method comprises displaying said severity over said GUI.

12. The method according to claim 1, wherein said calculating said score comprises calculating a distance between a segment of a curved surface and a plane defined by a first direction and a second direction, said distance being calculated at a point over said surface defined by first coordinate δ₀ along said first direction and a second coordinate δ₁ along said second direction; and
using said distance for calculation said score;
wherein each of said coordinates is defined by a different combination of said values.

13. The method according to claim 12, wherein for at least one of said coordinates, said combination of said values comprises a linear combination of said values.

14. The method according to claim 13, wherein for at least one of said coordinates, said combination of the values includes at least one nonlinear term corresponding to at least one of said values.

15. The method according to claim 12, comprising calculating an additional distance between a segment of an additional curved surface and said plane, and using said additional distance for calculation said score.

16. The method according to claim 1, comprising displaying on said GUI an output that summarizes scores calculated for previous blood samples.

17. A system for analyzing biological data, the system comprising a display device and a data processor, wherein said data processor is configured to display on said display device a graphical user interface (GUI), to receive values biomarkers in the blood of a subject, to automatically calculate a score based on said values, responsively to an activation of a calculation activation control by a user, said score being indicative of likelihood for an infection, and to display said likelihood over said GUI.

18. The system according to claim 17, wherein said data processor is configured to communicate with a machine that measures said values, and to receive said values from said machine.

19. The system according to claim 18, wherein said GUI further comprises a communication control, wherein said data processor is configured to communicate with said machine in response to an activation of said communication control by the user.

20. The system according to claim 17, wherein said GUI comprising a plurality of value input fields, and said data processor is configured to receive said values is via said input fields.


SEQUENCE LISTING (38 sequences)

SEQ ID NO: 1 - DNA, 1953 bp, Homo sapiens
  source 1..1953: mol_type = genomic DNA
  mRNA 1..1953
acagaacccagaaaaacaactcattcgctttcatttcctcactgactataaaagaatagagaaggaagggcttcagtgaccggctgcctggctgacttacagcagtcagactctgacaggatcatggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagtttttttggggcctttttagttggctaactgacctggaaagaaaaagcaataacctcaaagtgactattcagttttcaggatgatacactatgaagatgtttcaaaaaatctgaccaaaacaaacaaacagaaaacagaaaacaaaaaaacctctatgcaatctgagtagagcagccacaaccaaaaaattctacaacacacactgttctgaaagtgactcacttatcccaagagaatgaaattgctgaaagatctttcaggactctacctcatatcagtttgctagcagaaatctagaagactgtcagcttccaaacattaatgcaatggttaacatcttctgtctttataatctactccttgtaaagactgtagaagaaagagcaacaatccatctctcaagtagtgtatcacagtagtagcctccaggtttccttaagggacaacatccttaagtcaaaagagagaagaggcaccactaaaagatcgcagtttgcctggtgcagtggctcacacctgtaatcccaacattttgggaacccaaggtgggtagatcacgagatcaagagatcaagaccatagtgaccaacatagtgaaaccccatctctactgaaagtacaaaaattagctgggtgtgttggcacatgcctgtagtcccagctacttgagaggctgaggcaagagaattgtttgaacccgggaggcagaggttgcagtgtggtgagatcatgccactacactccagcctggcgacagagcgagacttggtttcaaaaaaaaaaaaaaaaaaaacttcagtaagtacgtgttatttttttcaataaaattctattacagtatgtcatgtttgctgtagtgctcatatttattgttgtttttgttttagtactcacttgtttcataatatcaagattactaaaaatgggggaaaagacttctaatctttttttcataatatctttgacacatattacagaagaaataaatttcttacttttaatttaatatga

SEQ ID NO: 2 - DNA, 1805 bp, Homo sapiens
  source 1..1805: mol_type = other DNA; note = cDNA
acagaacccagaaaaacaactcattcgctttcatttcctcactgactataaaagaatagagaaggaagggcttcagtgaccggctgcctggctgacttacagcagtcagactctgacaggatcatggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagtttttttggggcctttttagttggctaactgacctggaaagaaaaagcaataacctcaaagtgactattcagttttcaggatgatacactatgaagatgtttcaaaaaatctgaccaaaacaaacaaacagaaaacagaaaacaaaaaaacctctatgcaatctgagtagagcagccacaaccaaaaaattctacaacacacactgttctgaaagtgactcacttatcccaagagaatgaaattgctgaaagatctttcaggactctacctcatatcagtttgctagcagaaatctagaagactgtcagcttccaaacattaatgcaatggttaacatcttctgtctttataatctactccttgtaaagactgtagaagaaagagcaacaatccatctctcaagtagtgtatcacagtagtagcctccaggtttccttaagggacaacatccttaagtcaaaagagagaagaggcaccactaaaagatcgcagtttgcctggtgcagtggctcacacctgtaatcccaacattttgggaacccaaggtgggtagatcacgagatcaagagatcaagaccatagtgaccaacatagtgaaaccccatctctactgaaagtacaaaaattagctgggtgtgttggcacatgcctgtagtcccagctacttgagaggctgaggcaagagaattgtttgaacccgggaggcagaggttgcagtgtggtgagatcatgccactacactccagcctggcgacagagcgagacttggtttcaaaaaaaaaaaaaaaaaaaacttcagtaagtacgtgttatttttttcaataaaattctattacagtatgtcatgtttgctgtagtgctcatatttattgttgtttttgttttagtactcacttgtttcataatatcaagattactaaaaatgggggaaaagacttctaatctttttttcataatatctttgacacatattacagaagaaataaatttcttacttttaatttaatatga

SEQ ID NO: 3 - DNA, 1334 bp, Homo sapiens
  source 1..1334: mol_type = other DNA; note = cDNA
atttcctcactgactataaaagaatagagaaggaagggcttcagtgaccggctgcctggctgacttacagcagtcagactctgacaggatcatggctatgatggaggtccaggggggacccagcctgggacagacctgcgtgctgatcgtgatcttcacagtgctcctgcagtctctctgtgtggctgtaacttacgtgtactttaccaacgagctgaagcagtttgcagaaaatgattgccagagactaatgtctgggcagcagacagggtcattgctgccatcttgaagtctaccttgctgagtctaccctgctgacctcaagccccatcaaggactggttgaccctggcctagacaaccaccgtgtttgtaacagcaccaagagcagtcaccatggaaatccacttttcagaaccaagggcttctggagctgaagaacaggcacccagtgcaagagctttcttttcagaggcacgcaaatgaaaataatccccacacgctaccttctgcccccaatgcccaagtgtggttagttagagaatatagcctcagcctatgatatgctgcaggaaactcatattttgaagtggaaaggatgggaggaggcgggggagacgtatcgtattaattatcattcttggaataaccacagcacctcacgtcaacccgccatgtgtctagtcaccagcattggccaagttctataggagaaactaccaaaattcatgatgcaagaaacatgtgagggtggagagagtgactggggcttcctctctggatttctattgttcagaaatcaatatttatgcataaaaaggtctagaaagagaaacaccaaaatgacaatgtgatctctagatggtatgattatgggtactttttttcctttttatttttctatattttacaaattttctacagggaatgttataaaaatatccatgctatccatgtataattttcatacagatttaaagaacacagcatttttatatagtcttatgagaaaacaaccatactcaaaattatgcacacacacagtctgatctcacccctgtaaacaagagatatcatccaaaggttaagtaggaggtgagaatatagctgctattagtggttgttttgttttgtttttgtgatttacttatttagtttttggagggttttttttttcttttagaaaagtgttctttacttttccatgcttccctgcttgcctgtgtatcctgaatgtatccaggctttataaactcctgggtaataatgtagctacattaacttgttaacctcccatccacttatacccaggaccttactcaattttccaggttc

SEQ ID NO: 4 - protein, 281 aa, Homo sapiens [sequence omitted]

SEQ ID NO: 5 - protein, 101 aa, Homo sapiens
MAMMEVQGGPSLGQTCVLIVIFTVLLQSLCVAVTYVYFTNELKQMQDKYSKSGIACFLKEDDSYWDPNDEESMNSPCWQVKWQLRQLVRKTPRMKRLWAAK

SEQ ID NO: 6 - protein, 65 aa, Homo sapiens
MAMMEVQGGPSLGQTCVLIVIFTVLLQSLCVAVTYVYFTNELKQFAENDCQRLMSGQQTGSLLPS

SEQ ID NO: 7 - protein, 223 aa, Homo sapiens
QDHGYDGGPGGTQPGTDLRADRDLHSAPAVSLCGCNLRVLYQRAEAEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 8 - protein, 178 aa, Homo sapiens
KEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 9 - DNA, 2421 bp, Homo sapiens
  source 1..2421: mol_type = other DNA; note = cDNA
caccctcatgagccccgggtacgtttaactattgagggccaggaaattgccttcctcctggacactggcgcagccttctcagggttaatctcctgtcctggatgactgtcttcaaggtccgttaccacccgaggaatcctgggacagcctataaccaggtatttctcccacatcctcagttgtaattgagagactttaatcttttcacatgccttttttgttattcctgaaagtcccacacccttattaaggagggatatattagccaaggctggagctattatctacatgaatatggggaaaaagttacccatttgctgtcccctacttgaggagggaatcaaccctgaagtctgggcattggaaggacaatttggaagggcaaaaaatgcctgcccagtccaaatcaggttaaaagatcccaccacttttccgtatcaaaggcaatatcccttaaggcctgaagctcataaaggattatagaatattgttaaacatttaaaagctcaaggcttagtgaggaaatgcagcagtccctgcaacaccccagttctaggagtacaaaaaccaaacagtcagtggagactagtgcaagatcttagactcattaatgaggcagtaattcctctatatccagttgtacccaacccctataccctgctctctcaaataccaggggaagcagaatggttcacggttctggacctcaaggatgccttcttctttattcccctgcactctgactcccagtttctctttgcttttgaggatcccacagaccacacgtcccaacttacacagatggtcttgccccaagggtttagggatagccctcatctgtttggtcaggcactggcccaagatctataggccacttctcaagtccaggcactctggtccttcaatatgtggatgatttacttttggctaccagtttggaagcctcgtgccagcaggctactctggatctcttgaactttctggctaatcaagggtacaaggtgtctaggtcgaaggcccagctttgcctacagcaggttaaatatctaagcctaatcttagccaaagggaccagggccctcagcaaggaatgaatacagcctatactggcttatcctcaccctaagacattaaaacagttgcggggattccttggaattactggcttttgctgactatggatctccagatacagcgagacagccaggcccctctatactctaatcaaggaaacccagagggcaaatactcatctagtcgaaagggaaccagaggcagaaacagccttcaaaagcttaaagcaggctctagtacaagctccagctttaagccttcccacaggacagaacttctctttatacatcacagagagagccaagatagctcttggagtccttagactcgtgggacaaccccacaaccagtggcatacctaagtaaggaaattgatgtagtagcaaaaggctggcctcactgtttaagggtagttgcagcagcggccgtcttagcgtcagaggctatcagaataatacaaggaaaggatctcactgtctggactactcatgatgtaaatggcatactaggtgccaaaggaagtttatggctatcagacaaccgcctcttagatgccaggcactactccttgagggactggtgcttaaaatatgcacgtgcgtggccctcaaccctgccacttttctcccagaggatggggaaccaattgagcatgactgccaacaaattatagtccagacttatgccgcccgagatgatctcttagaagtccccttaactaatcctgaccttaacctatataccgacggaagttcatttgtggagaatgggatacgaagggcaggttacgccatagtgatgtaaccacacttgaaagcaagcctcttcccccagggaccagtgcccagttagcagaactagtggcacttacccgagccttagaactgggaaagggaaaaagaataaatgtgtatacagataacaagtatgcttatctaatcctacatgcccatgctgcaatatggaaagaatgggagttcctaacctctgggaacccccgctggatgccacagggaagttatggagttattgcacatggtgcaggaacccaaagaggtgggagtcttacactaccaaggccatcaaaatgggaaggagaggggagaacagcagcataagcggctggcagaggtagggaaagaccagcaagaaggaaagagagaaagagaaagtcagagaaagagacagagagaggaagagacagagagacagaacgttaaagagggtgtcagaaacagagacaaacaaaaggagtcagaaagaaggacagacacagaaagtcaaagagagagttaaaaagagaggaagagacaaagaagtcgaagagagaaagagagagatggaagt

SEQ ID NO: 10 - DNA, 2421 bp, Homo sapiens
  source 1..2421: mol_type = other DNA; note = cDNA
ctttgcagataaatatggcacactagccccacgttttctgagacattcctcaattgcttagacatattctgagcctacagcagaggaacctccagtctcagcaccatgaatcaaactgccattctgatttgctgccttatctttctgactctaagtggcattcaaggtaaggaacatcaaaggatacttaatttgtaaaatgagaaataggaataggtataaattctaaaaatacagaaataatgtatttgtaaaagtttcactgcatgcttataaataagagggaaataaatagagattccctcagatcataaaacttatatgaattgaagtgagagaaacaaatagaataagagaaagagaaggaaaaagggaaggaggacagaagagatggggaagagggaggatagagagagaaaatgtgagggaatgcggacagagatgagatacagatacttccttacctaactaagctcaatgaaccacatgaactgtgcttaagggtttgactttataatcaacaagctgcaattcttttcttccagataatcaactctttaatcatttacagttgtgttatgatgtgatccattcctcctcagattaagtgactatttgctgatatggggatataggttctgctaaataccaccagtctacattaaatgcctaaaatgaacactgtgctaaccttctctgctgttcctcttttcctacaggagtacctctctctagaactgtacgctgtacctgcatcagcattagtaatcaacctgttaatccaaggtctttagaaaaacttgaaattattcctgcaagccaattttgtccacgtgttgagatcatgtgagtgaaatcccatctgattatcacttccctggttgtaattatatactgtattaaatatgtaatgataataaaaaaagatcagtaaagggtttgtgatgattctaaaactaatgtacagcaaacaaaaacatgcagagtgaaacttaaatgtctgacttcagaactgcgtatgccatctgttttattgacccaacacagttttaaatattttcatccctatttatttctacagtgctacaatgaaaaagaagggtgagaagagatgtctgaatccagaatcgaaggccatcaagaatttactgaaagcagttagcaaggaaaggtaggtttgctgttgcctgcagaagaattgctctttaggaaacggcaatcttgggagtcagaaatacttgcattgtggtttgctgtgcaatcgctggtttaaaagtatgttaccaccacgccctcccctacctccatttatttaaatgctgaggcaccatcttgtgtgataagtatcagaagttaccctgattaccagtcaaccttgaagtacagctataactatctaagcaaaactgacaacattttccccaagtctttcatggttgaaaaaagcaacccctataatccataatgaatgcatagcagcaggaaagctcagttatctattctatgaactcggtactttccaaacacaacccaatctgaagccagagtcagactatcacacttttatatcccctttctcttcttacaggtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatgatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtagaagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaaataaaaatgaggtactctcctggaaatattaagaaagactatctaaatgttgaaagatcaaaaggttaataaagtaattataactaaga

SEQ ID NO: 11 - DNA, 2421 bp, Homo sapiens
  source 1..2421: mol_type = other DNA; note = cDNA
agatgacttttttctatttatatttaataagatgatgaacccttcttgcattcccgaaataaacctcaactgttacagtgttttattcttttaatatgtacgaagtacatgttaagcaagttatttcctaagcagccccacaaactgggcactactaccatcctgctctgcccctccctcactctacttcagccacttcagccacaatggcctctcctcactgcccctctgatgcaccaagcttgttctcacctcaggaatgtgcaacacctgccagacttgctgttccccggagcctccatccccagatatcctcatatatcatcctcctcttcatttgtgtctctgcttacatatgacctatttacagaagcctttcctgtctaccccccatgaaatagaaatcgcattccaatcttgtctctaccccaatgctgtttcattttgtctgtagcaattgtcatcatctcatatatattcacatgtggaaaatacacaaaatgtttaacttcttttaatttacattccatttccccatgaattgaagctccatgacagcggagatttttctctgctttccctgttgctcacttcccagcaccaagagcaggcctggcacatgggaagtacttactatttattgaatggatgaatgaacaaatgaatgaatggatacttattttacacataagaaaactgaagcttatagaaattaagtaactaaaatcacacagaagcacagctgaaactaaaacctacgtctaactttcaattcctgacccttaaccattaaaacaaatgacaggtgactttaggccactgaaaatgctcatataatcttatgaattctaaagcacaagttaatcacaccattgattgaaagtctgaggaatactgtatagacaagcccctgtacaaggtaagcaaaagaatcagaggatggcctccaaagaattccctggacattatgggaattacattgttagccttcctactgatacccataagcctcacagcaagcatcatgaagctgtgaccttcatctgcacatgcccttgtatacccaaaagataaaactggatgcttcagggccgaatggccaataaacacgtgtttattactggcatgggcagacacacatactgaaagtaccatttcccagcggactagccatattatgatcagtacagacactaaagatttagctttgaaaaaactatttgctcttccaaagctgaagaatcttctgtgatttcaacaggcaagttacagtcaggtattcttaatgttcttttcctcctctctcactgggatactttctttccttcagacaacgtcaagcgaaaaacaaaatttcacaaatctccatttctgacactaaacagtacagtatctttattttttttataatttaatcaaaccctgtattttagaactgtggggctgatccaacattgcaatgtgtcacatttaattccatcaatgtaaagcataatgagcaaagattaaggtagtgaggcataactaaatgttttgaacctgtgaatttcaaaagcaaggcccatttgtgttattttctaaatagtaaataaaatcattttccaacatttcactatcaaattacagtaatttttccaccagtacacacttgaggaaagccacaaaaagacttttccaacagttcattctgttattgctcataaccttctaaatacttctcctcattggcttctattcaaaggtaaatggaaagcagtaaaatttatggaaaatatattcaactgcttaaaatacatcaaccaaaaaaaagattttagagctgtattatgagttgtgaaattgcattgccttcacttacctttcagtttcactggtaggtaacaaaactgacagactggtcaagttccaaaacatcccctatatagagcctgactcttccatctcaaattctcaccttggtcaaggccagagtaaacacctgtccttcacatttttacacaacatcactttgtatgctacaaatataagctttcataccagggaggaagcaaattccaggacactggaaacatttctgctctcttaaaccagtctgttgattgttcccttgactttctcagctgtcaggatagtgaaaggaggaaaactgcaaaactgtaaagtataacctgataagtttgccctttaagcttttcacacagagagaggtaaaataaaactcaagtctaaggtttaaaattgagctatgaatattatattctagcactagacaaaaatgttgcaagattttaataaaataagattattaaaatcaatttttacatttcatgggccaaggagagacatcaaagaatgtttaactaacattttaaagatactatactttataaagttaagaagaaaaatgacaactgcaccagt

SEQ ID NO: 12 - DNA, 1227 bp, Homo sapiens
  source 1..1227: mol_type = other DNA; note = cDNA
ctttgcagataaatatggcacactagccccacgttttctgagacattcctcaattgcttagacatattctgagcctacagcagaggaacctccagtctcagcaccatgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatgatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtagaagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaaataaaaatgaggtactctcctggaaatattaagaaagactatctaaatgttgaaagatcaaaaggttaataaagtaattataactaagaaaaaaaaaaaa

SEQ ID NO: 13 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 14 - DNA, 2301 bp, Homo sapiens
  source 1..2301: mol_type = other DNA; note = cDNA
aaggcaagagatctaggacttctagcccctgaactttcagccgaatacatcttttccaaaggagtgaattcaggcccttgtatcactggcagcaggacgtgaccatggagaagctgttgtgtttcttggtcttgaccagcctctctcatgcttttggccagacaggtaagggccaccccaggctatgggagagatttgatctgaggtatgggggtggggtctaagactgcatgaacagtctcaaaaaaaaaaaaaaaagactgtatgaacagaacagtggagcatccttcatggtgtgtgtgtgtgtgtgtgtgtgtgtgtgtgtgtgtgtggtgtgtaactggagaaggggtcagtctgtttctcaatcttaaattctatacgtaagtgaggggatagatctgtgtgatctgagaaacctctcacatttgcttgtttttctggctcacagacatgtcgaggaaggcttttgtgtttcccaaagagtcggatacttcctatgtatccctcaaagcaccgttaacgaagcctctcaaagccttcactgtgtgcctccacttctacacggaactgtcctcgacccgtgggtacagtattttctcgtatgccaccaagagacaagacaatgagattctcatattttggtctaaggatataggatacagttttacagtgggtgggtctgaaatattattcgaggttcctgaagtcacagtagctccagtacacatttgtacaagctgggagtccgcctcagggatcgtggagttctgggtagatgggaagcccagggtgaggaagagtctgaagaagggatacactgtgggggcagaagcaagcatcatcttggggcaggagcaggattccttcggtgggaactttgaaggaagccagtccctggtgggagacattggaaatgtgaacatgtgggactttgtgctgtcaccagatgagattaacaccatctatcttggcgggcccttcagtcctaatgtcctgaactggcgggcactgaagtatgaagtgcaaggcgaagtgttcaccaaaccccagctgtggccctgaggcccagctgtgggtcctgaaggtacctcccggttttttacaccgcatgggccccacgtctctgtctctggtacctcccgcttttttacactgcatggttcccacgtctctgtctctgggcctttgttcccctatatgcattgcaggcctgctccaccctcctcagcgcctgagaatggaggtaaagtgtctggtctgggagctcgttaactatgctgggaaacggtccaaaagaatcagaatttgaggtgttttgttttcatttttatttcaagttggacagatcttggagataatttcttacctcacatagatgagaaaactaacacccagaaaggagaaatgatgttataaaaaactcataaggcaagagctgagaaggaagcgctgatcttctatttaattccccacccatgacccccagaaagcaggagggcattgcccacattcacagggctcttcagtctcagaatcaggacactggccaggtgtctggtttgggtccagagtgctcatcatcatgtcatagaactgctgggcccaggtctcctgaaatgggaagcccagcaataccacgcagtccctccactttctcaaagcacactggaaaggccattagaattgccccagcagagcagatctgctttttttccagagcaaaatgaagcactaggtataaatatgttgttactgccaagaacttaaatgactggtttttgtttgcttgcagtgctttcttaattttatggctcttctgggaaactcctccccttttccacacgaaccttgtggggctgtgaattctttcttcatccccgcattcccaatatacccaggccacaagagtggacgtgaaccacagggtgtcctgtcagaggagcccatctcccatctccccagctccctatctggaggatagttggatagttacgtgttcctagcaggaccaactacagtcttcccaaggattgagttatggactttgggagtgagacatcttcttgctgctggatttccaagctgagaggacgtgaacctgggaccaccagtagccatcttgtttgccacatggagagagactgtgaggacagaagccaaactggaagtggaggagccaagggattgacaaacaacagagccttgaccacgtggagtctctgaatcagccttgtctggaaccagatctacacctggactgcccaggtctataagccaataaagcccctgtttacttga

SEQ ID NO: 15 - DNA, 2301 bp, Homo sapiens
  source 1..2301: mol_type = other DNA; note = cDNA
aggaattgaactcagctctgccccaagcggacctaatagacatctacagaactctccaccccaaatcaacagaatatacatttttttcagcaccacaccacacctattccaaaattgatcacatagttggcagtaaagctctcctcagcaaatgtaaaggaacagaaattataacaaactatctctcagaccacagtgcaatcaaattagaactcagaattaagaatctcactcaaaaccgcacaactacatggaaactgaacaacctgcttctgaatgactactgggtacataatgaaatgaaggcagaaataaagatgttctttgaaatgaacaagaacaaacacacaacataccagaatctctgggacgcattcaaagcagtgtgtagagggaaatttatagcactaaatgcccacaagagaaagcaggaaacatccaaaattgacatcctaacatcacagttaaaagaactagaaaagcaagagcaaacacattcaaaagctagcagaaggcaagagataactaaaatcagagcagaactgaaggaaatagagacacaaaaacccttcaaaaaattaatgaatccaggagctggttttttgaaaggatcaacaaaatagatagaccactagcaagactaataaagaaaaaaagagagaagaatcaaatagacacaataaaaaaatgataaaggggatatcaccaccgatcccacggaaatacaaactaccatcagagaatactacaaacacctctacgcaaataaactagaaaatcaagaagaaatggataaattcctcgacacatacactctcccaagactaaaccaggaagaagttgaatctctgaatagaccaataacaggatatgaaattgtggcaataatcaataccttaccaacaaaaaagagtccaggaccagatggattcacagccgaattctaccagaggtacaaggaggaactggtaccattccttctgaaactattccaatcaatagaaaaagagggaatcctccctaactcattttatgaggccagcatcattctgataccaaagccgggcagagacacaaccaaaaaagagaattttagaccaatcaatatccttgatgaacattgatgcaaaaatcctcaataaaatactgccaaaccaaatccagcagcacatcaaaaagcttatccaccatgatcaagtgggcttcatccctgggatgcaaggctggttcaatatacgcaaatcaataaatgtaatccagcatataaacagagccaaagacaaaaaccacatgattatctcaatagatgcagaaaagacctttgacaaaattcaacaacccttcatgctcaaaactctcaataaattaggtattgatgggacgtatttcaaaataataagagctatctatgacaaacccacagccaatatcatactgaatgggcaaaaactggaagtattcactttgaaaactggcacaagacagggatgccctctctcaccactcctattcaacatagtgttggaagttctggccagggcaattaggcaggagaaggaaataaagggtattcaattaggaaaagaggaagtcaaattgtccctgtttgcagacgacatgattgtatatctagaaaaccccattgtctcagcccaaaatctccttaagcagataagcaacttcagcaaaatctcaggatacaaaatcaatgtacaaaaatcacaagcattcttatacaccaacaacagacaaacagagagccaaatcatgagtgaaatcccattcacaattgctttaaagagaataaaatacctaggaatccaacttacaagggatgtgaaggacctcttcaaggagaactacaaaccactgctcaatgaaataaaagaggataaaaacaaatggaagaacattccatgctcatgggtaggaagaatcaatatcatgaaaatggccatactgcccaaggtaatttacagattcaatgccatccccatcaagctaccaatgcctttcttcacagaattggaaaaaactatttttagttcatatggaaccaaaaaagagcccgcattgccaagtcaatcctaagccaaaagaacaaagctggaggcatcacactacctgacttcaaactatactacaaggctacagtaaccaaaacagcatggtactggaaccaaaacagagatatagatcaatggaacagaacagagccctcaaaattaatgccacatatctacaactatctgatctttgacaaacctgagaaaaaccagcaatggggaaaggattccccat

SEQ ID NO: 16 - DNA, 2024 bp, Homo sapiens
  source 1..2024: mol_type = other DNA; note = cDNA
aaggcaagagatctaggacttctagcccctgaactttcagccgaatacatcttttccaaaggagtgaattcaggcccttgtatcactggcagcaggacgtgaccatggagaagctgttgtgtttcttggtcttgaccagcctctctcatgcttttggccagacagacatgtcgaggaaggcttttgtgtttcccaaagagtcggatacttcctatgtatccctcaaagcaccgttaacgaagcctctcaaagccttcactgtgtgcctccacttctacacggaactgtcctcgacccgtgggtacagtattttctcgtatgccaccaagagacaagacaatgagattctcatattttggtctaaggatataggatacagttttacagtgggtgggtctgaaatattattcgaggttcctgaagtcacagtagctccagtacacatttgtacaagctgggagtccgcctcagggatcgtggagttctgggtagatgggaagcccagggtgaggaagagtctgaagaagggatacactgtgggggcagaagcaagcatcatcttggggcaggagcaggattccttcggtgggaactttgaaggaagccagtccctggtgggagacattggaaatgtgaacatgtgggactttgtgctgtcaccagatgagattaacaccatctatcttggcgggcccttcagtcctaatgtcctgaactggcgggcactgaagtatgaagtgcaaggcgaagtgttcaccaaaccccagctgtggccctgaggcccagctgtgggtcctgaaggtacctcccggttttttacaccgcatgggccccacgtctctgtctctggtacctcccgcttttttacactgcatggttcccacgtctctgtctctgggcctttgttcccctatatgcattgcaggcctgctccaccctcctcagcgcctgagaatggaggtaaagtgtctggtctgggagctcgttaactatgctgggaaacggtccaaaagaatcagaatttgaggtgttttgttttcatttttatttcaagttggacagatcttggagataatttcttacctcacatagatgagaaaactaacacccagaaaggagaaatgatgttataaaaaactcataaggcaagagctgagaaggaagcgctgatcttctatttaattccccacccatgacccccagaaagcaggagggcattgcccacattcacagggctcttcagtctcagaatcaggacactggccaggtgtctggtttgggtccagagtgctcatcatcatgtcatagaactgctgggcccaggtctcctgaaatgggaagcccagcaataccacgcagtccctccactttctcaaagcacactggaaaggccattagaattgccccagcagagcagatctgctttttttccagagcaaaatgaagcactaggtataaatatgttgttactgccaagaacttaaatgactggtttttgtttgcttgcagtgctttcttaattttatggctcttctgggaaactcctccccttttccacacgaaccttgtggggctgtgaattctttcttcatccccgcattcccaatatacccaggccacaagagtggacgtgaaccacagggtgtcctgtcagaggagcccatctcccatctccccagctccctatctggaggatagttggatagttacgtgttcctagcaggaccaactacagtcttcccaaggattgagttatggactttgggagtgagacatcttcttgctgctggatttccaagctgagaggacgtgaacctgggaccaccagtagccatcttgtttgccacatggagagagactgtgaggacagaagccaaactggaagtggaggagccaagggattgacaaacaacagagccttgaccacgtggagtctctgaatcagccttgtctggaaccagatctacacctggactgcccaggtctataagccaataaagcccctgtttacttgaaaaaaaaaa

SEQ ID NO: 17 - protein, 224 aa, Homo sapiens
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 18 - DNA, 26803 bp, Homo sapiens
  source 1..26803: mol_type = other DNA; note = cDNA
cttgctctgtcacccaggctggagtgcagtgctgtgatcatggttcactgcagccttgaactcctgggctctggcaatcctcctgcctgagccttctgagtagctgagactatagatatgggccaccacacctggctaatttttaattttttttagtagagatgaagtcttgctatgttgaccaggcttgtgggagttcagtcaggctggtggaaaaaattttaaagatagttataagaaatagacacaaaccttcttgtaaggctggagagggtttacattgcttcagtaacagatttggctgaaagcagcctaatcctctctacctttagctgatagcaaaaatgaaaataacaagggaatgtgaggaagtttatctaaatagcttgcttactcatgtggtcctaaaaccaaactttgatcaacctcaggtgcataattgctctctactcagggggtgagcaatgttaattaccctctagtggtgtttactcgagacctttgtcatttaatctgtattaaataaatgtgaactttgctagcttattgaggtgatgctccagatgcagagcagagccccttagccagactgacaggcaaaatatctgtgtcagtgtatgtctctcatccatcactggttcagggtctgcgggctggattcctgcacaggctggtcttgaactcctgggctcaagcaatcctcccgcctgagccttctgagtagctgagactacagatatgggccaccacacctggctatttttaacattttttagtagagatgaagtcttgctgtgttgcccaggctggtcttaaactcctgggctcaagagctcctcctgccttggcctcccaaagtgctgggagtacaggcatgagtcatggtgcccagacggacatttttttaaaaataaaggaatactcctgaaacgctgaagtcttctttgtacccctctgtgataacatgaatagcctcttaatgacaccaaggagcaagacaagttagtcccaaagtagctcacatagatgatgataaaggaatgaggggtgggtgtgatctatgcaaaaaacccttactctttaatgggttgctgtttctaacataattgcaacagtaccctacattgctataatgcattatagttttcaaagtgcttttgtggcctccataatagagaggttgtgaggtaagcttcaaccacggctgccctactacccaagagtggacacacaatgatgaataggtcaatttctgcctcttagtttcttagctagggaggggcacttactggaacagcacagaaaacagagtctttggcccagttggagaatctttatcaggttatgctacttcaagctttcctcctgttaaatgtgagaggaataaatccctgttccttggatcagtgtgactctgaaacagctggaggagagacagctttaggcaggttgaaacagagagctccagcatatgtactttttttttttttttgagatggagtctcgctctgtctcccaggctggagtgcagtggctcgatcttggctcactgcaagctctgcctcccaggttcatgccattctcccgcctcagcctccctagtagctgggactacaggctcccgccaccacgcccggctaatttttttgtatttttagtagagacagggtttcaccatgttggccaggccagtcttgaactcctgacctgaggtgatccacctgcctcagtctcccaaagtgctgggattacaggcgtgagccactgaacttggccaagtgcactacttttaaaagttaaagtattatgcagccatgagggaatattgtgcaagaagaaagcttttacaagaaaaacttgaaacattggtatttttgcctcctttttaacaactagagtcgttttgggagttgtttcttgtcgaagaaacaacccatgtttattttcccagtatggcaggaccatgtaggaaagcaaaattacccctcaggagaggaaattctctgacactctataaggctccataaccctcctctgaactgtggccaacaagattgggtagcactttttaaggtagtttaagaaaaataggctgtgcatggtggtttatgcctgtaatcccagcactttgggaggccgaggtgagtggatcacctgaggtcaggagtttgagaccagcctgaccaatatggtgaaaccccatctctactgaaaatacaaaaattagccgggtgtggtggcgggcacctgtagtcccagctactcgggaggctgaggcaggagaattgcttgaacctgggaggcggaggttgcagtgagccgagactgtgccactgcactccagcctgggtgacagagcaggactccatctcaaaaaaaaaaaaaaaaaaaagaaagaaagaaaaatatatagtgagcccaataaagctgtataatctaaatcaaacatgacttgcatgcctggagactttgcactagaaaaatatttctacctaaaaaatcaaattttattttctttctcacaaatattcaatctgctttcatctcagttcttcctaccttgtcaaacctctccccacatttcctattctttttctctccagcctgatatctctcattatactgcttaagagaaatgttatgttactattcttttctcccagaactgtttctctggttctttaaggtgtctgagtacacactgtgccttcttccttttagccctctcttctccttgttccctgagccttacctttatgaccttagaacttcaagttcccactacaattttaaatatagactattttgcttttcctcctactagggagcttaaattgcctctaattacactgttttcccgagtccttcctctcctttgcaatttagatatagcacagaagcacattttgcttgactgtcctctgaactgtcatgctgttttgatgtggttctattgtccaagagtcttggttaaataatagcccagcatcccacctgtgtttaaaagaactgcttcacaggcaaatcaaaaagcccatggatccgaagtcacaatgggcgttgctattcaaacagcaccagattgctattcaacgcttggttgaaaaataaatttcagtttcattcacctaatatatttccttctattttgtaggatggtatgctcttacttcaatttggacttgttcacaattgaacgttaacatagccttatagattagcctgttttcattggtccagagtattctccaaataaagcagggtctgtgcattttaagcaactcccctgaacaatttcatgggcattctcaaatttgagaactacctgaaaagctgtgttggagaaaagaacaaccaatgaatgtggcaggacagaatattgaacattaacttcccttttcctcttctcccatctgttctcttccattatccctacccgtccgctcagtctcgttattcaggcaacagttattttgccattatttcctcaagaaaggaacaaaagtaaacacaattgctttctgattttttttttttttttgcattttaaaatggactttgaaaccataagcaaagaggtgtttaagagtctttccaaagccaaaaatgaaggttttgaaatttcaaagtcactgccttgaagagactcgaggtttggagtgtgtacagtatgtcggagctggacttttctccttcctgagactagataacggtctgaatccaagacagttttcatgatttcagaggaagtggtcaagtggtctgtgaggtagaccttctgcttaagagcagtcaggaggccgggtgccgtggctcacgcctgtaatcccagcactttgggagaccgaggtgggcggatcacctgaggtcaggagttcaagacaagcctgaccaacatgcagaaaccctgtctctactaaaaatacaaaaaattggccgggcacggtggcacatgcactccagcctgggcaacaatagtgaaactccatctcaaaaaaaaaacaaaaaaaagagaagaaaagaaaagaaaaggagcagtcaggatgtgtgcctccaaagctgaggtagacaaaaagataccagagttctagaggcctgccaggcacagcagcagcagcagaaggaaggtgtgggcgagaacagggcagccaggcgtgtgccacctcccagacacaattattgggaatggagggcaagtggtgatgggagaaaatcttgacttaattgatgtcaagattaaagaaatgccacctggtggcatttaagttcacacataggtaaagaaagttatgcatttactgtgaaagtcatcccactatttagtagaaacaggagatctggattctggtcaagagtctcttttgccaactgtggcaccactgagcagcggcacagcttttgtgaatcctgggttcttcattattaaaatggggacattagcgttgggttgagtataagaaatggacatttttgcaggtcaaaaatggttgaatatttgcattttcatatgattcaaccgaatacttacttcacaggcataaggaaaaaaatagaataacatactaacaactgtccctggagtaagtacttaacaaatacatgatttataaagaagatatgtgaaagatatttgtaagtacatgatttatagaaagatatgaaagtatgtaaacccttgtggtctaatggtcacagaataatctgagcttaatatccctgctccctaccatacagaaggcaaaatgcctattaggggttttctttcttcaccctctccttctttttcctcctcctcttgactcctcctcatcctcctctttcttcttccccttattaatgtctaaaaaggggctgagcatggtggctcatgcctgtaatcctagcactctgggaagctgcggcaggtggatcacctgaggccaggagtttgaaaccagcctggtcaacatggcaaaaccccatccctactaaaaatacaaaaattacccaggtgtggtggcaggcacctgtaattccaactacccgggaggctgaggcaggagaatcgcttaaacccgggaggcagaggttgcagtgagccaagattgtgccactgttgtccatcctgggtgacagagggagactctgtctcaagataaataataataataataataatttctaaaaaggtaatacattttcatagttcaaaaaccaaaaggtataaaaggaaatacagtaaaaaatttcctatcatatcactgtctagagtactattccttatatattttcctgatttttgagtattttaaaatgtgagtgttggatatgagtgttggatttaaaaagttttatgataatttgtgtatatttgtgtgtgtgtgtgtgtgtgtgtgtgtgtatagtagtccaagactatcagtttatgaataataagaggagacccatggaaaaccagtccctttgaccaagttcactcagataatcagcagcagggcttggacattaattacagttatccaacatcctttgaggtctcacatgacaaattacaaatatggagtgtaaatgtaacccactttgctaggcaaaaaaagccctgtttttttaaaaaatatatatttttggcttatgggcaacagaagccagggagacgtacagtcaaacctcatttctcatggctttcatatctgcaaattctcctactcattaaaagttatttataactcccgaatcaatacccacagcacttttgtgatcattggcagacatgtgcagaaaagaaaaaaaaattgagttgttgattgcacacattcccagctgaatttcaacaaagcaacactctgccttcccacttcagctttcttactatatgtgtgtcctttttctgtttatttagtaccatgtttttcacactttcgttctttttggtggtgattttgctgtttaaaatggccaacaagtgtagtgctaagtgctgcgtagggttcttaagcacaagaaggctatgatgtgccttatggagaaaatacgtgtgttggatcagtttcactcaagcatgagttatggcgctattagctgtgagttcaatgttaacaaatcaacaatatatgctaaagtgtctttaaacagaaacacacataaaacaaggttatatgtttggttggcaaaaatgttataaccagaagcttgcagaaacctaaccctgtatttcccttaagagcaatggttcattattcactaattcaatgtttacagcaactttataaactataactaccatgaataatgagaattgactatgttttagatcataatacctgaagtgaagttttccatcatggtttttagttcttagagtccatttaagttttttaaaaatatgatagctatcctatcacatcccacattcttgagattaaaaaattaatcttttatttggtacagaaaaggctcctgcgtgcatttttggaggggctggaccgctgaggaagtcatggttggctcaagtggtttaaatgcattgctaatttcatagcctctgaaccacttatgaaacaatactgatgatatgtcagtagctatgtttgcagctccttcaggaatgcttatatcttttactgctggtaaagaagaaatttatagtaatagattttttttttttttagatagagtctcactctgtcgcctaggctggattacagcggcttgatcacggtttactgcagcctggatctcccaggctctcctactgtagcctccccagtaactagggcccccacacaggcatcaccacacccagctcatttttttatgttttatagagacagggtcttactatgttttccaggctggtctcaaacttctgggctcaagcaatcctcctactttggcctcccaaagtgctgaaattacaggcatgaaccactgcacctggtttatggtcatcttaagtagagacttatgagtgcgtatcattgtatcaccaatctgagactcaattttatctttctgttaatagacaatgcagtttttctgttaattacaatgaaaatcaagggttgcttagcaagtttttactcataactccaagttttgtgacttatagtgagaaatgggtataattctttgtgatttgttaaaatgcagtatttcgaggagggataacatataaaatttaacagtttcctatcattatttttatccttttgaaccactgtatactacagatagacaaaggcagctttggccacttactcctcagtgtgactaagttgaccagatgttttccagaccacaggttctgtcaagagacagtgtggagagaaatgggtaggcaggtatggaaagaagagtcagaaacagatagtgagctaatctctacaaatggctcgttatggcctgagctgtgttctcctccccgcaaaaagatatgttggagtcgtaatccccagtacctcagaatgtggccttatttggagattcgatatttacagaggtaagcaagttaaaatgagatcactatagtgggtcctaattcaatacgactggtatccttacaaaaaaggggaaatttaaccacagatacagacacacacatagggagagtgctagtgaaggtgaagtcagagattggaatgatgtagcagaagcctaggaatgccaaagattgccagcaaaccaccagaagctgggagagccacatggaacagattctccttcacagtcccctgaaagaaccaactctcctgacacctcgatttcagacttctagcctccagagctggggtcaatgagcttctctcattaagccatcatccagtgtatggtactttgttacagcagtcttagcaaatgcatggttcctcactggaaccatgaatttccatgcatttatttcatttaaataataaaacggatcccctttgtggcttgtagttctgttccatttcaaaaatccagaaaaaagatttgttcagaagctagaaatgatgaactggatcttgcccaaggtcataaaaccacaaaaccctttacagagcacaaaagtctgatttttcaaagcttctctcaaaagatgggtcactccttagtcatttaggccactgacaactgccctggactctttatttatttatttatttatttatttatttaatttttttgagacagagtcttgctttgtcgcccaggctggagtgcagtggcgtgatctgagctcactgcaagccctgcctcccgggttgacgccattctcctgcctcagcctcccaagtagctgggactacaggcacccgccaccacgcccggctaattttttgtattttgtttagtagagacagggtttcaccatgttagccaggatggtctcgaggtcctgaccttgtgatccacccgccttggcctcccaaagtgctgggattacaggcgtgagccactgcgcctggcctggactcttacatataataagcttgacttatcgtacaacttataattgatgctttcacgtcatgggaagatcaaattaatgcagagagcgactacgtttctggtgggagagcagcaggggtcttgagaggaacagcagtgttgactgtttcgctacctactctgggtgctgattacatctttctttcgtgcaggcaaatccacttggagctattttgggctcactgtgggcatcattctttgtatactctgctcctttcccccatagtttctctattgatgggggattactaaagaatgaagaaaagaacaaaatgaaagtggcttttgaaaaattttaaatgactgtcactatgtaacatattcattatttcatgtctccactggacattttggagatgtgagccttgcaatacctacatgaatgcttctatgattatgattattgttttgctcttcgcctaacaacttgccaagtattgtcaacctcagtgtgtgagatggggtccactcaaacatcagggccgaagtcaggtagttcagttaagtgaatttgataccaggaactagttacaaaggagttggaagggctggaaagccaaactggagaagaaggggaaccccagagtaacaatagcaggaagcctctaccgtctacctctagaactggggaggagctgagttaacagagtcctggagccattgctggggaagaagaaccccaactgcagaggaaaagtggccattgtgaagaaggtgatggtggagaagtcgtctgaatcaaaggggagaggatatgttggctcctttatctttttatctttcattgtcctaattccttctgggcatggttaaatgagcccaggaaatgcagttggcaggaatcagcttcctgtgacatagacagagtaagagaaggacaaaaataatgaatctgagagcaagcaggcaaatgaccagcacattaagcctagcacacagtatgttatacgggatttggggtagcaaaagatgaaggcaggtcagagaagccacaactaggggatgggcaaggtcaatgaggtatgacgggtattgtatacaggaagagggtcataaacaggagtggagtcaatacaggagactaacatatacacatcataaatattagtagagaagcataaaatagtctcctggagatcagggaaacaggaaattataggttttcaggataattcagccatatccaggaaacacacacagtgaagtaacaaagagtcaataggcttagagtggtacaattcattatgcacatgtaggaattcattccaaataataggctgaaatgtagacatgagaatccaaaaaagatgttttcttagtttttgccaatatcttagctacgttttttttggttcaacaaagtaagttaacagtcatatctgcttggaaattgtatttaggccaggtgcagtgtctcacgcctgtaatcccagcacttggggtggctgaggcaggaggatcccttgagcccaggagttagaggctgcagtgagcgacaactacaccactgcattccagtctgggtgacagaacaaaaacttattaaaaaaagaaaaaaaaaaggtcgggcgcggtggctcacgcctgtaatcccagcactttgggaggtcgaggtgggcggatcacgagtcaagagatggagaccatcttggtcaacatggtgaaaccccatctctactaaaaatacaaaaattagctgggcatggtggcatgcacctgtagtcccacctattcaggaggctgaggcaggagaatcgcttgaacccgggaagcggaggttgcagtgagctgagattgcaccactgcactccatcctgtcgagactctgtctcaaaaagaaaaaaaaggaaaagaaactattgaaatagctgatattagtttgcttacttgtcgttactctttttcatgatggattataaagaaaagttataactatttgaattttctgctgatttgaagtctctataaacagtacattcctttttggtacacagagggcacttatctgcaagaaaggcaaagaaaatggaaaagttaatgaaagaggaatcatccaatccacgaacagaatgaaaccacatacacagtgaagaaacttgtcttacattttcttccttatattacttatcattcatggtagtgactactttggggcttgagtaaagcttctctaatttattccatgtagcatcatatgtgaaaaagacaaatagatactttagacatgataataacactttattttttatttatttgtttattttgagacagagttttgctcttgttgcccaggctggagtgcaatggtgcaatctcagctcactgcaacctctgcctcctgggttcaagcgactctcctgactcagcctcccaagtagctgggattacaggcacgcatcaccacgcccagctaattttttgtaattttagtagagacagggtttctccgtgttggccgggctggcctcaaactcctgacctcaggtgatccacccaccttggtctcccgaagtgctgggattataggtgtgagccaccatgcctggcccataacaccttatttaaaaataatctgtctggatccatacaacttgtctggataactaaattggaaattattccttgttttaaagtaattcaattgaaaatttttaaatttttttgttaatcaagcactttttggtggaatctaaattaacacatgtaggagatgcctgtttcactaattacacaggcatcttgcagtaattaatgtctgggaggaaggaatgtcttttgcttactctcttcttcttcacaaaaatgtgaattttggaaagcaataatggaagcatgtagaattatagaaatacaaatgtatataactatcacaaaaaaatgaggccaaaggactattcagatataattaggctatggtagctgtaattatctaggaaattaataaaattcattcacctagaaattattagtgagcatcaaatatgtgtcagcactaggctagggtctcagaacgtacagataaatcatagttctggcttcagggagttatatagattagagataaaacctaactacaggggctgggcacggtagctcatacctgtaatcccaccactttgggaggccgaggcgggtggatcgcctgaggtcaaggagtttgagaccagcctggccaacatgataaaatcctgtcactactaaaaatacaaaagttagccgggaggtagtatgtgcacctgtaatcccagctactcgggaggctgaggcaggagaatctcttgagcctgcggtggaggttgtggtgagctgagatcacgacactgcactccaatctgggcgagagagtgagaccctatctcaaaaccccaaacaaaacaaacaaaaaaaccaaacctaactacagggctatgagagatgactactggcaaggagccacaggtagaacaaaggggatgtgtccccaggcaaagggtagtcagcaagcctgcaacctcagggttcccggatctgagcctctggctcttggctaggcaggccccaagcgttggcctcctgccatggcaagctccagcctggtctcccaccttgagctaacattcatatgttgtagacacagccacgcttcctgcttacctgtcacttccagttctcgaaggcacccttttcaaatgaaaatccgccccttttcacatcaaacagctcatctggtcctgtggattacatttctcagaaatgcctctgaacattcgcctcctctccacccccactgcctctgctacagtgcaggtgctcgccatttctgatttgttctgtcacacactcgtttatcaggtctctccatctcctgttttgacatgctgtaaagcaatttgccactggaaaaaagggctctctttttttttttgagacggagtctcgctctgctgcccaggctggagtgcaatggcatgatctcggctcactgcaacctctgcctcccaagttcaagtgattctcctgcctcagcctccctaaagtctgggattacaggtgcacgccaccaagcccggctaatttttgtatttttagtagacgtggggtttcaccatattggccaagctggtctcgaactcctgacctcaggtgatccacccgcctcggcctcccaaagtgctgggatttcaggtgtgaggcatcgtgcccggcctctctttccaaagtatgttattatttttcccaagaccctttgacggatcctcatttcctacacacagtggttctcaaacttggatgtactccagaattacataggaaattcttcatttttttagacgaggtcttgctatgtttctcaggttggtctcaaactcttgacctcgtgtgatcctccggccgcagtctcccaagtagctgagattataggcgtgccactgagcccagctggaaatttttttaaaacacagattcctctgaatcaaaatttctagaagtgaggtctgagcaatctgtatttttaacaagttatccagatggctctttactgtcagtctggttccagctgagggagttttagaattacaaggcaaagtccaaacttagcacacaaagtcattcacatcaactcttcccctgtacacaccctatgtcatagccagagttgtaacccatttcttaaacacccagggttcttttaagtctctgtgtcattgtatgttattttctctagaattgcctttaacctcctttccaccctggaaagcattccctaagccatctttgaagctttctttgatctctaacttagagtcctcctctccttctaaagccctgtgttaatcacttgtcatggtgtactttaatttttacttgcctgtttctctttacggtactttgacttcaaagggtggggctgcaaattagtcatctccttagagtccagccttgttcctagttcctaactggcacttaatataaacgaatgaatgaatggacaaatgaagagaatgctagttatgataaagaattggccgtgtatgagactacttctctttatgaactaaataattatatgcctttcaataaaatactagtacacgtagctagcacaagctcatcagcatttgagatgatatggaaaccaaaataaacaaatgctaccacaaaaacataatgactgctttccccagtgcaggactgatggaatcatcaaacattgagattaatgtaatgtttggcagatgtccagtgtttttatttttcatttgcctttgtgttcatttatggactaacaatataataaacacacacatactcacagtacatcttttttttttttttttttgcaaagcccagttttcttcatcgcatatctttgttttcttcaagtatccccttacttaatgttggtagtattttttttaatgaaatataaatccctaaccaccaagcaagaaatgagactcttaattgcatcagtttacagtgcaatgtgagtgtaaatagtgtaaattgaatttttaatggacttttttttttcccgtttttgcttgccttacattcattatccctccctgggtaataaacatttatttttccctttgtaaccactcctccccttctgtccatgtggttctttttagtggagctggtggtagggatgtggcatgagattcaagcttggccacctggagtcactgtgttgtgctccagaaggacacctgatccaagtcagccagtaagagtgagccccaggatttttgcttggaccactgaggaaaagtgtactctgccctgtggctgatcaaccattaggatataagccattgttgtagctgtgtgagtcaagctacctgagaatgaggacaacacagtggcaagcaacaccaagagagaaaaagaggcagattctgatgacatttttgagcctttgcatccagctatgcctgaatccaatttatcccctggaatttacacttacttgagctccacccacttgaaagaaaacatttctttttattcttagcctgatttgaatttggcctctctcatttactacccaaagtgtcttgaccactagaatattatgccagactttacagcatcattgaatttgccactttccagaagagttgtgtgaattttcaatgtagcttttaccttctatgagtatctagagatatatttaagtagaagtactccactagtttgttgtgagatcttaagtcacttaatttctctgtaccctagttccctcatttgctagacctagggagctataatgttccttctgcaaaattcttattttgtgaaatattctagaatgtctaactgatacactgctagaacaactgactgctatttaagaagagttgactgctatttaaggatcataattctctaggcataagtgctgtgacggcacagcgtgtgcatcggggctgaggggtggggtggagcagaaagtaggaggagaaagtttgataaacttccttttggataaattgaaaacagtcaaataatttataatttcttatattatcattattagcttcttctataattaggagacttgttccaaaatgtgagaattgtcacaagttgtcaaattcatcaaaggaagaaaatggatgtctcacaaaaaagtatgctcagtccaatttcttctcgtcacactggaacaaactgaacagttttacacagagatgagaagcctggacatttttcaaatatgttttgaagagaatggcaatgcctgagacagaagtaggaaaaagcaatgaatatttaaaaatctgagctggtgtaaaactagaaatagttttagtaagaacaatgtgatgtgctacactaagtgaaatgtatacattgggccacattataatcaaaaataagaatgtacttttattcatcttttatttaaacaataatcaaggtggcgggcgcggtggctcatgcctgtaatcccagcactttgggaggccaaggtgggtggatcatgaggtcaggagttcaagaccagcctggacaacgtggtgaaaccccgtctctactaaaaatacaaaaattagctgggtggggtggcatatgcctgtaataccagctgctcggggggctgaggcaggagaatcgcttgaacttgggaagtggaggttgcagtcagccaagattgtgccattgcgctccagcctgggtgacagagcaagagactctgtctcaaaaggaaaaaagaaaaaaaaaaaatcaaggtaccatttgtaccatttcctggaatttctccaaagtggcaaggtcacatgtttatacattagactcccagtttaacacacagcagacaataactttttttttttttttttgagatggagtctcgctctgtcacccaggctggagtgcagtggcacaatcttggctgactgcaacctccgcttcccgagttcaagcgattctcctgcctcagcctcccgagtagctgggattacaggcatatgccaccatgcccagctaatttttgtatttttagtggagatggggtttcaccacattgtccaggctggtctcaaactcctgacctcataatccagccacctcagcctcccgaagtgctggtccacccttccttcttttctcccttccatccttctcccttttattccatttttctaaatattagaccatagtacaaatcaaaagtcacaaactgataggctgcaatgtagatacagctgggaaaatgttttgtttgcagaacactggggaaatttaacatgaaaaactggaagatctcaatccacatggccacatggtaatattattaatgttgcaggggctttccaattcaacatgtcctctgcatccctactatttatactgccactcatccacctttctgtattactggcctatcacctatacacgtttgagtttatattcttctggctttacttagcaacttaccttttatatttaacattacaacatggtattatcaattagtattcgattcagttgcatataaccaaaaatgccaaattatactggcttaaacactaaggatttatttttctgtcatataaaacaagtctggaggtgggtagtccagggctaatatgacactccagtgtcacaaggtacctgggctccttctatttatcttttttttttttttttttgagacggagtcttgctctgtagcccaggctggagtgcagtggtgcgatctgggctcactgcaagcttcgcctcccgggttcacaccattctcctgcctcagcctccggagtagctgggactacaggcgcccgccaccacgcccggctaatttttttgttatttttaatagagacgaggtttacaccgtgttagccaggatggtctcgatctcctgacctcgtgatccactcatctcggcctcccaaagtgctgggattagaggcgtgagccaccgcgcctggccctatttatcttttataggacatggcttccagtctcaagttcagtttgtcacccataatggccaaagagcagtagtcaccttacctgtttcaggcaggaagggcagagggcaagaaacaaaatggtgctcctcctgattgagtcagcgttctttaaagatgttttccagatgttccacccagccgcgtttttttttttttgagagacagggtcttgctcttgtcacccaggctggagtacagtggcatgatcatggctcactgaggcctcaatctcccaggctcaagcgatccttccattttagcctcccaagtagctggaagtagctgggaccacaggcacatgccaccataccctgctaactttttcattttgtgtagagacaaggtgtcactgtgttctccaggctggtctgcagtttccaactcctgagtgcaagtgatcctcctgctttggcctcccaaagggctgggattacaggtgtgagccactgtgtctggccaagctacttctacttatatctcattggtcataacttgatcacacagccacatccagctacaatggagattgagaaatgtagtcttttggctgggtacacagcatctgaataaaatccaggcattgttactaaggaagaaggagtaagtgtcaatctctgctccataactctctaggttaatacacacagatggaggagattgctagttgcccctcaagatccagccttgccttctgggctaagaaagcccctgagatttacctggccatagtggcactgggaacaaacaatgtatttctaaatcttctgattttaaaatctttcagaatcacgatttctccgacatcagtatttttatgtctttagaattcaacaaaatgaaattcctaagtctaatatatgtgaatattaagttttagcagatactgctacataactttccagaagggtgtggcaattcacatctccaccagtgatcagcatgttcattttccatacagctctggatattttgtctattttaaaatatcttcttctaatctataatttaaaaatgtaacttagtaggaatttaattgttcatgtaaccaaatcttcccattaatggctatgggtttcttcttttacttcagaaagtcctccccaccttcagagtatataaacatttttttctaaattcccttctaatttcttataatttatagttttatttttgttatttgattcatttattctctcaacagatatttattgagcacttattatacgtcaggctctcttcaaactctggtgagagtattttctaactgggagagacaaccctagttgataagaaacaaacaaaccaataagtaaataagacatttcccccagataattaatacttggtgacgggggaagacagtgagacaggctactttacactggcaggtcaggaaaggcttctctgaggaggcaagtgattcaggattaattgatgactggtggagaagtccggggagtggacacaggtgggaacagcctggtgggtgtgaacagcaacaagaaggttaatgtggcttcatggaacagggtgaagatgagacaagctgaacactgaggtgggcaccagaacatggagggctttgtaggtcccaataaggagtatggattttattgtatactggagatttgtcatccatctagaatctgtttttatatataagaaaaggtgtatatgtttgcgcaagtgtgtgtgtgtgtgtttgggggggcggggtgggggcagacagggcgtaacttttctttaaattagagtcaaaatttaattaaactattcattctttacaggcagtgaggggattaggatcttatcccacagaatctcacctcatttcaaatgttgtacagatattatctgagatatattttcaggccgggtgcggtggctcacgcctgtaatctcagcactttgggaagccgaggcgggtggatcatttgaggtcaggagttcaagaccatcctggccaacatggtgaaaccccatctctactaaaaatacaaaaattagccgggtgtggtggtacacgtctataatcccagctacttgagaggctgaggcaggagaatcgcttgaatccaggaggtggagcttgcagtgagccgagaaaacgccactgcactccagcctgggcgacagggcaagactctgtctcaaaaaaaaaaacgaaaaaagaaatatattttcaatgaaatcaagcatataatgactatttttatcccagcatcttggcttcttcaggctgctataacaaagcatccctagtggagcatccctaatctgaaaatccaaaatccaaaacgttccaaaatctgaaaccttctgaacactgacatgacaccacaaatggaaaattctacatgtaaatacacgtaaatacaaactttgtttcatgcacaaattaaaaatattgtataaaattaccttcaggctatgcatataaggcatatatgaaatataaatgaattttgtgtttacccatgggtctcatccccaagatgtttcattttgtatatgtgcatactcccaaatctgaaaaaatccaaaatcttaaatatctgtagtcctaagcattttagataagggatattcaatcccttatccataaacaatttatttataaacaacacaaatttaggctttgtaaaaaatagaaatttggccgggtgtggtggctcacgcttgtaatcccagcactttgggaggccgaggcaggcacatcacttgagctcaggagtttgagaccagcctggtcaacatggtgaaaccttgtctctaccaaaaatacaaaaattagcagggcttcgtggcatgcgcctgttgtcccagctactcgggaggctgaggcaggagaattgcttgaacccggaaggcagaggttgcagtgagccaagattgagccactgcactccagcctgggggacaagtgagactccacctcaaaaaacaaacaatcaaacacaatataaatttatttctcatagttctgcaagctgagaagtccaagatcaagatgtgagcagatccacctttggtgaagggctgctttccatttgatagatggctgtcttcttgtgtcctcacatggtggaaagggtgaggcagttttttgaggtctcttttaaaagagaactaatcccattcatgtgggggtctgccctcatgacctaatcactttccaaaggccccacttcctaataccatcaccctgggggttaaattttcaacacacaaattggttggagagtgagcgaacatagacattcagtctatagcccccagggatgtagccactgaataaaataatctagaacttcatctagaggcagtttataagtcacataagaaaaccagttttacttatagtcacataaaacttttgataaaaaacaaccctagttgataagccatttgtttactggacttggctctgaagaaatgacttttggctaatcctcaaaatgaaatctactcacagagggcaagatctgccaccagtgaagatgtttaaaacaatgtgctgtaggctttgaaggcaagtctaaagaagagcttctaaaaatattttgcacaatggtagcattaaaagagtaggcacatgggccccaagataacatctgtgaagaggaggaccagtgtgtgtttccttttgtatgtttaagtactaagtcagtgattactttataattaaaccatattcctcttgatctgtccataataaggtcactctgtaatttataataggcttcttaccaaattcaagctttacaacaaccctataggttaaatattcctgtgaattttatagatgaagaaacagagtaagtgaaagagttcatactcaattcaagtctcttgagtctaaaatcagtgctctctctacataagaagttagttgctaagatcacacaacttgagaagagagaagacagttttcacagtccctcccctctgaccactaggctgcttggggtacttttacaacttacctgctctgaattaaggctctctgtgaatctgattcatctgccaatttgcaaacagtcagccctgtagtacgtacctaagttgatttttaaaatactatttctcatcttaaaataatggaaattgagtgacataaatttcctggacactttttaagatccaaagacaatttgctaatttgtcgttacaattaaagagccccccaaaaggcgtaagaactggattttgacttggacttctgttccttagtttctttaacatattgggggccgggagtggtggctcacacctgtaatcccagcactttgggaggccgaggcgggcagatcacgaggtcaggagattgagaccatcctggctaacacagtgaaaccccgtctctacaaaaaatacaaaaaaaaaaaaaaaaaattagccgggtatggtggcgggcgcctgtagtcccagctactcaggaggctgaggtaggagaatggcatgaacccaggaggcagagcttgcagtgagccgagatcgcactacactgcactccagcctgggcgacagagcaagattccatctcaaaaaaaaaaagaaaaagatattgggaaaactcacttaaactctatgggcttctgtaccactaaggcttctcaaatttgatgcaagtaatgggaacttgctgtggctaacttgagccacaggagaaggatactgcaagactcacaaatgcttgaaagtttcggaacaggtttggaaatgggtaagaaccaaggaccgggttgacctgaactgaaaggaaattgaggtgctattagggacaaatatgaaatgaaggatgaactgtctctgacatcctttgcttctgtgtttggagatgagtaagcagggagtgggtgcatatgcaaacagggtggtcagaaatgctttggccggatgtgtgactgaaggctatgtgcagctgaggaaggctgggccaccacagtagcagagaggctgaccaatttggcatgcagcaagcaggaagtagaagggatcttaagcaagcaaagtgtttgggtagacaagattatgaggaatgagcaccaatgacatttccaacaagcactgaaagcttccaaatgcttctaacagttactgcctgtacaaacacaggacagtcttgactatgtgagactgtgacagtttcctcatgcctatgaaatgagagacagtacttagcactgacatgagcttcatatttgaaggcagcaaactactgaaccacatgtagcttccgaaatcaaaagatgcaaactggcagaccacaggtcagatatgacatgcagaaaaaaattagttactatcttagcctagtttgtgctgcttacagaatatcagagactgggtactttatagagaacacaaattatttttcatagttttagaggctgggaagtccaagatcaaagggccatcatctcgtgaaggcgtccctgctgtgtcaaaacatggtggaaggcatcacatgggcaaaagagagagagggaagcgggggcaaactcctttttatcaggcacccagtcctgtgattcattcatgaagacagtgtcctcctggcctaatcacctcttaaagctcccacctctcagcactgttgcactggaaattatatttctcacacatgaactttggggaacacattcaaaccattacagttaccaaagataaaaatgcagatttcagatttctcttggggaagaaaaaaggctctggcaataccagggctgtatttccacatggcaacagttgtctgttgccttctgttgggttgtgggctctcacattgccataccctcctcatggcccccttcactcataacagtacctgcctagacctcgaagcactagtttccaaccctgcttgagttaactacttccatgacaactgtgcaaggccagagtgtgggcagctaccttgagggccaccggcatgggctgcccaagtgaccgagggcactaacctctgctctcgaaacttctttggtaggatctcttcactcctgacttactacttgtaactgctggaaacaaccatttattctacccctactaaaccacatctgtcttttctgtcagagccaatcttcaagcatatgcttcctcaaaagactttaattaccaggtagtagaagcggcagcaaagagtccccatctagcatcttctaaaatgtagtggagaaaccaacgacaacgaatctgagacaaccagttgtttgacttctctgaaaacaacctctaaaaaaagccttctctcgtgaatagctgcagacatgctgggcactaattgatgatgatgccctttagtgagtctcctctgcacatacctgattccatttggatgctggccctcacatacttctatctgcgaactccggaacacagaatatcatacaatgtatgattcaatctggactctgctggagccattctctacaaagcagacacaactttttcttgcctaaatcctttcaagttgctttatttttatttttaaactaattaccataaatctaatttatcaagctgcttttaagataaactttaaaatgcttagagcagtctacaaattcttgcactgtttcactcttccttatctctcagtcttcatctcccattgcatcttctctgactctttcatatggatcttctttcaatttgtggaagaagccaagcttgttcctgcctcaggtccttgacacttgctgttcactctcctgggagactcttccccccatcttagcttttaaataccagctggtcagactccttacctgccttccaccttcctctcattctcccactcctcgccttcatttctcagagtcctccgctctcttctttctattgctcttaacacaatttgtctgttttccgctaatctgtaaactctacaagagcacttaactattttgcatcccaccgaatattcggagtcagcacagaccccgtcataatagagtaggtaagcaatgcttacctgtcaaatgaatgaatgaaacacccgattcatcccccaagccagcttaatacattagatgatcttatggactgaataagacatccctccttttaaagaatatttttgttttagcttcacatcagcaaaccccgaacggttcatcctaagataagcctggtagagaggaaagatactaaccttctagggtcctaaagtggaactaggaacttattagatatgcacattctcaggcattatttccgatagcccagtcagagaattctgggggtatggcccagcaatctgttttaacaaacctttcaggtaattctgatacacactaaagtttgaaaggtttctttaaggctttccagttttcttttcttttttggagggatgggggggggtctccccatgttgcccaggctggtctcgaactcctagcctcaagctatcctatcctcccacctcggccttccaaagcattgaaattacaggcatgacccactgcggctcgcctccgtttctaacttaaaaaaattttctttggaggggagccttaatcctttctattatcctgctagtagaatcttactactcctgagggtaatctttgtcataatccaaaagccttgaataaaggcctatttgtatagttatgcagaatatattcctgggaggtttgccttcctaggagcacaggtcggccctgggaggtgggggttggggggcagggctgcattctaaagtcctgtaacaacgcacaagtacaactgaatagaactcggaacaaaggcatcagggccacggtgcaagtctttgttcatccgttccccgactgctcaccctgtctgataccgctcttttccacccagaaaagcagccactcaagttttaagaatggatgtatgccgcggacgttttcgattaggccgttttctctcaggcactggaggatattcgtggctgcaggaggcgcttccaccctttcctcaacctagcaaagaagtaactgaaactaacccaagggttacaaccgaaaagccccttccagcttcagaagcagaactggaagctcggatagacttctccgcctctacactcctggaaaacccgcagtggatttcaccaacttcaggatcggagcccaggcaggcgaacgtaccttattgcgcatgctcgctagcccctcccgctcggagcggaagggggagcgctgggggcctgggcctggcctggccggggcgtcggcaccggcggccatcttggcttcccggggaaaggcggcgtgaggggaagaagttgtagggtgggggcaggagtgaggaggagggaagagagagggggaggaggccgcggcggggcagggcggggactgcctgcctgcctgggttgcggaagtgatagccgccgaccgagcctgctgctttcttgctactgcttcggcttcccggctaccccccggacggtgaaggcggcccagctgtggatggtcagatagcccttgtctcccgccgccaatctctggcccctagcagcacggagcagacggcggcagcagcagcagcaggcgaggaggaagatggcgggacggctgccggcctgtgtggtggactgtggcacggggtaagggggcttacgggcgggggtggggaaactgaggcggaggaaggaagatggcgggggagggaggaggccgggaaatgaatggtgcggcgaggtgccgccgccggctgtcagtcctagacccgccggccagcgaggggtggggcccgcagccagggcctcgcgggtcccctcgtttctccctcctgggactggggcgggggcgcgggcccgagattcaacccccaaccctcccagcggctttctccgcgcgacccctcccggcccttcccccactacggtgggcagcgccgcccaaagggcgctggggacggtcgtcttgggggtggtccccgggcccgacccatccggctttcctttccctccgcgcccgtttttgccagtcggtttggggacccaggggccgagctcggggactggcctggcaggggagctagaaaagagaagcgctcctggtaggtttgacaagatcgctgtgacaacattctgcccagggtgtgggtggggaaagggaagaatcggactctgaaaatgggaacctacagtggggctttcatttgaccacccaccttctcctttcgactcctggtcatttccatctccctctgcgttttaaccggtgaaccaagtcacattttaattcgagggagaaagatgtcatgtgttacttctgtagcctcaaaaaagtcccccagtgagcaagcgcgctgcaacttccttagttttgtcaaagccgcttcctgctttcagtcttttatacccttataaggtagtttttagtttccaacctgagcacatcttactagaacttttaagcaggttttaaagagactgaattaccggtgcttggtgtccatttatatgacttaaaaaaatgtacttatgtttcatggagtgggggaaaggaagcaccctggaaaataaactaatttaagttgtattcgttcattctgtgatggtatcttgaaggaagcaggaaggtatagaggtatataggagggtgtttaagttgcaaatagttactcgtagatatgtagggtagtttgcgaaaatgtagagtggctttaaagtgtggtcgttcattttgtattaatagacgttttagagagttgtaccacactcataactgcatagagaatagactacccttatttttatgtagaatcactgcttcaggatgacatataggaaagtggtttttttttctgtgctgatgacatctctccaattcccagaacactcccagttttttttttttgagggggaagtc

SEQ ID NO: 19 - DNA, 26803 bp, Homo sapiens
  source 1..26803: mol_type = other DNA; note = cDNA
gccgcacctaccgctggcctcagacatcagcaccccaaagggtatgttggagtcccatggtggaggtgccggccgctcctccacgcacttgatgtataccgaggatcccatgcttgacgtcgcagggggcattagccaggagcacgttgctgggatgccagtccaggctgaggatggtggagccaatgggcttcttgatgtacttgcacacccaccagttattctcccgctggaaataacagatggcgatgacacacagctgccgcccacagcaaacttgtcctgtgggcccagcacacacagcaggcagtgggtgcgggcttaccctcctggcccgtcagcgtccacacagaggccttgtggcctgtgccataggtcacgatgaggttactcttggaggcccagttgatgccttttacctgcccgttgtgctccttgagctcgggcaccttggcccacttggccccactcttacagcttcgtggttgttgaggcagatggtggccatctgggtgcggcccttgctccaggtttggcagcaatgggttcctgcagtcagctgtgggaggctgtagttgctctccttttcagagtaccccagctgcagactctgggcaggacgcacccaaccagtgctctggttttttgaagtaaaaaatgcataatgcaattttaccatcttaaccattttttaattatgaaatccggtagtgttaagtataattgtgttgttgtgaaacagatctccacaactttttcattttgcaaaactaaataactcctctttccccctactcccaagctccaggcagctaccatttctgtttctatgaatttgactacttatattacctcatataaaggggttcataagtatttgtcttttcttgcttggttacatcacttagcatgatttcttaagtttcattcatgttgtagcacatgtcaagatttccttttttttttttaaaaaaaaggatgaagactattcgtttgtatgtatatgccacattttgtttatccatccatttttcttcaaatgtttgagttgtgtgatgtttaattatgtgtcaccttgacggggcaaagggttgcccagacagctggtaaaacattatttctgggtatgttagtgaggctgtttccagaagagattagcatttgaattggcaggctgaataatgaagatctgccctcatcaatgtggatgggccagaacaaaccaaaaaggtggagaaaggaccagttatctccccaccacccctgtgccctttagctgggacatccatcttctgccatcagacatcagagcttctggttctcaggccttcagactccagaagttataccagtggcttccctggcttttggactcagactggggtttcactgtatgtttccctgattctcaggcctttggacttgaattgaattacagcatgaactttcctagttctctagcttgcatatggcatattgtgggacttcttgacctccataatcatgtgagctaattcccataataaatctcctcttatgtatctataattcatatagatatgatatctatatatcaatcccatagataaatagatgtctcatatatatatcaatgtctctatatgtagatgtctcatatatggtatctatgtatctgtatcatctctatatatctaatcatctaatatagatgtctcatgatatctatatagatatatctatatcatagatgcagagatatcatatatatgtctgtttctttggagaattctgactgatacaggttgcttccgccttttggctatcatgagtaatgccgctgtaaccatgggtatgcaaatacttctttgagaccctgctctgagttcttttgggtaaatacccagaagtggattgctggatgatttggtggttctacttttaattttttgaggaaatgccatactgttttccataatggttgcaccattttataatctcaccaagagtgcacagggtttcaatttctctacatccataccaacacttattagtgtgtgtgtgtgtgtgtgtgtgtgtttaatggctgtcctaatgggtgtgaggtgacatttcactgtggttctgatttgcacatctctgataattgctggtgttgagcatccttccatatgcttgttggtcatttatatatcatctttggaaaaaatgtctattcaagtcttttgtctattttttttccttccaacttttattttaggtctgggggtacatgtgcaggtttgttacatggataaattgtgtgtcatgggggtttggtgtacagattatttggtcgcctaggtaaatgagcatagtacctgataggtagttttttgaccctcccccttctcccaccctccaccctgaagtaggccccagtgtttattgttcccttcttagcatctgtgtgtagtcaatgtttagctcccacttataagtgagaaaatgtggtatttggttttctgttcttacattaacttgcttagaatgatggcctccagcagcatccatgttgctgcaaaggacatgatttcgttcttcttatagctgtggtgtatatgtaccacattttctttacccagtccactgctgatgggcatctaggttgattccatgtctttgatattgtgaatagtgctatgatgaacatatgtgtgcatgtgtctttgtggcagaacaatttacatttctttgggtatatatccagtaatggggttgctaggtggaatgattaattcacttttaagttctttaagaaatctctaaacctctttccccagtggctgaactaacttacattcccaccagcagtgtccaagtgtttccttttctgcacaaccttactaacatctgttattttttgactttttaatgatagccattctgactcatatgagatcgtatctctctgtggttttgacttgcatttctctgatgattagtgatgtagagcattttttcaaatgcttgttggccgcatgtgtcttcttttgagaagtgtctgctcctgtcatttgcccactttttaatgggcttgtttgttttttgcttgttaatttaagtttcatgtagattctggatattagacctttgtcaaatgcatagtttgtgaatattttctcccattccataggttgtttactctgttgatagtttcttttgctatgcagaagctctttagtttagttaggtactacttgtcaatttttgtttttgttgctttgtccattttaaaaaatagagtaatttgattattttgttgttgaattgtaggaattctttatatattatggatgctaacatcttatcaaatatgatttgcaaaacattttccctcattctgtaggttgccttttcactattgtgtccttcaatgaacaaaagtttttatgtttgatgcagtcccatttgtccattttttttggttgcctgtgattttggtgtcatatccaagaatgtggaatgtgtcatatccacattcaatgtcctgacgatttttttctatgttttcttccagaagttttattgctttgggtctttggtttaagtctttagtccattttgagttaatttttgtatgtggtgtaagaaacaggtccaactgcattgttttgcatgtaaatatccagttttcccagcatcatttgttgaccagactgtccttttcccatttagtgctgttggagctcttcttggaggtcagttggccatgtgtacacaagtttatttctgggctctttattctgttctattgatttgtatatctgtctttattccagtaccacactgttttattcttctttttcaaaattgtttgactcttagggccctttgagattccatatcaattttaggatgaattcttctatttctgcaacaaatgctattggaattttgataaggattgaattgaatctgtagactgttttgagtgatattaacatcttaataatattaagtctaatccatgaatgtgagatgtttctatttatttatctcttctttgatttctttcagtaatgttttatagttttcattatataagtttttacttccttagtcaatttctaagaattgttttctttttgatgccattgcaacgggaatcattttcttaattttttcagatgctttcactatttgtgtatagaaatgtaactgattttttgtatgtgtgttgattttgtatctggtaactttactgaatttttaaatttgttctaacctgttttttttttttggtggaatctttaggattttctgcatataagatcatgccatctacaaacagaaatttttacttctttcttcccaatttggatgccttttattgctttttcttgtctaattgctttggactggagttccaatactctgttgaatagaagtgtccagaacagacatttttgccttgttcttaatcttagaggaaaagcttccagtttttcactgagtatgccgttagctgtggacttttcctaaacaacctttattatgtgcaggtaatttccttctctttccacttttgagtgttttttttcttttcttttctttttttgtgtgtgaaaggatattgaattttgccaaatgcttttcctgcatcagtggagatggtcatgtgggttttgtcctttattctgtaaatgtggtgtactacattgattttcatatgttgaaccatccttgcatcccagggataaatcccacttgatcatggtgaatgatcttttgagtgtgctgttgaatttattttgctagtatttttttgactacgttcatcagacatattgggtaattatttattttttcttgtagtatttttgcctagctttgatatcacactaatgctgcccctcaaagaatgaccttggaagtattcctttctcttcagtttttgggggattgatgtaaattcttttttttttttttttggagacagggtctcactgtcacccaggctggagtgcagcggcacaatcatagctcactgcagcctctaactcctgggctcaagcaattctgcttcagcctcctgagtagctgggactataagcatgtgccaccatgcacagctaatttatatatatataatataatagataaatatatatatatatatatatatatatatatattttttttttttttttttttttttttttttttttttgcagagacaaggtaggtcttgctatgttgcccaggctggtctcagactcctaggctcaagtgaacctctcacctcagtctcccaaagttctgggattacaggcatgagccaccacgtctggccagtgttaattcttcaatatttggtagaattcttcagtgaagtcttctagtcctgggcttttctttgttgggaggtttttaattaccaactcaatttccttactagttattggtctatttatatcttctatttcttcatgattcagtcttggcaggtttggtgtttctagaaatttatccatttcttataggtcatccagtttgttggtatagttcatagtcttctcttataatcctttcttatctgtagctcagtacctcctatctggagatgcagaattaggaattgaaactataataggccccccttttataatggggtgtccttatttttaccttctcaccttgcaatacccttgtcttgctagtaaagaaagaagggaaatttgattcaggaggtaatcatacttttcaatttgtacaagatctaatagccatgaactcctatgtcattcctcaccatcccataattcttagcccagccatcatccttacctcaatccctgctggtgcagcctggtttattctgttaatgtgctgtactgcattgaggtaaggacttctgctcagctttcttcttgataccattgtaccttgattcacacttcctcttgtgaaataaagtttagcatgaagctgctttcttacatattttaagttcggcttaaaggtttttctgtacatcgtgaactgtaacaagtggaatataaccagaccgtagcttacacttgtgccatttaccaagttttggccaatcaaatgtagccaactgtttgaactgtattcaaataagggaaatgctcagctgtaaccaagccaactgtttctgtacctcacttctgttttctgtatgtcactttcctttttctgtccataaatcatcttccatggcgtaggtgtgctggagtctcagagtctattctggctcaggaggctgcctgattttgaatcattcatggctcaattaaacttctttaattttttttttttttgagattgagtttcacccttgttgcccaagttggagtgcaatggtgcagtctctgctcactgcagcctctgcctcctgggttcaagcgattctcctgcctcagcctcctgagtagctgagattacaggcacatgccaccatgcccagctaatttttctatttttagtagagacagggtttcgccatgttggccaggctggtctcgaactcctgacctcagcctcccaaagtgctgagattacagatgtgagccactgcacctggcttcactccttcaaatttaattcagctaaagtttttatttttttctctttcttttttttttttttttttgagacagagtcactgtcacccaggctgaagtgcagtggcacaatctcggctcactgcaacctccgcctcccagactcaagcgatttctggctaatttttgtgtttttagtagagatgggttttcaccatattggccaggctgatcttgaactcctgacctcaggtgatctgcctgccttggcctcccaaagtgctaggattacaggcatgagccactgtgcccagcctcaaatttaagaagaaacagttaacatggacttgcatacctccaggatattgtgaatacctctcaatatttttctaagttctcaaagtcaaattagactctatgacaatcactcaaggttccctttgtttccaatatgttgatgacctgctactttgcaaccaaagcaaacagggtgctcttctggactcccttactcaccttaaggcactgactgactaaggttataaatcttccaggtccaaatgccaacaggtacaaaaaattcttacctacttaggccataaaatattttagggtactcaaaaactcgtcccaaaatgccttgaatcaattttattcattatctctcaaagacaaacaattacgtgaatttttaggagcaactggatattgccaatggattcccaattttgctgcccatgtctaacctttatatgctgtcctcttagatacaaccacagagcactttacctggtactctgaggcaccagcctccttggaagcattgtccacaccccagcccttcgactacccaactttgacaatcttttttacctatattactgtgaaaatgatgggattgttgtgggtatcttaggacaatctttttgtcccataacatatttctcatgtcaacctagatatgtcccacagtacctttctcatgttaagtagcatcaggcattcctccatgcttatatgcaatagacttagctgccatcctaattgacaaagcaagtattcttatactgccccaccattcacctctctgttctccatgctgttcctctacattggtatccctttcaaagtggatcttgcctcaactaacccttaactgttttttttttgtttttgttttttgttttttgacaattctggcactaccccaacacctgcttgttccaatctttcaaagcctggccaccttaaatagttaatctggttactggttaggttaacatctacataatgcagaagagtttttgttcctaccaatgtaaatatacctggtgaacccattctggacaatggatatataaaagcatatggtatccataagcaggaagacaaagtcactctgtctcctcctctggtagattaatttggcactgggaagcctccatggaagctcaaggtcagtgctttacccaagtaagactattggaaggaaatctctctacattgaaaataaatacagtactggaccttttctgggtaacatttcaaaaatgtattgcaatcaaattctgtgatttggcttcacagatggcactaaacaaccctccactgtcattgttacttggattataactaagtatttcaatgtgagtgaatgtcagattacaggataatattcctggtttgtaggaaaaagtagtgggatatggaatagctcaagtgttcccctagttggcttgaccagcacccatgactatagttggatggatggaccttaacttggtcaataaataaaatacatttctatagtaaccaaggctaaattaaagggtttctctgccaggtgtttcacaacctgttttaccctcatagcctaacagaagcacattggaagtgaagatgtgcagatgccaacatgattgatgataagggttgtgaaagacacagaattccaacttggttgttcacaggttccaccactatgaccttgtctgtaaacaacactggtctctctctctttttttttttttttagaaggaatctcactctgttacccaggctggagggcagtggcacaatctcagctcactgcaccctccaccttccggttcaagcgattctcctgcctcagtctcccgagtagctgggagtacaggtgcccaccaccatgcccggcttatttttgtagttttagtagagacaggatttctccatgttggccaggctggtcttcacctcaagtgatccgccagcctcggcctcccaaagtgctgggattacaagcgtgagccactgtgcctggcctggtctcatttttatgtagcaataagatatataaaaggttcccacctaagtggtcagagaaatgtgaagttggatatctggtgccttcccttaccagatatcccactttgaatgctagccacattacaactggggttcttttatatataaattaatgccatgtagatgcacctgatgagacatcatagacaacgcacttatgtattgcaaccctaagttctcagtaatgagaatgcttttccaaagcctggcaatgtacgatgtagaaagaacaatcctaagcattccaaagtaatgaacaagcatctggtgccactatacagactgggaagcctgccatttacaagttagcagtgtagcctctgttgcactccaaaattgtgtcctagatatgctgactgcccaacagagaggagcttgtacaatcactggcaacaatgctacttctatataaattagaaaggaaaaattgtgtctaatctatatcatttaaaaagaaaggttggccgggcacggtggctcacgcctgtaatcacagcacttcgggaggccgaagcgggcggatcatgaggtcaggggatcgagaccatcctagctaacatggtgaaaccctgtctctactaaaaatacaaaaaattagccaggcatggtggcgggcacctgtagtcccagctacttgggaggctgaggcaggagaatggcgtgaacatgggaggcggagcttgcagtgagcagagatcacaccactgcaccccagcctgggcgacagaacgagactccgtctcaaaaaaaaaaaaaaaaaaaaaaaaaaaaaaaaagaaaggtcaacattctacgttaggtaaataaggcatagtccaattaactgaactgaactgacctattcccacgactgggagactggttcaacagaatatggaccagtgtgttcagatttttctctgttgtaatctatgttcttctttcattttgcagatctcttacctcccagcacctaatatgagtcttttctacataggaaataatccaaatatttgtgaaacttcatgtgaagtttcaaatgggggaagtgaaggaacgaacaacccacctcctaaccccaatttatacccacctgtgttagttcatttgtattgctatgaagagatacctggaggctgggaaatttataaagaaaagaggcttaattggctcacagttctgcagggtgtataggcatggcatcagtatgtgctcagctcctggtgagggcttcagggagcttacaatcatagctgaaggtgaagggggagcatgcatcttacatggtgatagagggagcaagagagagggtcggggggtgccaggctctttaaacaaccagctctcatgtgaaataccagagccagaactcactcatcaccatggggatggcactaagccattcatgagggatctgcctcatcacccaaacacttcccagtaggccccacctccaacttggggattgtatttcaacatgacattttgaggggacagatatccaatattcaaaccatatcaccaccagaaaggcataaagattactttaaactgaaaatattggaacaaacaatcgctgcggaaagcagccttatctgaccgaaagcagacctgtccaagagttctgctatcatcaactccttctgagggactttccagacagcgaggtgacagatgtttacaaacgtgacgttaaaaaataaagttgtgtaaatgagtcttatcagaaccttttatactttctcactgaagcctcagttggtatataaacccttaccactggctgtttgggaagtgactccttacagagtgctccctcaggcatggagaataaacttttctcctgttaatgtattttcaactaattcgcaggccctagaccactcagatctaagttgacagatgaaatgttttcctcccaacatgaccaagcttatagtgcttttatggtatatctgtagagattgcggtcaggcataacattgtttgttagggttgcagtacctttcaggaaatgctcaaagtttaattgatttaggggactttcactggatgagatgcctctctgagtcatcataggttgcaaagtattagttttttgccactcactgtgtgttgtgatatgacatactcataatggggtaggacaagatgtcagttttgcttccaaactttccttacttgtttcctttttggttcaccactaatgttgaagaaatgcaagatggaaaatctcaatagttgacactgattgattgattgagatggagtctcactctgtcacctgggctggagtgcagtggcgtgatatcggctcactgcaacctccacctcccaggttcaagcagttctcctgcctcagcctcccgaacagctgggattacaggtgcccgccactactctcagctaattttttgtatttttagtagagatggggtttcaccatgttggccagactagtctcgaattcctaaccttgtgattcgcccgcctcggcctcccaaggtgctgggattacaggcatgagtagttgacatttattaagtgtttatatgtgataagcactgttagattatcccatgtaatacatcacttgattgatttaagagattgtcatttgaaagattaaagaatatttcccagaaaggatgcttggtcaagcctcatcatcctgggatggatgggaccttggttttgggcaagtttattacaatttaagactattctaggtcatggaagtgtgtgttaggacatataagtagttttcttgtccagaatactttgtaataatataccctaagtttgatcagctttttggcattcacttaattaaaatgttcacaagtaggtacatttttagaattgttaagtaatatactatttgtagtagcaaaacactacaaacaaatacctaatataataagagggagtggttgaataaactagggcatgtcaactaaaaaacatatttgaaatgcagggaaaatgcttactctaggcaatagttacaacttttgtccattataggtacataaaggaaaagagtacacaaagaattctattaatgatgaaattaggaacaatgttcccatccccaatttatacccaaaagaaaatatgttctggctgggcgcggtggctcatgcctgtaatcctagcagtttgggaggctgagacaggtggatcgcctgagctcaggagttcaaaaccagtctgggcaacatggcaaaaccccgtctccaccaaaaatacaaaaaattagccaggcgtgatggcacatgcctgtaggtccagctacttgggaggccgaggtgggaggatcgcttgagctcgggatgtggaggctgcagtgagcaaagctcacaccactgcactctagcatgggtgacagagtgagaccccgtctcaaataaataaataaataaatgaaaacttatattctataacatgtattcttatgtctttggctaaaacaaaagagaagaaaaataagtagagagctaagtgaagtgaagtgtcttgaaacagataagccataaagtgagatatatgagtaaatgagtagttgctggcctaggaattgtttctgatgttttcacctattgaaagaatttttttctttttgtaaatcttttttttggtttgttaaactgaaaaataaaagcaatgaagagataatattgctgtagtaatgtcccttagtgcatccttggagttagttgtcttgaatttggcctgctctgattgtcatttgctgtttgctgcattgaaaaaatttcagatttacaaaaatatgtgtgccagggtggaggaaatttttaacttgatgaattctcacctttaggacactgtctgctcttatatggcacttagcaagatgctgcaacaattgtccctgctcatactgttggaggaagagggtatttgcagggagattgctggagtcaagatgatccaaatttaaattgcagtggtgagcagtggccactagagggctgtgaagtacttaattctaaaatttccacattttaatctggagcagaggaaactctggaaggaccactgggactaagggagctcaatcccaaacatctttatctgagccctaaatgtcttctacgttattctttgacaatactttgttacatctcagaatttgtagttttgaggtggaggtttaagttagaaatagtttaatatattctattccagaatataattcgcttttttccaaatgaaactggcactaaaagctttttacttgaaagcaaatacacttcatatgctttatgtatttttgtccctatatcattgaaatagtagattcaaggctgtttgtaaaaataagtgactatactgcatataggaatagccccaccttaaagactttgattttaaggacattaagtgggagggtcttttttgcctttttttttttgactatcaaaatactttcacatttcttcaattcaaagaacaactgttctaaatttagaagacagacaagtgaaagagaatatgctgccatttccgatggttcttttttactcttgatccatcactctcctgtctttggctgactctttctccaaaaagtaccccttatttttcctatgcctttaacttctgttttacttactctattgtagaactttgagaagttgtacaccacattcactgctacctcacattaactctgcttactcattgcagtctgccttctgctcctcaatgattgctcctgtgaaggtcatctttggtttagcacttgtttgaaatctaatagcctaaatgcacatcgattactgtttagaattcaaacgacatttatttatttactttgagatggagtttcattcttgtcgcccagctggagtacaatggcatgatcttggctcactgcaacctccacctcccaggttcaagcgattctcctgcctcagcctcctgagtagctgggattacaggcaccctcaaccacgcctggctaagttttgtattttttttagtagagatggggtttcgccatcttggccaggctggtctcgaactcctgacctcaggtgatctgcctgcctcagccccacaaagtgctgggattacaagcgtgagccaccacgcccggcccaaatgacatttatttttaaaagcaactaaaccaggaaagaccttcactcattcaatcaaaaaacatgcattgaacacgtattcttggctagatccggtgctgcaaagatgaaagacgccactcctagcctcaaggagcttgcagtttagtggtggcactggatgtaaaataatcataagatgtgaggtgaagactgatgaggtctattttgttgacacaggtaaacattctcagagaatatttaattagcaacatgacaaaaattagttgatttatttgaaggatatttaagtgaatttagagatgagatttgtttttacttttttaatttcagagagttatgttggttgcaaaaatctttgttttctagtatctattttataagccatctgagaccaggatgttttctttttaacatgtaaacttgatgcagtctctttgtaaagtaatgaatccccccgtgaacctcaaggagaagaaagtttagcttgaacacttaatttcaagtaccttgagaagggaacaggctgtattgctcagagaagaaggctctattagctaaggtagaaggacaatgtaaaactcttgtgaagacacctaaatgtcattccactcaaagcccattattaataatttgaaactgtctgctctgtgaaggcaacgacctatctttactcatttttgactctgctaacatttctttttatagttgaactagaggcaaggcaaggaacttgacatgcttgttttccctttagaataaagtggccagaagcagtttcttctgtgtgatccaacactttcttatccccagagcaactctgcatagtctagatgtaccagagcccagcagcctgtgaggtgatgtgatgtgatgtgatgtgaacagaaaggctccatgttggatgatgcctatgaatctaatcagattctctttcttggtcattttgatttccagagaaggcagtagaagcaggttctgtgtgtataaccagagactagagggaggaactgacagggaagctgggtcatgagagtggtgtgctcagcagagtagggagtaagagagagctggctccagagaggatgacaaaatgcccatgtctccagcacttctttagcattctgagttcccatcctatcatgaataggaagagttttctagctctccatgaagccgaaataggtttcagactttcctttctttcccacacacataataaatacccatcaggtaacctgggcatggccttttatttaggcactaacaattctaaaaggaattaataaatgcttcactgccatttcacagtgtaacagcatgccagtttaccataccattaacatttagcctcgtggtatttctgtgttttttcagtcagtaatgatggcatttttgccaagataatatagtgttaaactgtattggttgaaatcctaggctttttaaatgtaaactagcactttacaaagtattacaaaggaaacgaaccagggaaacttgagaaggaactttcttatccacattgttataggttataaaatagttcctatttaaattaaaatacctactgttgcacttttatgtgtgtttttatatataaataacatctgggttggcaaatatgtgattttcgtcaaataggctttgattgaagatgaaatatttttgtaaaactagtccttttttaaagttaaaattttatgttaccacatgaattgatcagcagaaatttatttacattatgtaaattgatttttaagagtatccacaaggttttagctattatttttgaacttgcttcctagcccacacaattaagtaataagagtaagaaaaaagaaagcacacatccaaatcatctatcaaaacccagcttctacccaatgacacatcattttatttcttgatcttgtaatgttgagcacatgccttggacatattcctatatgtgggctctattctctgcagcacgtcactccctcttttatccgcccctcttatgtgctcccactcctatcctcttcaattaaaaatgcctctttcttttttttttaagttaattaattaattaatttttttttattgatcattcttgggtgtttctcgcagagggggatttggcagggtcataggataatagtggagggaaggtcagcagataaacaagtgaacaaaggtctctggttttcctaggcagaggaccctgcgggcttccacagtatttgtgtccctgggtacttgagattagggagtggtgatgactcttaacgagtctgctgccttcaagcatctgtttaacaaagcacatcttgcatggcccttaatccatttaaccctgagtggacacagcacatgtttcagagagcacagggttgggggtaaggtcatagatcaacagcatcccaaggcacaggaatttttcttagtacagaacaaaatgaagtctcccatgtctacttctttctacacagacacagcaacaatctgatttctctatcctttccccacctttccccctttcctattccacaaaaaccgccatcgtcatcatggcccgttctcaatgagctgttgggtacacctcccagacggggtggtggccgggcagaggggctcctcacttcccagaaggggcggccgggcagaggtgccccccacctcccggatggggcggcggctgggtggaggcgggcccccacctccctcccagacggggcggctggccgggcgggggctgaccccccacctgcctctgggacggggcggctggccgggcgggggctgaccccccacctgcctccgggacagggtggctgctgggcagaggggctcctcacttctcagacggggcagctgccgggcggaggggctcctcacttctcagacggggtggccgggcagagacgctcctcacctcccagacggggtcgcggctgggcagaggcgctcctcacatcccagacggggcggcggggcagaggcgcttcccgcatctcagacgatgggcggccgggcagagacgctcctcacttcctagacgtgatggtggccgggaagaggcgctcctcacttcccagactgggcagccaggcagaggggctcctcacatcccagacgatgggcggccaggcagagacgctcctcacttcccagacggggtggcggccaggcagaggctgcaatctcggcactttgggaggccaaggcaggctgctgggaggtggaggttgtagctagccgagatcacgccactgcactccagcctgggcaacattgagcactgagtgaaccagactccgtctgcaatcccggcacctcgggaggccgaggctggcggatcactcgcggttaggagctggagaccagcccggccaacacagcgaaaccctgtctccaccaaaaaaatacgaaaaccagtcaggcgtggcggcgcgcacctgcaatcccaggcactcggcaggctgaggcaggagaatcaggcagggaggttgcagtgagccgcgatggcagcagtacagtccagctttggctcggcatcagagggataccgtggaaagagagggagagggagactgtggggagagggagagggagagaaaaatgcctctttcaatatgtaaggtaagattgatagatgattgccttccgtagttcctcccaaaattcataggttgaaatcttagccctcagagtggtggtattaggaagtatggagcctttgggagataattagctcaggagggtggagccctcccgaataaatttaatgcctttataagcaagaccccagagagccctttcacccttttctaccctatgaggacacagcgagaagacagctgtctgtgaaccaggaagcaggccctcactacataccgaatctacaagcaacttgatctcagacttcccagtctcctgaactgcgagatataaatgtttgctgtttaagtcacccagtctaaggtatttttgttatagcagcctgaatggactcaggcggtaggcaagaggaggacagtgcacctgagttatacattgggcaagtcatttaccttcattggagcctcggtttacttgtgttagatatgggaataatgatagttacactgcagggtggttgtgaagattagagaaagtgcatatactctgatcagcacagtgcatggtacttcagtacacagtgttaactgaagacacagtgttaactgaagacacagttgaaaactaaagatagctagtgaaacacatgttctctgcacatcatacagtttcttgggcagttttcaaagtgtgactctgcagttcataccaccattatcacttagtctctaacaaataatcatatttgacacatttggctgggacttaataattttatttgggcagcagctggtttctggaagtgttcatgggaagaaaatgtatattaataaagttagcaggtaatgttctttgttgaaatgttggatattgtgggacacaaaagagtcagggtaaatgtacatttacagaattgagcgtcagagtgtcatagttctctaatgatttacaaagaaagaattcctggaacaagtgaaatacttaataattaattcagcctgtatttctgcaggtaatcaactgcagtgataaaaaaataaaaatggaactgtcttttgctcattggaaagatatttgctgcattcaaagtcatcaaaaaggcgaagcaatgaaacttaagacttttgggttttaataattaaaaatccatcttaaagagaaccttgggctctggcacggtggctcattcttgtaatcctagcactttcggaggcccaggtgggcggatcacttgaggtcaggagtttgagaccagcctggttaacatggtgaaaccccatctctactaaaaatacaaaaattagctgggtgtggtggtgtgcgcctgtaatcccagctacttgggaggctgaggcacgagaatcacttgaacctgggagatggaggttgcagtgagccaagattgtgccactgcattccagcctgggtgacacagtgagactctgtcttgaaaaaaaaaaaaagagagagagaccgtttgtgaaggtgtgttacacagagaattcctgattcctaatctccagaatagaaaaattcaaaggctcagaatcatcccaaatcaattatccagtggcatctgcattttccgatttcattctcactagccgtgtgaacatgggcatgaccccaggagccaaggaattctctaatgcctaaggacagccatgaggtcaatgaataacatgtgtcaggtttccctggcacagaggagcttcccagtaaatgttattttccttagctatttcccatttaacatgtcaattattcttagtgagtttccattcaatatgagagtttatataatttgtcatattgaataggaacttgctaaggaaagctgtgtagcagaggaagcagccccaagtgacaggacataaactttaaattcacaagatgcttgtatgtgaattatgatggtatctgaattaacagtgatatttacagtttagtaatacatttttcttcccttgtcttgggctttataaaaaccctgtaagtcatgtatgtcaggaattttatcctcataattgagagaaaaatactccatattttattggaaataagatcagtagattctctgtatatcatacaatgtcttgcctacttaaattatcaccccccttcctatttcaacaaagactttctctaagtacccctggcctcttgccctgatccatactgttctccttggtaatggagtgagtttcctgcctggtgggtgcagccattggccacatttctctagtgtatggaactcaccggataaaaggcctcagatgtaggtctctttctgctgtcttcacccaggtgttgtggagtgtgtctgtccttgagcactagctttcccctgtgtgggctgcctcaaggcctgcattcatcaggtagggactgttagctggtagacctaagccaccactccagtactgactgtcagtaaggaaggtgacatgctgacctccatctgctacatcattgtgtatatgtcttcattagttctgcatattggtgagaaaatggatgctctgattcctcttgaaccccaagagaatgaaggtaaaaaaattgaggtcctaatgcaaattttcttagtacacccaaatctttggatagagtcctgcactctttgagatatatcacacaatctgtctcactttcttttcgcatatgttatatattttaaaacttttgtttttggggccaccacttactggctctgtgattttaggaaagcttttcagaccctcagaaccccagtctcttcatctctaaagtaataaaatatatgtaaagaacctatttcagcatgtgaattctgttttcctttttcctctagatgtatcatatccagttaaaaaaaaatctacttgaactttgcaggtatcttcagtgtttattacttctcatttttaacatttattggaaattagcatattgtcagaattgatataatacaatatgtaacctctttcattaagcttatcatttttaaaatacaaataactattctttacttaaatttttatttctagacacagggtcctgctctctcgaacccaggctggagtgcagtggcacaatcatagctcactgcagcctcaaactcttgggcttaagtgatcctcctgcctcacactctggagtagctgggattatagacctgagccatggtgtccagcatgacttaaattttgaataagttgcacttactaattacacttacactgctgcatctgaaagagtaatacaacaatagaagccttttatagtaattatttgtggcaggtagactcctaaaatgactcccaaatgacccacacccttgtacaatcaataccctgtcccttgagtgacagtgacttgctctaatcagttggctttcagttaatcaaaagagagattatcttgggctgaactaaactaatcccagcaagataacagggacttcagtcttgcaaacagatatcatataaagggaatcatatctgcacatttgattcttggaccctgggtaggaatttgttagagagcaaggatgtgaataaaggtgggaaataaaggtgtatctggtgtctctgctcaaaagctcacatgtagctacaaggcagagatttaggggagtggtatccacagtcaagaagaattaatacatagtgtttgagtctggcttcttccaattagcataatgcttttgaggttcatccatattgttgcattaccagtagtttttatttatttattttatcagtagtttctaaattgttgagtagtatattacattgtgtagatatatcctgtgtgtttatccatacacatttatggatatttgggagttgctagtttttagctattctatgagcattcacatacatgtctttgtgtggaaataggttttcatatctcttgggtaaatatacgggagtggaattgctagatcatatggtaaatactctgtatacttaaaatgaagagaaactgtcaaactattttccataatggctctaccatacttgatgttttagaaaagaggaaaaacactagcaattttgatgctgtatgtctacaacctaagagaatctaataatagcgtcctggtctgtcagtgtaggaattgcctaggtaatacatggaaggtgtggagggagcaggtacacagaaaatgtgcgtcagaaagttttgcttttcctatcccacgtcatttctgagtctgatcctccctactcctcacaaataaattggtagattcatctgtttttggacccttttctgctctagttactgaactatcagagacttcaggaagagcttaagctaagctattccagggaagttttggtgttactctgtacttacggactcctgcgtgtctgtctctgtcaccccagctgccaactcaacttagagtttattagcctggcattgaccagcacactgtgatattctctgaggcacactgcatgctgctagtgtgccctggttcataaagctgggccttcagcattgatcccactccaagttccttgctctgtcttcccacagctgtccccttgggggatgtggggggtctgacatcatttgcattattagtctattccaagaaatgcacctgtgcactaatcagttcactttttctgtcttagtcggcagatctagccatagcccaatccctcaatttctatactaagccttttatattagtaaaactgttatgttatctagaaattcatcagtgggaaaactacttcaagggaggtagttagaggctgcaaggtgctaaatatttttctttctttctttttaaatgtaacttggatctaaagtaaggacgaatttccttttattatttgttcagaatataccagctggtcctgtgggcagaagtttgttaaggggcctggatgtgaaataacagtgtatctcgtgtctctgctcaagagctcacatgtagctacaaggcagagatttaggggagtggcatatacggccaactgagaaataacaatattgtcagggactccaaggcactttggaagatataaagaatttcttaaattttcttgtagaattgttgcaaaaggcagacatgcttaaatgtttaaataagcttcttgaaaactcatggaacattcaatcacaactccaacagttctccaaaatttgatttattctggccatattatgcagcaaagaaacacttggctatttatctaaatctcttctgtcttgtaaatgatcctagaaaataatctagaaatacattttattaaagtaatgcatgaggtacatatcgattaccagccaatagttgaacttaaacataatgcatgggtgtgagggctggcattgatgattcacaatgaacacagttgaatatcacttccctgctgagactcgggtatcatcgcaaaaccacagtgagcatcagtggggatctgcatgctctggtcaagacagatctccatgctgagtgtctgtttgccatgatgaagagaacagtcaacaggagtgtttgccatgatgtagagaacagtccgggaggagtgaatggaggatgggggcatgaggaaagatgaggctttctctagttatgaccctttgtgtagaagttaggagtaggggataaatgtttctgcctaatatttaacttggaagagaagatgtccatgtgaaaaactgctaaatactgaaacaagagagcatgtgactaagcaataccatgtgtggcacagaaaacaagtgcaatagcagtttagcaaagtgagttcagtgtaggctgaactggttagagcaggtttccgggtgaaggtgaggattggtgagggtttggattggtagaaagagacggggaggacatctcacatcaagaagttatgccagtggatttcaggagaattgagtgtttgtgtctgaaaagcctgtaagaatagggcatgacagtgtggagaaatgcatgaaagtatgaaggatcgtgatatccagtttgaggagtgctaactagaggctctgaagatttgagaagaaacataatgaaaggtgttctcagcctgatcttggtatgcaggactggtaaaccccaaacaccttaccattctattacctctccctcaaggaatgggtcccttgataagaatttagtgtaaaaaagattgtcaattcagtgctgctgctgagtgctagtttctttaaacacacacagttttcttttgagaattttttttctattagatagacgaactgttatttaaatgaaaaaggcacatagtcccataaaacaattacacattcgggtgataacttcaaaaggagaaattaaaatgttcttatgttttgagcaagcatttccacttccagactttgctgcataaacatctgtggtcatctagggaatgcctgacctggttcagaggtgtcagagcaatgtaaagtcacggaagtgctgcagttctattctgggctctcatctttttgcagcggtccattctctaattttcaaccacatattaccagacaatctcttaagtcatacacaacaaactgattctgtttcaatgcttagaattagaataaaaaagcctaagcaaaaatagcacaaacatttgaaaaacactccttttttctaccactcccttcttagaacagaaataaaagccctgtactttaagaaaatggatggaagaattttctttgtacttcttattctccaagttacatttactacctgatagtgttaatacctttttgtagtacctttctttaaaatatacagggaaatgatctgttccaagaaactgtgtttttaaatttaattatagtgtgctatgatgatttaaaaaatggccttttgagtaaaggacacgaacaagttatttaaaaagtgagcagtaagtgtggtcagtaaacacatcaaaaagttcccttcactaatattcaaagaaatgcaaattagaataataatacccttgttgcttgtcaaattaatactttaacaattattatgtaaattttatggataggattgcaaatcaacacacctttcctggccactggtgtacataatagaaattctaaaaatgttcttaggcttgaccaagcatttccacttctagactttgctgcacaaagatctgtggtcatctaggagaaggcttgacctggttcagaggtatcagagcaatgcaaagtcatgaaaatgctgcaattctatcctggactgtcatcttttttgtagtggtccattctctaatttttcaatgacgtagtaccagacagtctcttaagtcaaatgtatttgtagggctctggatttgaaattcaatctggctttcagccttgatgtggccactcccaacttgtaaaggacctgtgaagacttagacacagaataaaacacagaagataagaaaaccaactttgaaagaaaatttattgtcaaaattgtactgggtcaattgagcaaaaacttcaaagagcgttttcatattaaccaaagcatccctcctcaattgaaacatataaagatattgttcacccctatcctgactcagagatccacaaatctgggcattcatcatcaccatattctgctggcaatagttttaagcagctaaattactgttctctactcttgagtttcctccatctcttcagatacagaaagttctagaatatattcaaataatcaaatctttaattcattccatatatctggctcaagacagtgtcccaataaccttattctagagaaagtcagatttagacttgtgctcatcatcatactcatgttcttgacatcattttaagctctgaggagacatctcacataactctcattgatacgtgtatacatctgtcactctgtgtttgtgctgtgatggatggacttggatgtagatatgcatagatgatgaagcagatagctatatatatggtattgatgtagattcagtacagctgtataattgcatctgtgtttcacatatcttgtgtactgtatcttgactgtatctgtgtcaatacctaattgagttataaggcataaatatgtggagctcttagcacaatacctggcacactgcaatcagtcgatgtatgtgtttgtgtgtgtgcgtgtgtgtgtgtgtgatacagacagaacctgctaatatgcataatagtcaggttgggttgttataacaaaatactatagactgtgtggcttaaacggcagacatttgtttctcacagttctggagtctgggaaatccaaggtcaagattagtttctggtgagagcagtcttcctcttttgcagctccccatggtttgcaaatcacagatttctcgctgtattctcacatagtgtaggaggaacagagggagggaagggaggaagtgcctaaggacactaatcccatgatgagagttctatccaccatgacctaattacctcctaatgccaccatctccaaatactatcacactgggaattaggtctttaacatgtgaatcatttggggtggagaagacaaacattcagctcataacaggtaaggtagaaaatctcaagaatttatattttgtatatgaaagatcattaatgagcagaattttaagtttaatgccaattttaatctatttttatcttttctacgattcttttttactttttaatttttaaactttcatatacaaagggataatatttggctaaatgttctcttctttttgagctctggatcattgttacgatcaccccataattgtatgtgtgcagagatagtgctttatacatttttaaaacaattttatatacagtattaaattttatttttcaataaacaatccatgagatagaaagaacatatattttgttttacagataagctccagggagagtctagggtaccttagattgtatgggtaaaacatggtttatttaaatcataaataaacagtcttatttccctaaatgcacatacaggcatccacatcaaatgaaatcacatcacatgaattgaacaggcagttactgacttagaactttgcactcacaaaagacacactctcctaaatgtctccatcatactatactttttgtccccaaatgcctaatcactgaagcttcagactttgttgctttgattcccttaggaaaaattcctggtgtttcagaaaaaaagagccatttaactacattagaagttaaccctcctttaaaaatgtg

SEQ ID NO: 20 - DNA, 26803 bp, Homo sapiens
  source 1..26803: mol_type = other DNA; note = cDNA
cagttaaggtggggcaggaataaatcacaatggtggaatgtcatcagttaaggcaggaactggccattttcacttcttttgtgattcttcacttgcttcaggccatctggatgtatatgtgcaggtcacagggcatacattggcttagcttgggctcagaggcctgacaaggcacagaaggagctgggatagaggtgcaaagtactcatgctgacctgacatcccatgcccacggcagacatgactacttgatcggacaatactttcttggaatcaaagacacaacttcacagtcctcagcatcatagttcaggcagcctgtatcagtagatcagagtgggcacatggcctggtgctatcactccatcctgtgaggggagatgggattggaatggaggtggaggagtcacaggtggtcctgatatcacaggcctgggagtctgaaagaagtgcagtaggtagtattatggccctcaaagacatctgtgtcctactgtctagagtcaattaaagtgttacctaacatagaaaaaaggattttgcaggtgggattaaactaaggatctcaagatagaaagactctcctggagtatccagttgtgtccaatgtaagaacaagcgttctcaaaatagggacacaggagattgaattgcaaaggagatgtgctgataaacggagaggttggagtgatgccgccatgaggcaaggaatgtgggtggcctctagcagctagcaatgattcggaaatggattctcccccagagtccagaaagatcacagcagtgctgacaccttgatttgagcccagtgtcacctctgtcagacttctgccctatagaactgtaagaggataaatttgtgttttaaggcacaaagtttatggtaatttgttatagcagcaatgggaaactaatacaagaagggttgtgttatcaacagaaatacagcaatcagaagaaagatggttggggagagcagagggtggggtgggaaggtgatggattctgtttgggacattctgggtgtcatatgaatattattggggaactgtccatcaaaagctcagacttcaaagaaagaaaggaaggagcaacaatattttgaaagggagaaagtaggtaatattcaatatgtgttgagtacctacaaggtttttagttctttcaccaagttattttatttaatttttatggcatcaaggggtgttttttttttttcttaaaccaaattttctcagttaagcatcccatgtaagtggccaggggttaagagatgtgactaagactagacagagtgattttcatcattacatggtactatggttctactgaagcactgaggaaggaagtcaagagagttcctgagaaatgtctggagtcttagtaacatgcaaagtgaggcagaggtattgtcagtgttagctggtcactttgaaaggttagaggtaaataaagttcagggcaatggtacttggagaggagtgaatctttggtaacctggaggagatgcttctcagaggaaaatttcttcacagcatgaagtcaccagctctaagactagtgggtgttctgggggatgttagcagtgactcactgactgttggcccagtgaggggagggatgaaaggctgggtagtgggtagagaatctgactcctcctgcccccacatgtctattcctgggccttctcttggtgggggcaaaaggtatgcaaagggatttccgcagtgtctgagtgctcccaggagacggaaaagtaactgatgacttggagttggagtcttgttggctcaggcagctggggcgttttgggatttcaacacctccctttagtttctatgtatggtgctgagcaagtagtcaaagactttggtcttgctttaccccagtagcccacaagcctgagtacatggctaacatttattgggcacttagtgtgtgccaaagagctttatgagacaggttctttttgagtcccattatactgataatgggactgataagaaacagaaggtcagaaaggcccagtaacttgggcaaggtcatgtagctagtaagtggtccagctgaaatttaaaaccaggtttatccaactccaaatcccacatctgatttattttgccatgaattaaaacatagttctgattttgagaaactcccagtcccagtgggaaaacagatggaatcacaaattaccataacacagtgtagtgatgtagatataaaaacaaagacatgggaaccctgaaaggaagttgtaacaccaatagaggaatgcatggaggagatctttcagaggaggtagcctcgtagctgggctcaggggtggatgagtttctggctagaagcgcattgtgggtagagggagaacatatatagaaaggcatgaagatggtggacatgtttggaaagagtgagtactagactgatgaacacgatgagtcaggagcttatgacgtcaggggctactgctagcctagctaatgactgaagattctgtgaaaagaaaaccctaacatttccagctgagtactagggccatggtgattatatatcatctctcatgacaacacaaatgtttgggcttacttatcatcctgctcagtaatcattatgtcactcattaaagatgtatcagggtaaattttaggtgagaattgattagaaatgtaaactctatatatgtagttacaggcttcacagtttgccactcaaacaatatggtcatccaatcatgaggcacaaccttctgctcagaacttttttataattgggagaaagtgacatccgagttccatctcttctgagaactgtgcagctcagaaatcttcttcccttttttgatcccccagttgggtgagatgacattaacagtgtgttataagcacttttcatgtttactaccccatattcaaaattaagcatttcaaacaactgtataattgtctataaatgtagcattattagttaattttccttttaacacattgaggttacctctaatgttttgctatttttaactatgttgaaataatcatgtggtttttattctttgaattattttatattgtaatgttccctgaaatgaaattagttgatcaaaagatgagtaatcttttcatgacttttgatacatgtcactcaatcaccttctggaagagttggaacaagagcctattgctggcatcccatttcatagccatctcaccaatgttggaaaatgggcttttcctattttctgctaattcattagttgttaatggcttattttaactattaatcccttgttattgatgaaagttaagcattttctgtatttttattttctgtctaaattctactttttgcacaatttcctttgctcacttcttcactggaatttagtatttttctttccaatttgtatgactccaaaatcagtgataaaataagcaaagggtctttcttagaggctaaacaagggaatgaataaggaacaagaacagcaaagggatagtgaataaagatacaaaataataacttatttttatttttgaagctttccccaggcagtaattcttgacctttttaagattataaattattttgagatatgcattcttatcctcaaaatgaattagtacataaaatttaaatgggttgatagatcccctgaagtgtatccacaaaatgctctcaggttaagcaacattgttctataataactgctttaaccaaaatgtttaattattgcttgtgtttacctgtcctagcaaagagctaattgttaaattttgtgttagagggtcaatattataaataacttaatgtggcctctctttcctttcctttttttgagagttttgaggctatcactaagtgggctttagagtaccaggtgggaatctgacctcactgctcaaactttcaacttcaggcatgaagtggtcccagtgatgtgggaacctcagagtctaaaacaaaaatagtagactgaggccctcagaaaccagacttatcagattcagaatttaatataactatgtttgaaagatttaaagaaaataaatcaaattaaaaagtgagcaagaagcaagaataacaaatgaactggcatatttgagaaagaattttgagcttttagaaataatacatacacttattgaaataaaaactcaagtcaataggtttaaacagcaaaatgaatatagttaaagaaaactattgtgctgaaagatttaaaaaactattacacagaatgcaaaatgatgaaatagggtaatttaatatatggcagatatgttaagacactatgaggatagtatgagaagacctgatagacatctaatcacagcaccttaaggtgaaaatagagagagtgcaccaatattgaggagttaatgtctgagactattccagaactgataaatagatgaatctacagatctcagaaacacagcttttacaaaggataaatgaagagaaattcaccagagatacattgtaatgaatctgtaaatcaccaaagctaaagatattatttcacaagtggaatgacagctgacttctcaacaacaacgaaagcaaggagacagttgaaagacatcttgaaaatggtgagagaaaaactaactgttaatataaaattgtgtacctatcaaaaatatcttttggccaggcgtggtggctcacgcctataatcctagcactttgggaagccaaggaacgtggatcacttgaggtcaggagtttgagaccgagaccgtcctggccaacatggtgaaaccccatctctactaaaaatacaaaaaaaaaaaaaaaatagccatgtgtggtggtgtgcacctgtaatcccagctacttgggaggctgaggcaggagaatcgcttgaacccaggaggtagaggttgcagtgagctgagatcatgctgctgcactccagtctgggtgacagagtgagactgcctcaaaaccaaaccaaaccaaaccaaaccaatctttcaagaagtacaccataaagacatttccggataaacagtgaatgagattacttcctaaacgaatatgtaaggatgtttctttttaaaattttttttgggtgtattcatcacctcaagcatttattcttttgtgttgttacaactgataagaatatctaaggatgtttcaaaaggaagaaaaatgatccctaaagtctggtatattagtttcttattagttgctctaagaaattactgcaaacttagtggctttaaaaaacacgaaattatctttcagttctgtaggtcagaaatttaacctcctgatttctggtcagaagtcttactgggctaaaatcagggtatcagcagggctgctggaggctctggagatgatgtttctctaatttctagaggcatttgcattccttagctagtggcttcttcccacatcttcaaagctaacagtggcagtcgagtccttctcttatcttttttttttggggggggttataaattttttttattattattatactttaagttttagggtacatgtgcacaacgtgcaggtttgttacatatgtatacatgtgccacattggtgtgctgcacccattaactcatcatttagcattaggtatatctcctaatgctatccgtcccccctcccccaccccacaacagtccccagtgtgtgatgttccccttcccttctcttatcttatcactctgacctcttctgcctcctttctctacttttaaggacccttgtgattagattgggtccacctaaataatccaggataatctctttattttaaggttagctgattagcaacctcaattccatctgtaaccttaatttcttttttgccatgtaacctaacatagtcacaggcccaagggaataataggatgtgggaattgcgggtggtggagtaggggcattattctgcttaccacaggggaccttggatgtcagtaaaaaatggtgaggaagaaaacaggaaacgtagaataatctaaacaaatctttgaaaaataagttgaaatgtggggttagaaaaagggcagaactaaaatgttaagacaaattaaaatgttaagcctataagttgagagggagatgatcaaggttaaaaatactcataaatcatatttttttgggtagggggttagatatttattaattttagaccctgagaagtaaaatagtcatgacaaaaatttaatagtaaccactaaaacaagagaaacagagtatataacttctaaatcaatatgggaaggggaatggaataagaaaacaatagcaaccaccaacaccccacccaaaataaataataaggtgagagagaaaaagaagcattgaaaaggggctaaaataaaataaaaaattaagtcatgcccgtaatcccagcactttgagaggccaagatgggtggatcacctgaggtcaggagttcgagaccagcctgaccaacatggggaaaccccatctctactaaaaattcaaaattagctgggtgtgggggtgcatgcctgtaatcccagctactctggaggctgaggcaggagaatcgcttgaacccaggaggcagaggttgtggtgagcagagattgtgccattgcattccagcctgggcagcaagagcgaaacgccatctaaaaaaaaaaaaaaaaaaattaagatagtaaaacaaatccaaatacattaaagattacaaaaaatgaaaatgaactaaacttcctagttgaaagacatattgtcagattggattttaaaatctggcaatgatatttccaggacacagtcctaaaatattaaacacagaaaagttgaaagtaaagggatggaagaacacagtcaaactaactagaagagagctgggttcacccttttatatcagagcagaatttcactgagaaaacactaacaggaagggggaaaggcacaacatagtgatgaagcacttaattctcaacaaatgtatgacacttctaaactattatgactgcaaaaaaaaaaaaaacctcagcatatataaaattttggaagagccatggctatcagctgacatgcacttaccatatttgcattgtcctactacaaactttacccatatgaccaaggtgctgtcttcatccccattttacagatggggaaatttccccatagagaaaaagatttgggccatgccttggtttcttcttgactctgcttcctgctgattgatcctcacacctcacctggagacctacttcacattatccatcaggatgagaactggtgtgcctttaccaaaggaccacagtcaccttcaaaacgtgtttagatagatctggatggagacttatgtggttgcagatgaaaaaaaagtccccatggtatatctgactccatgtcattcactgcatgaacactcacacacatgcacacgtacacaggcaaacacaatcctgttctggtccggcagtagtgccaacactcaaatcccaggtcctactctgaagcctttgaggaaaccattgtcattctggggtttatgggaatatttcttcttggtgccccttgttcctcacagaagctttttttctgtgctgtgggcttgctatcctagctttccaagctatcctagttttgcaaatttctctgatcaatcacatcaggttcttggatcatcccaaaattaaagaggtacgttttgctgcaagagacaaagacattcacctctcacacagtttgaattcctgggtagaaacagatttcaatgctggccagcctctgtagctcccagtgccccctcaattatgactcccgaggcattggagcaggatcctcaccttgcgcaaagataattaggtgtgctgaagaaagccagacactgctctttcttagctctgcatccttaaaaagttaccactactccttctctttgaatctttgacttgtcaatggtaaagtgcctatgattcttaaagggataaaaacgcaacctatataaagcacctggcatagtgcctggcacaggtgggtgtttaacaaatgttaactcctttctatcccactgtgggtgtccatatcctcttgggtaggctgcctctggtttccattgtgtcatcagagccatactaaatcgatgtgttggacatggatcccaagaactaatttctggaatgagttatgcctaccaacttccctgatacttacttctgtctctccatttatcaaatggaaatgtttgcatttggcctctgccagttggagtaaccaaaaggcttagaggttttggagaggtcctacactgccatctgtttgaccacatactgcctttcacgtgtaataagcattgtttcttacatttgtctaggaattagcagttcacaaagcacattcgcatataagggcttgttttgaattgatcttggcagcaattctatgagacaagtaaaaggtaggtcaagcattataatcctcattttaaatctgggaaaactgaatctcaaaaaggttgaaagacttgtctaggggacagtgtgtgggtaaatgagaagttaagatttgctgaactggcatttcctgactacatatctagtgtttatttatggagaaggcactacggtggccaagtggctcagagtcacacagctctgtgcctcagtttatttgtctgtaaaatgaagataataatatattctgaatactgttgtggaatttcattgagatagttcacataatggcatggatactgtagtacaccgcctagattcacaaccccacgactatccatgagaatggcccatagctcaagaggtcacattctttccccaggtgaagcccgcatccaatgactgctcagtgtgggtatataaaggcctggctcccttgccagtgccaccctacctcctggcgaatggactgaggcttgcattatgactgcattgcagctcaactgttctttccattcagtctttcctttacactcacacaggtgaggacccagccattaaatgcctcacatgatgacctctattgcatgctaggcttcgtgtgcacacacaaaaaccatcctctatcagtcaggccccttacttgcagaggcaaagaggccagtctgggttagatgtgcaccctgtttttacaacagaacgagacaaacaggtcctccttaggtgtaagttcatggccttggccccacccctgaaactcagccatctgagacagttttaggactgaggcaaccccagactttgggttgttgtttgctgggcacagcctccttttgccaatgttgcaatcctttgtaagagaccacaggttgatcacccgttcctgttactgagcacagagaggtttgtaggccagtctctccaggaattctgacgcactgcaaaatcccatggtctgaatgcttctaccttccctgtgaaagcccctggcctcagaaaaggaagctggtttaagtgaccaacatttggggtggagcttccgagggcccagccaccgtgttacatgcaacaatcaacagagatattctttggctagagttgtttttctgggtataggaccctataattaaaatcagctctccaacttcccttcctccaaaaagtatacaatgtaaggaggaaaatgcaaattgaaaagttgctagcccttcccgccagaatgccaccccaagcctcccctgcaggaagttctggagtccccaactctgccccaagcctgaggcccttgataaggtaaagggatgtgagtgtggaggccggagcccccctcgccctgtaggctgctccccttgcttttccctttgaatgttacagtttagttctgtgacttacttataattgcctgaattccccgcccacgctttctcttactttggggcttctcaccgttcctcttcctccatcttcctcctctgaccccactcccaagcctgaatggggtgttcctcttccgtgctcccacaaaaccctgtgcctctttcatcgttgcatatattatgctgtaatgtagttttctggaggtgtgaagactatgaatcctatgaagacaaatacacacctaccttgctgaccgttgtatcctcagaatctaatactgcacttggcacacagtggttgggagtaaatattcatgaggcaaacacacaggaaggactcttttgttatggttgtttgttgttgttgcaagtaacataaccccaccttgatctagctgactgacggctctaccaccttgtcttttgcagcacaagaggaataggaactgcacctcttccttcagtttcagcttgaataatatcaggaagattcgtatcggtctgagttgggtcacgtacccgacgtgctatagctgaggatggggtaagctgattggagtttgcaacactgttcacatagccaagatatggaaagaacctaaatgtcaactggtggatgaatggataaagaaattgtggtatatacatacactggaatattattcaaccttaaaaagaaggaaatcctaacatttgtgacaacatggatggacctggagggaattatgctgagtgaaataagacagacacaaaaagacatttcttgcaggagctcacttatatgtggaatctaaaatagtcaagcttaaagaagagagtagactactggttgtcaggagcaggagaaaagtggaaatgaagaggtgatagttaaagggtacaaagtttcagttatacaagataaataagttctggaggtttactattaatatatcacatagtacctataagtaacaatactgtattgtatacttaaaattgctaagagggtatatcttatatgttcttaccaataataataataatggtaataattaaggggcaggaggacacttcaaaaggtgatggatatgtttatggccttgatggtggtgatggtttcatgagagtatacttatccccaaactaattgagatgtaaatattaaatatgtacagctttttgtatgtcaatcgtacctcagtaaagtagtttaaaaatggttggactgagaaaaggaggagctgctcagcaacatgaggctgggtgctgggcagacaaaacctcacacatgcattactgaacccacggactcatgtctgtgagctttgtggctgtggatgcaccatgccaatgtagtcaaggattcttcaatgtgtaccttactggattagttccctctgtttatttgtattgactcctcagttcccttatgggttgcttgttggctcttgtggaattatttaagtaaggctttggttgcaggagtcagacatgcttaagctggttttggctgtaatggggaatttgtgagatgtgacacagtgacaggaagtgcagccagacgttgtgagaggcagtaactgggaataggaaagttatgagaagttaaggcagtagtaattgtttctctatcgaaggccataatgttatcattcctgcctctatctgtttgcttgttcttctctctcagcagaatggtcttctctgcttctctgtgcacctgcagaaggtgaccaccctaaggcttatggattcaggagtcctcagtttgagagcagttaccaaatgcctcgcccctgtgaactctagtcccagtttcttattcctggtactggcttagtttgaggcagctgcccaccgtaagtccagtgagccatggcctggggacaaggtcaggtacatgtgtaatcagctgggtctatgagttgtggtggtggcgatggaggtagctcccagaaaaatggattacaggttaagaagagtctatatatcttgaccacatttgatgtgacttggaagtttcaatgtgtgaccacttacaaagttatgatcaggtgtctcatttatttatggtgatgtatgagtccgtgtgtgagtgtgtgtgtgtctgtgtttgtgtgtgtgtgtgagagagagagagagaatgagaatatgagtggtggtttcccatttatttttctatgggccaaatcctgggctatgagctggagatataagagtaaacaaaatagacacggtgcttgctgtgacctgccctgggagacagatattacacggaggatcacataaatgcagagtctgagctgaggtcttaagggtgaggataagtaactagtcagtggagcaggggagggaagggtgttccatgcctgtgtcaaggtcctgaggtgagagaaagaggtgaatttgaggaaccaaaaggagttcagggaacaagaggggctgtttcacaagctgagtctggaagctgagcaggctgagcagattcttaggaactctgcaaaaacttaagggcttgtctatgaccgtaaacaactacggtcgtagataaacaagttggcaaacaactcaaggatttacagtcatcagaattgtaaaactgatcccatgtattttcaaacctcaaatcccttgctctgaccttgtgtgtctaagctagaaggaagcaggatgcacctctcccctggctggaatgaagggattcacccaagctctcagtcttctcacggcatccagggcccccctgcttgtgtgtggtctagatttccattcccatagtaggaattcctttgggagccttggggtctctcctgctagagggcttcacctgtgactgtctcaattcaagggaggggttctaataacattaaacctcaatatcttgctcttccattcctgatgcctccctttccttctacccttcccctacctccttctttcctatgccacaggctggcaggttagtgccagagcaagtggccagtcaccatcattggggttgtggatcctcagggttcttcagaagccccttcaccatgatcaagagtctccagtcactcagaattccacggttcccaaatgccagctctcccactactcccagcgttctccatctctgggatgttgggctccaggcttctcagatgcactgagtaccctaggctaagccactgatccatcagaacttcatagcctgagaggagagggagacaggcttgcaaaggagagttctgactagacactggggtgctacagatgcccgtaagttgtccctttaccctcgatgtccccagtgttgggaccacctgggaatgcccagccttgtgtgctgattgacttgtagtcccctggccctgactggaagcctagttttctctctcatgaccagctaggcccctagctccccagggaagaaatccaatctatttcctctgatagtaatggctaatacttaaataatgccaaccacatgccaagcactttacagttgttagcccagtcttcacaagcaccttgtgaggtgggcaagagtcttatcttactctcattttatagatgtggcccaggatcccacagctatagttcatggtgctgggatttgaacctctggccaccagagcccaccttaatgtgtcctcctcctgttgtcataacagaaaagtacaacaccatgatgacacatcaggctatcctggcaggttcccaggctgccccaatgcccaactttctaggtttacaaagttgacatttacgaagtttccaggtttacaaatctagtttctgattctttagtcagcaggaatttctctacaaaagctgcttcgaaaatttccagccaaaccttacacaccttggcattacatcttggtgagccaaggcggaagagaacaggaagtgaaggccccatgggaagtccctgcggtcgggagcacccaggcggggcggggggtggggggctttcctgtggccggctccctgcccctcccacccccattcaggccctgtgagttgaatgaagagaccctgggaatgagtccaggtctgcagggttagaggaaattgaaggcccttaccagatccctgttgagaagtttatgaattatgagcccttctgcaaatgagagggttcttccctgtcaggagggacagattgtaggtggcaagattggtggcagccagtaggctggtctgctccttcctctctatttcatatgtgtatgaaggcattacctgcagcaagggcctgtgtaaatgcatgtgatttacagagcattttatgtactgcgtgtcattcatgcttccggtgagccctaagtctaagatagggcagatagcatcaggtccattttgcagctgtcaaaatgaggtctgaagggcagaagtggtgtgcccacacacacacaactggttggctgcagacctggggactagacccgggacttcgtcctgcccaggggtctcttgccactgctccccatcaacttggatggctttaagcatttgtgagttgtctgctccctgatggcagaatgcagagacatgaagctacaagcaggttcgctcccaacggcaaaaaggaggaggggtgttcagaacatcaggtgcttctagagaaagcagggagagagtatctggccttgtggacaatgtcacggcagaggccaggtatagggcatgggggtaactggaagcgggatggaccctgttattccctaagacatggcttccacgtagtgctcaaacaaggcctttgcccttgctgttccctccacctggaatattcttccccttccttgacattgctcaggtctccactcttatgtcaccctctcagagagggcttccctggccactttccctaaaatagccacccactcctaggtccctcaaaagcatatcctgctttggattttccctatagcaatatgccctatgaagttattttatttgctaacttgtttcttgtctgttttcctttgttagagcgttggggaccttgtctggcttgttcccaatgcctggaagagtgcctggcacacaggattaagccaacacatatgttttgaatgaatgtgtgcacacatgcatgagctggcggcagtcggggttggggtaagcacgaaggcccagctcagttctctgcatgtgacctcccatcttacgcagataagaaccagtttggtttctgctagcctgagtcaccctcctggaaactgggcctgcttggcatcaagtcagccatcagccggcccatctcctcatgctggccaaccctctgtgagtgtgtgggaggggaggctgggctcctccttgtactctctgaggtgctctggaaggaggggcagctccaccctgggagggactgtggcccaggtactgcccgggtgctactttatgggcagcagctcagttgagttagagtctggaagacctcagaagacctcctgtcctatgaggccctccccatggctttaggtaagctccttccactctcattttttcacctgagaaatgagagaggaaaatgtctacaattggtgtttatcaaatgctttcaggctctggtgagcaagcgtccaggaaaatgtcaagcgcatggagctccaggcctgtctgggggatctgggcacggggaggcatccatgggagaccatgcaggcactctgaggcaggggctgcaagcctagtgcctgctggggcagcaggtgaacagagaggtgtaactgctgtgacagaagtcatggagtccttggagtgtgagggtcattttccactgttgatagaatagggaaattggtgaaatagccctgttaaatgagagaaagaacagtgtgagctcaatgagaaatactaatagaatgtggcactgagccacaaggtctgaggcttgattgataaggaagggtggggactgtggagaattaagggcttggcacaggtcagttccaccagttgtcacaagagaatgcaggctcaggtggccagaacttctcgcttttccagaagagtccgatattctgatttcattatatatagtattctgattaaaccagacaataaagcaagcagataaaatatttaaagtataagctgccagtttgcaacctccggttaggatttgtgtggggcaaagaaaaaaactctcaggatcattggtatgtagactctaattttaagtttctaatttaaaattggcccctgaggctgggcgtggtggctcacacctgtaatcccagcattttgggaggccaaggtgggtggatctcttgaggtcaagagttcaaggcctgcctggccaacatggtgaaaccctgtctctattaaaaatacaaaaattagctgggcatggtggtgcatgtctgcaatcttagctacttgggtagctaaggcaggagaattgctggaacccgggaggtagaggttgcagtgaatggagatcacaccactgcactccagtctgggcaatagagagagacgctctctctaaaaaaaaatatgtaaagataaataaaatgaaataaaataggcctctaatgagcaggccattctcctttctgggtcttactttccttgcactcctttctgggtgttaagaggaggtctagaggaagctggacaactcttagcttgtagtaagcacagtggaagtatcagctcttaatgggtcatggacacgttacaagctaggcgccgtgctgagcactttacatggtttatcccactgaaccctctcaataaccctatgaggaagggctattattgctcacattttcagaagaggaaatggatatagagagattagataatttgcccatggccagacagctagtataagaggaggaggtggattgactgcagacattctgtcttcaaaccactacactatgctatggaggcacagagacttaatgaaatcatggagaggggaattgctttgtcaaccacaagcagttattccgggggcagcagatcctcccctgtcccccagtgggtacaatggtccctggtgggttgtgctacaatgttagcccatggtcttatgtgtttttcaaatgtgtaaagtaggatgctggaaccactcttagaaccagataccaatacattgtgaagaaataaatctctgtgcttaaaactggttcatcccaaaatattttgaactgacacacaataggtgctaaataaatgtgtgttaacttgaattggattgaattcgggaaaaaagtgcaataagcttagtgaagacaccatgttccctgggtagaggaaccacattctccatctaaggccaggagtatgggaggtatcaatgtttgcccagcacagaacagggtgccaagaagagaaaagttgacggggtgcatactctgactggaaactggaagggtgagaacagagggtaaaggatagagatggaaccatgtgcatacactttgtgttaccttggacaagtcattcatttctctggacctctgctttctctctacacaatggggtcccaccacttcccttacagctgacttgtatgaagaaggaggtggaggaggaggagaaggtgaagacaatgctgactcaaagggtaaattatttttaggatccaagtttgaaaacaattttaggctactagatatgaacaacatcttgattatgtagttgaaggaaattaaagatgaatggtttaattaaaaattaatcagaatgaaaacgattgattactaatatatctgcaatggtttattttcctgagtggcagactcactaaggtttttgaatactcctgtgtgattgctctatgtatgtatgtatgtatgtatgtatgcatgtatctatctatctgttgtctaataaaatggatcacatctctgctaataaaaacactacactggcagggtacaattataatcattaactgtgcctggaatttgcagcagcagccaccagaggtaccagtgccctttaagggttcataatttagaataatccaattatctgagtttttcagggactgaggggtttggcaaggtgtagaactttcagtaataaagtcaagaaagtcctggacaaaccaaggtagttggtcactctagtccataaccaggtaaagagctttccctgtaacctgtgtaaggttttagaatcatttctttccttattaccaaaaatcctccccaaattttcaagaaattatgaactaaatagttactctatgagataggagttcagcccaaaagaaacaccataagaacaaatataattcttgcttatgttaaccatgcaatgaagcagagagaaaaagtcagtggcctctttaggaggactgtagtgtgggaagaaataactaaactgggtttcaatcctggcctggccaggatctggagcaagtgagttaatctttctaagccttgagtagtttcttcttcttcttcttcttcttcctcccccttctcctcttcttcttcctcctccttctcctcttcttcttcttcttcttcttcctcttcttcttcttcctcctcctcctcctcctcttcttcttcttcctcttcctcttcctcttcctcttcttcctcttcctcttattcttcttcgtcgtcttcgtcttctttttattttcaaagtgaaagcaagtttattaagaaagtaaaggaataaaagaatggccactccatagacagagtagcctgaaccttgagttcttctataaagtcactatgaatttatactcattttgaaagtgggtgtcaatatgtctgtccactttgcacagctgttatgtggacaaaaggagatctgtgtgaaagtgtaacacagagcctaaactataacaggtaagcaacacagttgtccccttccccatggtgtctgttcttctccatttcctcctgtctgcagggggattataaaactaatcatcaaagccaagaaggcaagagcaagcatgtaccgctgaaaacacaagataactgcataagtaatgactttcagtgcagattcatagctaacccataaactgctggggcaaaaatcatcttggaaggctctgaacctcagaaaggattcacagtaagttaaccatgtagatctgagaggagagtagcttcttgtagataacagttggattatataccatgtcctgatccccttcatcatccaggagagcagaggtggtcaccctgatagcagcaagcctgggggctgcagcttggtgggtagaggtactcaggggtacagatgtctccaaacctgtcctgctgccttagggagcttctaataagttgatggatttggttaaaattaacttggctacttggcaggactgggtcagtgaggaccaacaaaaagaagacatcagattataccctgggggtttgtatttcttgtgtttctttctcttctttgtactaaaatatttacccatgactgggaaagagcaactggagtctttgtagcattatcttagcaaaaatttacaaagtttggaaaacaatattgcccatattgtgtggtgtgtcctgtgacactcaggattcaagtgttggccgaagccactaaatgtgagatgaagccattacaaggcagtgtgcacatctgtccacccaagctggatgccaacatttcacaaatagtgcttgcgtgacacaaatgcagttccaggaggcccaaatgaaaatgtttgtactgaaatttgttaaagcttcccgacaaactagatttatcagtaaggattgttttctgcaagggggatgaaacttgtggggtgagccatttgggctgaggaggagggaggttggagctgagaaatgtggagacaatttccctttagaaggactgaatctccctgcctctctggggtgcggcagccagcaggatccaatggtgtatatgtctccccagctccccattcagtgatatcatgtcagtagcttgaaattatccgtggtgggagtattatgtcatggaaattggcaaatggaaacttttattggagattcaattgttaaacttttaccagcacaacactgccctgccttcagagtcaatgaccctatccaagtttaatccatctgtccactgtctccaacacgatctttataaaacacacctgacaacattacccttttattcagttttttaaaagataagtttccagctcatcggggtggctttaaaggccatttctcctctggacctcacccaacttttcaaatcacttttcctacccctacctctaaatgctactcaaactccagccatcctgaataataagacttttgaaaagtagattatgggctgggcacagtggctcacacctgtaatcccagcactttgggaggccaagatgggtggatcacctgaggtcgggagttcgagaccagcctgactaacatagtgaaaccctgtctctactaaaaatacaaaattagttgggggtggtggcacaagcctgtaatcccagctactcaggaggttgaggcaggggaattgcttgaacctgggaggcggaggttgcggtgagcctagattgctccactgcactccagcctgggcaacaagagcgaaactccatctcaaaaaaataaataaataaataaagtagattacatcagatacctctggcctaggttgtttatgaccaactctcctgctgagaataactagaaaagctagacaaaacatatttccaaaagatctctttggaggcatcagagaatggccaaggctgtaaggaactgcctgagcccagagaggtggagcccagcactggtgccctttactcctggggacatgtgctggtttcaaaaacttcagctgagcttttgagcattcatggaacttggtgggggagatgaaatttgtaccttaaatcctgcctacagggagggtccctgataatccccacccaatttggaaatctgggtcagccttcacaggtactgaagccctcctctgaatgatctcaagtcctgctagggtagaggttacctgcttttgaaaggctcctggcctacctgtgcagcaggagcaaaagtgaaccatctcagggtacagataacaatcatccagagccttgaatgacctctactgtgcttaatatatagtattcagcagtcagtaaaaaggatttaggcacatgcaagatgacctgtgtatcagggagaaataggcaataaattgagatccagcagggatttgaatcatggatttgaatcaggggcagccttcgaaagaactatggagaatatactcagatttaaaacataagattggaatttttggcagagaactaacaactgtacaaaaaaggaaccaaatggaaatcctagaactgaaagatgcaattaaccgatgttgagaaatagccaacatctattgaacacttcccatgtggacagctgtgctaaacactttacaggcatcaacataagatgtgtccccttacagcagtgcagtgtccctcctaagacatggacagcctggtttccctatctctctgcttcatcaaaacccctttacgtggggcttagacactcctgttgtctctagtgtctagtagcacagggctcagcacatggaagccactagatacaatttgatgaccaggacctccgatgaaagccatgggtgctgattgggaaggcattgtcttttatgtgctatggtcttaaagcttcatccaggaagcagaactcggggggtgctgaggacccagaaccgagaataagattagtcagagatttcctgtgggcagaaatcataaggacgccaactgtttgggtgagataagacgaaaccaagagtggacttgtggccagaagcgtgaggaagagggagagagcttcccttgtcccctttcttcctctccctaagccacagtgattgacagccccccccctttggagtcagagcaggcttgagactggactgggaaaggagggtgggtcaggatacagagcaggaaggctgggagtgcagggcaggagcaaggggctggggcattcattgtgcctgatctctcccactttacctggggtaaagaagcatatgcaaaagccacggtgtgagtatttcccaagtgccagggtcagggcatgattcatcacgtgcagcatttcattcaatccttatagtaaccgatgatgtggcttctattattagctctatcagataatgaaactgagaccaagacaggctctgcacattgtgtggggtaatgacacagggggattcagacctagactccataactcctgccccagggaccacccccaccctcaccctgtgcatgtcgacaaaggacagactgggccacttctcaggacacagcggggaaatgacacagagcagggaggttccaggagccccgagcgtcttttctccaggagaatactctctgaattcagactggggtcagagaaacatttacccaggagccgcagtgtgggtggggctttttacttgaaacgctgtctgaaggcagtggccaggatggaactctccaccctaccttggcaagccacttctcttctgcaatctgtaaggacattgttgagagaattatggtcttccaattccggagggttgaagaaagacaaataggagagaacctatcatagtcaggtgctagctgccttctctttcagagagtgtgagaataaagtgatacacttgattattagcaaatactttggaaattttaaacgctaatattcaacacactctggaagaggcaaataagtagacaggttcatatacatcatctccttcagctagtcctcacaaaaacaaacaaatgaataaacaaaattcttctttggccctcataggaagacactgtttcttgaacgtgtttcaaaaaggatgggtgactcactcaaggtcacactgtttatgaggacagtacaggaatacagacatgccattttgcctgaaaaaatccatcacccagggaggtgacacaattttgcagaaatgttctatttcctctgaaggatacattctttaaacctttgggaaattcattcatagtcttcctcctttgaaggattaactctctggacacaaagtgtttgattctgatttgttggttggaagatgtgttggttgagagaaagattctgatttgttggttgaaaatagactcatcaagatcaactgctgtagtagtaaatattttgacattttgtctgtattcctgtgctgccctcacaagctgcatcaccttgagtgagtcattcatacttttttgtttgtttttgttttggagatggagtcttactctgttgcctaggctggagtgcggtggcgtgatcttggctcactgcgacctccatctcctgggttcaagtgatcctcctgcctcagcctcccgagtagctgggattacaggcacatgccaccatccctgctaatttttgcattttcagtagagacggagtttcaccatgttggtcaggttggtcttgaactcctgacctcaggtgatccgcccacctcagcctccccaagtgctgggattacaggtgtgagccaccgtgcccagcccagccatcatttttgaaacacgtttgagaaacagtgtcttcctttgagggccaaggagacattttttttgtttatttgtttgtttttgtgaggactagctgaagggggtgatgtatattaacctgcctacttatttgcctcttcccagagtgtgatgaatattagggtttaaagtttctgaagcatttgttaataaagcccggggctggaggtcagaagacctggatttctctgcatacttttgccatcagcaagctgtgtgaccttggacagatcccttttttgtctaaatctttctgagtcttcttgaaaacaatgccaggttgggacaggatgattgccaagctcccgtccagctctaaaacactgcaacgtatgcttctgcaccagcactgtccatcctgtagatcatgcagaaattctcttcaactttttcctacccataaaataggagcatgcttacctttttcctaatgttccaggccccgggtctagaatattgtaagtaaggaagttaatgtgtatcagagcccattatgggccagaagttctcctcttccttcctacacctgcttcctccctccctccctccctctttcccttccttccttccatccatttgtgaagaagacatgatcaccctcattctgagagtgaagagacagaggctcaactaatgaaatgatttgttcaaggtcacacgggtggcacaaggcaagtggcagaggttgaatttagacccattcctgtccaaatgctgagtttatgtcatcgtcccgagaccataactttaaagatgtaagatagtgggaaaagagttgatttcaaagcacctctcagaaggactcactttacatcaggggtcagcagactcaggccaaatccggtccattccccgcttttgcaaagaaagttgtagtggaacacagctaggcttattgatttatggattgccaacgtccttttgtgaaacagacagctgagctgagtaatcgtggcgcacaaaacctaaaatatttactatctcgtcctttacagaatgtttgccaatctatggtccggagtccaaggctgtccatttttcaaagaacacaaagtgacatgagactgtcccatgtgcagggagccctatcattttattatgaaaaaacggcctttctgctcaaatctgttttttaaaaagtcaacaaacagactctgggtacctgtcaggaacagtagggagtttggtttccattgtgctcttcttcccaggaactcaatgaaggggaaatagaaatcttaattttggggaaattgcacaggggaaaaaggggagggaatcagttacaacactccattgcgacacttagtggggttgaaagtgacaacagcaagggtttctctttttggaaatgcgaggagggtatttccgcttctcgcagtggggcagggtggcagacgcctagcttgggtgagtgactatttctttataaaccacaactctgggcccgcaatggcagtccactgccttgctgcagtcacagaatggaaatctgcagaggcctccgcagtcacctaatcactctcctcctcttcctgttccattcagagacgatctgccgaccctctgggagaaaatccagcaagatgcaagccttcaggtaaggctaccccaaggaggagaaggtgagggtggatcagctggagactggaaacatatcacagctgccaggggctgccaggccccagagggcctgagaactgggtttgggctggagaggatgtccattattcaagaaagaggctgttacatgcatgggcttcaggacttgtgtttcaaaatatcccagatgtggatagtgcgaccggagggctgtcttactttcccagagactcaggaacccagtgagtaatagatgcatgccaaggagtgggactgcgattcaggcctagttgaatgtgctgacagagaagcagagaggggcaccaggggcacagcccgaaggcccagactgatatgggcaaggcctgtctgtgctgacatgtcggagggtcccactctccagggaccttggtttccccgtctgtgacatctgtgacatgagagtcacgataactccttgtgtgccttacagggttgttgtgaaaattaaatgcacagataatagcgtaacagtattccgtgcattgtaaagagcctgaaaaccattatgatttgaaaatggaatcggctttgtgagaccatcactattgtaaagatgtgatgctgatagaaatgacaggactgcttgtgcatgccctctgcagtgtgacattccagcagtgaaatcatgttggggtgacttctcccccactctgacctttatgtttgtctgggccgaggctgcaagtcgggctctgtgggtgtatgagtgacaagtctctcccttccagatatggggactgtctgcttccctaggttgcctctccctgctctgatcagctagaagctccaggagatcctcctggaggccccagcaggtgatgtttatccctccagactgaggctaaatctagaaactaggataatcacaaacaggccaatgctgccatatgcaaagcactttggtttgcctggccacccctcgtcgagcatgtgggctcttcagagccacctgatgaggtgggtacagttagccacacttcacaggtgaagaggtgaggcacaggtcccaggtcaggctggccggagctctgtttattacgtctcacagctttgagtcctgctctcaaccagagaggccctttaccaagaagaaaggattgggacccagaatcaggtcactggctgaggtagagaggaagccgggttgttcccaagggtagctgctcctgcaggactctgagcaggtcaccagctaatggaggaaaggctctagggaaagacccttctggtctcagactcagagcgagttagctgcaaggtgttccgtctcttgaaacttctacctaggtgctatggtagccactagtctcaggtggctatttaaatttatacttaaatgaatgaaaatagaagaaaatttaaaatccagacccttggtcacactatccacatttaaagaggtcaatagccacatgtggttagtggccaccctattgggcagtgcagctacagaacatttttgcatcccagaaagttcttttggatgttgctgctctacagcatgctttgctgaaacagaagtgccttccctgggaatctcagatgggaagcaagtaaggaggggagtcaaatgtgggctcactgctcaccagctgtgagggttgggcctgcctcttaaccattgtcagcctcagtcttctcatccatgcatgccgtgggtatactaaaatactatacccctggaagagctggatgcaaatttgacaagttctgggggacacaggaaggtgccaagcacaaggctgggcacatggtggctgtgcactacagctgagtccttttccttttcagaatctgggatgttaacc

SEQ ID NO: 21 - protein, 177 aa, Homo sapiens
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 22 - protein, 159 aa, Homo sapiens
MALETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 23 - protein, 180 aa, Homo sapiens
MALADLYEEGGGGGGEGEDNADSKETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 24 - protein, 143 aa, Homo sapiens
MQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 25 - DNA, 3804 bp, Homo sapiens
  source 1..3804: mol_type = other DNA; note = cDNA
taatttcagtgaaattatataacttggttacatttaggttacttattacaaatgaaaataaatatactaacagaagctgcctcatatgcaaatgagtaacatgttagagactaaaatacacaaaaaaatgcatgtgctttagctcatataaacaatatacaggacaacttaccgggtagcataaacaaccaacaacggactatcaaaaggaacctcgtcatctagtaactttaaccttgttggtagatctcctttttccatctccatatacacaggattggaacttgccatttctgaaatataaagcatatccttttccaaactttatctcttaggcggaaatattcaatttatgaacatgttcattcagcaatcattagtcacgtacctactgtgtggacctattatgtgcaaggcccaatattaagcattacaagagaaacaaataaaagtacaagagatgtaatttctacttttaagaaccttaaaatctaattgggaggcaaagcatattaaaagttttaaataagacgaaggaacatataattaatacatcagtgactattactcttaaaataatgagtatctaggcctatatatagcaactgtccatataagcacacatcaaaaaccaatatacatagctatcatcaatgccaagttgctcagttttttctagaactagtattcctactccatcttttagaattaaattcaatgaaattaattttaaaatatttgacctttttttaaagttaccagtatagcaaatactcattctctgaagaacagaagatttgactttttggaatagccaaaagtcacttaaaatcaagtctggtgaaaggagtgatgatctaaaactatttgtggtttaaaaaaaaaaagtatgaatctaaagtaaagtgggtttttttccaagtagttttcttttttagttttttagaaacagggtctcattctgttgcccaggctggagtgcagtggtgatcactgcaaccttgaactcctggctgtgtgtgtgtgtgtgtgtgtgtgcagcgacggggtcttcctacgttgcctagctgcagtgcctgtgtgcacacatgcgcacatgcgtgtgtggagagacagggtcttgctacgttgcctagctgcaatacctgggtgtgtgcatgtgtgtgtgtgtagagacaaggtcttgaaacgttgtctaggctggcatgtgtgtgtgtgtttatatgtatgtgtgtgtagacagggtcttgctacattgcataggctggtgtgtgtgtttttgtgtgtgtagagacggggtcttgctacgttgcctaagctgggcaagtatttttcaactgcctctgaagacaaatcccaaataacagttccaaaagctgtttcccataattatcacatcattagaagggtgaggcctactcaccaggttctaagagccaacattcattttctgatacatgcttttttaaaaaagtcattttttccccagtctcattgtttcccatttgactgtgtcaggcaataagtactttaaaggaattcagaggaggaggccattcagaggtttggggaagcctgatgactgcgcgggagctaaaccagacatatccacctaaattcaagtaagcagccatatcactcaaattgcccaccatgcttcctctagccagcactggtagtaacaactatcactctggctgatggagactcttttctgctcttctgtgactgggtatgatcacataatagagacagatacagtaaatttccaatgagtaataatgtcacacatttgaacttacctgaggagaaatagcttgtttcttatttcacacaaaagacaatctacctcaactcagaaaaaaaaaaattattatgcttttaactgctatatttgaattaaagcagatctgtaactatagatccatgtttctagaaagctaaaatatctttaagtaagatgacataaaaatgtatctctattcacttttggtaatgaatgaaaagttgcttaaagtctaaagtattagaaatatggcatctgttattcaagtaggatttggaattaagaaaattcacttcttcaaaaacatgggactatggctgcagaaagggcaatgcatatagtttttagggtatgatagctggtttctattatatgtcaggatgacatatgcgaccttccgccaaggtagatactgcgggctatgcaccaaagtctctgaggcagacatgtaagcgagctcttcacctatattcattcttttcctcctggacaggttacatttcccagtttcctttgcagttagttgtggctatatgacagaattctcatcaatggaaatgtacacagaagagaggtaagccactaccaggccaggcctataagacagcactttctacatgctttccccagacatagcaacccaaacatgaccacatcccttaagggaagatggagctgaaaataatggaaggaacttggaatgctagaatgctgaattaccacctgggagacagctacccactgacctggaatacctgtcctggactgttacatgagcaagaaatacacttctatttatgtatgagttacttcattatcagatatttattacagcagtttagctacctaagatctctctctgcctcagactgcttatctataaaatggaataacaccatctactccaaacattattgtaaggatgaaatgagacaatgctgaaaagtgtttaccataatatctgccacacaataagtaccccatatagtatttctgtattagtaagttacatgagagattttcttcttttaatacatctgcatttataaacattttactttaacctcaacttccccagcactgctctaccattttctgaatgtcattatgagagaaataaaactaatttctagggccaggcatggtggctcacacctataatcccagcattttgggaggccaaggtgggaagactgctttgaggtcaggatttcaagaccaacctgggcaacacagtaagaccccatctctataaaaaaatgaagaaatcagagggtacagtggcacatgcctgtaatcccacgactcagaaaactgaggcaggaggatcgcttgaacccaagggatcaaggctacagtgagccatgatcacaccactgcattccagcctgggcacagagtgagaccctgtctctaaaaataaaaaatagggccaggcacagtggttgatgcctgtgatcccaacactttgggaggccaaggcaggtagatcacttgaggtcaggcgtttgagaccagcttggccgacatggcaaaaccctgtctctactaaaatacaaaaattagctgggcgtggtggtgcacgcctgcagtcccagctacttgggaggctgaggcaagacaatcacttgaacccaggaggcggaggttgcagtgagccaagatggcgccactgcactcaaacagaatgaaactctgtctcaaaaaaaataaaataaataaaaatttaaaaactaatttcttataatccagttgtgaatttaaccaatgtctgaaagaactattaaaagttaaaatgaatggaaaacagaataaagggttgaccagaacagatgtgattttctacttaaatcttttttttaaaccccaaaattcaaaactgctaatgttttttaatacgaatttctatctttgataaggcaatctgagtattacctttcaatccttcaataaaagtatcccaaacagaagggctattactgtaactaagcttgatactctccttcgctcttttcaag

SEQ ID NO: 26 - DNA, 3804 bp, Homo sapiens
  source 1..3804: mol_type = other DNA; note = cDNA
  misc_difference 2122..2131: note = n is a, c, g, or t
ggcttctggctctgagtgaggtcctgctgcaaggtttcctagatgagccactgagactctaataagatccagtggaaataaccaggctctcgtcggaatataagtcccaagggaagctgtgccagtcttgtgggcgactgcctgacttctcctttcatttcagcaccatgaagcttctcacgggcctggttttctgctccttggtcctgggtgtcagcagccgaagcttcttttcgttccttggcgaggcttttgatggtaaggcttcagaaggtttgcaggatttctgaagagaaacatcaccctggacctgataaactggggaaaatgatgctttcggaaggctgcttttgaaccacagagttgctagtgtctgcgttgctgaggcctgccaggaactagggtttgctgggttgcctgtctcgagtctttcagagctgctgggaatatcccctttccccgtagtgcagcttctcaggatgtgttaagtggatggatcacatttcagaagccgctgcaaggtgtatcaaaaacacatctcctgagccgtaagggacggggcatccagtaacaacgcacacggggtatttttgggcttccttaagatttgagccgctgccttaggttgtgctgcccaatgtgcctggggagctgctaaacagattagagagtcgaggattgttgtcagttactcagagaaagaacaatcatcctttccaggagcacctgagctgtttgttttgcgtagaagatgcaaaataaggcctgcaatgggtataaaatgtccctcagcataaatcgcataggagtatgactaaggctgttgactcttctgtcttctttctccttcctccttcgatttcctagttggataatgtacagggctctttagcctcgctctgtcaggggctcccttcctggtttgttctgtttccattcttccttctccagccttcttgacaagagctgggaactaacgtgcctcaagcccccacaaggaccacagcattttctcatttagtttcagaatgactctgtgacgcaatcttcctctcttggaaggtgagaaagctgatcttggaaggtgagaaagctgagacttagagcagctgaagccaatgcccagggacttactgccagtcagcaggtggcagggcagaggtttgagcccggctgtgcttgaggtcagggctcttgccaggtagacgcatcactgaccacctcctagaggttgatggttatgaatctcaggcacaccttggcatcacctgaaatacccatgccttcaactccccagcagagtctgcagaaactggcctggggtgtggcctgggcactgggactttcagtttctctctgggtgattagaaagtgcagccaaggctcacgcctgtaattccagcactttgggaggccaaggtggatgaatcacttgaggtcatgagttccggagcagcctggccaacatggtgaaaccccgtctctactaaaaatactaaaatgtagccaggcgtggtggcaggcacctgtaatcccagctactcaggaggctgaagcacgagaatcacttgaacccgagaagcagaggttgcagtgactagagatcgcaccagtgtcctccaacctgggtgacagagcgagactccatctaaaaaaaatgaaaaagaaagtgcagccaaggcagagcaccactgccctattgcttcctcaagcaacccacagcatcagtacagcctactaagaaagtatttagggacttttatgctcctaacagtcactggaactcacgtcacaatgacgtgtattccatttgcaagaatatatactttaggtcggggtgcggtggctcacgcctgtaatcccagcactttgggaggccaaggcagggggatcacgaggtcaggagttcgagaccagcctgaccaacatggtgaaatccccgtctctactaaaaatacaaaaattagccaggcgtgatggcgcatgcctgtaatctcagctactcaggaggctgaggcagaagaatctcttgaacctgggaggtggaggttgcgatgagctgagatagcaccactgcactccagcctgggcgacagagcaagactctgtctaaaaaaaaaannnnnnnnnnaaaaaaaaaaaaaaaaaaaaaaaaaaaaaaaagaatataaactttagtagtcagggcagaagtactctgtgtctgccacctttctcagcatcagtattccatgtcactacctcattcatacacactcctggatcttatcataggcagcttcattctatagcagtggctcttcaccagggcacttgaagaagccaactaggataaaggaatgtgcttctcaacccatggtatccaaggctgctatgatcacaggctgaaagcttgaagtcagtggaagatttgtccttcctcattcccctctaaggtgttgttggagtctttatgttctcctgatgtcccttctgcctttcctttcctttccaggggctcgggacatgtggagagcctactctgacatgagagaagccaattacatcggctcagacaaatacttccatgctcgggggaactatgatgctgccaaaaggggacctgggggtgcctgggctgcagaagtgatcaggtaactggagctcctgggacgttagggctgggtgagcagagcttgcctgccttggacagtcaggagggagacgagctccttgtggagaagttagaggctgcggcccctcctcctcttgccctctctctgcctctgtgctcagtgtgaggtctgagtggatggtaggagtgagtgattcctcatcctccctctctgggtgctgttcatccagcctaggggtgcccagcctggctgaatggggtggtgcccagtgttttcatccctccttccttggcctttctgggctcctctctgagccctcccttggaacagggagaatgggagggtgggctattgctcactggcctgattattaatctccttcttgcctgccttgattacagcgatgccagagagaatatccagagattctttggccatggtgcggaggactcgctggctgatcaggctgccaatgaatggggcaggagtggcaaagaccccaatcacttccgacctgctggcctgcctgagaaatactgagcttcctcttcactctgctctcaggagatctggctgtgaggccctcagggcagggatacaaagcggggagagggtacacaatgggtatctaataaatacttaagaggtggaatttgtggaaactgggtgttatactttgtggtatagactgcctgtttagtatgaaggggcgatccatgcacatctaagtgaacgtggaggctgggtgggtgggagacgactcctgggcacacagggcatcctgggcatccctgaggcaaggacatgatgagttcagtggccacccccacaggatcccaggggcttcagcagatcccaccccttaccccatgtgagcagctgcccagtgagtctgtaggaacccgagccacattcccagtgagttcaactgcaccccggcacgttttgctagcacctcaatggagagctccttgcttgcagctttggcttgtggcacccagcaaaagcttcctgccacccagtggctacagccacacactctccagcaagatttaatctcagccttgtgaggagccctttcccaaatttatttctttctgtgttttttatcccttagtagctaatctcatgttagccattaataactctctatgttaaacccttccttttgtatctgcggctacattga

SEQ ID NO: 27 - DNA, 3804 bp, Homo sapiens
  source 1..3804: mol_type = other DNA; note = cDNA
gtataaatgcagaggtgcagtaactgggcttttcaggatatccagatggagttgtggtgttgttttgcttgtggtttttaacgttaacttttttttcccctttattttaagagaagcacaaaatgaacaaactagtgagcccagcaacatggatggaaattccggagatgcagactgctttcagccagcagtcaaaagggtatgggcaaaaaaatatgaaccatttggggctcaagtttctccaaatactttatgtgactgcaagtactgtatacgcttatttcctgtgactcagttcttctaactaagatgttaagcatttggcttaaagtgtatagcattacaaagagtatttccccagctttggcttgccagccaactttccattgactctagcctgttagccatttttattgttttttgtttgtttgtttgtttgttttcctcacatgtacacatacatacagattgttcttatatgtgattttgttctctgggaataaaatcttcattcaacagaggcaatatgacagaaaaaccgaagtttcatgtatatgatttttcaaagaaagtgaattggccctcatgttaaacctagcatttcagagctgaaagtgtcttctcattaaatattgaagaaatcatttgagggtgtggagaaggatggacagaattagctgcttgtgtatttattcttctcctgcaactttgccacgctattttgtacctcccttcctaattatgataaagctttcttagagagcagtcaggcaatgtgtattaaatgtttaaagctttacacccttagttctacttgtggacatttatttcttaagaaagatgtatactaagatttagatagaaatattcattacagtatcataataaagacagtagcaaggattctgttatctgtgttgcattaatacaatagagattgatgcaactgttcattattttgaaagcattaatgataatattgcatcaaagagttcactgaacagattgtacagtacagttttacacacaaaaaaaatagtttgtgagctttaaaagggccttcaaatgaatatgctaaacggttatctttcaataaagagagtatgggtaagtcttaatttctttcatttatttcagtaatttaatgtttttgttgttgttattgtttgtttttttgagacagagtcttgctctgtcacccaggctggagtgcagtggtgcaatctcagctcactgcaatctcggctcactgcaacgtctgcctcctggattcaagcaattcttctgcctcagcctccggagtagctgggattacaggcgtgcaccaccatacctagctaatttttgtatttttagtagagacggagttttgccatattggccaggctggtgttgaactcctggcctcatgtgatccgcccatctcagcctcccaaagtgctggggttacaggcatgagccactgtgccagcctatttcagtaacttaatgtttttacaggcatgtattacctataaaattaataaagccagtgaggtatttcttttttgaactaaagcaaagctaataataagttatagagaagttagagaagaaatctattaagtgatactttctttgtatactgttgggctgagtacccttgattcttggtggtgaacaagttatcagaaatttcttggccaggagccgtggctcacacctgtagtcccagcactttgggagtccaaggtgggtggatcacttgaggtcagaatttctagatcagcctggccagcatggtgaaaccctgtctctattaaaaatacagaaattagccaggcatggtgacgtgcgcctgtaattccagctacctgggaggctgaggcagcaaaatcacttgaacctggcaggcggagttcgcagtgagctgggatcgcgtcgctgcactccagcctggatgacagagcaagactccatctcaaagaaaaaataaaaaagaaatttatttacttgtgtgaatttttacaatacagatgcttctcgacttaaatggggctacatcccaataaactcataagttgcaaatactgtaaatcaaaaatgcattgaatacacctaatgtatggaacaccatagtttagcctatcctactttaaatgtgttccgaacacttagattagcctgtagttgggcagcattacctactataaagtgtattttctaataaaatgttgaatatctcatgtaactcattgaatactgaaagtgaaaaacaatgtatgggtactcaaaatatggtttctctactgaatgtgaatcactttgacaccatcataaagttgaaaaattccaagtcaaaccattgtaagtcaggggctatcagtattcagtggtaaatgctggctctaactattcttccaagtcagtggttgactgctgtttattctataaagggttacaatttatagattctctcacttgtagaatgagagattcagaattaatagcagacagagtccctaccttgatggagctttcatttaagtgtgaaagtcaggtgacctaacaaggccttggcataagtttaggatttggattgttatgggagcttgggtagggacatgtcataggtaaggcaacagcaggggtagagataagcttgacatatgtcaaaaatcatgaagacatcagtaatccttgaagttggctgaaaggtatagagttgagaaagtagttaaaaaaaaaaaagtcaggctgagtctaggtaaggatgtgtttctctgaggtcagatttgttcctgtaccataaagggactatttagaatcttaaagctggagcaatttaaaacgttaagttttcagattgaggtcagatttgtgacttcatgtgaggtcagatttgttcctgtaccataaagggactatttagaatcttaaagctggagcaatttaaaacgttaagttttcagattgacgttttttgaggtatagttaataacctgaatgttctgattctagtcttggtagtcaataagagttgaccagatgaatttcatagctttgtagaggatgaaatatttcaaggctgatttgcacaaatgtttacatagatcatgtatctttcataagtaatatgtttgtattattacaaggctgtaaaaatttaagcaggttgttaatagcacagggggtaacagattaataaaattaatgaataaaattactaaaagagtccagaagtaaacccaaatacgtggaggaattaagcatatgtatgatacacatgacattttaaaaatcagtgggaaaaggtaaattattttacaaatggtgttagaagcactgattgataattttgttaaaagaaacttagattccctattttactcctaatccaaaataaattctgagtggatctaagattaagcaaaaattaagccggaagctgagcatggtagcatgtgtctgtaatctccgcaatttaggagactgagtttggctggggaggtggtgatatgcgcctagaaaaaaaaaattttttaagccacagatgtataagcaaaaagcgggcaaagaggcggaatttttttttttttttttgatgaagtctcacttgtcgcccaggctggaacgcagtggcgtgatctcagctcactgcaacctctgcctcccgggttcaagcgattctcctgcctcagcctcccaagtagctgggattacaggcacctgccgaggagaggatttttttataattaaaacaaaacaaaacaaaaaaacaccaaactggaagataaagtatttacaacatgtaaaagactgttt

SEQ ID NO: 28 - protein, 122 aa, Homo sapiens
MKLLTGLVFCSLVLGVSSRSFFSFLGEAFDGARDMWRAYSDMREANYIGSDKYFHARGNYDAAKRGPGGAWAAEVISDARENIQRFFGHGAEDSLADQAANEWGRSGKDPNHFRPAGLPEKY

SEQ ID NO: 29 - protein, 122 aa, Homo sapiens; identical to SEQ ID NO: 28

SEQ ID NO: 30 - protein, 122 aa, Homo sapiens; identical to SEQ ID NO: 28

SEQ ID NO: 31 - DNA, 5687 bp, Homo sapiens
  source 1..5687: mol_type = other DNA; note = cDNA
ctctctggttgcccttaacattttttccttcatttcaactttggtgggtctgatgattatgtgtcttggggttgctcttctcgaggagtatcttagtagtattctctgtatttcctgaatttgaatgttggcctgtctttctaggttggggaagttctcctggataatatcctgaagagtatttttcaacttggttctattctccttgttactttcaggtacaccaatcaaacgtagatttggtcttgtcacatagtcccatatttcttggaggttttattcgttcctttttattcttttttctctagttttgtcttctcgctttatttcactaagttgatcttcaatctctgatatccttgcttctgcttgattgattcagctatcccccgctcgatattacaaaccatgtcacgaggcgtggacaccccccatgatatggggagtattatcacccccctcttcccccactggatattacaaaccatgtcatagggaggtggacatcccccacaatatgaggagtaatatcacacccctttccccgcagtggatattatgaaccatgtcacaggcggttaaacacccccaacgatatggggagtaatatcacactcctctcccccctggatattacgaaccatgtcatggggggtggacaccctttgcaatatggggagtaaaatcacccccctctcccccaactggatattatgaaccatgtcacagtgggggaaaaatcctctgtgatatgcagagtaatatcaccccactctcaccacctggatattacgaaccatgtcacaggggggtggacaccccccaagatgggggagtaatatcacctcactctctgccaccagatattacaaactgtgtcacaggggggtgaacaacccccacaatatggggagcactatcacccccctcccccagggtattatgatccatgtcacaggggggtggataccacccactatatggggagtaatatcacctttctctcccgccctggtttttatgaaccgtgtcacaggggggtggacaccccttgcgatatggggagtaatatcacccccctctccaccatctggatattacgaaccatgtcacaggggggtggacacccctgcgatatggggaggaatatgcccctctccccacctggatattacaaatcatgtcacgggggacggacatcccccacaatatggggagtaatatcaccacactctcccctgctggatattacgaaccatatcacaggcggctggagacacaaggcattaacaatatttcgagtaatattatctttccctttgaacattatgaacaatatgacagaggggtgtacacctcctgcgatattgggagtaatgtcatcccctcccccactggatattaggaaccatattactgggggatgtattcccccttctagattgggaggaagatcatacttgccctccctgaatatttgaaacaatatcataggggtttgtacacttttacgatattgggagtaatatcatcctttctccccctggaaattaggaacaatatcacaggggtggtgtacacccctgcaatatttagggtaatattattgtcttctcccctcgatattaggaacaatattacaaggacggtgtaaagtacctgccaaattgggaaaaatactatcctctccctcttgtatattagaaacaataacacagggggaatgtacacccactgccatattgggagtaatatcatactcgccccatcccccagatattaggaacaatatcacagcaggggtgtacacttttacgatattgggagtaatatcatactctctccccctggaaattaggaataatatcacagagatggtgtagaccctctgcaatacttaggataatattatcatctcccccctcgatattaggaacaatattacggggagtgtaaattacctgccaaattggaggtaatcctctcctctctctccctgtattttagaaaatataacacacaggaaatgtacaacactgcgatattcggagtaatatcttcttctccccacctggatattaggaacaataacacggacggggcgtacacccctcgcgatattgaatgtaatgtcatcctctccctccctttatattacgaacaatatcacaggggggtgtacaacccctgcaatattggaagtagtatcatccattctcccatgaatattaggaacaatatcacaggggtagagtacaccctctgcaatttcgggagtaacatcatcctctcgttccctggatattataaacaacaccacgggggggtgggggtgtacacacccttcgatattgggagtaatataatcctttccctccctatatattagaagcaatatcacaggggttggtgtaaacttcttgcgatatggggattaatatcacccccctctcctgccctggatattatgaaccatatcacagggaggtggacacactttgcgatatggggagtaatatcacgcccctctcccccccgatattacgaaccatatcacaagggagtggaccccccccacgatatggggagtaatatcacccccctctcccgccctggatattacgaaccatatcacagggggatggacaccccccgcgatgcggggagtaatgtcacccccttctgccccctaggatattacgaaccatatcatggacaccctccacgatattggaaataatatcatcctctcccccttggatattaggaacaatatcacagggggttgtacacctcctatgatattggaagtaatatcatcctctccctcctggatattagcaacaatatcacagggagtgtgtacaaccccagcgatatttggagtaatatcaccctctcaccccatggatatgagaaacaatatcacaggggaggtgtacatcccacgtgatattgtgtgtaatatcattcttccccaacccctgcaatattgtggtgtaatataattctctcccttcctggacattatgaacaatatcactactaggtgatacattaggagtaatgtatccataggatattatgaggaatatcacagggtgtacacccactgtgatattagaggtaatatctccctaaaatattaagaagaatatcttacacccactgtgactttagaagtaatatctccctaaaacgttacaaataacatcgcagggtgtacactcacagtgatattaggagtaatatctccctagaatattacaaatacacatggtgtaaacccactgtgactttagaagaactatctccctaaaataatacaaaaatatcgcagtgtataccataatatcccctagaatatcataaataatatcacagggtgtacacccactgtgataataggaataataccaccccaggatattatgaataatgtcacaggctgtacacccactatgacattaggagtaatatctccctaggacactatgaataacatcacagattttacacccatggtgtgcacccactatgatattaggagtaatatctgcacaggatataacaaataatagtacagggtgtacacatatgatatacacccactgtgatattaggagaaatatatccctaggatattatgaataacctctcagagtacacacacatggtatacaccctctgtggcattaggaacaataactttctaaaacattacgaataacatcacagaatgtacacacatggtttacacccactgtgacaggtgcaatatctcccttggatattatgaataacaacaaactatcactgtcatattaggagtaatttctccctagaatattacaaataacatcacagggtgttcatttatggtgcacacccactgtgatattaggagtaatatctccctaggatattacttttcatataaaagtgtgtacatccactgtgatattgggaaaaatatttctctaggatattatgaataatatcacagagcgtacacccactgtgatattaggagtaataattccctgggttattatgaataatatcacaggatgtacaaccactgtgatattaggagcaatatcttcctaggatattacaaataatatcacagggtgtacacccactgtgatattaaagtaatttttaggttattgtgaataatatcaccaagtgtacaaacatggtgtacactcactgtgatatcaggagtaatatctcagtaaaatattatgaataatatcacagggtatacacccactgtgatattagcagtagtatctttgtaggatattacaaataatatcacagggtgtatgcccactatgacattagaagcaatatctccctaggatatcaaaaataatatcacagggtgtacaacttctacatcccaggttctaagggattctcctgcttcagcctcctgagtggctgggattacagatgcccaccaccacacctggctaatttcgtattttcagtagagatggggtatcaccatgctggtcaggctggtctggaacttctgacctcaggtgatccaccagcctcggccttccaaagtgctgggaatacaggtgtgagccaacgtgcttggcagagagttatatattaaataaatctggaaacatagctcccatgtttgagtgtgcatttacttttatgaagaaattatgtcagaaaacctaaggatgataataaatatgaaaagtaactggcatgtaaaaaggtcttttgattaagaactataaggttcgatttcatttttagataacgtgatcctagctcttgtatagtgcttataaatattctacatcaaaggaatttgttgcacagtgtcagaataaaataaagtgtatttcactgcttcttaatttttaaattagactgagtttgttttcctagagagagaagaacattttttattttttctgaaaagagtaggccatattttactgagatcttagatttgttatatattaggttttggtcttctaacattctccagtggattttctctaaagtaggtatgcacagaaagagttgaatagcaaaaaagtaaatcatgtaataattctgagatttttgggtttgtcacaactgagaaatattgctgagggtgtatggtcctcaagtgtgaaaatgttccttgtgaattgcttgtatccgaaatatacacacaacattaagtcctggtttttatcttttattttttccaatccttttttcttctcaaggtgtccaagtcacacagagccacagaatctcacaggtgtctcagaattcctcctcctgggactctcagaggatccagaactgcagccactccttgctgggctgttcctatccatgtgcctggtcacgatgctggggaacctgctcatcatcctggccgtcagccctgactcccacctccacatccccatgtacttcttcctctccaacctgtccttgcctgacattggtttcaccttggccacggtccccaagatgattgtagacatgcaatcacatagcagagtcatctcccatgcaggctgtctgacacagatacctttctttgtcctttttgtatgtatagatgacatgctcctgactgtgatggcctatgactgatttgtggccatctgtcaccccctgcactacccagtcatcatgaatcctca

SEQ ID NO: 32 - DNA, 5687 bp, Homo sapiens
  source 1..5687: mol_type = other DNA; note = cDNA
attgaattttatctcagagcccacatgaagcaggatcaaagtcagtacacatgaaaactagagcccaaagactataaagcatgaaataaggatttaagctaaccctatcttgtaaggggtttgtaaagcccagcttgcatctgagctacactagcaccaggacagccactcagtaatggggtttctcaaggttattgcttttcattcagttgaaatgagagtcatttcttacccttatgccctgtgagatttcactggaggttgttcactgaaacattttcatatcattgcatcaaccctcttgaactcactgtgcctgcccccagttcagtctgtgactcacaagtaccctgcagcaaaagaaatccaatagagggcaaatccctcaccttaccttcctttctaagacctttgatgttctcatgtgtcatttcataattgggattgtcaattagtcgcctcatctctggtcctcactttcctctctcccagccaaactcaaccttcagcccacacaatggaattcaacaaaatgaggtaacagttttctgtgtgagtcactctgggcaactctgttcacagagcactgtgaggtgagcagccagaacccaggcaagtgtttcagccatccaagaactggcaggcagcccaagagacactctcacctgatgacagactagcaggatgagtcctggaggaaatggttcccaacagctgcagaaggagtctcttggctcatgcacagcaatgctcttctcaattaaaaacgttgtcattattgacactgcagtgtaaaatccttttacactgtgctcacatttctacaggccttcacctgctctgcccattaaagacaagacccttccatgagatgatgacatctctaagttactgttccacccaaacagtcctatataatgaagagaaaaattttgctggccctcaaaaggcaaacacaaggagaaagatttccacaagctgtttctctttgctgagcacttagaggaaaactgtaagtggttggaagaaggctttgttttctacaagacttttagttattcctcagaaattttcctgcttattcccagaggaggtcatctcttagatgctgtcagtcagatagggattggcagccaagcagaggtgctcagagaggtttccaactaatgtggccagcggaaaactgccaaagaagcagggatccttaggacaaataaactggaagatattttggggataaaaataaatccttttgaaaatgaaagatggagagatgctgtatatacaaattgccctgttctgaacaatgttgtcactaggactggccctggagaccaatgatacaaaccaaaatgttctcagacatgctttgatggtctttttctccaaagttatctattctgtttccatttcattctcacaggacttgccatggggttctcataagatttcacattggtcataatccaggtggccctggactgcaacctctgagttggcaacatcagaataggaattacgaaaaaccaatttaaagttaaatacagacacaggcaaaagagagatgggttgtcgaagctagtgcctaggtggacactgcctcacatttttaattccagaagccatcagtactgagtgtcagatctcattagtcaaacacagtgatcaggaatcctgttttcctggaggatttccttgagggagggaccactcaagagtctgaaatatttcacgtcatagagtatggatctcaccccaacacccaatcagaaaatagggagaactggaaaccaaaattccccctcccgctgtggaaggatgaaaaccagagtgttggagttctgtcctgataatggagcagacagctaggcagcaatcaatgagggccagtacaggaattcagtgctaagtattgggtcataacagaagtagggaaggtattaatccagtgctatatgaggatcctgggacactggctcctagtaatctagttataactattagcaaaaaagaaaaaaaaaatcagtgatgtgaagagatggcctaaaggagctccagcaatatagctaagcagctggcaagtggtctgaggagcattgcaattccaggcctcctaaggtggcagtacgggcactggtaagacattctgctgtggtgaaactagtttaccatagaggattcacaattaaaataggcaaacaggaaatgcaagacagaggctaacaaagggttttttttttggtgggggggagttgtttgtttgtttgtttgttttctgagacggagtcttcctctgttgcccaggctggaatgcagtggcacgatctcagctcactacaacctctgcctcccaggttcaagcagttcttctgcctcagcctcccaggttcaagcagttcttctgcctcagcctcccaagtacctgggactatagctgtgtgccaccacattgactaatttttgtacttttagtacagactgggtttcaccatgttggccaggctggtgtcgaactcctgacctcaagtaatccccccgccttggcctcccaaagtgctggaattacaggcatgagccaccacacctggccagtttttggtaattcttaaagaactcaatgagcaacactcaaacaaccataaagactatagagctcatggttgaattttagatagctaaacagacaggagtttttgtaagttttgtaagtcttgctcatccttccctcttccatcctctatctcaactattctgtctaccattaaagcaccttagaccttgagtttggcaatgcaacaagtgtgtgctcaacacgaaataggtaattcaatagcaaagccctaaaacagcctggcttgattatttctcagggcatgcagttcctttgaagcaggatcattttaataataataataatagaaataataatagaaattgaagacaattatttcacaatttccatacacctaagagctatacatatgaatgataatgcataattgtaaagcatgcatattacaggtaataaatatgttagctaattataaacaatgcccattttcatatagtttatccttgccaaataaaactgtaaaaaaaagacacctttcaaatgctgctaaggagtaatacctgaatgaggttgatttaatggagtcttagttcctgcatgtgttctaattgaatagactatgtagtaattcccttacatacccatccatgtccaagaacagtgaagatctttatttaatatgaattattgcagatgattagcacagtctagccaaaccattccagtaattgtttttacttgttatattaatatataaattctcaaaggatataacagtgatgttgggtgaatttcactgaatgatagctcaaacacctgaaatattgactaagaaaactaatttatcaatactgataatcaattttaatatgttaattgattgtaatacaggattctgtggttcaaaaaaaaagaacaagcaaaaaaactttcttccatttccaaataccaattaatagatctctacttccccttggatttcttcttaccacctaccacctccaatcttcattctttcctcacaaactaaacataaaagttacctacaaagcatagaatctgtgttaaaggatattcttgcttgttttaagtccaaaattaaacagctctgaattattaaaaagcacatgaattcaaatgtcctattctaataagaaaatggtttacatttctctatgttcaaggaaaaaaatagtcaagggtgtacaagtggggtaaaaattatttccagtaggttatgtgatttaagttatagaaacgaaccaggcaattcaattaaatgtcatggaaagtaggttttttcttttcctctttttttctaatatgtacactttgtgagaagataaatccatagtgtgataatttgtccactgggtccatcagacactggagacagcttcctaagaattataaggcttctaaaggcttctaaagcctaaattgcctagagcattttgtgtgccaggcactttgctaggtgccctagggatgcaagaagtataaatgttttatgagaatacaggctggaaatgtattcttgattattcctgtggaatttctaggcagaaaagagtctaatggggtataggtatattttctcaacacaattttctgagcctttaccagatgcagttctatggtttgaatgtgtccctcagagtttgtgtgttggaaacttaatccccaatgcaataatgttggtgaggcctaatgaaagcctaataatgtaggtgaggcctaatgagagctgtttagaccatgaaggctcttccctcatggatggattaatgctgttatggtgggaatgggttcattattactggggtgggttcataataaaaagatgagtttggcctgctattctctctctttctcatcctctcttccaccatgggatgacacagcaagaaggcccctgcaagatgccctcccctcagtattggacttcacagcctccaggaccataagccaataaatttttgttcattataaatttcccagtctgtggtattctgttatagcaacactcaatttatgcattacttccagattcttatggctatacctacttctcacagtttgtattcacccctccttcaaccaagtacccttaacacagttcccatagtcacaaagccaggtcactgaagctgccctctctccaaccacacacatatagatcaaatgaccccagacatagagctgattgagaaggagggaccagtacgagctctgcttccccagcagcttcctggaaagaagaggcaatacaacccaacccaaaagtgcaagagaagtaacacctcatgggatgagcttaattaatcaatgggagaggacactagaagacactagaggatctcccttcctccctttctttcccacttcaccccctccagtctctgaaccatgagctatttcaaaggtgcagtaatgctatatttggcttctctgaagatatcctatgaggccaagtcatcagctttgttcattatctaagagtggtggccagctcaccagcacttcccatcatgtttgccctccctctttcccttgtgttacttcccattttcccttacttctgctttcttggcattaaattctactctgcaatgttaggatataagtttttgcctcagattctgttttctaggaaacccatgctaagacaacactggcagtggccctggaaaagtaaacctcatgatggatttggagttggattgttcactgatctgaaggacagaggactccacttaagtggtaagcagtgtagctatgaactctgccacgcaggcctcacaattactgaggcttcttttacctgtggttaactgggacacagaacagcaggaaattgagtgtagaggttatcaagtagctgcttcacttaattggtataattttatggagttaacctggtttagagtccagagaacattccacatagcctagaaagggtagttatttgtccttaccataatcaagtcatactttgaatatgagttttccttccctgttcagcaccacttctcttagacttaagaatgcctgatctgttgatattatgtcccatgtaacattgcctgagacaaagatatccatgtaccttaaa

SEQ ID NO: 33 - protein, 140 aa, Homo sapiens
MGFQKFSPFLALSILVLLQAGSLHAAPFRSALESSPADPATLSEDEARLLLAALVQDYVQMKASELEQEQEREGSSLDSPRSKRCGNLSTCMLGTYTQDFNKFHTFPQTAIGVGAPGKKRDMSSDLERDHRPHVSMPQNA

SEQ ID NO: 34 - protein, 128 aa, Homo sapiens
MGFQKFSPFLALSILVLLQAGSLHAAPFRSALESSPADPATLSEDEARLLLAALVQDYVQMKASELEQEQEREGSRIIAQKRACDTATCVTHRLAGLLSRSGGVVKNNFVPTNVGSKAFGRRRRDLQA

SEQ ID NO: 35 - protein, 141 aa, Homo sapiens
MGFQKFSPFLALSILVLLQAGSLHAAPFRSALESSPADPATLSEDEARLLLAALVQDYVQMKASELEQEQEREGSSLDSPRSKRCGNLSTCMLGTYTQDFNKFHTFPQTAIGVGAPGKKRDMSSDLERDHRPHVSMPQNAN

SEQ ID NO: 36 - protein, 116 aa, Homo sapiens
APFRSALESSPADPATLSEDEARLLLAALVQDYVQMKASELEQEQEREGSSLDSPRSKRCGNLSTCMLGTYTQDFNKFHTFPQTAIGVGAPGKKRDMSSDLERDHRPHVSMPQNAN

SEQ ID NO: 37 - protein, 187 aa, Homo sapiens
TSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 38 - protein, 168 aa, Homo sapiens
VRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG